(19) United States
(12) Patent Application Publication
(10) Pub. No.: US 2025/0243171 A1
(43) Pub. Date: Jul. 31, 2025

(54) QUINAZOLINE COMPOUNDS FOR TREATMENT OF DISEASE

(71) Applicant: Arthrosi Therapeutics, Inc., San Diego, CA (US)

(72) Inventors: Shunqi YAN, San Diego, CA (US); Litain YEH, San Diego, CA (US)

(21) Appl. No.: 18/685,798

(22) PCT Filed: Aug. 23, 2022
(86) PCT No.: PCT/US2022/041218

§ 371 Date: Feb. 22, 2024

Related U.S. Application Data
(60) Provisional application No. 63/236,581, filed on Aug. 24, 2021.

(51) Int. Cl. C07D 239/94; C07D 401/04; C07D 401/12; C07D 403/04; C07D 403/14; C07D 405/14; C07D 407/14; C07D 413/14; C07D 451/02; C07D 451/14; C07D 471/06; C07D 487/04; C07D 487/06; C07D 491/048; C07D 498/14

(52) U.S. Cl. CPC C07D 239/94; C07D 401/04; C07D 401/12; C07D 403/04; C07D 403/14; C07D 405/14; C07D 407/14; C07D 413/14; C07D 451/02; C07D 451/14; C07D 471/06; C07D 487/04; C07D 487/06; C07D 491/048; C07D 498/14

(57) ABSTRACT

Described herein are inhibitors of EGFR exon 20 insertion mutants and methods of treating cancer comprising the administration of said inhibitors.

### CROSS-REFERENCE

This application claims the benefit of U.S. Provisional Application No. 63/236,581 filed on Aug. 24, 2021, which is incorporated by reference in its entirety.

### BACKGROUND

Lung cancer is the leading cause of cancer-related mortality in the United States. The 5-year relative survival rate from 2010 to 2016 for patients with lung cancer was 21%. About 80% to 85% of lung cancers are non-small-cell lung cancer (NSCLC). As such, there is a need for improved treatment options for NSCLC patients.

### SUMMARY OF THE INVENTION

In one aspect, described herein is a compound of Formula (I), or a pharmaceutically acceptable salt or solvate thereof:

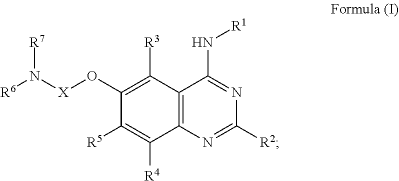

wherein:
X is C₃₋₈cycloalkylene optionally substituted with one, two, three, or four R⁸;
R¹ is selected from C₃₋₈cycloalkyl, C₂₋₉heterocycloalkyl, C₆₋₁₀aryl, and C₁₋₉heteroaryl, wherein C₃₋₈cycloalkyl, C₂₋₉heterocycloalkyl, C₆₋₁₀aryl, and C₁₋₉heteroaryl, are optionally substituted with one, two, three, or four R⁹;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from

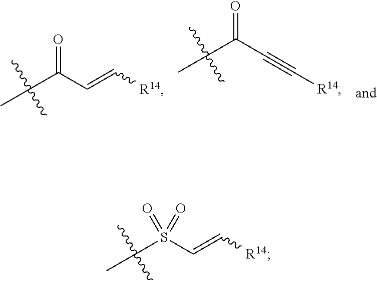

R⁷ is selected from hydrogen, C₁₋₆alkyl, and C₁₋₆haloalkyl;
each R⁸ is independently selected from halogen, oxo, C₁₋₆alkyl, C₁₋₆haloalkyl, C₃₋₆cycloalkyl, C₂₋₉heterocycloalkyl, —OR¹⁰, —SR¹⁰, —N(R¹⁰)(R¹¹), —C(O)OR¹⁰, —C(O)R¹³, —C(O)N(R¹⁰)(R¹¹), —S(O)₂R¹³, —S(O)₂N(R¹⁰)(R¹¹)—, wherein C₁₋₆alkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, and —N(R¹⁰)(R¹¹); each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

In some embodiments is a compound of Formula (I), or a pharmaceutically acceptable salt or solvate thereof, wherein X is cyclobutylene optionally substituted with one, two, three, or four R⁸. In some embodiments is a compound of Formula (I), or a pharmaceutically acceptable salt or solvate thereof, wherein X is cyclopentylene optionally substituted with one, two, three, or four R⁸. In some embodiments is a compound of Formula (I), or a pharmaceutically acceptable salt or solvate thereof, wherein X is cyclohexylene optionally substituted with one, two, three, or four R⁸.

In another aspect, described herein is a compound of Formula (Ia), or a pharmaceutically acceptable salt or solvate thereof:

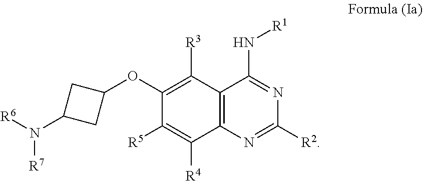

In some embodiments is a compound of Formula (I) or (Ia), or a pharmaceutically acceptable salt or solvate thereof, wherein R⁷ is hydrogen. In some embodiments is a compound of Formula (I) or (Ia), or a pharmaceutically acceptable salt or solvate thereof, wherein at least three of R⁷ is C₁₋₆alkyl.

In another aspect, described herein is a compound of Formula (II), or a pharmaceutically acceptable salt or solvate thereof:

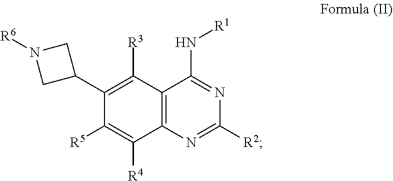

wherein:
R¹ is selected from C₃₋₈cycloalkyl, C₂₋₉heterocycloalkyl, C₆₋₁₀aryl, and C₁₋₉heteroaryl, wherein C₃₋₈cycloalkyl, C₂₋₉heterocycloalkyl, C₆₋₁₀aryl, and C₁₋₉heteroaryl, are optionally substituted with one, two, three, or four R⁹;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from

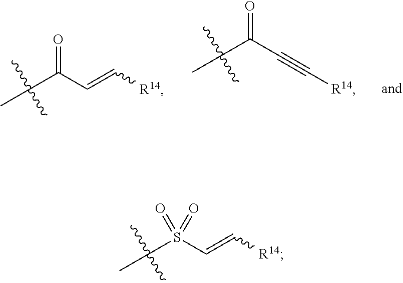

each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

In some embodiments is a compound of Formula (I), (Ia), or (II), or a pharmaceutically acceptable salt or solvate thereof, wherein R¹ is C₆₋₁₀aryl optionally substituted with one, two, three, or four R⁹. In some embodiments is a compound of Formula (I), (Ia), or (II), or a pharmaceutically acceptable salt or solvate thereof, wherein R¹ is phenyl optionally substituted with one, two, three, or four R⁹. In some embodiments is a compound of Formula (I), (Ia), or (II), or a pharmaceutically acceptable salt or solvate thereof, wherein R¹ is C₁₋₉heteroaryl optionally substituted with one, two, three, or four R⁹. In some embodiments is a compound of Formula (I), (Ia), or (II), or a pharmaceutically acceptable salt or solvate thereof, wherein each R⁹ is independently selected from halogen, C₁₋₆alkyl, and C₁₋₆haloalkyl; or two R⁹ substituents are combined to form a cycloalkyl or heterocycloalkyl ring. In some embodiments is a compound of Formula (I), (Ia), or (II), or a pharmaceutically acceptable salt or solvate thereof, wherein each R⁹ is independently selected from halogen. In some embodiments is a compound of Formula (I), (Ia), or (II), or a pharmaceutically acceptable salt or solvate thereof, wherein R¹ is unsubstituted C₆₋₁₀aryl. In some embodiments is a compound of Formula (I), (Ia), or (II), or a pharmaceutically acceptable salt or solvate thereof, wherein R¹ is unsubstituted C₁₋₉heteroaryl.

In another aspect, described herein is a compound of Formula (III), or a pharmaceutically acceptable salt or solvate thereof:

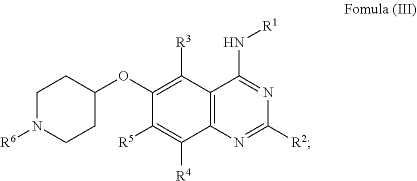

wherein:
R¹ is selected from naphthyl and C₁₋₉heteroaryl, wherein naphthyl and C₁₋₉heteroaryl are optionally substituted with one, two, three, or four R⁹, and C₁₋₉heteroaryl is not indazolyl;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from

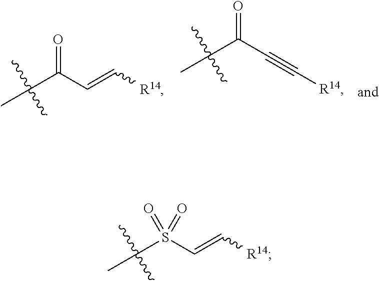

each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

In some embodiments is a compound of Formula (III), or a pharmaceutically acceptable salt or solvate thereof, wherein R¹ is naphthyl optionally substituted with one, two, three, or four R⁹. In some embodiments is a compound of Formula (III), or a pharmaceutically acceptable salt or solvate thereof, wherein R¹ is unsubstituted naphthyl. In some embodiments is a compound of Formula (III), or a pharmaceutically acceptable salt or solvate thereof, wherein R¹ is C₁₋₉ heteroaryl optionally substituted with one, two, three, or four R⁹. In some embodiments is a compound of Formula (III), or a pharmaceutically acceptable salt or solvate thereof, wherein R¹ is unsubstituted C₁₋₉heteroaryl.

In some embodiments is a compound of Formula (I), (Ia), (II), or (III), or a pharmaceutically acceptable salt or solvate thereof, wherein R⁵ is selected from hydrogen, halogen, C₁₋₆alkyl, C₁₋₆haloalkyl, and —OR¹⁰. In some embodiments is a compound of Formula (I), (Ia), (II), or (III), or a pharmaceutically acceptable salt or solvate thereof, wherein R⁵ is —OR¹⁰. In some embodiments is a compound of Formula (I), (Ia), (II), or (III), or a pharmaceutically acceptable salt or solvate thereof, wherein R⁵ is —OR¹⁰ and R¹⁰ is C₁₋₆alkyl. In some embodiments is a compound of Formula (I), (Ia), (II), or (III), or a pharmaceutically acceptable salt or solvate thereof, wherein R⁵ is hydrogen. In some embodiments is a compound of Formula (I), (Ia), (II), or (III), or a pharmaceutically acceptable salt or solvate thereof, wherein R⁴ is hydrogen. In some embodiments is a compound of Formula (I), (Ia), (II), or (III), or a pharmaceutically acceptable salt or solvate thereof, wherein R³ is hydrogen. In some embodiments is a compound of Formula (I), (Ia), (II), or (III), or a pharmaceutically acceptable salt or solvate thereof, wherein R² is hydrogen. In some embodiments is a compound of Formula (I), (Ia), (II), or (III), or a pharmaceutically acceptable salt or solvate thereof, wherein R⁶ is

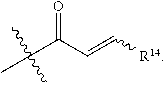

In some embodiments is a compound of Formula (I), (Ia), (II), or (III), or a pharmaceutically acceptable salt or solvate thereof, wherein R⁶ is

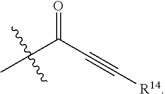

In some embodiments is a compound of Formula (I), (Ia), (II), or (III), or a pharmaceutically acceptable salt or solvate thereof, wherein R⁶ is

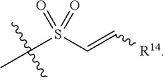

In some embodiments is a compound of Formula (I), (Ia), (II), or (III), or a pharmaceutically acceptable salt or solvate thereof, wherein R¹⁴ is hydrogen. In some embodiments is a compound of Formula (I), (Ia), (II), or (III), or a pharmaceutically acceptable salt or solvate thereof, wherein R¹⁴ is C₁₋₆alkyl optionally substituted with —N(R¹⁰)(R¹¹). In some embodiments is a compound of Formula (I), (Ia), (II), or (III), or a pharmaceutically acceptable salt or solvate thereof, wherein R¹⁴ is unsubstituted C₁₋₆ alkyl. In some embodiments is a compound of Formula (I), (Ia), (II), or (III), or a pharmaceutically acceptable salt or solvate thereof, wherein R¹⁴ is C₁₋₆alkyl substituted with —N(R¹⁰)(R¹¹). In some embodiments is a compound of Formula (I), (Ia), (II), or (III), or a pharmaceutically acceptable salt or solvate thereof, wherein R¹⁴ is C₁₋₆alkyl substituted with —N(R¹⁰)(R¹¹), R¹⁰ is C₁₋₆alkyl, and R¹¹ is C₁₋₆alkyl.

In another aspect, described herein is a pharmaceutical composition comprising a compound of Formula (I), (Ia), (II), or (III) described herein, or a pharmaceutically acceptable salt or solvate thereof, and at least one pharmaceutically acceptable excipient.

In another aspect, described herein is a method of treating cancer in an individual in need thereof, comprising administering to the individual a therapeutically effective amount of a compound of Formula (I), (Ia), (II), or (III) described herein, or a pharmaceutically acceptable salt or solvate thereof. In some embodiments is a method of treating cancer in an individual in need thereof, comprising administering to the individual a therapeutically effective amount of a compound of Formula (I), (Ia), (II), or (III) described herein, or a pharmaceutically acceptable salt or solvate thereof, wherein the cancer is characterized by the overexpression of EGFR/Erb-B2 or mutations of EGFR/Erb-B2. In some embodiments is a method of treating cancer in an individual in need thereof, comprising administering to the individual a therapeutically effective amount of a compound of Formula (I), (Ia), (II), or (III) described herein, or a pharmaceutically acceptable salt or solvate thereof, wherein the cancer comprises exon 20 insertions. In some embodiments is a method of treating cancer in an individual in need thereof, comprising administering to the individual a therapeutically effective amount of a compound of Formula (I), (Ia), (II), or (III) described herein, or a pharmaceutically acceptable salt or solvate thereof, wherein the cancer is lung cancer. In some embodiments is a method of treating cancer in an individual in need thereof, comprising administering to the individual a therapeutically effective amount of a compound of Formula (I), (Ia), (II), or (III) described herein, or a pharmaceutically acceptable salt or solvate thereof, wherein the cancer is non-small-cell lung cancer.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### DETAILED DESCRIPTION OF THE INVENTION

EGFR Exon 20 insertions were initially identified between 2004 and 2005 and are mostly present in non-small-cell lung cancer (NSCLC). However, Exon 20 insertions also commonly appear in many other tumor types such as head and neck cancer, glioblastoma and urothelial cancer.

Metastatic NSCLC (mNSCLC) patients harboring EGFR Exon 20 insertions account for about 2% of all mNSCLC patients. These individuals have a poor prognosis. Due to the lack of effective approved targeted treatments, these patients are at increased risk for swift disease progression. Unfortunately, current EGFR TKIs on market offer less than 10% overall response rate and less than 3 months of progression-free survival. Therefore, there exists an unmet need for patients harboring Exon 20 insertions in mNSCLC and other cancers, requiring new innovative and effective targeted treatments.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features. The term “about” when referring to a number or a numerical range means that the number or numerical range referred to is an approximation within experimental variability (or within statistical experimental error), and thus the number or numerical range may vary between 1% and 15% of the stated number or numerical range.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to a straight or branched hydrocarbon chain radical consisting solely of carbon and hydrogen atoms, containing no unsaturation. In some embodiments, the “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆alkyl” or similar designations. By way of example only, “C₁-C₆alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, and hexyl. Alkyl groups can be substituted or unsubstituted. Depending on the structure, an alkyl group can be a monoradical or a diradical (i.e., an alkylene group).

An “alkoxy” refers to a “—O-alkyl” group, where alkyl is as defined herein.

The term “alkenyl” refers to a straight or branched hydrocarbon chain radical group consisting solely of carbon and hydrogen atoms, containing at least one carbon-carbon double bond. Non-limiting examples of an alkenyl group include —CH═CH₂, —C(CH₃)═CH₂, —CH═CHCH₃, —CH═C(CH₃)₂ and —C(CH₃)═CHCH₃. In some embodiments, an alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a straight or branched hydrocarbon chain radical group consisting solely of carbon and hydrogen atoms, containing at least one carbon-carbon triple bond. Non-limiting examples of an alkynyl group include —C≡CH, —C≡CCH₃, —C≡CCH₂CH₃ and —C≡CCH₂CH₂CH₃. In some embodiments, an alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

The term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂ group, where alkyl is as defined herein.

The term “aromatic” refers to a planar ring having a delocalized π-electron system containing 4n+2π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group).

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

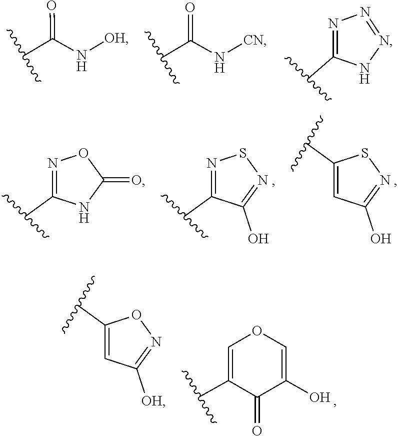

and the like.

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). In some embodiments, cycloalkyl groups include groups having from 3 to 10 ring atoms.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom.

A “heterocycloalkyl” group or “heteroalicyclic” group refers to a cycloalkyl group, wherein at least one skeletal ring atom is a heteroatom selected from nitrogen, oxygen and sulfur. The radicals may be fused with an aryl or heteroaryl. The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The term “halo” or, alternatively, “halogen” means fluoro, chloro, bromo and iodo.

The term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

“Optional” or “optionally” means that a subsequently described event or circumstance may or may not occur and that the description includes instances when the event or circumstance occurs and instances in which it does not.

The term “optionally substituted” or “substituted” means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L^{S}R^{S}, wherein each L^{S} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(C₁-C₆alkyl)-, or -(C₂-C₆alkenyl)-; and each R⁵ is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder, or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of a compound dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which a compound is absorbed into the general circulation when the pharmaceutical composition is taken orally as compared to intravenous injection.

“Blood plasma concentration” refers to the concentration of a compound in the plasma component of blood of a subject. It is understood that the plasma concentration of a compound may vary significantly between subjects, due to variability with respect to metabolism and/or possible interactions with other therapeutic agents. In some embodiments, the blood plasma concentration of a compound may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of a compound may vary from subject to subject.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effective amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder, or condition. An “enhancing-effective amount,” as used herein, refers to an amount adequate to enhance the effect of a therapeutic agent in the treatment of a disease, disorder, or condition. When used in a patient, amounts effective for this use will depend on the severity and course of the disease, disorder, or condition, previous therapy, the patient's health status and response to the drugs, and the judgment of the treating physician.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like. As an example, one can determine such prophylactically effective amounts by a dose escalation clinical trial.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts, and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins, and the like. See Berge et al., supra.

The term “subject” as used herein, refers to an animal which is the object of treatment, observation or experiment. By way of example only, a subject may be, but is not limited to, a mammal including, but not limited to, a human.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

### Compounds

The compounds of Formula (I), (Ia), (II), or (III), or a pharmaceutically acceptable salt or solvate thereof, described herein are inhibitors of EGFR exon 20 insertion mutants. The compounds of Formula (I), (Ia), (II), or (III), described herein, and compositions comprising these compounds, are useful for the treatment of lung cancer including, but not limited to, non-small-cell lung cancer.

In some embodiments, described herein is a compound of Formula (I), or a pharmaceutically acceptable salt or solvate thereof:

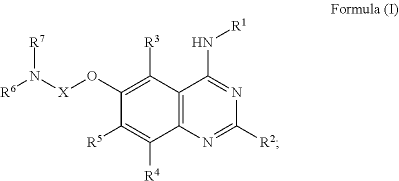

wherein:
X is C₃₋₈cycloalkylene optionally substituted with one, two, three, or four R⁸;
R¹ is selected from C₃₋₈cycloalkyl, C₂₋₉heterocycloalkyl, C₆₋₁₀aryl, and C₁₋₉heteroaryl, wherein C₃₋₈cycloalkyl, C₂₋₉heterocycloalkyl, C₆₋₁₀aryl, and C₁₋₉heteroaryl, are optionally substituted with one, two, three, or four R⁹;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from

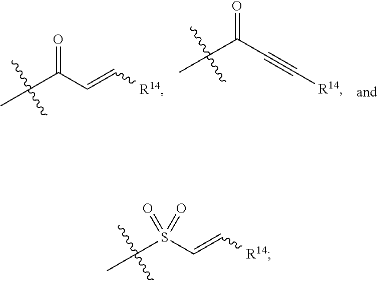

R⁷ is selected from hydrogen, C₁₋₆alkyl, and C₁₋₆haloalkyl;
each R⁸ is independently selected from halogen, oxo, C₁₋₆alkyl, C₁₋₆haloalkyl, C₃₋₆cycloalkyl, C₂₋₉heterocycloalkyl, —OR¹⁰, —SR¹⁰, —N(R¹⁰)(R¹¹), —C(O)OR¹⁰, —C(O)R¹³, —C(O)N(R¹⁰)(R¹), —S(O)₂R¹³, —S(O)₂N(R¹⁰)(R¹¹)—, wherein C₁₋₆alkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, and —N(R¹⁰)(R¹¹);
each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

In some embodiments is a compound of Formula (I), or a pharmaceutically acceptable salt or solvate thereof, wherein X is cyclobutylene optionally substituted with one, two, three, or four R⁸. In some embodiments is a compound of Formula (I), or a pharmaceutically acceptable salt or solvate thereof, wherein X is unsubstituted cyclobutylene. In some embodiments is a compound of Formula (I), or a pharmaceutically acceptable salt or solvate thereof, wherein X is cyclopentylene optionally substituted with one, two, three, or four R⁸. In some embodiments is a compound of Formula (I), or a pharmaceutically acceptable salt or solvate thereof, wherein X is unsubstituted cyclopentylene. In some embodiments is a compound of Formula (I), or a pharmaceutically acceptable salt or solvate thereof, wherein X is cyclohexylene optionally substituted with one, two, three, or four R⁸. In some embodiments is a compound of Formula (I), or a pharmaceutically acceptable salt or solvate thereof, wherein X is unsubstituted cyclohexylene. In some embodiments is a compound of Formula (I), or a pharmaceutically acceptable salt or solvate thereof, wherein X is cycloheptylene optionally substituted with one, two, three, or four R⁸. In some embodiments is a compound of Formula (I), or a pharmaceutically acceptable salt or solvate thereof, wherein X is unsubstituted cycloheptylene. In some embodiments is a compound of Formula (I), or a pharmaceutically acceptable salt or solvate thereof, wherein X is cyclooxylene optionally substituted with one, two, three, or four R⁸. In some embodiments is a compound of Formula (I), or a pharmaceutically acceptable salt or solvate thereof, wherein X is unsubstituted cyclooxylene. In some embodiments is a compound of Formula (I), or a pharmaceutically acceptable salt or solvate thereof, wherein X is cyclopropylene optionally substituted with one, two, three, or four R⁸. In some embodiments is a compound of Formula (I), or a pharmaceutically acceptable salt or solvate thereof, wherein X is unsubstituted cyclopropylene.

In some embodiments, described herein is a compound of Formula (I), or a pharmaceutically acceptable salt or solvate thereof, having the structure of Formula (Ia):

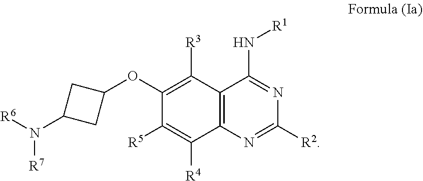

In some embodiments is a compound of Formula (I) or (Ia), or a pharmaceutically acceptable salt or solvate thereof, wherein R¹ is C₆₋₁₀aryl optionally substituted with one, two, three, or four R⁹. In some embodiments is a compound of Formula (I) or (Ia), or a pharmaceutically acceptable salt or solvate thereof, wherein R¹ is phenyl optionally substituted with one, two, three, or four R⁹. In some embodiments is a compound of Formula (I) or (Ia), or a pharmaceutically acceptable salt or solvate thereof, wherein R¹ is C₁₋₉heteroaryl optionally substituted with one, two, three, or four R⁹. In some embodiments is a compound of Formula (I) or (Ia), or a pharmaceutically acceptable salt or solvate thereof, wherein R¹ is C₂₋₉heterocycloalkyl optionally substituted with one, two, three, or four R⁹. In some embodiments is a compound of Formula (I) or (Ia), or a pharmaceutically acceptable salt or solvate thereof, wherein R¹ is C₃₋₈cycloalkyl optionally substituted with one, two, three, or four R⁹. In some embodiments is a compound of Formula (I) or (Ia), or a pharmaceutically acceptable salt or solvate thereof, wherein each R⁹ is independently selected from halogen, C₁₋₆alkyl, and C₁₋₆ haloalkyl; or two R⁹ substituents are combined to form a cycloalkyl or heterocycloalkyl ring. In some embodiments is a compound of Formula (I) or (Ia), or a pharmaceutically acceptable salt or solvate thereof, wherein each R⁹ is independently selected from halogen, C₁₋₆alkyl, and C₁₋₆haloalkyl. In some embodiments is a compound of Formula (I) or (Ia), or a pharmaceutically acceptable salt or solvate thereof, wherein two R⁹ substituents are combined to form a cycloalkyl or heterocycloalkyl ring. In some embodiments is a compound of Formula (I) or (Ia), or a pharmaceutically acceptable salt or solvate thereof, wherein each R⁹ is independently selected from halogen.

In some embodiments is a compound of Formula (I) or (Ia), or a pharmaceutically acceptable salt or solvate thereof, wherein R¹ is unsubstituted C₆₋₁₀aryl. In some embodiments is a compound of Formula (I) or (Ia), or a pharmaceutically acceptable salt or solvate thereof, wherein R¹ is unsubstituted phenyl. In some embodiments is a compound of Formula (I) or (Ia), or a pharmaceutically acceptable salt or solvate thereof, wherein R¹ is unsubstituted C₁₋₉ heteroaryl. In some embodiments is a compound of Formula (I) or (Ia), or a pharmaceutically acceptable salt or solvate thereof, wherein R¹ is unsubstituted C₂₋₉heterocycloalkyl. In some embodiments is a compound of Formula (I) or (Ia), or a pharmaceutically acceptable salt or solvate thereof, wherein R¹ is unsubstituted C₃₋₈cycloalkyl.

In some embodiments is a compound of Formula (I) or (Ia), or a pharmaceutically acceptable salt or solvate thereof, wherein R² is hydrogen, halogen, C₁₋₆alkyl, or C₁₋₆haloalkyl. In some embodiments is a compound of Formula (I) or (Ia), or a pharmaceutically acceptable salt or solvate thereof, wherein R² is hydrogen. In some embodiments is a compound of Formula (I) or (Ia), or a pharmaceutically acceptable salt or solvate thereof, wherein R² is halogen. In some embodiments is a compound of Formula (I) or (Ia), or a pharmaceutically acceptable salt or solvate thereof, wherein R² is C₁₋₆alkyl. In some embodiments is a compound of Formula (I) or (Ia), or a pharmaceutically acceptable salt or solvate thereof, wherein R² is C₁₋₆haloalkyl.

In some embodiments is a compound of Formula (I) or (Ia), or a pharmaceutically acceptable salt or solvate thereof, wherein R³ is hydrogen, halogen, C₁₋₆alkyl, or C₁₋₆haloalkyl. In some embodiments is a compound of Formula (I) or (Ia), or a pharmaceutically acceptable salt or solvate thereof, wherein R³ is hydrogen. In some embodiments is a compound of Formula (I) or (Ia), or a pharmaceutically acceptable salt or solvate thereof, wherein R³ is halogen. In some embodiments is a compound of Formula (I) or (Ia), or a pharmaceutically acceptable salt or solvate thereof, wherein R³ is C₁₋₆alkyl. In some embodiments is a compound of Formula (I) or (Ia), or a pharmaceutically acceptable salt or solvate thereof, wherein R³ is C₁₋₆haloalkyl.

In some embodiments is a compound of Formula (I) or (Ia), or a pharmaceutically acceptable salt or solvate thereof, wherein R⁴ is hydrogen, halogen, C₁₋₆alkyl, or C₁₋₆haloalkyl. In some embodiments is a compound of Formula (I) or (Ia), or a pharmaceutically acceptable salt or solvate thereof, wherein R⁴ is hydrogen. In some embodiments is a compound of Formula (I) or (Ia), or a pharmaceutically acceptable salt or solvate thereof, wherein R⁴ is halogen. In some embodiments is a compound of Formula (I) or (Ia), or a pharmaceutically acceptable salt or solvate thereof, wherein R⁴ is C₁₋₆alkyl. In some embodiments is a compound of Formula (I) or (Ia), or a pharmaceutically acceptable salt or solvate thereof, wherein R⁴ is C₁₋₆haloalkyl.

In some embodiments is a compound of Formula (I) or (Ia), or a pharmaceutically acceptable salt or solvate thereof, wherein R⁵ is selected from hydrogen, halogen, C₁₋₆alkyl, C₁₋₆ haloalkyl, and —OR¹⁰. In some embodiments is a compound of Formula (I) or (Ia), or a pharmaceutically acceptable salt or solvate thereof, wherein R⁵ is hydrogen. In some embodiments is a compound of Formula (I) or (Ia), or a pharmaceutically acceptable salt or solvate thereof, wherein R⁵ is halogen. In some embodiments is a compound of Formula (I) or (Ia), or a pharmaceutically acceptable salt or solvate thereof, wherein R⁵ is C₁₋₆alkyl. In some embodiments is a compound of Formula (I) or (Ia), or a pharmaceutically acceptable salt or solvate thereof, wherein R⁵ is C₁₋₆haloalkyl. In some embodiments is a compound of Formula (I) or (Ia), or a pharmaceutically acceptable salt or solvate thereof, wherein R⁵ is —OR¹⁰.

In some embodiments is a compound of Formula (I) or (Ia), or a pharmaceutically acceptable salt or solvate thereof, wherein R⁵ is —OR¹⁰ and R¹⁰ is selected from hydrogen, C₁₋₆alkyl, C₁₋₆ haloalkyl, C₃₋₆cycloalkyl, and C₂₋₉heterocycloalkyl, wherein C₁₋₆alkyl, C₁₋₆ haloalkyl, C₃₋₆cycloalkyl, and C₂₋₉heterocycloalkyl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (I) or (Ia), or a pharmaceutically acceptable salt or solvate thereof, wherein R⁵ is —OR¹⁰ and R¹⁰ is selected from hydrogen, C₁₋₆alkyl, C₁₋₆ haloalkyl, C₃₋₆cycloalkyl, and C₂₋₉heterocycloalkyl, wherein C₁₋₆alkyl, C₁₋₆ haloalkyl, C₃₋₆cycloalkyl, and C₂₋₉heterocycloalkyl are unsubstituted. In some embodiments is a compound of Formula (I) or (Ia), or a pharmaceutically acceptable salt or solvate thereof, wherein R⁵ is —OR¹⁰ and R¹⁰ is C₁₋₆alkyl optionally substituted with one, two, or three groups selected from C₁₋₆alkoxy, C₃₋₆cycloalkyl, and C₂₋₉heterocycloalkyl. In some embodiments is a compound of Formula (I) or (Ia), or a pharmaceutically acceptable salt or solvate thereof, wherein R⁵ is —OR¹⁰ and R¹⁰ is C₁₋₆alkyl substituted with one, two, or three groups selected from C₁₋₆alkoxy, C₃₋₆cycloalkyl, and C₂₋₉heterocycloalkyl. In some embodiments is a compound of Formula (I) or (Ia), or a pharmaceutically acceptable salt or solvate thereof, wherein R⁵ is —OR¹⁰ and R¹⁰ is C₁₋₆alkyl substituted with one group selected from C₁₋₆alkoxy, C₃₋₆cycloalkyl, and C₂₋₉heterocycloalkyl. In some embodiments is a compound of Formula (I) or (Ia), or a pharmaceutically acceptable salt or solvate thereof, wherein R⁵ is —OR¹⁰ and R¹⁰ is unsubstituted C₁₋₆alkyl. In some embodiments is a compound of Formula (I) or (Ia), or a pharmaceutically acceptable salt or solvate thereof, wherein R⁵ is —OR¹⁰ and R¹⁰ is —CH₃. In some embodiments is a compound of Formula (I) or (Ia), or a pharmaceutically acceptable salt or solvate thereof, wherein R⁵ is —OR¹⁰ and R¹⁰ is C₁₋₆ haloalkyl. In some embodiments is a compound of Formula (I) or (Ia), or a pharmaceutically acceptable salt or solvate thereof, wherein R⁵ is —OR¹⁰ and R¹⁰ is C₃₋₆cycloalkyl. In some embodiments is a compound of Formula (I) or (Ia), or a pharmaceutically acceptable salt or solvate thereof, wherein R⁵ is —OR¹⁰ and R¹⁰ is C₂₋₉heterocycloalkyl.

In some embodiments is a compound of Formula (I) or (Ia), or a pharmaceutically acceptable salt or solvate thereof, wherein R⁶ is

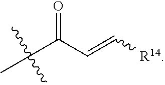

In some embodiments is a compound of Formula (I) or (Ia), or a pharmaceutically acceptable salt or solvate thereof, wherein R⁶ is

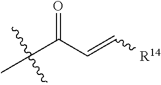

and R¹⁴ is hydrogen. In some embodiments is a compound of Formula (I) or (Ia), or a pharmaceutically acceptable salt or solvate thereof, wherein R⁶ is

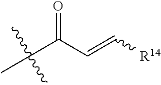

and R¹⁴ is C₁₋₆alkyl optionally substituted with —N(R¹⁰)(R¹¹).

In some embodiments is a compound of Formula (I) or (Ia), or a pharmaceutically acceptable salt or solvate thereof, wherein R⁶

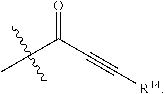

In some embodiments is a compound of Formula (I) or (Ia), or a pharmaceutically acceptable salt or solvate thereof, wherein R⁶

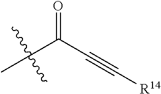

and R¹⁴ is hydrogen. In some embodiments is a compound of Formula (I) or (Ia), or a pharmaceutically acceptable salt or solvate thereof, wherein R⁶ is

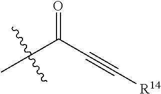

and R¹⁴ is C₁₋₆alkyl optionally substituted with —N(R¹⁰)(R¹¹).

In some embodiments is a compound of Formula (I) or (Ia), or a pharmaceutically acceptable salt or solvate thereof, wherein R⁶ is

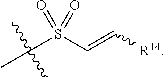

In some embodiments is a compound of Formula (I) or (Ia), or a pharmaceutically acceptable salt or solvate thereof, wherein R⁶ is

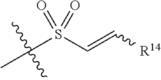

and R¹⁴ is hydrogen. In some embodiments is a compound of Formula (I) or (Ia), or a pharmaceutically acceptable salt or solvate thereof, wherein R⁶ is

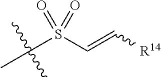

and R¹⁴ is C₁₋₆alkyl optionally substituted with —N(R¹⁰)(R¹¹).

In some embodiments is a compound of Formula (I) or (Ia), or a pharmaceutically acceptable salt or solvate thereof, wherein R⁷ is hydrogen. In some embodiments is a compound of Formula (I) or (Ia), or a pharmaceutically acceptable salt or solvate thereof, wherein R⁷ is C₁₋₆ alkyl. In some embodiments is a compound of Formula (I) or (Ia), or a pharmaceutically acceptable salt or solvate thereof, wherein R⁷ is —CH₃. In some embodiments is a compound of Formula (I) or (Ia), or a pharmaceutically acceptable salt or solvate thereof, wherein R⁷ is C₁₋₆haloalkyl.

In some embodiments, described herein is a compound of Formula (II), or a pharmaceutically acceptable salt or solvate thereof:

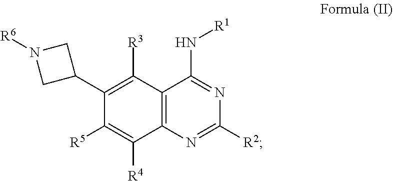

wherein:
R¹ is selected from C₃₋₈cycloalkyl, C₂₋₉heterocycloalkyl, C₆₋₁₀aryl, and C₁₋₉heteroaryl, wherein C₃₋₈cycloalkyl, C₂₋₉heterocycloalkyl, C₆₋₁₀aryl, and C₁₋₉heteroaryl, are optionally substituted with one, two, three, or four R⁹;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from

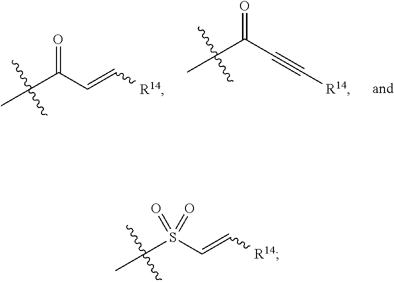

each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

In some embodiments is a compound of Formula (II), or a pharmaceutically acceptable salt or solvate thereof, wherein R¹ is C₆₋₁₀aryl optionally substituted with one, two, three, or four R⁹. In some embodiments is a compound of Formula (II), or a pharmaceutically acceptable salt or solvate thereof, wherein R¹ is phenyl optionally substituted with one, two, three, or four R⁹. In some embodiments is a compound of Formula (II), or a pharmaceutically acceptable salt or solvate thereof, wherein R¹ is C₁₋₉heteroaryl optionally substituted with one, two, three, or four R⁹. In some embodiments is a compound of Formula (II), or a pharmaceutically acceptable salt or solvate thereof, wherein R¹ is C₂₋₉heterocycloalkyl optionally substituted with one, two, three, or four R⁹. In some embodiments is a compound of Formula (II), or a pharmaceutically acceptable salt or solvate thereof, wherein R¹ is C₃₋₈cycloalkyl optionally substituted with one, two, three, or four R⁹. In some embodiments is a compound of Formula (II), or a pharmaceutically acceptable salt or solvate thereof, wherein each R⁹ is independently selected from halogen, C₁₋₆alkyl, and C₁₋₆haloalkyl; or two R⁹ substituents are combined to form a cycloalkyl or heterocycloalkyl ring. In some embodiments is a compound of Formula (II), or a pharmaceutically acceptable salt or solvate thereof, wherein each R⁹ is independently selected from halogen, C₁₋₆alkyl, and C₁₋₆haloalkyl. In some embodiments is a compound of Formula (II), or a pharmaceutically acceptable salt or solvate thereof, wherein two R⁹ substituents are combined to form a cycloalkyl or heterocycloalkyl ring. In some embodiments is a compound of Formula (II), or a pharmaceutically acceptable salt or solvate thereof, wherein each R⁹ is independently selected from halogen.

In some embodiments is a compound of Formula (II), or a pharmaceutically acceptable salt or solvate thereof, wherein R¹ is unsubstituted C₆₋₁₀aryl. In some embodiments is a compound of Formula (II), or a pharmaceutically acceptable salt or solvate thereof, wherein R¹ is unsubstituted phenyl. In some embodiments is a compound of Formula (II), or a pharmaceutically acceptable salt or solvate thereof, wherein R¹ is unsubstituted C₁₋₉heteroaryl.

In some embodiments is a compound of Formula (II), or a pharmaceutically acceptable salt or solvate thereof, wherein R¹ is unsubstituted C₂₋₉heterocycloalkyl. In some embodiments is a compound of Formula (II), or a pharmaceutically acceptable salt or solvate thereof, wherein R¹ is unsubstituted C₃₋₈cycloalkyl.

In some embodiments is a compound of Formula (II), or a pharmaceutically acceptable salt or solvate thereof, wherein R² is hydrogen, halogen, C₁₋₆alkyl, or C₁₋₆haloalkyl. In some embodiments is a compound of Formula (II), or a pharmaceutically acceptable salt or solvate thereof, wherein R² is hydrogen. In some embodiments is a compound of Formula (II), or a pharmaceutically acceptable salt or solvate thereof, wherein R² is halogen. In some embodiments is a compound of Formula (II), or a pharmaceutically acceptable salt or solvate thereof, wherein R² is C₁₋₆alkyl. In some embodiments is a compound of Formula (II), or a pharmaceutically acceptable salt or solvate thereof, wherein R² is C₁₋₆haloalkyl.

In some embodiments is a compound of Formula (II), or a pharmaceutically acceptable salt or solvate thereof, wherein R³ is hydrogen, halogen, C₁₋₆alkyl, or C₁₋₆haloalkyl. In some embodiments is a compound of Formula (II), or a pharmaceutically acceptable salt or solvate thereof, wherein R³ is hydrogen. In some embodiments is a compound of Formula (II), or a pharmaceutically acceptable salt or solvate thereof, wherein R³ is halogen. In some embodiments is a compound of Formula (II), or a pharmaceutically acceptable salt or solvate thereof, wherein R³ is C₁₋₆alkyl. In some embodiments is a compound of Formula (II), or a pharmaceutically acceptable salt or solvate thereof, wherein R³ is C₁₋₆haloalkyl.

In some embodiments is a compound of Formula (II), or a pharmaceutically acceptable salt or solvate thereof, wherein R⁴ is hydrogen, halogen, C₁₋₆alkyl, or C₁₋₆haloalkyl. In some embodiments is a compound of Formula (II), or a pharmaceutically acceptable salt or solvate thereof, wherein R⁴ is hydrogen. In some embodiments is a compound of Formula (II), or a pharmaceutically acceptable salt or solvate thereof, wherein R⁴ is halogen. In some embodiments is a compound of Formula (II), or a pharmaceutically acceptable salt or solvate thereof, wherein R⁴ is C₁₋₆alkyl. In some embodiments is a compound of Formula (II), or a pharmaceutically acceptable salt or solvate thereof, wherein R⁴ is C₁₋₆haloalkyl.

In some embodiments is a compound of Formula (II), or a pharmaceutically acceptable salt or solvate thereof, wherein R⁵ is selected from hydrogen, halogen, C₁₋₆alkyl, C₁₋₆haloalkyl, and —OR¹⁰. In some embodiments is a compound of Formula (II), or a pharmaceutically acceptable salt or solvate thereof, wherein R⁵ is hydrogen. In some embodiments is a compound of Formula (II), or a pharmaceutically acceptable salt or solvate thereof, wherein R⁵ is halogen. In some embodiments is a compound of Formula (II), or a pharmaceutically acceptable salt or solvate thereof, wherein R⁵ is C₁₋₆alkyl. In some embodiments is a compound of Formula (II), or a pharmaceutically acceptable salt or solvate thereof, wherein R⁵ is C₁₋₆haloalkyl. In some embodiments is a compound of Formula (II), or a pharmaceutically acceptable salt or solvate thereof, wherein R⁵ is —OR¹⁰.

In some embodiments is a compound of Formula (II), or a pharmaceutically acceptable salt or solvate thereof, wherein R⁵ is —OR¹⁰ and R¹⁰ is selected from hydrogen, C₁₋₆alkyl, C₁₋₆ haloalkyl, C₃₋₆cycloalkyl, and C₂₋₉heterocycloalkyl, wherein C₁₋₆alkyl, C₁₋₆ haloalkyl, C₃₋₆cycloalkyl, and C₂₋₉heterocycloalkyl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (II), or a pharmaceutically acceptable salt or solvate thereof, wherein R⁵ is —OR¹⁰ and R¹⁰ is selected from hydrogen, C₁₋₆alkyl, C₁₋₆ haloalkyl, C₃₋₆cycloalkyl, and C₂₋₉heterocycloalkyl, wherein C₁₋₆alkyl, C₁₋₆ haloalkyl, C₃₋₆cycloalkyl, and C₂₋₉heterocycloalkyl are unsubstituted. In some embodiments is a compound of Formula (II), or a pharmaceutically acceptable salt or solvate thereof, wherein R⁵ is —OR¹⁰ and R¹⁰ is C₁₋₆alkyl optionally substituted with one, two, or three groups selected from C₁₋₆alkoxy, C₃₋₆cycloalkyl, and C₂₋₉heterocycloalkyl. In some embodiments is a compound of Formula (II), or a pharmaceutically acceptable salt or solvate thereof, wherein R⁵ is —OR¹⁰ and R¹⁰ is C₁₋₆alkyl substituted with one, two, or three groups selected from C₁₋₆alkoxy, C₃₋₆ cycloalkyl, and C₂₋₉heterocycloalkyl. In some embodiments is a compound of Formula (II), or a pharmaceutically acceptable salt or solvate thereof, wherein R⁵ is —OR¹⁰ and R¹⁰ is C₁₋₆alkyl substituted with one group selected from C₁₋₆alkoxy, C₃₋₆cycloalkyl, and C₂₋₉heterocycloalkyl. In some embodiments is a compound of Formula (II), or a pharmaceutically acceptable salt or solvate thereof, wherein R⁵ is —OR¹⁰ and R¹⁰ is unsubstituted C₁₋₆alkyl. In some embodiments is a compound of Formula (II), or a pharmaceutically acceptable salt or solvate thereof, wherein R⁵ is —OR¹⁰ and R¹⁰ is —CH₃. In some embodiments is a compound of Formula (II), or a pharmaceutically acceptable salt or solvate thereof, wherein R⁵ is —OR¹⁰ and R¹⁰ is C₁₋₆ haloalkyl. In some embodiments is a compound of Formula (II), or a pharmaceutically acceptable salt or solvate thereof, wherein R⁵ is —OR¹⁰ and R¹⁰ is C₃₋₆cycloalkyl. In some embodiments is a compound of Formula (II), or a pharmaceutically acceptable salt or solvate thereof, wherein R⁵ is —OR¹⁰ and R¹⁰ is C₂₋₉heterocycloalkyl.

In some embodiments is a compound of Formula (II), or a pharmaceutically acceptable salt or solvate thereof, wherein R⁶ is

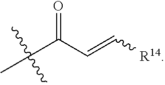

In some embodiments is a compound of Formula (II), or a pharmaceutically acceptable salt or solvate thereof, wherein R⁶ is

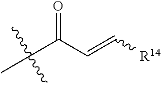

and R¹⁴ is hydrogen. In some embodiments is a compound of Formula (II), or a pharmaceutically acceptable salt or solvate thereof, wherein R⁶ is

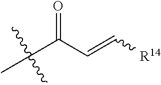

and R¹⁴ is C₁₋₆alkyl optionally substituted with —N(R¹⁰)(R¹¹).

In some embodiments is a compound of Formula (II), or a pharmaceutically acceptable salt or solvate thereof, wherein R⁶ is

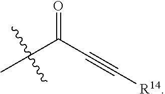

In some embodiments is a compound of Formula (II), or a pharmaceutically acceptable salt or solvate thereof, wherein R⁶ is

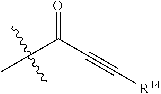

and R¹⁴ is hydrogen. In some embodiments is a compound of Formula (II), or a pharmaceutically acceptable salt or solvate thereof, wherein R⁶ is

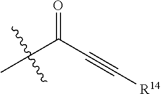

and R¹⁴ is C₁₋₆alkyl optionally substituted with —N(R¹⁰)(R¹¹).

In some embodiments is a compound of Formula (II), or a pharmaceutically acceptable salt or solvate thereof, wherein R⁶ is

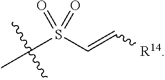

In some embodiments is a compound of Formula (II), or a pharmaceutically acceptable salt or solvate thereof, wherein R⁶ is

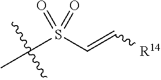

and R¹⁴ is hydrogen. In some embodiments is a compound of Formula (II), or a pharmaceutically acceptable salt or solvate thereof, wherein R⁶ is

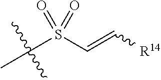

and R¹⁴ is C₁₋₆alkyl optionally substituted with —N(R¹⁰)(R¹¹).

In some embodiments, described herein is a compound of Formula (III), or a pharmaceutically acceptable salt or solvate thereof:

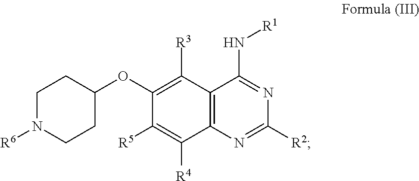

wherein:
R¹ is selected from naphthyl and C₁₋₉heteroaryl, wherein naphthyl and C₁₋₉heteroaryl are optionally substituted with one, two, three, or four R⁹, and C₁₋₉heteroaryl is not indazolyl;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from

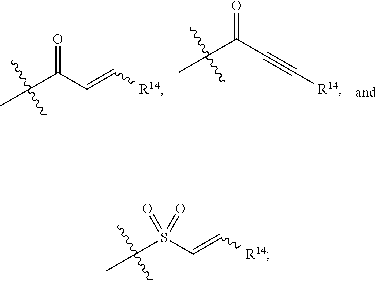

each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

In some embodiments is a compound of Formula (III), or a pharmaceutically acceptable salt or solvate thereof, wherein naphthyl optionally substituted with one, two, three, or four R⁹. In some embodiments is a compound of Formula (III), or a pharmaceutically acceptable salt or solvate thereof, wherein R¹ is C₁₋₉heteroaryl optionally substituted with one, two, three, or four R⁹. In some embodiments is a compound of Formula (III), or a pharmaceutically acceptable salt or solvate thereof, wherein R¹ is C₁₋₉heteroaryl optionally substituted with one, two, three, or four R⁹, wherein C₁₋₉heteroaryl is a bicyclic heteroaryl. In some embodiments is a compound of Formula (III), or a pharmaceutically acceptable salt or solvate thereof, wherein R¹ is C₁₋₉ heteroaryl optionally substituted with one, two, three, or four R⁹, wherein C₁₋₉heteroaryl is a quinolinyl, indolyl, or benzothiazolyl. In some embodiments is a compound of Formula (III), or a pharmaceutically acceptable salt or solvate thereof, wherein each R⁹ is independently selected from halogen, C₁₋₆alkyl, and C₁₋₆haloalkyl; or two R⁹ substituents are combined to form a cycloalkyl or heterocycloalkyl ring. In some embodiments is a compound of Formula (III), or a pharmaceutically acceptable salt or solvate thereof, wherein each R⁹ is independently selected from halogen, C₁₋₆alkyl, and C₁₋₆haloalkyl. In some embodiments is a compound of Formula (III), or a pharmaceutically acceptable salt or solvate thereof, wherein two R⁹ substituents are combined to form a cycloalkyl or heterocycloalkyl ring. In some embodiments is a compound of Formula (III), or a pharmaceutically acceptable salt or solvate thereof, wherein each R⁹ is independently selected from halogen.

In some embodiments is a compound of Formula (III), or a pharmaceutically acceptable salt or solvate thereof, wherein R¹ is unsubstituted naphthyl. In some embodiments is a compound of Formula (III), or a pharmaceutically acceptable salt or solvate thereof, wherein R¹ is unsubstituted C₁₋₉heteroaryl. In some embodiments is a compound of Formula (III), or a pharmaceutically acceptable salt or solvate thereof, wherein R¹ is unsubstituted C₁₋₉heteroaryl, wherein C₁₋₉heteroaryl is a bicyclic heteroaryl. In some embodiments is a compound of Formula (III), or a pharmaceutically acceptable salt or solvate thereof, wherein R¹ is C₁₋₉heteroaryl, wherein C₁₋₉heteroaryl is a quinolinyl, indolyl, or benzothiazolyl.

In some embodiments is a compound of Formula (III), or a pharmaceutically acceptable salt or solvate thereof, wherein R² is hydrogen, halogen, C₁₋₆alkyl, or C₁₋₆haloalkyl. In some embodiments is a compound of Formula (III), or a pharmaceutically acceptable salt or solvate thereof, wherein R² is hydrogen. In some embodiments is a compound of Formula (III), or a pharmaceutically acceptable salt or solvate thereof, wherein R² is halogen. In some embodiments is a compound of Formula (III), or a pharmaceutically acceptable salt or solvate thereof, wherein R² is C₁₋₆alkyl. In some embodiments is a compound of Formula (III), or a pharmaceutically acceptable salt or solvate thereof, wherein R² is C₁₋₆haloalkyl.

In some embodiments is a compound of Formula (III), or a pharmaceutically acceptable salt or solvate thereof, wherein R³ is hydrogen, halogen, C₁₋₆alkyl, or C₁₋₆haloalkyl. In some embodiments is a compound of Formula (III), or a pharmaceutically acceptable salt or solvate thereof, wherein R³ is hydrogen. In some embodiments is a compound of Formula (III), or a pharmaceutically acceptable salt or solvate thereof, wherein R³ is halogen. In some embodiments is a compound of Formula (III), or a pharmaceutically acceptable salt or solvate thereof, wherein R³ is C₁₋₆alkyl. In some embodiments is a compound of Formula (III), or a pharmaceutically acceptable salt or solvate thereof, wherein R³ is C₁₋₆haloalkyl.

In some embodiments is a compound of Formula (III), or a pharmaceutically acceptable salt or solvate thereof, wherein R⁴ is hydrogen, halogen, C₁₋₆alkyl, or C₁₋₆haloalkyl. In some embodiments is a compound of Formula (III), or a pharmaceutically acceptable salt or solvate thereof, wherein R⁴ is hydrogen. In some embodiments is a compound of Formula (III), or a pharmaceutically acceptable salt or solvate thereof, wherein R⁴ is halogen. In some embodiments is a compound of Formula (III), or a pharmaceutically acceptable salt or solvate thereof, wherein R⁴ is C₁₋₆alkyl. In some embodiments is a compound of Formula (III), or a pharmaceutically acceptable salt or solvate thereof, wherein R⁴ is C₁₋₆haloalkyl.

In some embodiments is a compound of Formula (III), or a pharmaceutically acceptable salt or solvate thereof, wherein R⁵ is selected from hydrogen, halogen, C₁₋₆alkyl, C₁₋₆haloalkyl, and —OR¹⁰. In some embodiments is a compound of Formula (III), or a pharmaceutically acceptable salt or solvate thereof, wherein R⁵ is hydrogen. In some embodiments is a compound of Formula (III), or a pharmaceutically acceptable salt or solvate thereof, wherein R⁵ is halogen. In some embodiments is a compound of Formula (III), or a pharmaceutically acceptable salt or solvate thereof, wherein R⁵ is C₁₋₆alkyl. In some embodiments is a compound of Formula (III), or a pharmaceutically acceptable salt or solvate thereof, wherein R⁵ is C₁₋₆haloalkyl. In some embodiments is a compound of Formula (III), or a pharmaceutically acceptable salt or solvate thereof, wherein R⁵ is —OR¹⁰.

In some embodiments is a compound of Formula (III), or a pharmaceutically acceptable salt or solvate thereof, wherein R⁵ is —OR¹⁰ and R¹⁰ is selected from hydrogen, C₁₋₆alkyl, C₁₋₆ haloalkyl, C₃₋₆cycloalkyl, and C₂₋₉heterocycloalkyl, wherein C₁₋₆alkyl, C₁₋₆ haloalkyl, C₃₋₆cycloalkyl, and C₂₋₉heterocycloalkyl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments is a compound of Formula (III), or a pharmaceutically acceptable salt or solvate thereof, wherein R⁵ is —OR¹⁰ and R¹⁰ is selected from hydrogen, C₁₋₆alkyl, C₁₋₆ haloalkyl, C₃₋₆cycloalkyl, and C₂₋₉heterocycloalkyl, wherein C₁₋₆alkyl, C₁₋₆ haloalkyl, C₃₋₆cycloalkyl, and C₂₋₉heterocycloalkyl are unsubstituted. In some embodiments is a compound of Formula (III), or a pharmaceutically acceptable salt or solvate thereof, wherein R⁵ is —OR¹⁰ and R¹⁰ is C₁₋₆alkyl optionally substituted with one, two, or three groups selected from C₁₋₆alkoxy, C₃₋₆cycloalkyl, and C₂₋₉heterocycloalkyl. In some embodiments is a compound of Formula (III), or a pharmaceutically acceptable salt or solvate thereof, wherein R⁵ is —OR¹⁰ and R¹⁰ is C₁₋₆alkyl substituted with one, two, or three groups selected from C₁₋₆alkoxy, C₃₋₆ cycloalkyl, and C₂₋₉heterocycloalkyl. In some embodiments is a compound of Formula (III), or a pharmaceutically acceptable salt or solvate thereof, wherein R⁵ is —OR¹⁰ and R¹⁰ is C₁₋₆alkyl substituted with one group selected from C₁₋₆alkoxy, C₃₋₆cycloalkyl, and C₂₋₉heterocycloalkyl. In some embodiments is a compound of Formula (III), or a pharmaceutically acceptable salt or solvate thereof, wherein R⁵ is —OR¹⁰ and R¹⁰ is unsubstituted C₁₋₆alkyl. In some embodiments is a compound of Formula (III), or a pharmaceutically acceptable salt or solvate thereof, wherein R⁵ is —OR¹⁰ and R¹⁰ is —CH₃. In some embodiments is a compound of Formula (III), or a pharmaceutically acceptable salt or solvate thereof, wherein R⁵ is —OR¹⁰ and R¹⁰ is C₁₋₆ haloalkyl. In some embodiments is a compound of Formula (III), or a pharmaceutically acceptable salt or solvate thereof, wherein R⁵ is —OR¹⁰ and R¹⁰ is C₃₋₆cycloalkyl. In some embodiments is a compound of Formula (III), or a pharmaceutically acceptable salt or solvate thereof, wherein R⁵ is —OR¹⁰ and R¹⁰ is C₂₋₉heterocycloalkyl.

In some embodiments is a compound of Formula (III), or a pharmaceutically acceptable salt or solvate thereof, wherein R⁶ is

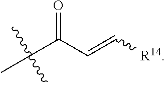

In some embodiments is a compound of Formula (III), or a pharmaceutically acceptable salt or solvate thereof, wherein R⁶ is

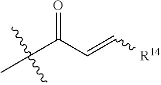

R¹⁴ and R¹¹ is hydrogen. In some embodiments is a compound of Formula (III), or a pharmaceutically acceptable salt or solvate thereof, wherein R⁶ is

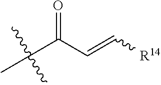

and R¹⁴ is C₁₋₆alkyl optionally substituted with —N(R¹⁰)(R¹¹).

In some embodiments is a compound of Formula (III), or a pharmaceutically acceptable salt or solvate thereof, wherein R⁶ is

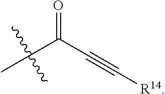

In some embodiments is a compound of Formula (III), or a pharmaceutically acceptable salt or solvate thereof, wherein R⁶ is

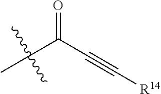

and R¹⁴ is hydrogen. In some embodiments is a compound of Formula (III), or a pharmaceutically acceptable salt or solvate thereof, wherein R⁶ is

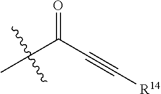

and R¹⁴ is C₁₋₆alkyl optionally substituted with —N(R¹⁰)(R¹¹).

In some embodiments is a compound of Formula (III), or a pharmaceutically acceptable salt or solvate thereof, wherein R⁶ is

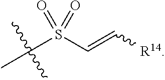

In some embodiments is a compound of Formula III or a pharmaceutically acceptable salt or solvate thereof, wherein R⁶ is

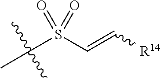

and R¹⁴ is hydrogen. In some embodiments is a compound of Formula (III), or a pharmaceutically acceptable salt or solvate thereof, wherein R⁶ is

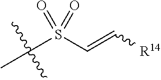

and R¹⁴ is C₁₋₆alkyl optionally substituted with —N(R¹⁰)(R¹¹).

In some embodiments, is a compound selected from:
1) 1-(3-(4-((3,4-dichloro-2-fluorophenyl)amino)-7-methoxyquinazolin-6-yl)azetidin-1-yl)prop-2-en-1-one
3) 1-(3-(4-((3,4-dichloro-2-fluorophenyl)amino)-7-methoxyquinazolin-6-yl)pyrrolidin-1-yl)prop-2-en-1-one
4) 1-(3-(7-methoxy-4-(naphthalen-1-ylamino)quinazolin-6-yl)azetidin-1-yl)prop-2-en-1-one
5) 1-(3-(7-methoxy-4-(naphthalen-2-ylamino)quinazolin-6-yl)azetidin-1-yl)prop-2-en-1-one
6) 1-(3-(4-((3-chloro-4-fluorophenyl)amino)-7-methoxyquinazolin-6-yl)azetidin-1-yl)prop-2-en-1-one
7) 1-(3-(4-((4-chloronaphthalen-1-yl)amino)-7-methoxyquinazolin-6-yl)azetidin-1-yl)prop-2-en-1-one
8) 1-(3-(4-((3,4-dichloro-2-fluorophenyl)amino)quinazolin-6-yl)azetidin-1-yl)prop-2-en-1-one
9) 1-(4-(4-((3,4-dichloro-2-fluorophenyl)amino)quinazolin-6-yl)piperidin-1-yl)prop-2-en-1-one
10) 1-(4-(4-((3,4-dichloro-2-fluorophenyl)amino)-7-methoxyquinazolin-6-yl)piperidin-1-yl)prop-2-en-1-one
11) 1-(3-(4-((1H-indol-5-yl)amino)-7-methoxyquinazolin-6-yl)azetidin-1-yl)prop-2-en-1-one
13) 1-(3-(4-((4-fluorobenzyl)amino)-7-methoxyquinazolin-6-yl)azetidin-1-yl)prop-2-en-1-one
14) 1-(3-(4-((4-fluoronaphthalen-1-yl)amino)-7-methoxyquinazolin-6-yl)azetidin-1-yl)prop-2-en-1-one
15) 1-(3-(4-((3-chlorobenzyl)amino)-7-methoxyquinazolin-6-yl)azetidin-1-yl)prop-2-en-1-one
16) 1-(3-(4-((3-chloro-2,4-difluorophenyl)amino)-7-methoxyquinazolin-6-yl)azetidin-1-yl)prop-2-en-1-one
17) 1-(3-(4-((3,4-dichloro-2-fluorophenyl)amino)-7-(2-methoxyethoxy)quinazolin-6-yl)azetidin-1-yl)prop-2-en-1-one
18) 1-(3-(4-((3,4-dichloro-2-fluorophenyl)amino)-7-ethoxyquinazolin-6-yl)azetidin-1-yl)prop-2-en-1-one
19) 1-(3-(4-((3,4-dichloro-2-fluorophenyl)amino)-7-(2-hydroxyethoxy)quinazolin-6-yl)azetidin-1-yl)prop-2-en-1-one
20) 1-(3-(4-((3,4-dichloro-2-fluorophenyl)amino)-7-(2,2,2-trifluoroethoxy)quinazolin-6-yl)azetidin-1-yl)prop-2-en-1-one
21) 1-(3-(4-((3,4-dichloro-2-fluorophenyl)amino)-7-(2-(4-methylpiperazin-1-yl)ethoxy)quinazolin-6-yl)azetidin-1-yl)prop-2-en-1-one
22) (S)-1-(3-(4-((3,4-dichloro-2-fluorophenyl)amino)-7-((tetrahydrofuran-3-yl)oxy)quinazolin-6-yl)azetidin-1-yl)prop-2-en-1-one
23) 1-(3-(4-((3,4-dichloro-2-fluorophenyl)amino)-7-(difluoromethoxy)quinazolin-6-yl)azetidin-1-yl)prop-2-en-1-one
24) (S)-1-(3-(4-((3,4-dichloro-2-fluorophenyl)amino)-7-((tetrahydrofuran-3-yl)oxy)quinazolin-6-yl)azetidin-1-yl)prop-2-en-1-one
25) 1-(3-(4-((3,4-dichloro-2-fluorophenyl)amino)-7-(oxetan-3-yloxy)quinazolin-6-yl)azetidin-1-yl)prop-2-en-1-one
26) 1-(3-(4-((3,4-dichloro-2-fluorophenyl)amino)-7-(2-(dimethylamino)ethoxy)quinazolin-6-yl)azetidin-1-yl)prop-2-en-1-one
27) 1-(3-(4-((3,4-dichloro-2-fluorophenyl)amino)-7-((tetrahydro-2H-pyran-4-yl)oxy)quinazolin-6-yl)azetidin-1-yl)prop-2-en-1-one
28) 1-(3-(4-((3,4-dichloro-2-fluorophenyl)amino)-7-(2-morpholinoethoxy)quinazolin-6-yl)azetidin-1-yl)prop-2-en-1-one
29) (R)-1-(3-(4-((3,4-dichloro-2-fluorophenyl)amino)-7-((tetrahydrofuran-3-yl)oxy)quinazolin-6-yl)azetidin-1-yl)prop-2-en-1-one
30) 1-(3-(4-((3,4-dichloro-2-fluorophenyl)amino)-7-(3-morpholinopropoxy)quinazolin-6-yl)azetidin-1-yl)prop-2-en-1-one
31) 1-(3-(4-((3,4-dichloro-2-fluorophenyl)amino)-7-(3-(4-methylpiperazin-1-yl)propoxy)quinazolin-6-yl)azetidin-1-yl)prop-2-en-1-one
32) 1-(3-(4-((3,4-dichloro-2-fluorophenyl)amino)-7-(methoxy-d3)quinazolin-6-yl)azetidin-1-yl)prop-2-en-1-one
33) (S)-1-(3-(4-((3-chloro-2,4-difluorophenyl)amino)-7-((tetrahydrofuran-3-yl)oxy)quinazolin-6-yl)azetidin-1-yl)prop-2-en-1-one
34)N-((4-((3,4-dichloro-2-fluorophenyl)amino)-6,7-dihydrofuro[3,2-g]quinazolin-6-yl)methyl)acrylamide
37) (S)-1-(3-(4-((3,4-dichloro-2-fluorophenyl)amino)-7-((tetrahydrofuran-3-yl)oxy)quinazolin-6-yl)azetidin-1-yl)-2-fluoroprop-2-en-1-one
38) 1-(3-(4-((3,4-dichloro-2-fluorophenyl)amino)-7-methoxyquinazolin-6-yl)azetidin-1-yl)-2-fluoroprop-2-en-1-one
39) 1-(3-(4-((3,4-dichloro-2-fluorophenyl)amino)-7-(methoxy-d3)quinazolin-6-yl)azetidin-1-yl)-2-fluoroprop-2-en-1-one
40)N-((1s,3s)-3-((4-((3,4-dichloro-2-fluorophenyl)amino)-7-methoxyquinazolin-6-yl)oxy)cyclobutyl)-2-fluoroacrylamide
41) 1-(3-(4-((4-(benzyloxy)-3-chloro-2-fluorophenyl)amino)-7-methoxyquinazolin-6-yl)azetidin-1-yl)prop-2-yn-1-one
42) (E)-N-(3-((4-((3,4-dichloro-2-fluorophenyl)amino)-7-methoxyquinazolin-6-yl)oxy)cyclobutyl)-4-(dimethylamino)but-2-enamide
43)N-(3-((4-((3,4-dichloro-2-fluorophenyl)amino)-7-methoxyquinazolin-6-yl)oxy)cyclobutyl)but-2-ynamide
44)N-((1r,3r)-3-((4-((2,3-dihydro-1H-inden-4-yl)amino)-7-methoxyquinazolin-6-yl)oxy)cyclobutyl)propiolamide
45)N-((1s,3s)-3-((4-((2,3-dihydro-1H-inden-4-yl)amino)-7-methoxyquinazolin-6-yl)oxy)cyclobutyl)propiolamide
46)N-(3-((7-methoxy-4-(naphthalen-1-ylamino)quinazolin-6-yl)oxy)cyclobutyl)propiolamide
47)N-((1s,3s)-3-((7-methoxy-4-(naphthalen-1-ylamino)quinazolin-6-yl)oxy)cyclobutyl)propiolamide
48)N-((1r,3r)-3-((7-methoxy-4-(naphthalen-2-ylamino)quinazolin-6-yl)oxy)cyclobutyl)propiolamide
49)N-(3-((4-((3,4-dichloro-2-fluorophenyl)amino)-7-methoxyquinazolin-6-yl)oxy)cyclobutyl)propiolamide
50) 1-(4-((4-((3,4-dichloro-2-fluorophenyl)amino)-7-methoxyquinazolin-6-yl)oxy)piperidin-1-yl)-2-fluoroprop-2-en-1-one
51) 1-(4-((4-(3,4-dichloro-2-fluorobenzoyl)-7-methoxyquinazolin-6-yl)oxy)piperidin-1-yl)prop-2-yn-1-one
52) 1-(4-((4-((3,4-dichloro-2-fluorophenyl)amino)-7-methoxyquinazolin-6-yl)oxy)piperidin-1-yl)prop-2-yn-1-one
53) 1-(4-((7-methoxy-4-(naphthalen-2-ylamino)quinazolin-6-yl)oxy)piperidin-1-yl)prop-2-yn-1-one
54) 1-(4-((7-methoxy-4-(naphthalen-1-ylamino)quinazolin-6-yl)oxy)piperidin-1-yl)prop-2-yn-1-one
56) 1-(3-((4-((2,3-dihydro-1H-inden-4-yl)amino)-7-methoxyquinazolin-6-yl)oxy)-8-azabicyclo[3.2.1]octan-8-yl)prop-2-yn-1-one
57) 1-(3-((7-methoxy-4-(naphthalen-1-ylamino)quinazolin-6-yl)oxy)-9-azabicyclo[3.3.1]nonan-9-yl)prop-2-yn-1-one
58) 1-(3-((7-methoxy-4-(naphthalen-2-ylamino)quinazolin-6-yl)oxy)-9-azabicyclo[3.3.1]nonan-9-yl)prop-2-yn-1-one
59) 1-(3-((7-methoxy-4-(naphthalen-2-ylamino)quinazolin-6-yl)oxy)-8-azabicyclo[3.2.1]octan-8-yl)prop-2-yn-1-one
60) 1-(3-((7-methoxy-4-(naphthalen-1-ylamino)quinazolin-6-yl)oxy)-8-azabicyclo[3.2.1]octan-8-yl)prop-2-yn-1-one
61) 1-(3-(4-((3,4-dichloro-2-fluorophenyl)amino)quinazolin-6-yl)piperidin-1-yl)prop-2-yn-1-one
62) 1-(5-(4-((3,4-dichloro-2-fluorophenyl)amino)quinazolin-6-yl)-3,6-dihydropyridin-1(2H)-yl)prop-2-yn-1-one
63) 1-(5-(4-((3,4-dichloro-2-fluorophenyl)amino)-7-methoxyquinazolin-6-yl)-3,6-dihydropyridin-1(2H)-yl)prop-2-yn-1-one
64) 1-(4-((7-methoxy-4-(naphthalen-2-ylamino)quinazolin-6-yl)oxy)piperidin-1-yl)prop-2-yn-1-one
66)N-((1s,3s)-3-((4-((3,4-dichloro-2-fluorophenyl)amino)-7-methoxyquinazolin-6-yl)oxy)cyclobutyl)acrylamide
67)N-(3-((7-methoxy-4-((7-methyl-2,3-dihydro-1H-inden-4-yl)amino)quinazolin-6-yl)oxy)cyclobutyl)acrylamide
72)N-(3-((4-((3,4-dichloro-2-fluorophenyl)amino)-7-methoxyquinazolin-6-yl)oxy)cyclobutyl)-N-methylacrylamide
73)N-(3-((4-((3,4-dichlorophenyl)amino)-7-methoxyquinazolin-6-yl)oxy)cyclobutyl)acrylamide
74)N-(3-((4-((3-chloro-2-fluorophenyl)amino)-7-methoxyquinazolin-6-yl)oxy)cyclobutyl)acrylamide
75)N-(3-((4-((2,4-dichloro-3-fluorophenyl)amino)-7-methoxyquinazolin-6-yl)oxy)cyclobutyl)acrylamide
76)N-(3-((4-((2,3-dichloro-4-fluorophenyl)amino)-7-methoxyquinazolin-6-yl)oxy)cyclobutyl)acrylamide
77)N-(3-((4-((2-fluorophenyl)amino)-7-methoxyquinazolin-6-yl)oxy)cyclobutyl)acrylamide
78)N-(3-((4-((4-chloro-2-fluorophenyl)amino)-7-methoxyquinazolin-6-yl)oxy)cyclobutyl)acrylamide
79)N-((1r,3r)-3-((4-((3,4-dichloro-2-fluorophenyl)amino)-7-methoxyquinazolin-6-yl)oxy)cyclobutyl)acrylamide
80)N-(3-((7-methoxy-4-(naphthalen-2-ylamino)quinazolin-6-yl)oxy)cyclobutyl)acrylamide
81)N-(3-((7-methoxy-4-(naphthalen-1-ylamino)quinazolin-6-yl)oxy)cyclobutyl)acrylamide
82)N-((1s,3s)-3-((4-((3,4-dichlorophenyl)amino)-7-methoxyquinazolin-6-yl)oxy)cyclobutyl)acrylamide
83)N-((1s,3s)-3-((7-methoxy-4-(naphthalen-1-ylamino)quinazolin-6-yl)oxy)cyclobutyl)acrylamide
84)N-(3-((4-((4-fluorobenzyl)amino)-7-methoxyquinazolin-6-yl)oxy)cyclobutyl)acrylamide
85)N-(3-((4-((3-chloro-4-fluorophenyl)amino)-7-methoxyquinazolin-6-yl)oxy)cyclobutyl)acrylamide
86)N-(3-((4-((3-chloro-4-cyanophenyl)amino)-7-methoxyquinazolin-6-yl)oxy)cyclobutyl)acrylamide
87)N-(3-((4-((3-cyano-4-fluorophenyl)amino)-7-methoxyquinazolin-6-yl)oxy)cyclobutyl)acrylamide
88)N-(3-((4-((2,3-dihydro-1H-inden-4-yl)amino)-7-methoxyquinazolin-6-yl)oxy)cyclobutyl)acrylamide
89)N-(3-((7-methoxy-4-((5,6,7,8-tetrahydronaphthalen-1-yl)amino)quinazolin-6-yl)oxy)cyclobutyl)acrylamide
90)N-((1s,4s)-4-((4-((3,4-dichloro-2-fluorophenyl)amino)-7-methoxyquinazolin-6-yl)oxy)cyclohexyl)acrylamide
91)N-((1r,4r)-4-((4-((3,4-dichloro-2-fluorophenyl)amino)-7-methoxyquinazolin-6-yl)oxy)cyclohexyl)acrylamide
92)N-(3-((4-((3,4-dichloro-2-fluorophenyl)amino)-7-methoxyquinazolin-6-yl)amino)cyclobutyl)acrylamide
94) 6-((1-acryloylpiperidin-4-yl)oxy)-N-(4-fluorobenzyl)-7-methoxyquinazoline-4-carboxamide
98)N-(6-((1-acryloylpiperidin-4-yl)oxy)-7-methoxyquinazolin-4-yl)-4-fluorobenzamide
99)N-((1R,3s)-3-((4-((3,4-dichloro-2-fluorophenyl)amino)-7-(((S)-tetrahydrofuran-3-yl)oxy)quinazolin-6-yl)oxy)cyclobutyl)acrylamide
100)N-((1s,3s)-3-((4-((3,4-dichloro-2-fluorophenyl)amino)-7-(2-hydroxyethoxy)quinazolin-6-yl)oxy)cyclobutyl)acrylamide
101)N-((1s,3s)-3-((4-((3,4-dichloro-2-fluorophenyl)amino)-7-(2-(dimethylamino)ethoxy)quinazolin-6-yl)oxy)cyclobutyl)acrylamide
102)N-((1s,3s)-3-((4-((3,4-dichloro-2-fluorophenyl)amino)-7-(methoxy-d3)quinazolin-6-yl)oxy)cyclobutyl)acrylamide
103)N-((1s,3s)-3-((4-((3,4-dichloro-2-fluorophenyl)amino)-7-(methylamino)quinazolin-6-yl)oxy)cyclobutyl)acrylamide
104)N-((1s,3s)-3-((4-((3,4-dichloro-2-fluorophenyl)amino)-7-(oxetan-3-yloxy)quinazolin-6-yl)oxy)cyclobutyl)acrylamide
105)N-((1s,3s)-3-((4-((3,4-dichloro-2-fluorophenyl)amino)-7-ethoxyquinazolin-6-yl)oxy)cyclobutyl)acrylamide
106) 1-(4-((4-((3-chlorobenzyl)amino)-7-methoxyquinazolin-6-yl)oxy)piperidin-1-yl)prop-2-en-1-one
107) 1-(4-((4-((3-chlorobenzyl)oxy)-7-methoxyquinazolin-6-yl)oxy)piperidin-1-yl)prop-2-en-1-one
108) 1-(4-((4-((4-chlorobenzyl)amino)-7-methoxyquinazolin-6-yl)oxy)piperidin-1-yl)prop-2-en-1-one
109) 1-(4-((4-((2,4-dichlorobenzyl)amino)-7-methoxyquinazolin-6-yl)oxy)piperidin-1-yl)prop-2-en-1-one
110) 1-(4-((4-((2-chlorobenzyl)amino)-7-methoxyquinazolin-6-yl)oxy)piperidin-1-yl)prop-2-en-1-one
111) 1-(4-((4-((3-fluorobenzyl)amino)-7-methoxyquinazolin-6-yl)oxy)piperidin-1-yl)prop-2-en-1-one
112) 1-(4-((4-((2-fluorobenzyl)amino)-7-methoxyquinazolin-6-yl)oxy)piperidin-1-yl)prop-2-en-1-one
113) 1-(4-((4-((4-chloro-3-fluorobenzyl)amino)-7-methoxyquinazolin-6-yl)oxy)piperidin-1-yl)prop-2-en-1-one
114) 1-(4-((4-((3-chloro-4-fluorobenzyl)amino)-7-methoxyquinazolin-6-yl)oxy)piperidin-1-yl)prop-2-en-1-one
115) 1-(4-((4-((3,4-dichlorobenzyl)amino)-7-methoxyquinazolin-6-yl)oxy)piperidin-1-yl)prop-2-en-1-one
116) 1-(4-((4-((3-chloro-2-fluorobenzyl)amino)-7-methoxyquinazolin-6-yl)oxy)piperidin-1-yl)prop-2-en-1-one
117) 1-(4-((4-((3,5-dichlorobenzyl)amino)-7-methoxyquinazolin-6-yl)oxy)piperidin-1-yl)prop-2-en-1-one
118) 1-(4-((4-((5-chloro-2-fluorobenzyl)amino)-7-methoxyquinazolin-6-yl)oxy)piperidin-1-yl)prop-2-en-1-one
119) 1-(4-((4-((2,5-dichlorobenzyl)amino)-7-methoxyquinazolin-6-yl)oxy)piperidin-1-yl)prop-2-en-1-one
120) 1-(4-((4-((3-bromobenzyl)amino)-7-methoxyquinazolin-6-yl)oxy)piperidin-1-yl)prop-2-en-1-one
121) 1-(4-((7-methoxy-4-((3-methylbenzyl)amino)quinazolin-6-yl)oxy)piperidin-1-yl)prop-2-en-1-one
122) 1-(4-((4-((3-chloro-5-fluorobenzyl)amino)-7-methoxyquinazolin-6-yl)oxy)piperidin-1-yl)prop-2-en-1-one
123) 1-(4-((4-(benzylamino)-7-methoxyquinazolin-6-yl)oxy)piperidin-1-yl)prop-2-en-1-one
124) 3-(((6-((1-acryloylpiperidin-4-yl)oxy)-7-methoxyquinazolin-4-yl)amino)methyl)benzonitrile
125) 1-(4-((4-((3-iodobenzyl)amino)-7-methoxyquinazolin-6-yl)oxy)piperidin-1-yl)prop-2-en-1-one
127) 2-((6-((1-acryloylpiperidin-4-yl)oxy)-7-methoxyquinazolin-4-yl)amino)-2-(3-chlorophenyl)acetamide
130) 3-((6-((1-acryloylpiperidin-4-yl)oxy)-7-methoxyquinazolin-4-yl)amino)-3-(3-chlorophenyl) propanamide
131) 1-(4-((4-((1-(3-chlorophenyl)-3-hydroxypropyl)amino)-7-methoxyquinazolin-6-yl)oxy)piperidin-1-yl)prop-2-en-1-one
132) 1-(3-((4-((3,4-dichloro-2-fluorophenyl)amino)-7-methoxyquinazolin-6-yl)oxy)piperidin-1-yl)prop-2-en-1-one
133) 1-(4-((4-((3,4-dichloro-2-fluorophenyl)amino)-7-methoxyquinazolin-6-yl)oxy)piperidin-1-yl)prop-2-en-1-one
134) 1-(4-((7-methoxy-4-((1-(methylsulfonyl)indolin-5-yl)amino)quinazolin-6-yl)oxy)piperidin-1-yl)prop-2-en-1-one
135) 1-(4-((7-methoxy-4-(quinolin-3-ylamino)quinazolin-6-yl)oxy)piperidin-1-yl)prop-2-en-1-one
136) 1-(4-((4-((5-chlorobenzo[d]oxazol-2-yl)amino)-7-methoxyquinazolin-6-yl)oxy)piperidin-1-yl)prop-2-en-1-one
137) 1-(4-((4-((1H-indazol-6-yl)amino)-7-methoxyquinazolin-6-yl)oxy)piperidin-1-yl)prop-2-en-1-one
138) 1-(4-((7-methoxy-4-((1-methyl-1H-indazol-6-yl)amino)quinazolin-6-yl)oxy)piperidin-1-yl)prop-2-en-1-one
139) 1-(4-((4-((1H-indazol-7-yl)amino)-7-methoxyquinazolin-6-yl)oxy)piperidin-1-yl)prop-2-en-1-one
141) 1-(4-((7-methoxy-4-(quinolin-7-ylamino)quinazolin-6-yl)oxy)piperidin-1-yl)prop-2-en-1-one
142) 1-(4-((4-(benzo[d]thiazol-5-ylamino)-7-methoxyquinazolin-6-yl)oxy)piperidin-1-yl)prop-2-en-1-one
143) 1-(4-((7-methoxy-4-(quinolin-6-ylamino)quinazolin-6-yl)oxy)piperidin-1-yl)prop-2-en-1-one
144) 1-(4-((7-methoxy-4-(quinoxalin-6-ylamino)quinazolin-6-yl)oxy)piperidin-1-yl)prop-2-en-1-one
145) 1-(4-((4-(benzo[d]thiazol-6-ylamino)-7-methoxyquinazolin-6-yl)oxy)piperidin-1-yl)prop-2-en-1-one
146) 1-(4-((7-methoxy-4-(quinolin-8-ylamino)quinazolin-6-yl)oxy)piperidin-1-yl)prop-2-en-1-one
148) 1-(4-((7-methoxy-4-(naphthalen-2-ylamino)quinazolin-6-yl)oxy)piperidin-1-yl)prop-2-en-1-one
149) 1-(4-((7-methoxy-4-(naphthalen-1-ylamino)quinazolin-6-yl)oxy)piperidin-1-yl)prop-2-en-1-one
150) 1-(4-((4-((1H-indazol-5-yl)amino)-7-methoxyquinazolin-6-yl)oxy)piperidin-1-yl)prop-2-en-1-one
152) 1-(4-((4-(benzo[d][1,3]dioxol-5-ylamino)-7-methoxyquinazolin-6-yl)oxy)piperidin-1-yl)prop-2-en-1-one
153) 1-(4-((4-((2,3-dihydro-1H-inden-5-yl)amino)-7-methoxyquinazolin-6-yl)oxy)piperidin-1-yl)prop-2-en-1-one
154) 1-(4-((4-((5-chlorobenzo[d]oxazol-2-yl)amino)-7-methoxyquinazolin-6-yl)oxy)piperidin-1-yl)prop-2-en-1-one
155) 1-(4-((7-methoxy-4-((2-methylbenzo[d]thiazol-5-yl)amino)quinazolin-6-yl)oxy)piperidin-1-yl)prop-2-en-1-one
156) 1-(4-((4-((2,3-dihydro-1H-inden-4-yl)amino)-7-methoxyquinazolin-6-yl)oxy)piperidin-1-yl)prop-2-en-1-one
157) 1-(4-((4-(benzo[d]thiazol-5-ylamino)-7-methoxyquinazolin-6-yl)oxy)piperidin-1-yl)prop-2-en-1-one
158) 1-(4-((7-methoxy-4-(quinolin-2-ylamino)quinazolin-6-yl)oxy)piperidin-1-yl)prop-2-en-1-one
159) 1-(4-((4-(isoquinolin-3-ylamino)-7-methoxyquinazolin-6-yl)oxy)piperidin-1-yl)prop-2-en-1-one
160) 1-(4-((4-((1-chloronaphthalen-2-yl)amino)-7-methoxyquinazolin-6-yl)oxy)piperidin-1-yl)prop-2-en-1-one
161) 1-(4-((4-(isoquinolin-1-ylamino)-7-methoxyquinazolin-6-yl)oxy)piperidin-1-yl)prop-2-en-1-one
162) 1-(4-((7-methoxy-4-(quinolin-4-ylamino)quinazolin-6-yl)oxy)piperidin-1-yl)prop-2-en-1-one
164) 1-(4-((4-((4-chloronaphthalen-1-yl)amino)-7-methoxyquinazolin-6-yl)oxy)piperidin-1-yl)prop-2-en-1-one
165) 1-(4-((4-((1H-indol-4-yl)amino)-7-methoxyquinazolin-6-yl)oxy)piperidin-1-yl)prop-2-en-1-one
166) 1-(4-((4-((1H-indol-7-yl)amino)-7-methoxyquinazolin-6-yl)oxy)piperidin-1-yl)prop-2-en-1-one
167) 1-(4-((7-methoxy-4-((4-methylnaphthalen-1-yl)amino)quinazolin-6-yl)oxy)piperidin-1-yl)prop-2-en-1-one
168) 1-(4-((4-(indolin-5-ylamino)-7-methoxyquinazolin-6-yl)oxy)piperidin-1-yl)prop-2-en-1-one
169) 1-(4-((4-((1H-indol-5-yl)amino)-7-methoxyquinazolin-6-yl)oxy)piperidin-1-yl)prop-2-en-1-one
170) 1-(4-((7-methoxy-4-((1-methyl-1H-indol-5-yl)amino)quinazolin-6-yl)oxy)piperidin-1-yl)prop-2-en-1-one
171) 1-(4-((4-(indolin-5-ylamino)-7-methoxyquinazolin-6-yl)oxy)piperidin-1-yl)prop-2-en-1-one
172) 1-(4-((4-((4-fluorobenzyl)amino)-7-methoxyquinazolin-6-yl)oxy)piperidin-1-yl)prop-2-en-1-one
173) (R)-1-(4-((4-((1-(4-fluorophenyl)ethyl)amino)-7-methoxyquinazolin-6-yl)oxy)piperidin-1-yl)prop-2-en-1-one
175) 1-(4-((4-(3,4-dichloro-2-fluorobenzoyl)-7-methoxyquinazolin-6-yl)oxy)piperidin-1-yl)prop-2-en-1-one
176) 1-(3-((4-((3,4-dichloro-2-fluorophenyl)amino)-7-methoxyquinazolin-6-yl)oxy)-9-azabicyclo[3.3.1]nonan-9-yl)prop-2-en-1-one
177) 1-(3-((4-((3,4-dichloro-2-fluorophenyl)amino)-7-methoxyquinazolin-6-yl)oxy)-8-azabicyclo[3.2.1]octan-8-yl)prop-2-en-1-one
178) 1-(3-((7-methoxy-4-(naphthalen-1-ylamino)quinazolin-6-yl)oxy)-9-azabicyclo[3.3.1]nonan-9-yl)prop-2-en-1-one
179) 1-(3-((7-methoxy-4-(naphthalen-2-ylamino)quinazolin-6-yl)oxy)-9-azabicyclo[3.3.1]nonan-9-yl)prop-2-en-1-one
180) 1-(3-((7-methoxy-4-(naphthalen-2-ylamino)quinazolin-6-yl)oxy)-8-azabicyclo[3.2.1]octan-8-yl)prop-2-en-1-one
181) 1-(3-((7-methoxy-4-(naphthalen-1-ylamino)quinazolin-6-yl)oxy)-8-azabicyclo[3.2.1]octan-8-yl)prop-2-en-1-one
182) 1-(3-((4-((2,3-dihydro-1H-inden-4-yl)amino)-7-methoxyquinazolin-6-yl)oxy)-9-azabicyclo[3.3.1]nonan-9-yl)prop-2-en-1-one
183) 1-(3-((4-((2,3-dihydro-1H-inden-4-yl)amino)-7-methoxyquinazolin-6-yl)oxy)-8-azabicyclo[3.2.1]octan-8-yl)prop-2-en-1-one
184) 1-(3-(4-((3,4-dichloro-2-fluorophenyl)amino)quinazolin-6-yl)piperidin-1-yl)prop-2-en-1-one
185) 1-(3-(4-((3,4-dichloro-2-fluorophenyl)amino)-7-methoxyquinazolin-6-yl)piperidin-1-yl)prop-2-en-1-one
186) 1-(5-(4-((3,4-dichloro-2-fluorophenyl)amino)-7-methoxyquinazolin-6-yl)-3,6-dihydropyridin-1(2H)-yl)prop-2-en-1-one
187) 1-(5-(4-((3,4-dichloro-2-fluorophenyl)amino)quinazolin-6-yl)-3,6-dihydropyridin-1(2H)-yl)prop-2-en-1-one
188)N-(3,4-dichloro-2-fluorophenyl)-6-(1-(vinylsulfonyl)piperidin-3-yl)quinazolin-4-amine
190)N-(3,4-dichloro-2-fluorophenyl)-6-(1-(vinylsulfonyl)-1,2,5,6-tetrahydropyridin-3-yl)quinazolin-4-amine
191)N-(3-((4-((3,4-dichloro-2-fluorophenyl)amino)-7-methoxyquinazolin-6-yl)oxy)cyclobutyl)ethenesulfonamide
192)N-(3,4-dichloro-2-fluorophenyl)-7-methoxy-6-((1-(vinylsulfonyl)piperidin-4-yl)oxy)quinazolin-4-amine
193) (3,4-dichloro-2-fluorophenyl)(7-methoxy-6-((1-(vinylsulfonyl)piperidin-4-yl)oxy)quinazolin-4-yl)methanone
194) 4-((4-((3,4-dichloro-2-fluorophenyl)amino)-7-methoxyquinazolin-6-yl)oxy)piperidine-1-carbonitrile
195) 3-(4-((3,4-dichloro-2-fluorophenyl)amino)-7-methoxyquinazolin-6-yl)azetidine-1-carbonitrile
196)N-(3-((4-((3,4-dichloro-2-fluorophenyl)amino)-7-methoxyquinazolin-6-yl)oxy)cyclobutyl)-N-methylcyanamide
197) 5-(4-((3,4-dichloro-2-fluorophenyl)amino)-7-methoxyquinazolin-6-yl)-3,6-dihydropyridine-1(2H)-carbonitrile
198) 3-((4-((3,4-dichloro-2-fluorophenyl)amino)-7-methoxyquinazolin-6-yl)oxy)piperidine-1-carbonitrile
199) 9-((1-acryloylpiperidin-4-yl)oxy)-3-(3,4-dichloro-2-fluorophenyl)-8-methoxy-1H-pyrimido[4,5,6-de]quinazolin-2(3H)-one
200) 9-((1-acryloylpiperidin-4-yl)oxy)-3-(3,4-dichloro-2-fluorophenyl)-8-methoxy-1-methyl-1H-pyrimido[4,5,6-de]quinazolin-2(3H)-one
205) 9-((1-acryloylpyrrolidin-3-yl)oxy)-3-(3,4-dichloro-2-fluorophenyl)-8-methoxy-1H-pyrimido[4,5,6-de]quinazolin-2(3H)-one
206) 9-(1-acryloyl-1,2,3,6-tetrahydropyridin-4-yl)-3-(3,4-dichloro-2-fluorophenyl)-8-methoxy-1-methyl-1H-pyrimido[4,5,6-de]quinazolin-2(3H)-one
207)N-(3,4-dichloro-2-fluorophenyl)-7-methoxy-5-nitro-6-(piperidin-4-yloxy)quinazolin-4-amine hydrochloride 208) 1-(4-((4-((3,4-dichloro-2-fluorophenyl)amino)-7-methoxy-5-nitroquinazolin-6-yl)oxy)piperidin-1-yl)prop-2-en-1-one
215) 9-((1-acryloylpiperidin-4-yl)oxy)-3-(3,4-dichlorophenyl)-8-methoxy-1H-pyrimido[4,5,6-de]quinazolin-2(3H)-one
216) 9-((1-acryloylazetidin-3-yl)oxy)-3-(3,4-dichloro-2-fluorophenyl)-8-methoxy-1H-pyrimido[4,5,6-de]quinazolin-2(3H)-one
217) 1-(4-((4-(3,4-dichloro-2-fluorophenyl)-8-methoxy-5-methyl-4H-pyrido[2,3,4-de]quinazolin-7-yl)oxy)piperidin-1yl)prop-2-en-1-one
218) 1-(4-((4-(3,4-dichloro-2-fluorophenyl)-8-methoxy-4H-pyrido[2,3,4-de]quinazolin-7-yl)oxy) piperidin-1-yl)prop-2-en-1-one
225) 3-(3,4-dichloro-2-fluorophenyl)-8-methoxy-9-((1-(vinylsulfonyl)piperidin-4-yl)oxy)-1H-pyrimido[4,5,6-de]quinazolin-2(3H)-one
226) 3-(3,4-dichlorophenyl)-8-methoxy-9-((1-(vinylsulfonyl)piperidin-4-yl)oxy)-1H-pyrimido[4,5,6-de]quinazolin-2(3H)-one
227) 3-(3,4-dichlorophenyl)-8-methoxy-9-((1-propioloylpiperidin-4-yl)oxy)-1H-pyrimido[4,5,6-de]quinazolin-2(3H)-one; and
228)N-(4-amino-5-(quinolin-3-yl)-8,9-dihydro-7H-pyrimido[5′,4′:4,5]pyrrolo[2,1-b][1,3]oxazin-8-yl)acrylamide

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁷O, ¹⁸O, ³¹P, ³²p, ³⁵, ¹⁸F, and ³⁶Cl, respectively. Compounds described herein, and pharmaceutically acceptable salts, esters, solvate, hydrates, or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry₄^{th}Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein.

### Pharmaceutical Compositions/Formulations

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference in their entirety.

A pharmaceutical composition, as used herein, refers to a mixture of a compound of Formula (I), (Ia), (II), or (III), or a pharmaceutically acceptable salt or solvate thereof, and at least one pharmaceutically acceptable excipient. A pharmaceutical composition, as used herein, refers to a mixture of a compound of Formula (I), (Ia), (II), or (III), or a pharmaceutically acceptable salt or solvate thereof, with other chemical components, such as carriers, stabilizers, diluents, dispersing agents, suspending agents, and/or thickening agents. The pharmaceutical composition facilitates administration of the compound to a mammal. In practicing the methods of treatment or use provided herein, therapeutically effective amounts of a compound of Formula (I), (Ia), (II), or (III), or a pharmaceutically acceptable salt or solvate thereof, are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors.

In some embodiments is a pharmaceutical composition comprising a compound of Formula (I), or a pharmaceutically acceptable salt or solvate thereof, and at least one pharmaceutically acceptable excipient.

In some embodiments is a pharmaceutical composition comprising a compound of Formula (Ia), or a pharmaceutically acceptable salt or solvate thereof, and at least one pharmaceutically acceptable excipient.

In some embodiments is a pharmaceutical composition comprising a compound of Formula (II), or a pharmaceutically acceptable salt or solvate thereof, and at least one pharmaceutically acceptable excipient.

In some embodiments is a pharmaceutical composition comprising a compound of Formula (III), or a pharmaceutically acceptable salt or solvate thereof, and at least one pharmaceutically acceptable excipient.

In some embodiments is a pharmaceutical composition comprising a compound of Formula (I), (Ia), (II), or (III), or a pharmaceutically acceptable salt or solvate thereof, and at least one pharmaceutically acceptable excipient, for the treatment of cancer. In some embodiments is a pharmaceutical composition comprising a compound of Formula (I), (Ia), (II), or (III), or a pharmaceutically acceptable salt or solvate thereof, and at least one pharmaceutically acceptable excipient, for the treatment of cancer, wherein the cancer is characterized by the overexpression of EGFR/Erb-B2 or mutations of EGFR/Erb-B2. In some embodiments is a pharmaceutical composition comprising a compound of Formula (I), (Ia), (II), or (III), or a pharmaceutically acceptable salt or solvate thereof, and at least one pharmaceutically acceptable excipient, for the treatment of cancer, wherein the cancer comprises exon 20 insertions. In some embodiments is a pharmaceutical composition comprising a compound of Formula (I), (Ia), (II), or (III), or a pharmaceutically acceptable salt or solvate thereof, and at least one pharmaceutically acceptable excipient, for the treatment of lung cancer. In some embodiments is a pharmaceutical composition comprising a compound of Formula (I), (Ia), (II), or (III), or a pharmaceutically acceptable salt or solvate thereof, and at least one pharmaceutically acceptable excipient, for the treatment of non-small-cell lung cancer.

In some embodiments, a compound of Formula (I), (Ia), (II), or (III), or a pharmaceutically acceptable salt or solvate thereof, is incorporated into pharmaceutical compositions to provide solid oral dosage forms. In other embodiments, a compound of Formula (I), (Ia), (II), or (III), or a pharmaceutically acceptable salt or solvate thereof, is used to prepare pharmaceutical compositions other than oral solid dosage forms. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

### Dosage Forms

The pharmaceutical compositions described herein can be formulated for administration to a mammal via any conventional means including, but not limited to, oral, parenteral (e.g., intravenous, subcutaneous, or intramuscular), buccal, intranasal, rectal, or transdermal administration routes. As used herein, the term “subject” or “individual” is used to mean an animal, preferably a mammal, including a human or non-human. The terms individual, patient and subject may be used interchangeably.

Moreover, the pharmaceutical compositions described herein, which include a compound of Formula (I), (Ia), (II), or (III), or a pharmaceutically acceptable salt or solvate thereof, can be formulated into any suitable dosage form, including but not limited to, solid oral dosage forms, controlled release formulations, fast melt formulations, effervescent formulations, tablets, powders, pills, capsules, delayed release formulations, extended release formulations, pulsatile release formulations, multiparticulate formulations, and mixed immediate release and controlled release formulations.

Pharmaceutical preparations for oral use can be obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations described herein may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing particles of a compound of Formula (I), (Ia), (II), or (III), or a pharmaceutically acceptable salt or solvate thereof, with one or more pharmaceutical excipients to form a bulk blend composition. When referring to these bulk blend compositions as homogeneous, it is meant that the particles of a compound of Formula (I), (Ia), (II), or (III), or a pharmaceutically acceptable salt or solvate thereof, are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can include a compound of Formula (I), (Ia), (II), or (III), or a pharmaceutically acceptable salt or solvate thereof, and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol, and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In some embodiments, in order to release a compound of Formula (I), (Ia), (II), or (III), or a pharmaceutically acceptable salt or solvate thereof, from a solid dosage form matrix as efficiently as possible, disintegrants are used in the formulation, especially when the dosage forms are compressed with binder. Suitable disintegrants for use in the solid dosage forms described herein include, but are not limited to, natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like. In some embodiments provided herein, the disintegrating agent is selected from the group consisting of natural starch, a pregelatinized starch, a sodium starch, methylcrystalline cellulose, methylcellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, a clay, or a gum. In some embodiments provided herein, the disintegrating agent is croscarmellose sodium.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as calcium, magnesium, stearic acid, sodium stearates, magnesium stearate, zinc stearate, waxes, Stearowet*, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like. In some embodiments provided herein, the lubricant is selected from the group consisting of stearic acid, calcium hydroxide, talc, corn starch, sodium stearyl fumarate, stearic acid, sodium stearates, magnesium stearate, zinc stearate, and waxes. In some embodiments provided herein, the lubricant is magnesium stearate.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like. In some embodiments provided herein, the diluent is selected from the group consisting of lactose, sucrose, dextrose, dextrates, maltodextrin, mannitol, xylitol, sorbitol, cyclodextrins, calcium phosphate, calcium sulfate, starches, modified starches, microcrystalline cellulose, microcellulose, and talc. In some embodiments provided herein, the diluent is microcrystalline cellulose.

The term “non water-soluble diluent” represents compounds typically used in the formulation of pharmaceuticals, such as calcium phosphate, calcium sulfate, starches, modified starches, microcrystalline cellulose, microcellulose (e.g., having a density of about 0.45 g/cm³, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS, and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. In some embodiments provided herein, the surfactant is selected from the group consisting of sodium lauryl sulfate, sorbitan monooleate, polyoxyethylene sorbitan monooleate, polysorbates, polaxomers, bile salts, glyceryl monostearate, copolymers of ethylene oxide and propylene oxide. In some embodiments provided herein, the surfactant is sodium lauryl sulfate.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone, and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

### Methods

In some embodiments is a method for treating cancer comprising administering to the individual in need thereof a therapeutically effective amount of a compound of Formula (I), (Ia), (II), or (III), or a pharmaceutically acceptable salt or solvate thereof, described herein. In some embodiments is a method for treating cancer comprising administering to the individual in need thereof a therapeutically effective amount of a compound of Formula (I), (Ia), (II), or (III), or a pharmaceutically acceptable salt or solvate thereof, described herein, wherein the cancer is characterized by the overexpression of EGFR/Erb-B2 or mutations of EGFR/Erb-B2. In some embodiments is a method for treating cancer comprising administering to the individual in need thereof a therapeutically effective amount of a compound of Formula (I), (Ia), (II), or (III), or a pharmaceutically acceptable salt or solvate thereof, described herein, wherein the cancer is characterized by the overexpression of EGFR/Erb-B2. In some embodiments is a method for treating cancer comprising administering to the individual in need thereof a therapeutically effective amount of a compound of Formula (I), (Ia), (II), or (III), or a pharmaceutically acceptable salt or solvate thereof, described herein, wherein the cancer is characterized by mutations of EGFR/Erb-B2. In some embodiments is a method of treating cancer in an individual in need thereof, comprising administering to the individual a therapeutically effective amount of a compound of Formula (I), (Ia), (II), or (III) described herein, or a pharmaceutically acceptable salt or solvate thereof, wherein the cancer comprises exon 20 insertions. In some embodiments is a method for treating cancer comprising administering to the individual in need thereof a therapeutically effective amount of a compound of Formula (I), (Ia), (II), or (III), or a pharmaceutically acceptable salt or solvate thereof, described herein, wherein the cancer is lung cancer. In some embodiments is a method for treating cancer comprising administering to the individual in need thereof a therapeutically effective amount of a compound of Formula (I), (Ia), (II), or (III), or a pharmaceutically acceptable salt or solvate thereof, described herein, wherein the cancer is non-small-cell lung cancer.

### Methods of Dosing and Treatment Regimens

In some embodiments, a compound of Formula (I), (Ia), (II), or (III), is used in the preparation of medicaments for the treatment of diseases or conditions that would benefit from inhibition of EGFR exon 20 insertion mutants. In some embodiments, a compound of Formula (I), (Ia), (II), or (III), is used in the preparation of medicaments for the treatment of cancer that would benefit from inhibition of EGFR exon 20 insertion mutants. In addition, a method for treating any of the diseases or conditions described herein in an individual in need of such treatment, involves administration of pharmaceutical compositions containing a compound of Formula (I), (Ia), (II), or (III), or pharmaceutically acceptable solvate thereof, in therapeutically effective amounts to said individual.

In some embodiments, compositions containing a compound of Formula (I), (Ia), (II), or (III) are administered for prophylactic, therapeutic, or maintenance treatment. In some embodiments, compositions containing a compound of Formula (I), (Ia), (II), or (III) are administered for therapeutic applications. In some embodiments, compositions containing a compound of Formula (I), (Ia), (II), or (III) are administered for prophylactic applications.

In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder, or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered, if necessary. Subsequently, the dosage or the frequency of administration, or both, can be reduced, as a function of the symptoms, to a level at which the improved disease, disorder, or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Kits/Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Packaging materials for use in packaging pharmaceutical products include, e.g., U.S. Pat. No. 5,323,907. Examples of pharmaceutical packaging materials include, but are not limited to, blister packs, bottles, tubes, bags, containers, bottles, and any packaging material suitable for a selected formulation and intended mode of administration and treatment.

In some embodiments, the compounds or compositions described herein, are presented in a package or dispenser device which may contain one or more unit dosage forms containing the active ingredient. The compound or composition described herein is packaged alone, or packaged with another compound or another ingredient or additive. In some embodiments, the package contains one or more containers filled with one or more of the ingredients of the pharmaceutical compositions. In some embodiments, the package comprises metal or plastic foil, such as a blister pack. In some embodiments, the package or dispenser device is accompanied by instructions for administration, such as instructions for administering the compounds or compositions for treating a neoplastic disease. In some embodiments, the package or dispenser is accompanied with a notice associated with the container in form prescribed by a governmental agency regulating the manufacture, use, or sale of pharmaceuticals, which notice is reflective of approval by the agency of the form of the drug for human or veterinary administration. In some embodiments, such notice, for example, is the labeling approved by the U.S. Food and Drug Administration for prescription drugs, or the approved product insert. In some embodiments, compositions include a compound described herein formulated in a compatible pharmaceutical carrier are prepared, placed in an appropriate container, and labeled for treatment of an indicated condition.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

### List of Abbreviations

As used throughout the description of the invention, the following abbreviations, unless otherwise indicated, shall be understood to have the following meanings:
ACN or MeCN acetonitrile
Bn benzyl
BOC or Boc tert-butyl carbamate
t-Bu tert-butyl
Cy cyclohexyl
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
eq or equiv equivalent(s)
Et ethyl
Et₂O diethyl ether
EtOH ethanol
EtOAc ethyl acetate
HPLC high performance liquid chromatography
Me methyl
MeOH methanol
MS mass spectroscopy
GC gas chromatography
h or hr hour(s)
KF Karl Fischer
min minutes
MsOH methanesulfonic acid
NMR nuclear magnetic resonance
RP-HPLC reverse phase-high performance liquid chromatography
RT or r.t. room temperature
TFA trifluoroacetic acid
THE tetrahydrofuran
TLC thin layer chromatography
V volumes

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: Preparation of 1-(3-(4-((3,4-dichloro-2-fluorophenyl)amino)-7-methoxyuinazolin-6-yl)azetidin-1-yl)prop-2-en-1-one (Compound 1)

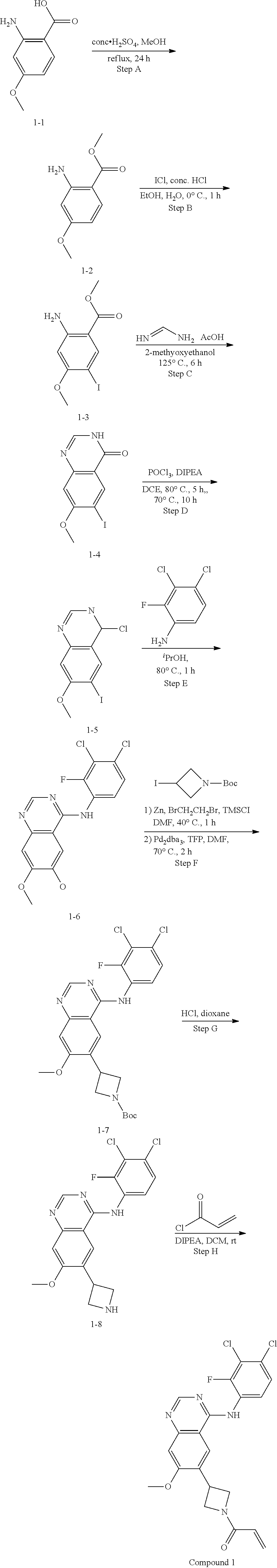

Step A: To a solution of 2-amino-4-methoxybenzoic acid (50 g, 0.3 mol) in MeOH (500 mL) was added concentrated sulfuric acid (50 mL) dropwise at 0° C. After addition, the mixture was heated to reflux for 2 days. The reaction mixture was cooled to room temperature. MeOH was removed in vacuo and the residue was poured into water (1 L). Na₂CO₃ was added with stirring until the pH>8. The resulting mixture was extracted with EtOAc (3×1 L). The combined organic layers were washed with brine (500 mL), dried over Na₂SO₄, filtered and concentrated in vacuum. The residue was purified by column chromatography eluting with EA/PE=5:1 to give methyl 2-amino-4-methoxybenzoate (45.6 g, 84% yield) as a yellow solid. ¹HNMR (400 MHz, CDCl₃) δ 7.78 (d, J=8.9 Hz, 1H), 6.23 (dd, J=2.4, 8.9 Hz, 1H), 6.10 (d, J=2.4 Hz, 1H), 5.62 (br. s., 2H), 3.83 (s, 3H), 3.78 (s, 3H); LC/MS (ESI, m/z): [M+H]⁺=182.0.

Step B: Methyl 2-amino-4-methoxybenzoate (45.6 g, 252 mmol) was dissolved in EtOH (350 mL), H₂O (450 mL) and concentrated HCl (22 mL). A solution of ICl (45 g, 277 mmol) in 40 mL of conc. HCl and 140 mL of H₂O was added dropwise at 5° C. with stirring. After addition, the mixture was stirred at room temperature overnight and a precipitate was filtered to give the crude compound. The crude compound was dissolved in EA (500 mL) and washed with saturated NaHCO₃ solution. The organic layer was concentrated in vacuum and the residue was purified by silica column chromatography eluting with PE/EA=4:1 to afford methyl 2-amino-5-iodo-4-methoxybenzoate (58.1 g, 75%) as a yellow solid. ¹H NMR (400 MHz, DMSO-d₆) δ 7.99 (s, 1H), 6.85 (br. s., 2H), 6.38 (s, 1H), 3.79 (s, 3H), 3.75 (s, 3H). LC/MS (ESI, m/z): [M+H]⁺=308.1.

Step C: A mixture of methyl 2-amino-5-iodo-4-methoxybenzoate (58.1 g, 189.3 mmol) and formamidine acetate (59 g, 567.7 mmol) in 2-methoxyethanol (200 mL) was warmed to 125° C. overnight. The mixture was cooled to room temperature and concentrated in vacuo. To the residue was added water (300 mL). A precipitate was filtered and washed with water. The solid was triturated with EtOAc (200 mL), filtered and dried to afford 6-iodo-7-methoxyquinazolin-4(3H)-one (54 g, 95% yield) as a white solid. LC/MS (ESI, m/z): [M+H]⁺=303.1.

Step D: To a solution of 6-iodo-7-methoxyquinazolin-4(3H)-one (54 g, 178.8 mmol) in SOCl₂ (500 mL) was added DMF (1 mL). The mixture was stirred at 80° C. for 1 h. The mixture was cooled to room temperature, concentrated in vacuum, and the residue was dried to give 4-chloro-6-iodo-7-methoxyquinazoline (57 g, 99% yield) as a yellow solid. LC/MS (ESI, m/z): [M+H]⁺=321.07.

Step E: A mixture of 4-chloro-6-iodo-7-methoxyquinazoline (57 g, 178 mmol) and 3,4-dichloro-2-fluoroaniline (32 g, 178 mmol) in iPrOH (500 mL) was stirred at 80° C. for 1 h. The reaction mixture was cooled to room temperature, then a precipitate was filtered. The filtrate cake was washed with iPrOH and dried to give the crude compound which was triturated with EtOAc to afford N-(3,4-dichloro-2-fluorophenyl)-6-iodo-7-methoxyquinazolin-4-amine (76.5 g, 92.8 yield) as a white solid. ¹H NMR (400 MHz, DMSO-d₆) δ 12.04 (br. s., 1H), 9.44 (s, 1H), 8.92 (s, 1H), 7.68 (dd, J=1.5, 8.9 Hz, 1H), 7.61 (dd, J=7.6, 8.7 Hz, 1H), 7.42 (s, 1H), 4.06 (s, 3H). LC/MS (ESI, m/z): [M+H]⁺=463.9.

Step F: A mixture of Zn (950 mg, 14.83 mmol) in dry DMF (20 mL) was degassed with nitrogen. 1,2-dibromoethane (183 mg, 0.98 mmol) in DMF (5 mL) was added to the mixture at RT. The mixture was stirred at 70° C. for 10 min and then cooled to RT. Trimethyl chlorosilane (106 mg, 0.98 mmol) was added and the mixture was stirred at RT for 1 hour. A solution of tert-butyl 3-iodoazetidine-1-carboxylate (3.50 g, 12.36 mmol) in DMF (10 mL) was added dropwise to the reaction mixture. The mixture was heated to 40° C. at stirred for 1 hour to give the (1-(tert-butoxycarbonyl)azetidin-3-yl)zinc(II) iodide which was used without any purification and work up.

A mixture of N-(3,4-dichloro-2-fluorophenyl)-6-iodo-7-methoxyquinazolin-4-amine (1.5 g, 4.12 mmol), Pd₂(dba)₃ (75 mg, 0.08 mmol), TFP (20 mg, 0.08 mmol) in DMF was degassed with nitrogen and stirred at RT. A solution of (1-(tert-butoxycarbonyl)azetidin-3-yl)zinc(II) iodide was added dropwise and then the reaction mixture was heated to 70° C. and stirred for 2 hours under nitrogen atmosphere. The reaction was cooled to r.t. and quenched with NH₄Cl (aq). H₂O (50 mL) was added and the mixture was extracted with EA. The combined organic layers were washed with brine, dried over anhydrous sodium sulfate, filtered and concentrated in vacuum. The residue was purified by column chromatography (PE/EA) to give the tert-butyl 3-(4-((3,4-dichloro-2-fluorophenyl)amino)-7-methoxyquinazolin-6-yl)azetidine-1-carboxylate (1.2 g, 75% yield) as a yellow solid. LC/MS (ESI, m/z): [M+H]⁺=493.2.

Step G: A solution of tert-butyl 3-(4-((3,4-dichloro-2-fluorophenyl)amino)-7-methoxyquinazolin-6-yl)azetidine-1-carboxylate (500 mg, 1.01 mmol) in DCM (10 mL) was slowly added trifluoroacetic acid and stirred at RT for 1 h. The reaction was then concentrated in vacuum to give the 6-(azetidin-3-yl)-N-(3,4-dichloro-2-fluorophenyl)-7-methoxyquinazolin-4-amine (398 mg, 99% yield) as a yellow oil. LC/MS (ESI, m/z): [M+H]⁺=393.2

Step H: To a solution of 6-(azetidin-3-yl)-N-(3,4-dichloro-2-fluorophenyl)-7-methoxyquinazolin-4-amine (398 mg, 1.01 mmol) in DCM (30 mL) was added DIPEA (261 mg, 2.02 mmol), acryloyl chloride (90 mg, 1.01 mmol). The mixture was stirred at RT for 1 hour. The reaction mixture was quenched by water and extracted with DCM. The organic layer was washed with brine, dried over anhydrous sodium sulfate, filtered and concentrated in vacuum. The residue was purified via reverse phase column chromatography (CH₃CN/H₂O) to give 1-(3-(4-((3,4-dichloro-2-fluorophenyl)amino)-7-methoxyquinazolin-6-yl)azetidin-1-yl)prop-2-en-1-one (Compound 1) as a white solid (162 mg, 37% yield). LC/MS (ESI, m/z): [M+H]⁺=447.0, 449.0. ¹H NMR (400 MHz, DMSO-d₆) δ 9.94 (s, 1H), 8.46 (s, 1H), 8.37 (s, 1H), 7.65-7.54 (m, 2H), 7.24 (s, 1H), 6.36 (dd, J=16.8, 10.4 Hz, 1H), 6.13 (dd, J=16.8, 2.4 Hz, 1H), 5.69 (dd, J=10.4, 2.4 Hz, 1H), 4.69 (t, J=8.8 Hz, 1H), 4.34 (d, J=8.0 Hz, 2H), 4.28 (dd, J=8.4, 6.6 Hz, 1H), 4.22-4.13 (m, 1H), 3.96 (s, 3H).

### Example 2: Preparation of 1-(3-(4-((4-(benzyloxy)-3-chloro-2-fluorophenyl)amino)-7-methoxyuinazolin-6-yl)azetidin-1-yl)prop-2-en-1-one (Compound 2)

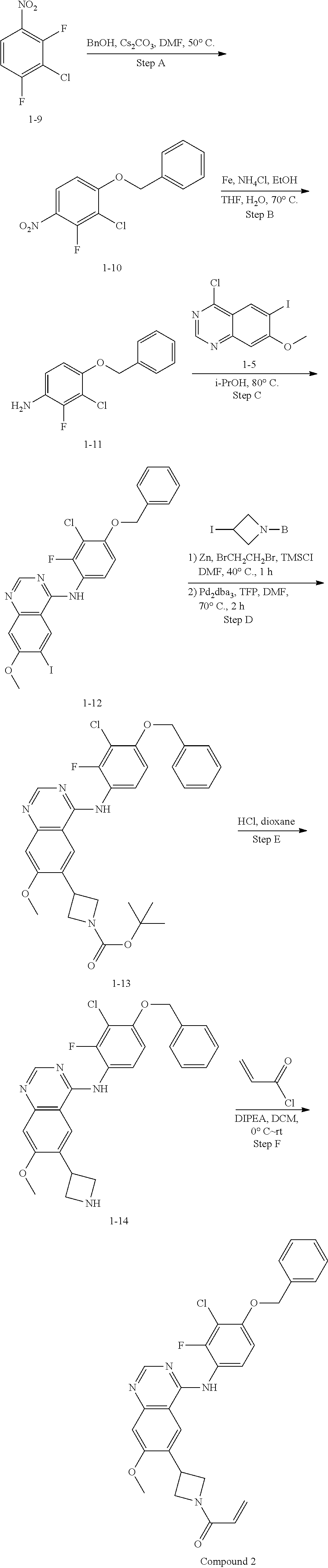

Step A: To a solution of 2-chloro-1,3-difluoro-4-nitrobenzene (970 mg, 4 mmol) and phenylmethanol (600 mg, 4.4 mmol) in DMF (15 mL) was added K₂CO₃ (1.33 g, 8 mmol). The mixture was stirred at room temperature for 16 hours. H₂O was added and the mixture extracted with EA. The organic layer was washed with brine, dried over Na₂SO₄, filtered, concentrated and purified by column (PE 100%) to give 1-(benzyloxy)-2-chloro-3-fluoro-4-nitrobenzene (660 mg, 47% yield) as a white solid.

Step B: To a suspension of 1-(benzyloxy)-2-chloro-3-fluoro-4-nitrobenzene (560 mg, 2 mmol), Fe (1.1 g, 20 mmol) in THF/EtOH/H₂O (2:2:1, 15 mL) was added NH₄Cl (1 g, 20 mmol). The mixture was heated to 70° C. for 2 hours. The mixture was cooled to RT, filtered and washed with MeOH. The organic solution was concentrated to give 4-(benzyloxy)-3-chloro-2-fluoroaniline (400 mg, 80% yield) as a yellow solid. LC/MS (ESI, m/z): [M+H]⁺=252.1.

Steps C, D, E, F were completed in a similar manner as described in Example 1 steps E, F, G, H to afford 1-(3-(4-((4-(benzyloxy)-3-chloro-2-fluorophenyl)amino)-7-methoxyquinazolin-6-yl)azetidin-1-yl)prop-2-en-1-one (Compound 2). LC/MS (ESI, m/z): [M+H]⁺=519.1. ¹H NMR (400 MHz, DMSO-d₆) δ 9.68 (s, 1H), 8.39 (s, 1H), 8.36 (s, 1H), 7.60 (dd, J=9.1, 6.0 Hz, 1H), 7.32 (t, J=8.9 Hz, 1H), 7.25-7.06 (m, 6H), 6.38 (dd, J=17.0, 10.3 Hz, 1H), 6.14 (dd, J=17.0, 2.2 Hz, 1H), 5.69 (dd, J=10.3, 2.2 Hz, 1H), 4.91 (s, 2H), 4.68 (t, J=8.6 Hz, 1H), 4.42-4.09 (m, 4H), 3.95 (s, 3H).

### Example 3: Preparation of 1-(3-(4-((3,4-dichloro-2-fluorophenyl)amino)-7-methoxyuinazolin-6-yl)pyrrolidin-1-yl)prop-2-en-1-one (Compound 3)

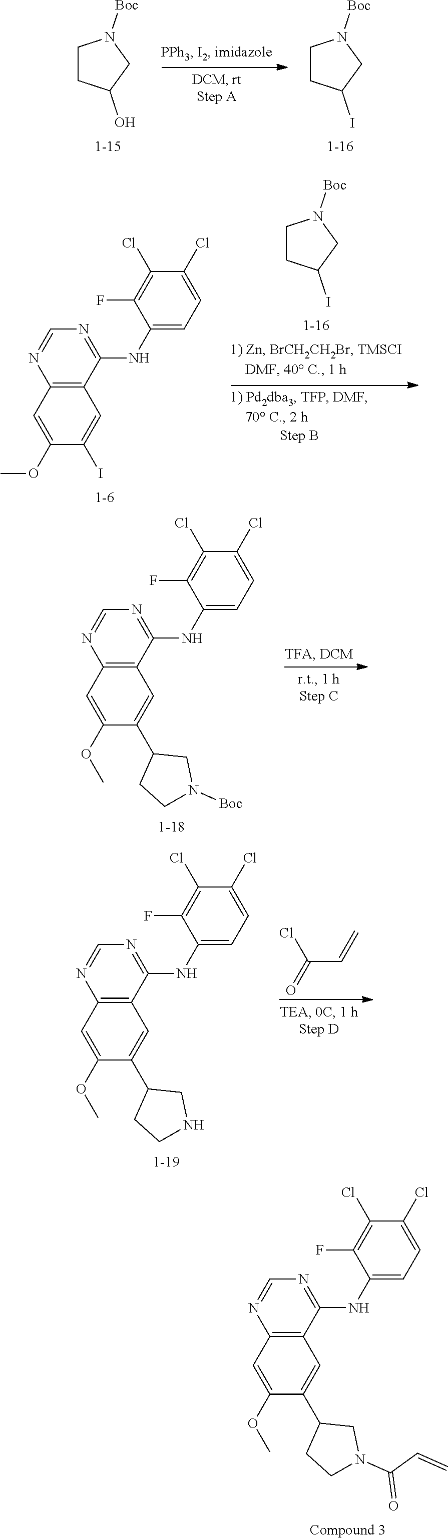

Step A: A mixture of tert-butyl 3-hydroxypyrrolidine-1-carboxylate (1.87 g, 10 mmol), imidazole (1.02 g, 15 mmol), PPh₃ (2.91 g, 11.1 mmol) in THF was added I₂ (2.82 g, 11.1 mmol) by portion at 0° C. After addition, the mixture was warmed to RT and stirred overnight. The mixture was quenched with 1% Na₂S₂O₃ (aq) and extracted with EA. The combined organic layer was concentrated in vacuo and the residue was purified by column chromatography to give tert-butyl 3-iodopyrrolidine-1-carboxylate (2.4 g, 99% yield) as a yellow oil. LC/MS (ESI, m/z): [M−55]⁺=241.8.

Steps B, C, D were completed in a similar manner as described in Example 1 steps F, G, H to afford 1-(3-(4-((3,4-dichloro-2-fluorophenyl)amino)-7-methoxyquinazolin-6-yl)pyrrolidin-1-yl)prop-2-en-1-one (Compound 3). LC/MS (ESI, m/z): [M+H]⁺=461.2, 463.2. ¹H NMR (400 MHz, DMSO-d₆) δ 9.92 (d, J=24.4 Hz, 1H), 8.45 (s, 1H), 8.32 (d, J=10.0 Hz, 1H), 7.59 (s, 2H), 7.24 (s, 1H), 6.70-6.56 (m, 1H), 6.23-6.10 (m, 1H), 5.74-5.62 (m, 1H), 4.20-4.07 (m, 1H), 3.99 (s, 3H), 3.85-3.75 (m, 1H), 3.73-3.61 (m, 1H), 3.57-3.38 (m, 2H), 2.36-2.24 (m, 1H), 2.23-2.08 (m, 1H).

Examples 4-10 were prepared using similar procedures as described in Examples 1-3.

| EX. | | Compound | | |
|---|---|---|---|---|
| NO. | IUPAC Name | structure | salt | Compound characterization |
| 4 | 1-(3-(7-methoxy- 4-(naphthalen-1- ylamino)quinazolin- 6-yl)azetidin-1- yl)prop-2-en-1- one | 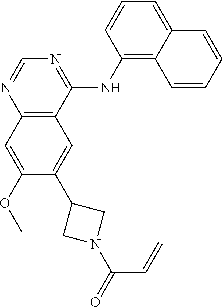 [SMILES: C=CC(=O)N1CC(c2cc3c(Nc4cccc5ccccc45)ncnc3cc2OC)C1] | None | LC/MS (ESI, m/z): [M + H]⁺ = 411.2. ¹H NMR (400 MHz, DMSO-d₆) δ 11.55 (s, 1 H), 8.75 (s, 1 H), 8.67 (s, 1 H), 8.11-8.01 (m, 2 H), 7.92 (d, J = 8.2 Hz, 1 H), 7.66- 7.57 (m, 4 H), 7.26 (s, 1 H), 6.37 (dd, J = 17.0, 10.3 Hz, 1 H), 6.14 (dd, J = 17.0, 2.2 Hz, 1 H), 5.70 (dd, J = 10.3, 2.2 Hz, 1 H), 4.73 (t, J = 8.7 Hz, 1 H), 4.41-4.31 (m, 3 H), 4.27-4.20 (m, 1 H), 4.04 (s, 3 H). |
| 5 | 1-(3-(7-methoxy- 4-(naphthalen-2- ylamino)quinazolin- 6-yl)azetidin-1- yl)prop-2-en-1- one | 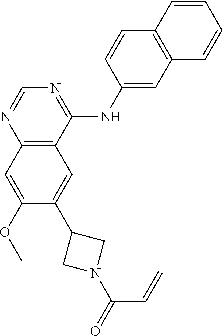 [SMILES: C=CC(=O)N1CC(c2cc3c(Nc4ccc5ccccc5c4)ncnc3cc2OC)C1] | None | LC/MS (ESI, m/z): [M + H]⁺ = 411.2. ¹H NMR (400 MHz, DMSO-d₆) δ 11.27 (s, 1 H), 8.87 (s, 1 H), 8.65 (s, 1 H), 8.21 (s, 1 H), 8.09-7.90 (m, 3 H), 7.83 (dd, J = 8.8, 2.0 Hz, 1 H), 7.64-7.50 (m, 2 H), 7.32 (s, 1 H), 6.38 (dd, J = 17.0, 10.3 Hz, 1 H), 6.15 (dd, J = 17.0, 2.2 Hz, 1 H), 5.71 (dd, J = 10.3, 2.2 Hz, 1 H), 4.72 (t, J = 8.7 Hz, 1 H), 4.46-4.29 (m, 3 H), 4.25-4.20 (m, 1 H), 4.02 (s, 3 H). |
| 6 | 1-(3-(4-((3-chloro- 4- fluorophenyl)amino- 7- methoxyquinazolin- 6-yl)azetidin-1- yl)prop-2-en-1- one | 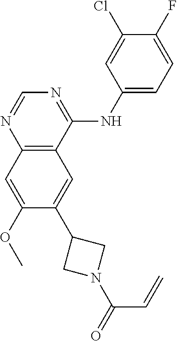 [SMILES: C=CC(=O)N1CC(c2cc3c(Nc4ccc(F)c(Cl)c4)ncnc3cc2OC)C1] | None | LC/MS (ESI, m/z): [M + H]⁺ = 413.1 ¹H NMR (400 MHz, DMSO-d₆ ) δ 10.82 (br s, 1 H), 8.83 (s, 1 H), 8.51 (s, 1 H), 8.02- 7.99 (m, 1 H), 7.69-7.53 (m, 1 H), 7.55 (t, J = 9.1 Hz, 1 H), 7.26 (s, 1 H), 6.37 (dd, J = 17.0, 10.3 Hz, 1 H), 6.14 (dd, J = 17.0, 2.2 Hz, 1 H), 5.70 (dd, J = 10.3, 2.2 Hz, 1 H), 4.71 (t, J = 8.7 Hz, 1 H), 4.38-4.32 (m, 2), 4.31-4.27 m, 1 H), 4.21-4.16 (m, 1 H), 4.01 (s, 3 H). |
| 7 | 1-(3-(4-((4- chloronaphthalen- 1-yl)amino)-7- methoxyquinazolin- 6-yl)azetidin-1- yl)prop-2-en-1- one | 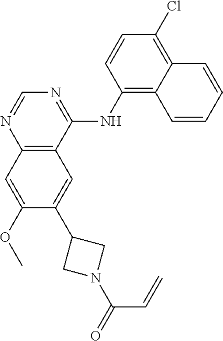 [SMILES: C=CC(=O)N1CC(c2cc3c(Nc4ccc(Cl)c5ccccc45)ncnc3cc2OC)C1] | None | LC/MS (ESI, m/z): [M +1]⁺ = 445.0.¹H NMR (400 MHz, DMSO-d₆) δ 11.53 (br s, 1 H), 8.74 (s, 1 H), 8.70 (s, 1 H), 8.30 (d, J = 8.4 Hz, 1 H), 8.03 (d, J = 8.4 Hz, 1 H), 7.87 (d, J = 7.9 Hz, 1 H), 7.79 (t, J = 7.2 Hz, 1 H), 7.70-7.65 (m, 2 H), 7.28 (s, 1 H), 6.38 (dd, J = 17.0, 10.3 Hz, 1 H), 6.14 (dd, J = 17.0, 2.2 Hz, 1 H), 5.70 (dd, J = 10.3, 2.2 Hz, 1 H), 4.74 (t, J = 8.6 Hz, 1 H), 4.42-4.27 (m, 3 H), 4.24 (dd, J = 14.1, 7.7 Hz, 1 H), 4.04 (s, 3 H). |
| 8 | 1-(3-(4-((3,4- dichloro-2- fluorophenyl)amino) quinazolin-6- yl)azetidin-1- yl)prop-2-en-1- one | 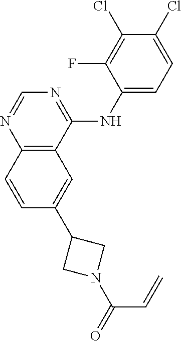 [SMILES: C=CC(=O)N1CC(c2ccc3ncnc(Nc4ccc(Cl)c(Cl)c4F)c3c2)C1] | None | LC/MS (ESI, m/z): [M + H]⁺ = 416.9, 418.9. ¹H NMR (400 MHz, DMSO-d₆) δ 10.09 (s, 1 H), 8.50 (d, J = 14.1 Hz, 2 H), 7.85 (dd, J = 23.2, 8.4 Hz, 2 H), 7.60 (s, 2 H), 6.39 (dd, J = 17.0, 10.3 Hz, 1 H), 6.16 (dd, J = 17.0, 2.2 Hz, 1 H), 5.71 (dd, J = 10.3, 2.2 Hz, 1 H), 4.73 (t, 1 H), 4.50- 4.27 (m, 2 H), 4.26-4.07 (m, 2 H). |
| 9 | 1-(4-(4-((3,4- dichloro-2- fluorophenyl)amino) quinazolin-6- yl)piperidin-1- yl)prop-2-en-1- one | 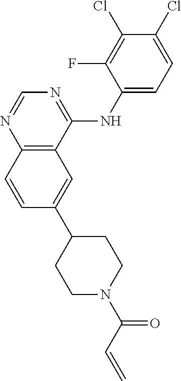 [SMILES: C=CC(=O)N1CCC(c2ccc3ncnc(Nc4ccc(Cl)c(Cl)c4F)c3c2)CC1] | None | LC/MS (ESI, m/z): [M + H]⁺ = 445.1, 447.1. ¹H NMR (400 MHz, DMSO-d₆) δ 10.02 (s, 1 H), 8.49 (s, 1 H), 8.31 (s, 1 H), 7.92-7.70 (m, 2 H), 7.59 (s, 2 H), 6.88 (dd, J = 16.7, 10.5 Hz, 1 H), 6.14 (dd, J = 16.7, 2.4 Hz, 1 H), 5.70 (dd, J = 10.4, 2.4 Hz, 1 H), 4.66 (d, J = 12.3 Hz, 1 H), 4.25 (d, J = 12.4 Hz, 1 H), 3.22 (t, J = 12.3 Hz, 1 H), 3.03 (t, J = 11.9 Hz, 1 H), 2.77 (t, J = 12.5 Hz, 1 H), 2.02-1.89 (m, 2 H), 1.74-1.55 (m, 2 H). |
| 10 | 1-(4-(4-((3,4- dichloro-2- fluorophenyl)amino)- 7- methoxyquinazolin- 6-yl)piperidin-1- yl)prop-2-en-1- one | 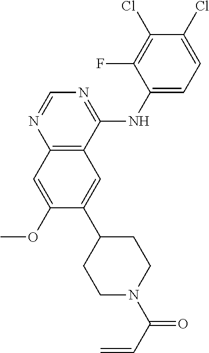 [SMILES: C=CC(=O)N1CCC(c2cc3c(Nc4ccc(Cl)c(Cl)c4F)ncnc3cc2OC)CC1] | None | LC/MS (ESI, m/z): [M +1]⁺ = 475.2, 477.2. ¹H NMR (400 MHz, DMSO-d₆) δ 9.86 (s, 1 H), 8.43 (s, 1 H), 8.22 (s, 1 H), 7.58 (s, 2 H), 7.22 (s, 1 H), 6.88 (dd, J = 16.6, 10.4 Hz, 1 H), 6.13 (dd, J = 16.7, 2.4 Hz, 1 H), 5.69 (dd, J = 10.4, 2.4 Hz, 1 H), 4.66 (d, J = 12.8 Hz, 1 H), 4.24 (d, J = 13.7 Hz, 1 H), 3.98 (s, 3 H), 3.28-3.19 (m, 2 H), 2.77 (t, J = 12.8 Hz, 1 H), 1.89 (t, J = 9.1 Hz, 2 H), 1.71-1.59 (m, 2 H). |

### Example 11: Preparation of 1-(3-(4-((1H-indol-5-yl)amino)-7-methoxygiuinazolin-6-yl)azetidin-1-yl)prop-2-en-1-one (Compound 11)

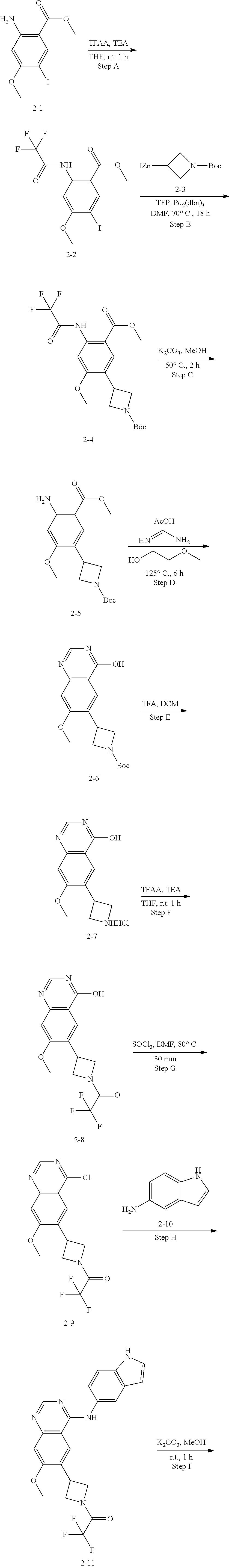

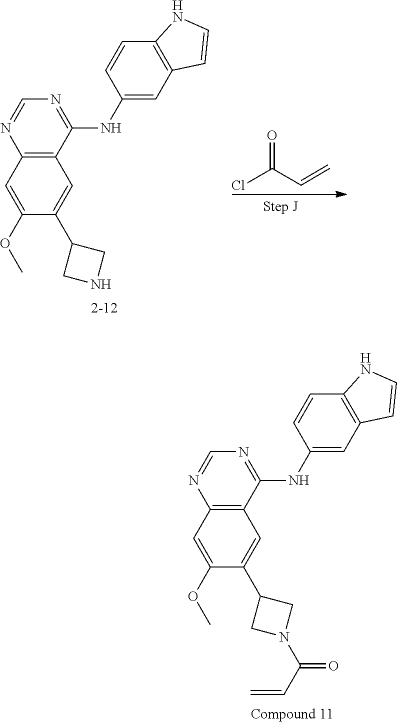

Step A: To a solution of methyl 2-amino-5-iodo-4-methoxybenzoate (5 g, 16.3 mmol), TEA (3.5 mL, 24 mmol) in THF (100 mL) was dropwise added a solution of TFAA (3.8 g, 17.9 mmol) in THF (10 mL) over 10 min. The mixture was stirred at RT for 1.5 h. Then the solvent was evaporated and purified by column chromatography (PE/EA=8:1) to give methyl 5-iodo-4-methoxy-2-(2,2,2-trifluoroacetamido)benzoate (5.4 g, 84% yield) as a white solid. LC/MS (ESI, m/z): [M+H]⁺=404.2.

Step B: A mixture of Zn (2.9 g, 44.64 mmol) in dry DMF (50 mL) was degassed with nitrogen, 1,2-Dibromoethane (668 mg, 3.57 mmol) in DMF (5 mL) was added in at RT, the mixture was stirred at 70° C. for 10 min and then cooled to RT. Then, trimethyl chlorosilane (386 mg, 3.57 mmol) was added and the mixture was stirred at RT for 1 hour. A solution of tert-butyl 3-iodoazetidine-1-carboxylate (10.53 g, 37.2 mmol) in DMF (20 mL) was added dropwise to the reaction mixture, the mixture was heated to 40° C. at stirred for 1 hour to give the (1-(tert-butoxycarbonyl)azetidin-3-yl)zinc(II) iodide, and use the result Zn reagent without any purification and work up.

To a solution of methyl 5-iodo-4-methoxy-2-(2,2,2-trifluoroacetamido)benzoate (5 g, 12.4 mmol), Pd₂(dba)₃ (227 mg, 12.4 mmol), and TFP (115 mg, 0.25 mmol) in DMF (60 mL) was added (1-(tert-butoxycarbonyl)azetidin-3-yl)zinc(II) iodide (74 mL, 37.2 mmol). The mixture was stirred at 70° C. for 2 h under N₂. After cooled to RT, the mixture was quenched by NH₄Cl, extracted with EtOAc (100 mL×3). The organic layers were washed with brine, dried over Na₂SO₄, filtered and concentrated to give tert-butyl3-(2-methoxy-5-(methoxycarbonyl)-4-(2,2,2-trifluoroacetamido)phenyl)azetidine-1-carboxylate (3.4 g, 64% yield) as a white solid. LC/MS (ESI, m/z): [M−55]⁺=377.1.

Step C: A mixture of tert-butyl 3-(2-methoxy-5-(methoxycarbonyl)-4-(2,2,2-trifluoroacetamido) phenyl)azetidine-1-carboxylate (1.7 g, 3.94 mmol) and K₂CO₃ (2.7 g, 19.68 mmol) in MeOH (70 mL) was stirred at 50° C. for 5 h. After being concentrated, the residue was dissolved in EtOAc and H₂O. The organic layer was washed with brine, dried over Na₂SO₄, filtered and concentrated to give tert-butyl 3-(4-amino-2-methoxy-5-(methoxycarbonyl)phenyl)azetidine-1-carboxylate (0.9 g, 69% yield) as white solid. LC/MS (ESI, m/z): [M+H]⁺=337.2.

Step D: A mixture of tert-butyl 3-(4-amino-2-methoxy-5-(methoxycarbonyl)phenyl)azetidine-1-carboxylate (1.8 g, 5.36 mmol), formamidine acetate (1.7 g, 16.34 mmol) in 2-metholyethenol (12 mL) was stirred at 125° C. for 6 h under N₂. After being cooled to RT, the mixture was evaporated, the residue was washed with water and the solid was filtered and dried to give tert-butyl 3-(4-hydroxy-7-methoxyquinazolin-6-yl)azetidine-1-carboxylate (1.7 g, 94% yield) as a brown solid. LC/MS (ESI, m/z): [M+H]⁺=332.2.

Step E: To a solution of tert-butyl 3-(4-hydroxy-7-methoxyquinazolin-6-yl)azetidine-1-carboxylate (1.7 g, 5.1 mmol) in DCM (20 mL) was added TFA (10 mL) at 0° C. The mixture was stirred at 0° C. for 1 h. After concentrated, the crude compound (1.2 g, 100% yield) was used in the next step without any purification. LC/MS (ESI, m/z): [M+H]⁺=232.2.

Step F: To a solution of 6-(azetidin-3-yl)-7-methoxyquinazolin-4-ol hydrochloride (1.2 g, 5.11 mmol) and TEA (4.8 g, 76.59 mmol) in DCM 50 mL) was added TFAA (7.7 g, 30.63 mmol) in DCM (5 mL) at 0° C. The mixture was stirred at RT for 3 h. After concentration, the residue was purified by column chromatography on silica gel (100% EA) to give 2,2,2-trifluoro-1-(3-(4-hydroxy-7-methoxyquinazolin-6-yl)azetidin-1-yl)ethan-1-one (1.6 g, 94 yield) as brown solid. LC/MS (ESI, m/z): [M+H]⁺=328.1.

Step G: A mixture of 2,2,2-trifluoro-1-(3-(4-hydroxy-7-methoxyquinazolin-6-yl)azetidin-1-yl)ethan-1-one (500 mg, 1.53 mmol) in SOCl₂ (12 mL) and DMF (2 drop) was stirred at 80° C. for 30 min. After concentration, the residue (529 mg, 100% yield) was used in the next step without any purification. LC/MS (ESI, m/z): [M+H]⁺=346.2.

Step H: A mixture of 1-(3-(4-chloro-7-methoxyquinazolin-6-yl)azetidin-1-yl)-2,2,2-trifluoroethan-1-one (150 mg, 0.43 mmol) and 1H-indol-5-amine (57 mg, 0.43 mmol) in i-PrOH (4 mL) was stirred at 80° C. for 2 h. After being cooled to RT, the 1-(3-(4-((1H-indol-5-yl)amino)-7-methoxyquinazolin-6-yl) azetidin-1-yl)-2,2,2-trifluoroethan-1-one was obtained by reduced pressure as yellow solid (195 mg, 75% yield). LC/MS (ESI, m/z): [M+H]⁺=442.2.

Step I: A mixture of 1-(3-(4-((1H-indol-5-yl)amino)-7-methoxyquinazolin-6-yl)azetidin-1-yl)-2,2,2-trifluoroethan-1-one (105 mg, 0.24 mmol) and K₂CO₃ (98 mg, 0.71 mmol) in MeOH (6 mL) and H₂O (0.6 mL) was stirred at RT for 1 h. After concentration, the crude compound was purified by Prep-TLC (PE:EA=10:1), then the desired product 6-(azetidin-3-yl)-N-(1H-indol-5-yl)-7-methoxyquinazolin-4-amine was obtained (45 mg, 55% yield) was obtained as a white solid. LC/MS (ESI, m/z): [M+H]⁺=346.2.

Step J: To a solution of 6-(azetidin-3-yl)-N-(1H-indol-5-yl)-7-methoxyquinazolin-4-amine (35 mg, 0.10 mmol) and TEA (9.1 mg, 0.30 mmol) in DCM (1.5 mL) was added acryloyl chloride (3.9 mg, 0.10 mmol) at 0° C. The reaction was stirred at 0° C. for 30 min. After concentration, the mixture was purified by Prep-TLC (DCM:MeOH=10:1) to give the 1-(3-(4-((1H-indol-5-yl)amino)-7-methoxyquinazolin-6-yl) azetidin-1-yl)prop-2-en-1-one (Compound 11) as a brown solid (5 mg, 13% yield). LC/MS (ESI, m/z): [M+H]⁺=400.1. ¹H NMR (400 MHz, DMSO-d₆) δ 11.31 (d, J=11.9 Hz, 2H), 8.78 (s, 1H), 8.64 (s, 1H), 7.76 (d, J=1.8 Hz, 1H), 7.50 (d, J=8.6 Hz, 1H), 7.45-7.40 (m, 1H), 7.29-7.21 (m, 2H), 6.55-6.48 (m, 1H), 6.37 (dd, J=17.0, 10.3 Hz, 1H), 6.14 (dd, J=17.0, 2.2 Hz, 1H), 5.70 (dd, J=10.3, 2.2 Hz, 1H), 4.71 (t, J=8.7 Hz, 1H), 4.40-4.28 (m, 3H), 4.25-4.11 (m, 1H), 4.01 (s, 3H).

### Example 12: Preparation of 1-(3-(4-(indolin-5-ylamino)-7-methoxyquinazolin-6-yl)azetidin-1-yl)prop-2-en-1-one (Compound 12)

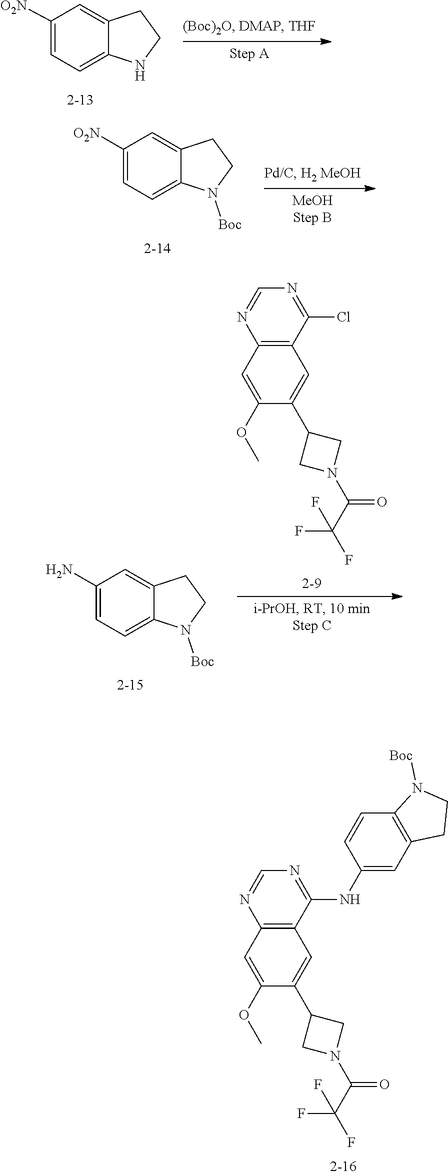 [SMILES: O=[N+]([O-])c1ccc2c(c1)CCN2 ; CC(C)(C)OC(=O)N1CCc2cc([N+](=O)[O-])ccc21 ; COc1cc2ncnc(Cl)c2cc1C1CN(C(=O)C(F)(F)F)C1 ; CC(C)(C)OC(=O)N1CCc2cc(N)ccc21 ; COc1cc2ncnc(Nc3ccc4c(c3)CCN4C(=O)OC(C)(C)C)c2cc1C1CN(C(=O)C(F)(F)F)C1]

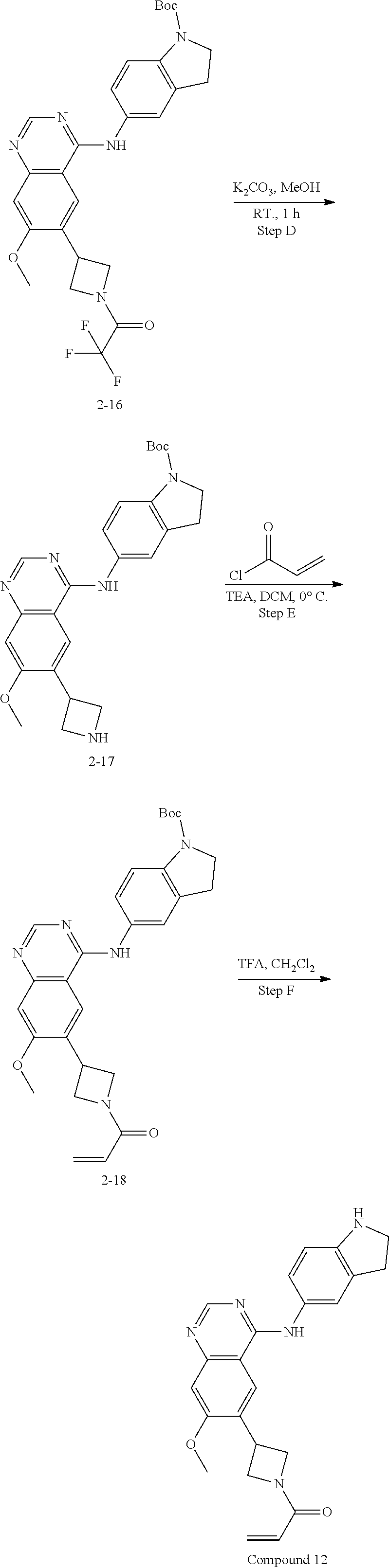

Step A: To a solution of 5-nitroindoline (200 mg, 1.22 mmol) in THE (5 mL) was added (Boc)₂C (531 mg, 2.44 mmol) and DMAP (15 mg, 0.122 mmol). The reaction was stirred at RT for 1 h. After concentrated, the residue was purified by silica gel chromatography to give tert-butyl 5-nitroindoline-1-carboxy late (250 mg, 71% yield) as a yellow solid. LC/MS (ESI, m/z): [M+H]⁺=265.2.

Step B: A mixture of tert-butyl 5-nitroindoline-1-carboxylate (188 mg, 0.68 mmol) and Pd/C (20 mg) in MeOH (5 mL) was stirred at RT under H₂ for 2 h. The Pd/C was removed by reduced pressure and then concentrated to give tert-butyl 5-aminoindoline-1-carboxylate (166 mg, 100% yield) as a white solid. LC/MS (ESI, m/z): [M+H]⁺=235.2.

Steps C, D, E were completed in a similar manner as described in Example 11 steps H, I, J to afford tert-butyl 5-((6-(1-acryloylazetidin-3-yl)-7-methoxyquinazolin-4-yl)amino)indoline-1-carboxylate (Compound 2-18).

Step F: To a solution of tert-butyl 5-((6-(1-acryloylazetidin-3-yl)-7-methoxyquinazolin-4-yl)amino)indoline-1-carboxylate (48 mg, 0.096 mmol) in DCM (1 mL) was added TFA (1 mL) at 0° C. The mixture was stirred at 0° C. for 30 mi. After concentration, the residue was purified by prep-TIPLC to give 1-(3-(4-(indolin-5-ylamino)-7-methoxyquinazolin-6-yl)azetidin-1-yl)prop-2-en-(-one (Compound 12) (4 mg, 10% yield). LC/MS (ESI, m/z): [M+H]⁺=402.2. ¹H NMR (400 z, DMO-d₆) δ 11.15 (s, 1H), 8.80 (s, 1H), 8.57 (s, 1H), 7.42 (s, 1H), 7.30 (s, 1H), 7.25 (d, J=8.0 Hz, 1H), 6.86 (d, J=7.9 Hz, 1H), 6.36 (dd, J=17.0, 10.3 Hz, 1H), 6.14 (dd, J 17.0, 2.1 Hz, 1H), 5.70 (dd, J 10.3, 2.1 Hz, 1H), 4.70 (t, J 8.6 Hz, 1H), 4.40-4.23 (m, 3H), 4.19 (m, 1H), 4.00 (s, 3H), 3.59 (t, J=8.2 Hz, 2H), 3.06 (t, J=8.2 Hz, 2H).

Examples 13-16 were prepared using similar procedures as described in Examples 11 and 12.

| EX. | | Compound | | |
|---|---|---|---|---|
| NO. | IUPAC Name | structure | salt | Compound characterization |
| 13 | 1-(3-(4-((4- fluorobenzyl)ami- no)-7- methoxyquinazo- lin-6- yl)azetidin-1- yl)prop-2-en-1- one | 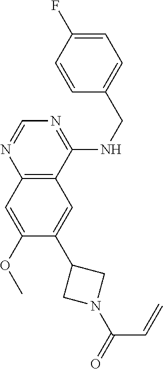 [SMILES: C=CC(=O)N1CC(c2cc3c(NCc4ccc(F)cc4)ncnc3cc2OC)C1] | None | LC/MS (ESI, m/z): [M + H]⁺ = 393.2. ¹H NMR (400 MHz, DMSO-d₆) δ 8.72 (t, J = 6.0 Hz, 1 H), 8.39 (s, 1 H), 8.20 (s, 1 H), 7.40 (dd, J = 8.6, 5.6 Hz, 2 H), 7.19- 7.05 (m, 3 H), 6.35 (dd, J = 17.0, 10.3 Hz, 1 H), 6.12 (dd, J = 17.0, 2.2 Hz, 1 H), 5.67 (dd, J = 10.3, 2.2 Hz, 1 H), 4.77 (d, J = 5.7 Hz, 2 H), 4.64 (t, J = 8.7 Hz, 1 H), 4.34-4.19 (m, 3 H), 4.14-4.10 (m, 1 H), 3.92 (s, 3 H). |
| 14 | 1-(3-(4-((4- fluoronaphthalen- 1-yl)amino)-7- methoxyquinazo- lin-6- yl)azetidin-1- yl)prop-2-en-1- one | 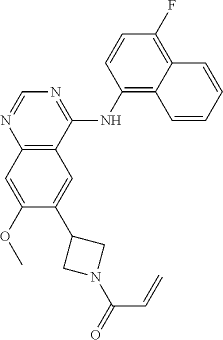 [SMILES: C=CC(=O)N1CC(c2cc3c(Nc4ccc(F)c5ccccc45)ncnc3cc2OC)C1] | None | LC/MS (ESI, m/z): [M + H]⁺ = 429.1 1H NMR (400 MHz, DMSO-d₆) δ 10.04 (s, 1 H), 8.54 (s, 1 H), 8.25 (s, 1 H), 8.12 (d, J = 8.3 Hz, 1 H), 7.90 (d, J = 8.3 Hz, 1 H), 7.62-7.54 (m, 3 H), 7.43 (dd, J = 10.5, 8.2 Hz, 1 H), 7.21 (s, 1 H), 6.37 (dd, J = 17.0, 10.3 Hz, 1 H), 6.13 (dd, J = 17.0, 2.2 Hz, 1H), 5.68 (dd, J = 10.3, 2.2 Hz, 1 H), 4.69 (t, J = 8.8 Hz, 1 H), 4.37-4.32 (m, 3 H), 4.22-4.18 (m, 1 H), 3.97 (s, 3 H). |
| 15 | 1-(3-(4-((3- chlorobenzyl)ami- no)-7- methoxyquinazo- lin-6- yl)azetidin-1- yl)prop-2-en-1- one | 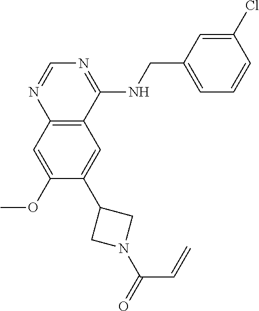 [SMILES: C=CC(=O)N1CC(c2cc3c(NCc4cccc(Cl)c4)ncnc3cc2OC)C1] | None | LC/MS (ESI, m/z): [M + H]⁺ = 409.4. ¹H NMR (400 MHz, DMSO-d₆) δ 8.74 (t, J = 6.0 Hz, 1 H), 8.38 (s, 1 H), 8.21 (s, 1 H), 7.40-7.29 (m, 4 H), 7.13 (s, 1 H), 6.35 (dd, J = 17.2, 10.3 Hz, 1 H), 6.12 (dd, J = 17.2, 2.2 Hz, 1 H), 5.67 (dd, J = 10.4, 2.4 Hz, 1 H), 4.79 (d, J = 5.9 Hz, 2 H), 4.65 (t, J = 8.7 Hz, 1 H), 4.32-4.23 (m, 3 H), 4.15-4.10 (m, 1 H), 3.92 (s, 3 H). |
| 16 | 1-(3-(4-((3- chloro-2,4- difluorophenyl)a- mino)-7- methoxyquinazo- lin-6- yl)azetidin-1- yl)prop-2-en-1- one | 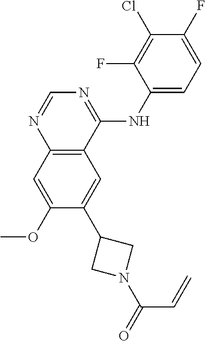 [SMILES: C=CC(=O)N1CC(c2cc3c(Nc4ccc(F)c(Cl)c4F)ncnc3cc2OC)C1] | TFA | LC/MS (ESI, m/z): [M + H]⁺ = 431.1 ¹H NMR (400 MHz, DMSO-d₆) δ: 11.04 (br s, 1 H), 8.78 (s, 1 H), 8.55 (s, 1 H), 7.58- 7.69 (m, 1 H), 7.49 (td, J = 8.8, 2.0 Hz, 1 H), 7.29 (s, 1 H), 6.36 (dd, J = 17.2, 10.4 Hz, 1 H), 6.14 (dd, J = 17.2, 2.4 Hz, 1 H), 5.70 (dd, J = 10.4, 2.4 Hz, 1 H), 4.71 (t, J = 8.6 Hz, 1 H), 4.40-4.17 (m, 4 H), 4.01 (s, 3 H). |

### Example 17: Preparation of 1-(3-(4-((3,4-dichloro-2-fluorophenyl)amino)-7-(2-methoxyethoxy)ciuinazolin-6-yl)azetidin-1-yl)prop-2-en-1-one (Compound 17)

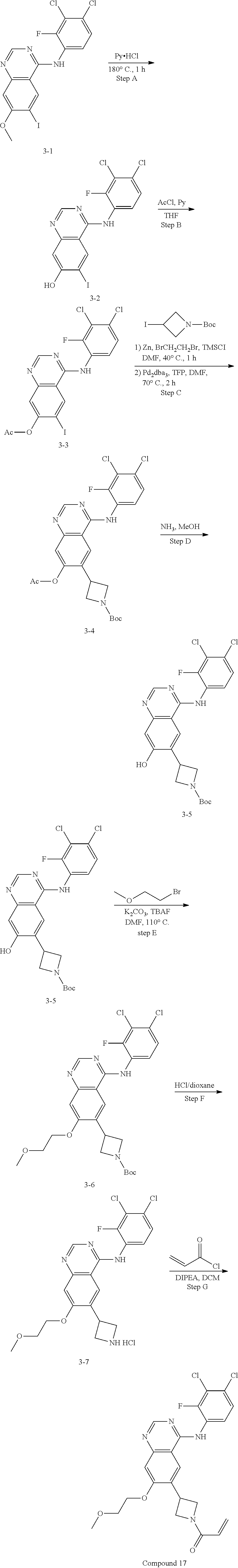

Step A: A mixture of N-(3,4-dichloro-2-fluorophenyl)-6-iodo-7-methoxyquinazolin-4-amine (3.3 g, 7.11 mmol) and pyridine hydrochloride (38 g, 33.04 mmol) was stirred at 180° C.˜185° C. for 1 hour. The mixture was cooled to room temperature. H₂O (50 mL) was added, followed by extraction with EtOAc (60 mL×4). The organic layer was washed with brine and concentrated under vacuum. The residue was purified by silica gel chromatography (eluted with DCM\MeOH=100/4) to give 4-((3,4-dichloro-2-fluorophenyl)amino)-6-iodoquinazolin-7-ol as a yellow solid (2.3 g, 72% yield). LC/MS (ESI, m/z): [M+H]⁺=449.9.

Step B: The solution of 4-((3,4-dichloro-2-fluorophenyl)amino)-6-iodoquinazolin-7-ol (2.3 g, 5.10 mmol) in DCM (40 mL) was stirred at 0° C., then triethylamine (1.15 mL, 7.69 mmol) was added dropwise. After the mixture stirred at 0° C. for 5 min, acetyl chloride (486 mg, 6.19 mmol) was added dropwise. The resulting mixture was stirred at 0° C. for 30 min. The mixture was quenched with water (80 mL) and extracted with DCM (50 mL×3). The combined organic layers were dried over anhydrous Na₂SO₄ and concentrated under vacuum. The residue was purified by chromatography on silica gel (eluted with DCM/MeOH=10/1) to give 4-((3,4-dichloro-2-fluorophenyl)amino)-6-iodoquinazolin-7-yl acetate as a yellow solid (1.5 g, 60%).

LC/MS (ESI, m/z): [M+H]⁺=491.9.

Step C: To a solution of activated Zinc powder (1.14 g, 17.50 mmol) in DMF (25 mL) was added 1,2-Dibromoethane (220 mg, 1.18 mmol). The mixture was stirred at 0° C. for 10 min under N₂, and then warmed to room temperature. After trimethyl chlorosilane (127 mg, 1.18 mmol) was added to the mixture, the resulting mixture was stirred at room temperature for 1 hour. Then the solution of tert-butyl 3-iodoazetidine-1-carboxylate (4.18 g, 14.77 mmol) in DMF (11 mL) was added dropwise over 20 min. The mixture was stirred at 40° C. for 1 hour, and then cooled to room temperature. The suspension was used in the next step directly. To the mixture of 4-((3,4-dichloro-2-fluorophenyl)amino)-6-iodoquinazolin-7-yl acetate (1.7 g, 3.45 mmol), Pd₂(dba)₃ (101 mg, 0.11 mmol) and Tri(2-furyl)phosphine (26 mg, 0.11 mmol) in DMF (10 mL), the (1-(tert-butoxycarbonyl)azetidin-3-yl)zinc iodide solution was added, and the mixture was stirred at 70° C. for 1.5 hours under N₂. After reaction was completion, NH₄Cl (2 mol/L, 8 mL) was added to quench, then extracted with EtOAc (20 mL*3), the combined organic layers were dried with anhydrous Na₂SO₄ and purified by silica gel chromatography (eluted with DCM/MeOH=10/1) to provide a mixture of tert-butyl 3-(4-((3,4-dichloro-2-fluorophenyl)amino)-7-hydroxyquinazolin-6-yl) azetidine-1-carboxylate and tert-butyl 3-(7-acetoxy-4-((3,4-dichloro-2-fluorophenyl)amino)quinazolin-6-yl)azetidine-1-carboxylate as a yellow solid (1 g mixture). LC/MS (ESI, m/z): [M+H]⁺=521.1.

Step D: The mixture of tert-butyl 3-(4-((3,4-dichloro-2-fluorophenyl)amino)-7-hydroxyquinazolin-6-yl) azetidine-1-carboxylate and tert-butyl 3-(7-acetoxy-4-((3,4-dichloro-2-fluorophenyl)amino)quinazolin-6-yl)azetidine-1-carboxylate (1 g) and NH₃ (7 mol/L in MeOH, 10 mL, 70 mmol) was stirred at room temperature for 20 min. Then the mixture was concentrated under vacuum and the residue was purified by silica gel chromatography (eluted with DCM/MeOH=10/1) to provide tert-butyl 3-(4-((3,4-dichloro-2-fluorophenyl)amino)-7-hydroxyquinazolin-6-yl) azetidine-1-carboxylate as a yellow solid (670 mg, 41.1%). LC/MS (ESI, m/z): [M+H]⁺=479.1.

Step E: A mixture of tert-butyl 3-(4-((3,4-dichloro-2-fluorophenyl)amino)-7-hydroxyquinazolin-6-yl)azetidine-1-carboxylate (200 mg, 0.42 mmol, 1.0 equiv), 1-bromo-2-methoxyethane (64 mg, 0.46 mmol, 1.1 equiv), and K₂CO₃ (290 mg, 2.1 mmol, 5.0 equiv) in DMF (10 mL) was stirred at 80° C. for 2 hours. The mixture was diluted with water (20 mL) and extracted with DCM (30 mL×3). The combined organic phases were concentrated in vacuo. The residue was purified by reverse phase prep-HPLC (eluted with CH₃CN/H₂O=5%˜90% with 0.1% TFA) to give tert-butyl 3-(4-((3,4-dichloro-2-fluorophenyl)amino)-7-(2-methoxyethoxy)quinazolin-6-yl)azetidine-1-carboxylate (105 mg, 47% yield) as a yellow solid.

LC/MS (ESI, m/z): [M+H]⁺=537.1.

Step F: The mixture of tert-butyl 3-(4-((3,4-dichloro-2-fluorophenyl)amino)-7-(2-methoxyethoxy)quinazolin-6-yl)azetidine-1-carboxylate (100 mg, 0.186 mmol) and HCl (4 mol/L in 1,4-dioxane, 2 mL) was stirred at room temperature for 30 min. Then the mixture was concentrated in vacuo to give 6-(azetidin-3-yl)-N-(3,4-dichloro-2-fluorophenyl)-7-(2-methoxyethoxy)quinazolin-4-amine hydrochloride as a white solid (86 mg crude). LC/MS (ESI, m/z): [M+H]⁺=437.1.

Step G: To a solution of 6-(azetidin-3-yl)-N-(3,4-dichloro-2-fluorophenyl)-7-(2-methoxyethoxy)quinazolin-4-amine hydrochloride (86 mg, 0.182 mmol) in DCM (2 mL) was added DIPEA (1 mL), followed by dropwise addition of acryloyl chloride (17 mg, 0.18 mmol) in DCM (1 mL). The resulting mixture was stirred at 0° C. for 20 min. Then the reaction mixture was diluted with water (10 mL) and extracted with DCM (10 mL×3). The combined organic phases were washed with brine (30 mL), dried over anhydrous Na₂SO₄ and concentrated in vacuo. The residue was purified by reverse phase prep-HPLC (eluted with CH₃CN/H₂O=5% 90% with 0.1% TFA) to give 1-(3-(4-((3,4-dichloro-2-fluorophenyl)amino)-7-(2-methoxyethoxy)quinazolin-6-yl)azetidin-1-yl)prop-2-en-1-one as a white solid (15 mg, 17% yield). LC/MS (ESI, m/z): [M+H]⁺=491.0.

¹H NMR (400 MHz, DMSO-d₆) δ: 9.94 (s, 1H), 8.45 (s, 1H), 8.34 (s, 1H), 7.59 (s, 2 H), 7.25 (s, 1H), 6.36 (dd, J=17.0, 10.3 Hz, 1H), 6.13 (dd, J=16.9, 2.3 Hz, 1H), 5.69 (dd, J=10.3, 2.3 Hz, 1H), 4.67 (t, J=8.8 Hz, 1H), 4.26-4.38 (m, 5H), 4.22-4.17 (m, 1H), 3.74 (t, J=4.4 Hz, 2H), 3.35 (s, 3H).

### Example 18: Preparation of 1-(3-(4-((3,4-dichloro-2-fluorophenyl)amino)-7-ethoxyquinazolin-6-yl)azetidin-1-yl)prop-2-en-1-one (Compound 18)

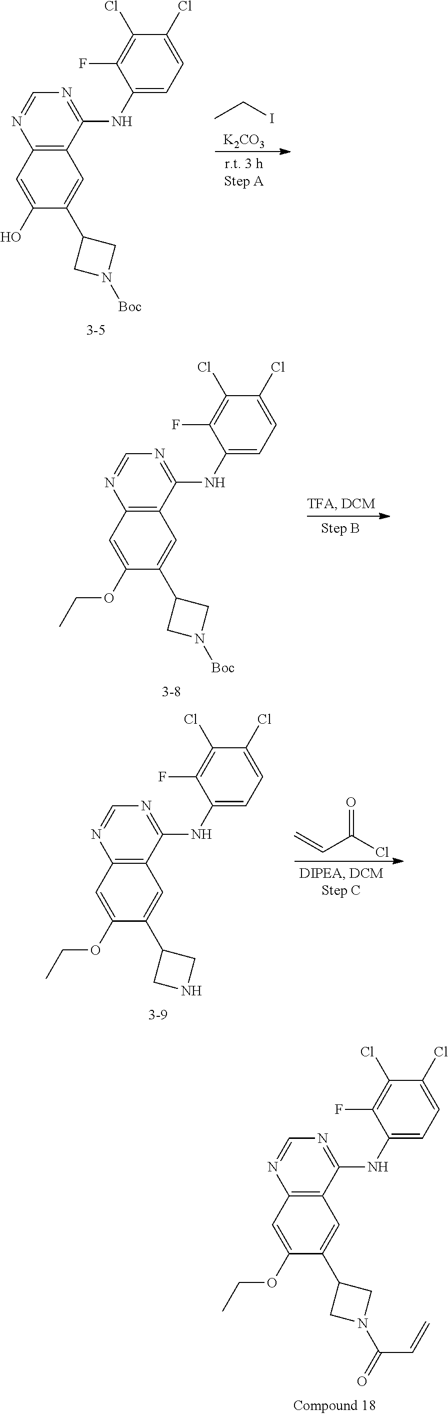

Step A: A solution of tert-butyl 3-(4-((3,4-dichloro-2-fluorophenyl)amino)-7-hydroxyquinazolin-6-yl)azetidine-1-carboxylate (150 mg, 0.31 mmol) in DMF(5 mL) was added K₂CO₃ (87 mg, 0.63 mmol) and iodoethane (49 mg, 0.31 mmol). The mixture was stirred at RT for 3 h. The mixture was quenched by water and extracted with EA. The organic layer was washed with brine, dried by Na₂SO₄, filtered, and concentrated in vacuum. The residue was purified by column chromatography to give tert-butyl 3-(4-((3,4-dichloro-2-fluorophenyl)amino)-7-ethoxyquinazolin-6-yl)azetidine-1-carboxylate (70 mg, 44% yield) as a yellow solid. LC/MS (ESI, m/z): [M+H]⁺=507.2, 509.1.

Steps B and C were completed in a similar manner as described in Example 17 steps F and G to afford 1-(3-(4-((3,4-dichloro-2-fluorophenyl)amino)-7-ethoxyquinazolin-6-yl)azetidin-1-yl)prop-2-en-1-one (Compound 18). LC/MS (ESI, m/z): [M+H]⁺=461.2, 463.2. ¹H NMR (400 MHz, DMSO-d₆) δ 11.00 (br s, 1H), 8.76 (s, 1H), 8.51 (s, 1H), 7.73-7.51 (m, 2H), 7.30-7.21 (m, 1H), 6.37 (dd, J=16.8, 10.0 Hz, 1H), 6.13 (dd, J=17.2, 2.4 Hz, 1H), 5.70 (dd, J=10.4, 2.4 Hz, 1H), 4.70 (t, J=8.8 Hz, 1H), 4.40-4.11 (m, 6H), 1.44 (t, J=7.2 Hz, 3H).

### Example 19: Preparation of 1-(3-(4-((3,4-dichloro-2-fluorophenyl)amino)-7-(2-hydroxyethoxy)quinazolin-6-yl)azetidin-1-yl)prop-2-en-1-one (Compound 19)

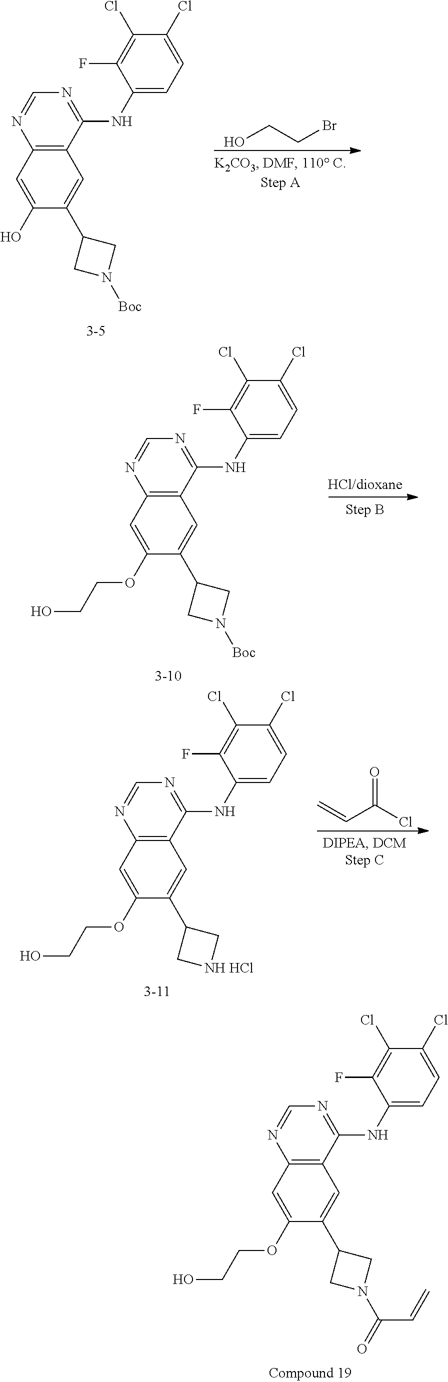

Step A: A mixture of 3-(4-((3,4-dichloro-2-fluorophenyl)amino)-7-hydroxyquinazolin-6-yl) azetidine-1-carboxylate (200 mg, 0.42 mmol), 2-bromoethan-1-ol (58 mg, 0.46 mmol) and K₂CO₃ (173 mg, 1.26 mmol) in DMF (2 mL) was stirred at 110° C. for 3 hours. The reaction mixture was diluted with water (50 mL) and extracted with EtOAc (20 mL×3). The combined organic phases were washed with brine (30 mL), dried over anhydrous Na₂SO₄ and concentrated in vacuo. The residue was purified by silica gel chromatography (eluted with DCM/MeOH=20/1) to give tert-butyl 3-(4-((3,4-dichloro-2-fluorophenyl)amino)-7-(2-hydroxyethoxy)quinazolin-6-yl)azetidine-1-carboxylate as yellow solid (58 mg, 26%). LC/MS (ESI, m/z): [M+H]⁺=523.2.

Steps B and C were completed in a similar manner as described in Example 17 steps F and G to afford 1-(3-(4-((3,4-dichloro-2-fluorophenyl)amino)-7-(2-hydroxyethoxy)quinazolin-6-yl)azetidin-1-yl)prop-2-en-1-one (Compound 19). LC/MS (ESI, m/z): [M+H]⁺=477.1. ¹H NMR (400 MHz, DMSO-d₆) δ: 10.98 (br s, 1H), 8.76 (s, 1H), 8.51 (s, 1H), 7.56-7.72 (m, 2H), 7.27 (s, 1H), 6.36 (dd, J=17.2, 10.4 Hz, 1H), 6.14 (dd, J=17.2, 2.0 Hz, 1H), 5.70 (dd, J=10.4, 2.0 Hz, 1H), 4.72 (t, J=8.8 Hz, 1H), 4.36-4.42 (m, 1H), 4.25-4.35 (m, 3H), 4.21-4.25 (t, 2H), 3.81-3.84 (t, 2H)

### Example 20: Preparation of 1-(3-(4-((3,4-dichloro-2-fluorophenyl)amino)-7-(2,2,2-trifluoroethoxy)quinazolin-6-yl)azetidin-1-yl)prop-2-en-1-one (Compound 20)

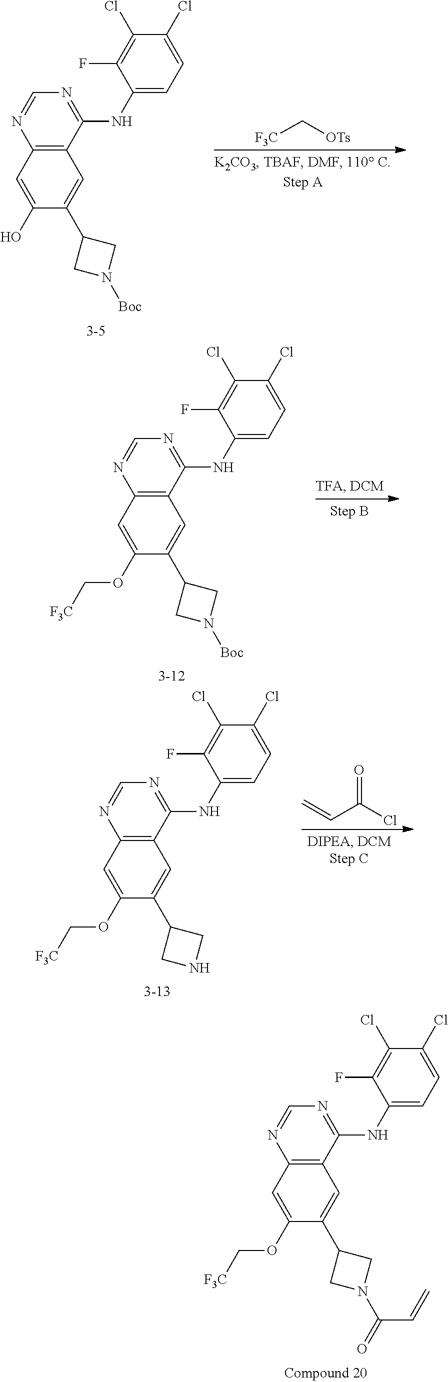

Step A: To a solution of tert-butyl 3-(4-((3,4-dichloro-2-fluorophenyl)amino)-7-hydroxyquinazolin-6-yl)azetidine-1-carboxylate (150 mg, 0.31 mmol, 1 equiv), 2,2,2-trifluoroethyl 4-methylbenzenesulfonate (94 mg, 0.37 mmol, 1.2 equiv) and potassium carbonate (86 mg, 0.62 mmol, 2 equiv) in DMF (5 mL) was added TBAF (0.31 mL, 0.31 mmol, 1.0 equiv) (1M in THF). The reaction mixture was stirred at 110° C. for 12 hours. The mixture was diluted with EtOAc (30 mL) and washed by water (30 mL×2). The organic layer was dried over Na₂SO₄ and concentrated and purified by chromatography (eluted with DCM/MeOH=10/1) to afford the title compound tert-butyl 3-(4-((3,4-dichloro-2-fluorophenyl)amino)-7-(2,2,2-trifluoroethoxy)quinazolin-6-yl)azetidine-1-carboxylate (45 mg, 36% yield). LC/MS (ESI, m/z): [M+H]⁺=561.2.

Steps B and C were completed in a similar manner as described in Example 17 steps F and G to afford 1-(3-(4-((3,4-dichloro-2-fluorophenyl)amino)-7-(2,2,2-trifluoroethoxy)quinazolin-6-yl)azetidin-1-yl)prop-2-en-1-one (Compound 20). LC/MS (ESI, m/z): [M+H]⁺=515.0. ¹H NMR (400 MHz, DMSO-d₆) δ: 10.03 (s, 1H), 8.50 (s, 1H), 8.41 (s, 1H), 7.60 (s, 2H), 7.42 (s, 1H), 6.34 (dd, J=17.0, 10.3 Hz, 1H), 6.12 (dd, J=17.0, 2.2 Hz, 1 H), 5.69 (dd, J=10.3, 2.2 Hz, 1H), 5.02 (q, J=8.6 Hz, 2H), 4.66 (t, J=8.7 Hz, 1H), 4.40-4.28 (m, 3H), 4.24-4.13 (m, 1H).

### Example 21: Preparation of 1-(3-(4-((3,4-dichloro-2-fluorophenyl)amino)-7-(2-(4-methylpiperazin-1-yl)ethoxy)quinazolin-6-yl)azetidin-1-yl)prop-2-en-1-one (Compound 21)

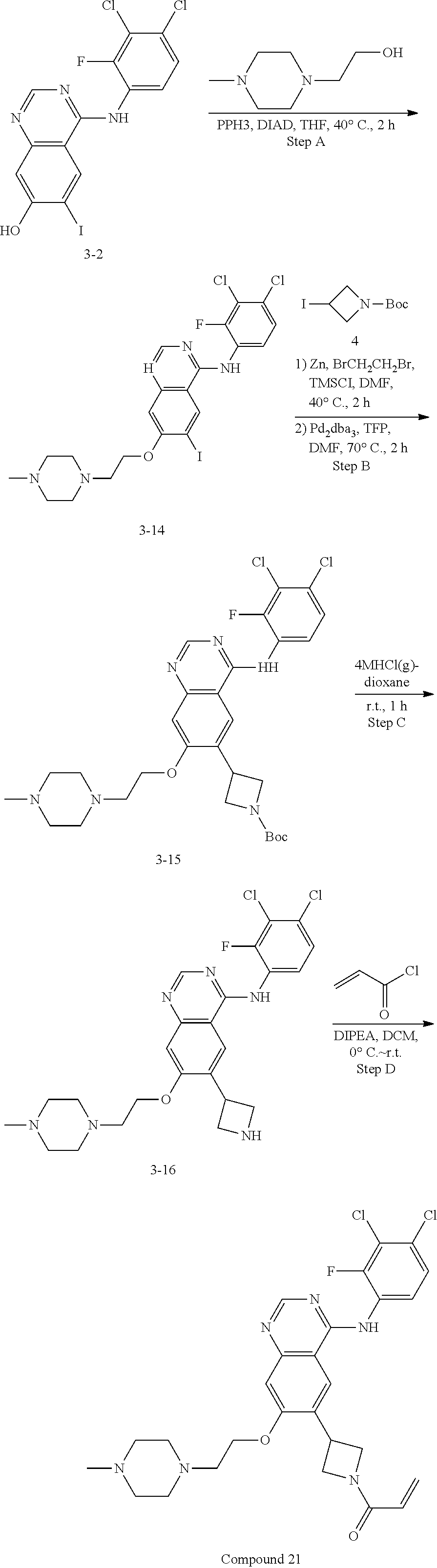

Step A: To a solution of 4-((3,4-dichloro-2-fluorophenyl)amino)-6-iodoquinazolin-7-ol (200 mg, 0.446 mmol, 1 equiv), 2-(4-methylpiperazin-1-yl)ethan-1-ol (84 mg, 0.58 mmol, 1.3 equiv), PPh₃ (175 mg, 0.669 mmol) in dry THE (30 mL) was added DIAD (135 mg, 0.669 mmol) at room temperature under N₂. The reaction mixture was stirred at 40° C. for 2 h. H₂O (30 mL) was added. The mixture was extracted with EtOAc (30 mL×3). The organic layers were washed with brine and dried over Na₂SO₄ and concentrated and purified by chromatography to afford the title compound N-(3,4-dichloro-2-fluorophenyl)-6-iodo-7-(2-(4-methylpiperazin-1-yl)ethoxy)quinazolin-4-amine (134 mg, 52% yield). LC/MS (ESI, m/z): [M+H]⁺=576.2.

Steps B, C, and D were completed in a similar manner as described in Example 17 steps E, F, and G to afford 1-(3-(4-((3,4-dichloro-2-fluorophenyl)amino)-7-(2-(4-methylpiperazin-1-yl)ethoxy)quinazolin-6-yl)azetidin-1-yl)prop-2-en-1-one (Compound 21). LC/MS (ESI, m/z): [M+H]⁺=559.4, 561.4. ¹H NMR (400 MHz, DMSO-d₆) δ 9.92 (s, 1H), 8.45 (s, 1H), 8.33 (s, 1H), 7.59 (dd, J=6.0, 4.4 Hz, 2H), 7.25 (s, 1H), 6.36 (dd, J=17.0, 10.3 Hz, 1H), 6.13 (dd, J=17.0, 2.2 Hz, 1H), 5.69 (dd, J=10.3, 2.2 Hz, 1H), 4.78-4.58 (m, 1H), 4.35-4.32 (m, 3H), 4.27 (t, J=5.4 Hz, 2H), 4.19-4.12 (m, 1H), 2.80-2.75 (m, 2H), 2.49-2.33 (m, 8H), 2.21 (s, 3 H).

### Example 22: Preparation of (S)-1-(3-(4-((3,4-dichloro-2-fluorophenyl)amino)-7-((tetrahydrofuran-3-yl)oxy)quinazolin-6-yl)azetidin-1-yl)prop-2-en-1-one (Compound 22)

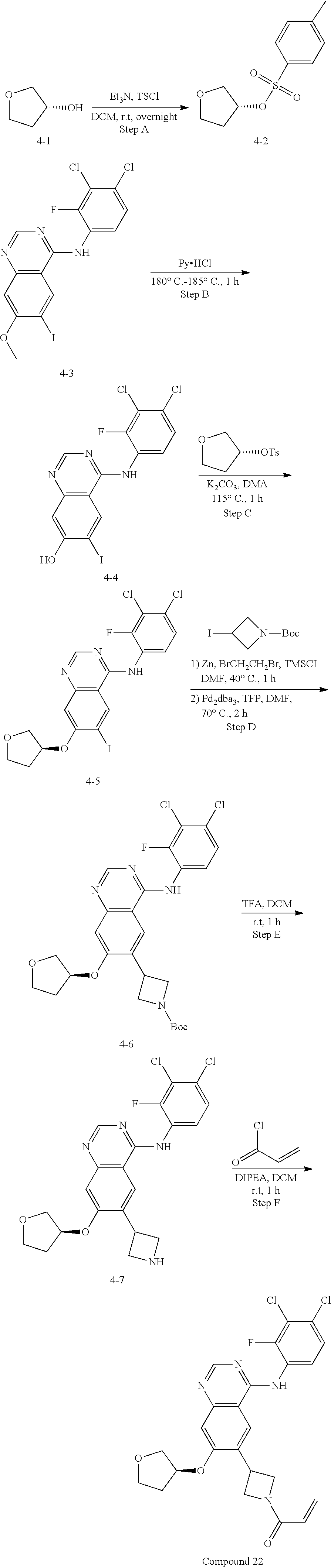

Step A: To a mixture of (R)-tetrahydrofuran-3-ol (10 g, 113.6 mmol), tosyl chloride (26 g, 136.4 mmol) in DCM (200 mL) was added Et₃N (23 g, 227 mmol) dropwise. After addition, the mixture was stirred at room temperature under N₂ overnight. The reaction mixture was washed with water (200 mL), brine (200 mL), dried over Na₂SO₄, filtered and concentrated in vacuo. The residue was purified by flash chromatography eluting with EA/PE=20% to afford (R)-tetrahydrofuran-3-yl 4-methylbenzenesulfonate (19.1 g, 70% yield) as a colorless oil. ¹H NMR (400 MHz, DMSO-d₆) δ 7.85-7.77 (m, 2H), 7.56-7.45 (m, J=8.0 Hz, 2H), 5.17-5.06 (m, 1H), 3.80-3.62 (m, 4H), 2.43 (s, 3H), 2.13-2.03 (m, 1H), 1.94-1.85 (m, 1H). LC/MS (ESI, m/z): [M+H]⁺=243.2.

Step B: A mixture of N-(3,4-dichloro-2-fluorophenyl)-6-iodo-7-methoxyquinazolin-4-amine (26 g, 56.15 mmol) and pyridine hydrochloride (129.8 g, 1120 mmol) was warmed to 185° C. The mixture was heated gradually under magnetic stirring. Then the reaction mixture was maintained at 185° C. for 1 h. The reaction mixture was cooled to room temperature. Water (1 L) was added to quench the reaction and the precipitate was filtered. The filtrate cake was washed with water and dried to give the crude compound. The crude compound was triturated with EA to afford 4-((3,4-dichloro-2-fluorophenyl)amino)-6-iodoquinazolin-7-ol (21.4 g, 84.7 yield) as a white solid. LC/MS (ESI, m/z): [M+H]⁺=450.1. ¹H NMR (400 MHz, DMSO-d₆) δ 11.46 (br. s., 1H), 9.92 (s, 1H), 8.92 (s, 1H), 8.41 (s, 1H), 7.57 (br. s., 2H), 7.14 (s, 1H).

Step C: A mixture of 4-((3,4-dichloro-2-fluorophenyl)amino)-6-iodoquinazolin-7-ol (21.4 g, 47.66 mmol), (R)-tetrahydrofuran-3-yl 4-methylbenzenesulfonate (11.53 g, 47.66 mmol) and K₂CO₃ (13 g, 95.32 mmol) in DMA (200 mL) was warmed to 115° C. and stirred for 1 h. The reaction mixture was cooled to room temperature, then poured into water, extracted with EA (3×200 mL). The combined organic layers were washed with water (2×300 mL), brine (300 mL), dried over Na₂SO₄, filtered and concentrated in vacuum. The residue was purified by silica gel column chromatography eluting with MeOH/DCM=1/50 to afford (S)—N-(3,4-dichloro-2-fluorophenyl)-6-iodo-7-((tetrahydrofuran-3-yl)oxy)quinazolin-4-amine (16 g, 64.7 yield) as a yellow solid. LC/MS (ESI, m/z): [M+H]⁺=520.1. ¹H NMR (400 MHz, DMSO-d₆) δ 10.01 (s, 1H), 9.00 (s, 1H), 8.49 (s, 1H), 7.64-7.49 (m, 2H), 7.21 (s, 1H), 5.35 (t, J=4.9 Hz, 1H), 4.07-3.97 (m, 1H), 3.97-3.87 (m, 2H), 3.83 (dt, J=4.4, 8.2 Hz, 1H), 2.40-2.27 (m, 1H), 2.13-2.02 (m, 1H).

Step D: A mixture of Zn (7.1 g, 110.98 mmol) in dry DMF (160 mL) was degassed with nitrogen, 1,2-Dibromoethane (1.65 g, 8.88 mmol) was added in at RT, the mixture was stirred at 70° C. for 10 min and then cooled to RT. Then, trimethyl chlorosilane (959 mg, 8.88 mmol) was added and the mixture was stirred at RT for 1 hour. A solution of tert-butyl 3-iodoazetidine-1-carboxylate (26.1 g, 92.5 mmol) in DMF (63 mL) was added dropwise to the reaction mixture, the mixture was heated to 40° C. at stirred for 2 hour to give the (1-(tert-butoxycarbonyl)azetidin-3-yl)zinc(II) iodide, which was used without further purification.

A mixture of N-(3,4-dichloro-2-fluorophenyl)-6-iodo-7-methoxyquinazolin-4-amine (16 g, 30.83 mmol), Pd₂(dba)₃ (564 mg, 0.617 mmol), TFP (143 mg, 0.617 mmol) in DMF (200 mL) was degassed with nitrogen and stirred at RT, A solution of (1-(tert-butoxycarbonyl)azetidin-3-yl)zinc(II) iodide was added dropwise and then the reaction mixture was heated to 70° C. and stirred for 2 hour under nitrogen atmosphere. The reaction was cooled to r.t, quenched with NH₄Cl (aq) solution, the mixture was extracted with EA (3×500 mL). The combined organic layers were washed with brine, dried over anhydrous sodium sulfate, filtered and concentrated in vacuum. The residue was purified by column chromatography (MeOH/DCM=3%-5%) to give the (S)-tert-butyl 3-(4-((3,4-dichloro-2-fluorophenyl)amino)-7-((tetrahydrofuran-3-yl)oxy)quinazolin-6-yl)azetidine-1-carboxylate (15.4 g, 91.2% yield) as a yellow solid. LC/MS (ESI, m/z): [M+H]⁺=549.1.

Step E: A solution of (S)-tert-butyl 3-(4-((3,4-dichloro-2-fluorophenyl)amino)-7-((tetrahydrofuran-3-yl)oxy)quinazolin-6-yl)azetidine-1-carboxylate (15.4 g, 28.1 mmol) in DCM (30 mL) was slowly added trifluoroacetic acid (10 mL) and stirred at RT for 1 h. The reaction mixture was then concentrated in vacuum. The residue was dissolved in 100 mL of 1M HCl solution, and then washed with EA (2×100 mL). To the aqueous solution was added saturated NaHCO₃ solution until pH>8. The solution was extracted with DCM/iPrOH=3:1 (3×200 mL), and the combined organic layers were washed with brine, dried over Na₂SO₄, filtered and concentrated in vacuum. The residue was dried to afford (S)-6-(azetidin-3-yl)-N-(3,4-dichloro-2-fluorophenyl)-7-((tetrahydrofuran-3-yl)oxy)quinazolin-4-amine (10.8 g, 84%), which was used directly in the next step without further purification. LC/MS (ESI, m/z): [M+H]⁺=449.2.

Step F: To a solution of (S)-6-(azetidin-3-yl)-N-(3,4-dichloro-2-fluorophenyl)-7-((tetrahydrofuran-3-yl)oxy)quinazolin-4-amine (10.8 g, 24.1 mmol) in DCM (200 mL) was added DIPEA (6.21 g, 48.2 mmol), acryloyl chloride (2.18 g, 24.1 mmol), The mixture was stirred at RT for 1 hour. Then, the reaction mixture was quenched by water, extracted with DCM (3×200 mL). The combined organic layers were washed with brine, dried over anhydrous sodium sulfate, filtered and concentrated in vacuum. The residue was purified by silica gel column chromatography eluting with MeOH/DCM=5% to give the crude compound. The crude compound was dissolved in EA (75 mL). After standing for 5 min, a precipitate was collected by filtration and dried to give (S)-1-(3-(4-((3,4-dichloro-2-fluorophenyl)amino)-7-((tetrahydrofuran-3-yl)oxy)quinazolin-6-yl)azetidin-1-yl)prop-2-en-1-one (5.5 g, 45.4% yield) as a white solid. LC/MS (ESI, m/z): [M+H]⁺=503.2. ¹H NMR (400 MHz, DMSO-d₆) δ 9.92 (s, 1H), 8.46 (s, 1H), 8.34 (s, 1H), 7.66-7.51 (m, 2H), 7.21 (s, 1H), 6.37 (dd, J=10.3, 17.0 Hz, 1H), 6.13 (dd, J=2.2, 16.9 Hz, 1H), 5.69 (dd, J=1.7, 10.2 Hz, 1H), 5.31 (br. s., 1H), 4.67 (t, J=8.8 Hz, 1H), 4.41-4.25 (m, 3H), 4.17-4.10 (m, 1H), 4.02-3.96 (m, 1H), 3.91-3.78 (m, 3H), 2.37-2.24 (m, 1H), 2.11-2.01 (m, 1H).

### Example 23: Preparation of 1-(3-(4-((3,4-dichloro-2-fluorophenyl)amino)-7-(difluoromethoxy)quinazolin-6-yl)azetidin-1-yl)prop-2-en-1-one (Compound 23)

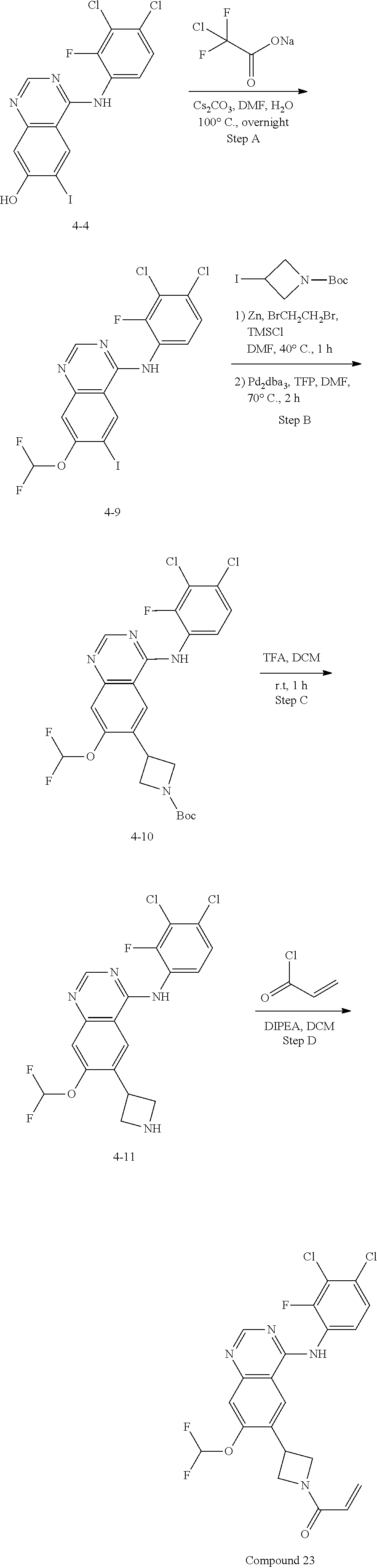

Step A: To a solution of 4-((3,4-dichloro-2-fluorophenyl)amino)-6-iodoquinazolin-7-ol (500 mg, 1.11 mmol) in DMF (10 mL) and H₂O (1 mL) was added Sodium 2-chloro-2,2-difluoroacetate (508 mg, 3.34 mmol) and Cs₂CO₃ (726 mg, 2.22 mmol). The mixture was warmed to 100° C. and stirred under N₂ for 8 h. The reaction mixture was cooled to room temperature, poured into water, extracted with EA (3×20 mL). The combined organic layers were washed with water, brine dried over anhydrous sodium sulfate, filtered and concentrated in vacuum. The residue was purified by silica gel column chromatography eluting with PE/EA=2:1 to give N-(3,4-dichloro-2-fluorophenyl)-7-(difluoromethoxy)-6-iodoquinazolin-4-amine (210 mg, 38% yield). LC/MS (ESI, m/z): [M+H]⁺=500.1.

Steps B, C, and D were completed in a similar manner as described in Example 22 steps D, E, and F to afford 1-(3-(4-((3,4-dichloro-2-fluorophenyl)amino)-7-(difluoromethoxy)quinazolin-6-yl)azetidin-1-yl)prop-2-en-1-one (Compound 23). LC/MS (ESI, m/z): [M+H]⁺=483.2, 485.2. ¹H NMR (400 MHz, DMSO-d₆) δ 10.16 (s, 1H), 8.54 (d, J=3.8 Hz, 2H), 7.75-7.39 (m, 4H), 6.37 (dd, J=10.3, 17.0 Hz, 1H), 6.14 (dd, J=2.3, 17.0 Hz, 1 H), 5.70 (dd, J=2.2, 10.3 Hz, 1H), 4.71 (t, J=8.6 Hz, 1H), 4.42-4.30 (m, 3H), 4.30-4.17 (m, 1H).

Examples 24-33 were prepared using similar procedures as described in Examples 22 and 23.

| EX. | | | | |
|---|---|---|---|---|
| NO. | IUPAC Name | Compound structure | salt | Compound characterization |
| 24 | (S)-1-(3-(4- ((3,4-dichloro- 2- fluorophenyl)a- mino)-7- ((tetrahydrofuran- 3- yl)oxy)quinazo- lin-6- yl)azetidin-1- yl)prop-2-en-1- one | 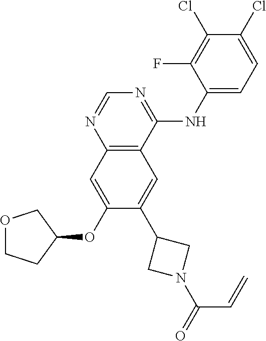 [SMILES: C=CC(=O)N1CC(c2cc3c(Nc4ccc(Cl)c(Cl)c4F)ncnc3cc2O[C@H]2CCOC2)C1] | None | LC/MS (ESI, m/z): [M + H]⁺ = 503.2. ¹H NMR (400 MHz, DMSO-d₆) δ 9.92 (s, 1 H), 8.46 (s, 1 H), 8.34 (s, 1 H), 7.66-7.51 (m, 2 H), 7.21 (s, 1 H), 6.37 (dd, J = 10.3, 17.0 Hz, 1 H), 6.13 (dd, J = 2.2, 16.9 Hz, 1 H), 5.69 (dd, J = 1.7, 10.2 Hz, 1 H), 5.31 (br. s., 1 H), 4.67 (t, J = 8.8 Hz, 1 H), 4.41-4.25 (m, 3 H), 4.17-4.10 (m, 1 H), 4.02- 3.96 (m, 1H), 3.91-3.78 (m, 3 H), 2.37-2.24 (m, 1 H), 2.11-2.01 (m, 1 H). |
| 25 | 1-(3-(4-((3,4- dichloro-2- fluorophenyl)a- mino)-7- (oxetan-3- yloxy)quinazolin- 6- yl)azetidin-1- yl)prop-2-en-1- one | 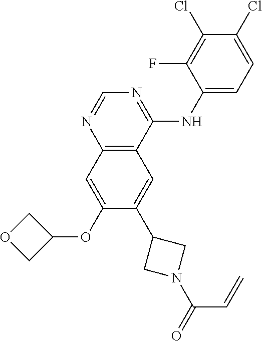 [SMILES: C=CC(=O)N1CC(c2cc3c(Nc4ccc(Cl)c(Cl)c4F)ncnc3cc2OC2COC2)C1] | None | LC/MS (ESI, m/z): [M + H]⁺ = 489.0, 491.2. ¹H NMR (400 MHz, DMSO-d₆) δ 9.96 (s, 1 H), 8.45 (s, 1 H), 8.39 (s, 1 H), 7.63-7.53 (m, 2 H), 6.86 (s, 1 H), 6.39 (dd, J = 16.8, 10.4 Hz, 1 H), 6.14 (dd, J = 17.2, 2.4 Hz, 1 H), 5.70 (dd, J = 10.0, 2.0 Hz, 1 H), 5.58-5.46 (m, 1 H), 5.02 (t, J = 6.8 Hz, 2 H), 4.76 (t, J = 9.2 Hz, 1 H), 4.66-4.54 (m, 2 H), 4.45-4.33 (m, 3H), 4.28-4.16 (m, 1 H). |
| 26 | 1-(3-(4-((3,4- dichloro-2- fluorophenyl)a- mino)-7-(2- (dimethylamino) ethoxy)quina- zolin-6- yl)azetidin-1- yl)prop-2-en-1- one | 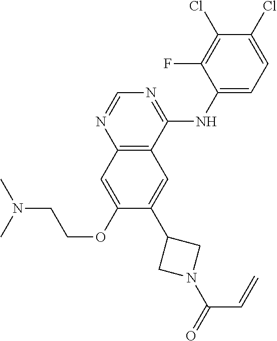 [SMILES: C=CC(=O)N1CC(c2cc3c(Nc4ccc(Cl)c(Cl)c4F)ncnc3cc2OCCN(C)C)C1] | None | LC/MS (ESI, m/z): [M + H]⁺ = 504.0, 506.0. ¹H NMR (400 MHz, DMSO-d₆) δ 8.43 (s, 1 H), 8.34 (s, 1 H), 7.58 (d, J = 1.6 Hz, 2 H), 7.24 (s, 1 H), 6.36 (dd, J = 17.0, 10.3 Hz, 1 H), 6.13 (dd, J = 17.0, 2.2 Hz, 1 H), 5.69 (dd, J = 10.3, 2.2 Hz, 1 H), 4.66 (t, J = 8.7 Hz, 1 H), 4.41- 4.29 (m, 3 H), 4.24 (t, J = 5.3 Hz, 2 H), 4.19-4.10 (m, 1 H), 2.76-2.66 (t, 2 H), 2.24 (s, 6 H). |
| 27 | 1-(3-(4-((3,4- dichloro-2- fluorophenyl)a- mino)-7- ((tetrahydro- 2H-pyran-4- yl)oxy)quinazo- lin-6- yl)azetidin-1- yl)prop-2-en-1- one | 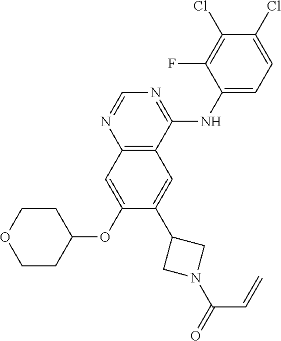 [SMILES: C=CC(=O)N1CC(c2cc3c(Nc4ccc(Cl)c(Cl)c4F)ncnc3cc2OC2CCOCC2)C1] | TFA | LC/MS (ESI, m/z): [M + H]⁺ = 517.1 ¹H NMR (400 MHz, DMSO-d₆) δ: 11.04 (br s, 1 H), 8.78 (s, 1 H), 8.52 (s, 1 H), 7.58-7.69 (m, 2 H), 7.39 (d, J = 2.9 Hz, 1 H), 6.39 (dd, J = 17.0, 10.3 Hz, 1 H), 6.14 (dd, J = 17.0, 2.2 Hz, 1 H), 5.70 (dd, J = 10.3, 2.2 Hz, 1 H), 4.94-4.89 (m, 1H), 4.72 (t, J = 8.7 Hz, 1H), 4.29-4.41 (m, 3 H), 4.16- 4.26 (m, 1 H), 3.88-3.83 (m, 2 H), 3.63-3.57 (m, 2 H), 2.10-2.04 (m, 2 H), 1.78-1.69 (m, 2 H). |
| 28 | 1-(3-(4-((3,4- dichloro-2- fluorophenyl)a- mino)-7-(2- morpholinoeth- oxy)quinazolin- 6-yl)azetidin- 1-yl)prop-2-en- 1-one | 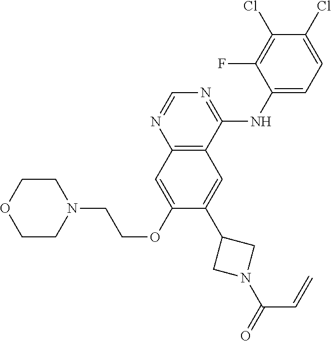 [SMILES: C=CC(=O)N1CC(c2cc3c(Nc4ccc(Cl)c(Cl)c4F)ncnc3cc2OCCN2CCOCC2)C1] | | LC/MS (ESI, m/z): [M + H]⁺ = 546.2. ¹H NMR (400 MHz, DMSO-d₆) δ 9.92 (s, 1 H), 8.45 (s, 1 H), 8.33 (s, 1 H), 7.62-7.55 (m, 2 H), 7.26 (s, 1 H), 6.36 (dd, J = 17.2, 10.4 Hz, 1 H), 6.13 (dd, J = 16.8, 2.0 Hz, 1 H), 5.70 (dd, J = 10.4, 2.4 Hz, 1 H), 4.69 (t, J = 9.2 Hz, 1 H), 4.35-4.33 (m, 3 H), 4.28 (t, J = 5.2 Hz, 2 H), 4.20-4.09 (m, 1 H), 3.60 (t, J = 4.8 Hz, 4 H), 2.77 (t, J = 5.6 Hz, 2 H), 2.60- 2.51 (m, 4 H) |
| 29 | (R)-1-(3-(4- ((3,4-dichloro- 2- fluorophenyl)a- mino)-7- ((tetrahydrofuran- 3- yl)oxy)quinazo- lin-6- yl)azetidin-1- yl)prop-2-en-1- one | 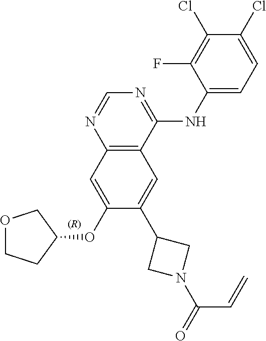 | TFA | LC/MS (ESI, m/z): [M + H]⁺ = 503.2, 505.2. ¹H NMR (400 MHz, DMSO-d₆) δ 10.87 (br, 1 H), 8.74 (s, 1 H), 8.49 (s, 1 H), 7.72-7.53 (m, 2 H), 7.27 (s, 1 H), 6.37 (dd, J = 17.0, 10.3 Hz, 1 H), 6.13 (dd, J = 17.0, 2.1 Hz, 1 H), 5.70 (dd, J = 10.2, 1.9 Hz, 1 H), 5.40-5.20 (m, 1 H), 4.70 (t, J = 8.5 Hz, 1 H), 4.39- 4.28 (m, 3 H), 4.21-4.11 (m, 1 H), 4.04-3.96 (m, 1 H), 3.93-3.80 (m, 3 H), 2.40-2.19 (m, 1 H), 2.12 - 2.01 (m, 1 H). |
| 30 | 1-(3-(4-((3,4- dichloro-2- fluorophenyl)a- mino)-7-(3- morpholinopro- poxy)quinazolin- 6- yl)azetidin-1- yl)prop-2-en-1- one | 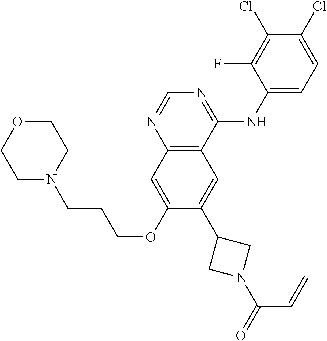 [SMILES: C=CC(=O)N1CC(c2cc3c(Nc4ccc(Cl)c(Cl)c4F)ncnc3cc2OCCCN2CCOCC2)C1] | | LC/MS (ESI, m/z): [M + 1]⁺ = 560.2, 563.2. ¹H NMR (400 MHz, DMSO- d₆) δ 9.93 (s, 1 H), 8.46 (s, 1 H), 8.35 (s, 1 H), 7.66-7.52 (m, 2 H), 7.23 (s, 1H), 6.37 (dd, J = 17.0, 10.3 Hz, 1H), 6.14 (dd, J = 17.0, 2.2 Hz, 1 H), 5.70 (dd, J = 10.3, 2.2 Hz, 1 H), 4.69 (t, J = 8.7 Hz, 1 H), 4.43-4.07 (m, 6 H), 3.83-3.42 (m, 4 H), 2.50-2.38 (m, 6 H), 2.21-1.86 (m, 2 H). |
| 31 | 1-(3-(4-((3,4- dichloro-2- fluorophenyl)a- mino)-7-(3-(4- methylpiperazin- 1- yl)propoxy)quin- azolin-6- yl)azetidin-1- yl)prop-2-en-1- one | 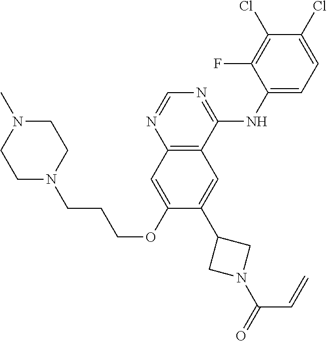 [SMILES: C=CC(=O)N1CC(c2cc3c(Nc4ccc(Cl)c(Cl)c4F)ncnc3cc2OCCCN2CCN(C)CC2)C1] | None | LC/MS (ESI, m/z): [M + H]⁺ = 574.2 ¹H NMR (400 MHz, CDCl₃) δ 8.63 (s, 1 H), 8.21-8.11 (m, 1 H), 7.98 (s, 1 H), 7.84 (s, 1 H), 7.26 (dd, J = 9.0, 2.0 Hz, 1 H), 7.20 (s, 1 H), 6.24-6.10 (m, 2 H), 5.58 (dd, J = 10.0, 2.1 Hz, 1 H), 4.60 (t, J = 8.7 Hz, 1 H), 4.43 (t, J = 7.5 Hz, 2H), 4.29-4.22 (m, 1H), 4.18-4.10 (m, 3 H), 2.61-2.33 (m, 10 H), 2.26 (s, 3 H), 2.01-1.96 (m, 2 H). |
| 32 | 1-(3-(4-((3,4- dichloro-2- fluorophenyl)a- mino)-7- (methoxy- d3)quinazolin- 6-yl)azetidin- 1-yl)prop-2-en- 1-one | 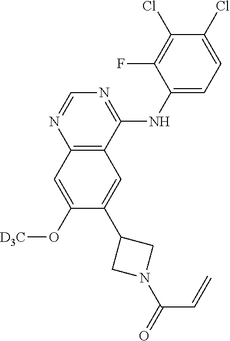 [SMILES: [2H]C([2H])([2H])Oc1cc2ncnc(Nc3ccc(Cl)c(Cl)c3F)c2cc1C1CN(C(=O)C=C)C1] | None | LC/MS (ESI, m/z): [M + 1]⁺ = 450.2, 452.2. ¹H NMR (400 MHz, DMSO-d₆) δ 9.93 (s, 1H), 8.46 (s, 1H), 8.37 (s, 1H), 7.68-7.53 (m, 2H), 7.24 (s, 1H), 6.36 (dd, J = 17.0, 10.3 Hz, 1H), 6.13 (dd, J = 17.0, 2.2 Hz, 1H), 5.69 (dd, J = 10.3, 2.2 Hz, 1H), 4.69 (t, J = 8.7 Hz, 1H), 4.39-4.25 (m, 3H), 4.23-4.13 (m, 1H). |
| 33 | (S)-1-(3-(4-((3- chloro-2,4- difluorophenyl) amino)-7- ((tetrahydrofuran- 3- yl)oxy)quinazo- lin-6- yl)azetidin-1- yl)prop-2-en-1- one | 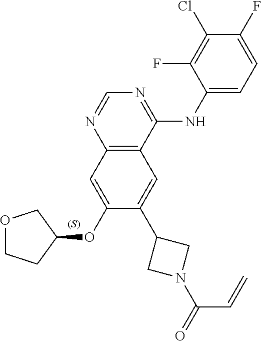 | TFA | LC/MS (ESI, m/z): [M + H]⁺ = 487.1. ¹H NMR (400 MHz, DMSO-d₆) δ 11.12 (br s, 1 H), 8.81 (s, 1 H), 8.53 (s, 1 H), 77.66-7.60 (m, 1 H), 7.50 (td, J = 8.8, 2.0 Hz, 1 H), 7.29 (s, 1 H), 6.37 (dd, J = 16.8, 10.0 Hz, 1 H), 6.13 (dd, J = 16.8, 2.0 Hz, 1 H), 5.70 (dd, J = 10.0, 2.0 Hz, 1 H), 5.30 (s, 1 H), 4.70 (t, J = 8.6 Hz, 1 H), 4.38-4.28 (m, 1 H), 4.20-4.14 (m, 1 H), 4.02-3.98 (m, 1 H), 3.93-3.81 (m, 3 H), 2.36-2.31 (m, 1 H), 2.12-2.09 (m, 1 H). |

### Example 34: Preparation of N-((4-((3,4-dichloro-2-fluorophenyl)amino)-6,7-dihydrofuro[3,2-2]quinazolin-6-yl)methyl)acrylamide (Compound 34)

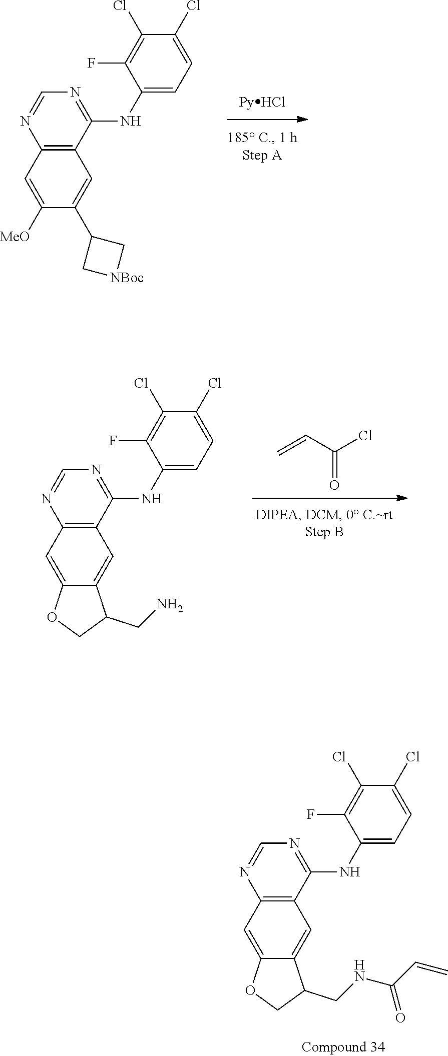

Step A: A mixture of tert-butyl 3-(4-((3,4-dichloro-2-fluorophenyl)amino)-7-methoxyquinazolin-6-yl)azetidine-1-carboxylate (3 g, 6.1 mmol) and pyridine hydrochloride (35 g, 305 mmol) was warmed to 185° C. and maintained at 185° C. for 1 h. The reaction mixture was cooled to room temperature, and water (1 L) was added to quench the reaction. Saturated NaHCO₃ solution was added to the mixture until pH>8. The mixture was extracted with EA (3×50 mL). The combined organic layers were washed with brine, dried over dried over Na₂SO₄, filtered and concentrated in vacuum. The residue was dried to give 6-(aminomethyl)-N-(3,4-dichloro-2-fluorophenyl)-6,7-dihydrofuro[3,2-g]quinazolin-4-amine (2 g, 87% yield) as a white solid. LC/MS (ESI, m/z): [M+1]⁺=379.1. ¹H NMR (400 MHz, DMSO-d₆) δ 8.58 (d, J=4.13 Hz, 1H), 8.38 (d, J=7.00 Hz, 2H), 7.43-7.64 (m, 2H), 7.32-7.43 (m, 1H), 6.99 (s, 1H), 4.69-4.83 (m, 1H), 4.58 (dd, J=6.00, 8.88 Hz, 1H), 3.58-3.72 (m, 1H), 2.97 (dd, J=5.44, 12.57 Hz, 1H), 2.85 (dd, J=7.44, 12.44 Hz, 1H).

Step B: To a mixture of 6-(aminomethyl)-N-(3,4-dichloro-2-fluorophenyl)-6,7-dihydrofuro[3,2-g]quinazolin-4-amine (79 mg, 0.209 mmol) and DIPEA (54 mg, 0.418 mmol) in DCM (5 mL) was added acryloyl chloride (19 mg, 0.21 mmol). The mixture was stirred at RT for 1 hour. Then the reaction mixture was quenched by MeOH and concentrated in vacuum. The residue was purified by prep-HPLC to give N-((4-((3,4-dichloro-2-fluorophenyl)amino)-6,7-dihydrofuro[3,2-g]quinazolin-6-yl)methyl)acrylamide (Compound 34) (17 mg, 18.8% yield) as a white solid. ¹HNMR (400 MHz, DMSO-d₆) δ 9.85 (s, 1H), 8.46-8.39 (m, 3H), 7.64-7.46 (m, 2H), 7.03 (s, 1H), 6.25 (dd, J=10.0, 17.1 Hz, 1H), 6.13 (dd, J=2.3, 17.1 Hz, 1H), 5.63 (dd, J=2.3, 10.0 Hz, 1H), 4.76 (t, J=8.8 Hz, 1H), 4.53 (dd, J=5.4, 9.0 Hz, 1H), 3.86-3.84 (m, 1 H), 3.76-3.63 (m, 1H), 3.31-3.26 (m, 1H). LC/MS (ESI, m/z): [M+H]⁺=433.0, 435.0.

### Example 35: Preparation of (Z)—N-((4-((3,4-dichloro-2-fluorophenyl)imino)-1-trideuteriomethyl-1,4,6,7-tetrahydrofuro[3,2-2]quinazolin-6-yl)methyl)acrylamide (Compound 35)

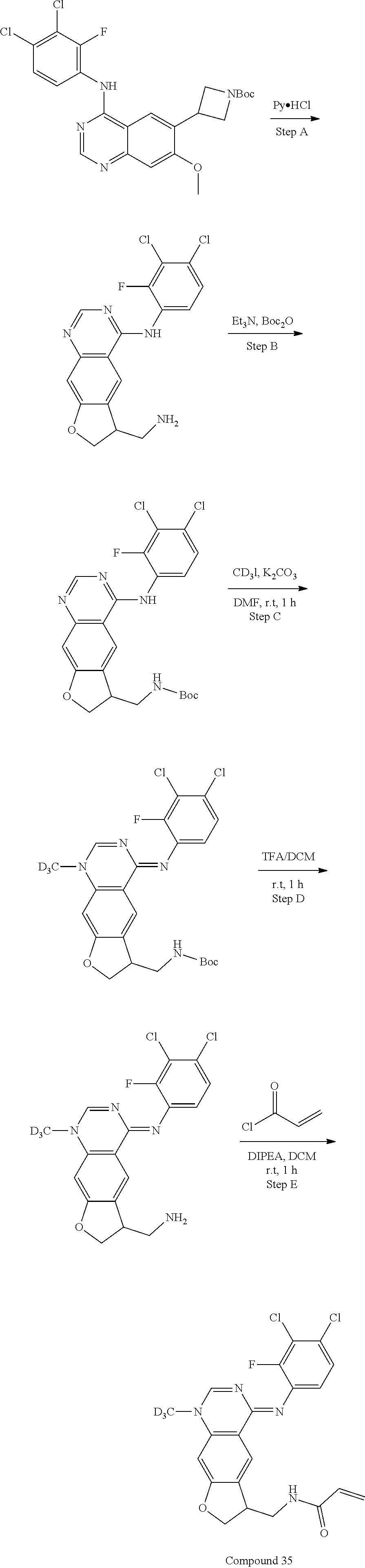

Step A: A mixture of tert-butyl 3-(4-((3,4-dichloro-2-fluorophenyl)amino)-7-methoxyquinazolin-6-yl)azetidine-1-carboxylate (3 g, 6.1 mmol) and pyridine hydrochloride (35 g, 305 mmol) was warmed to 185° C. and maintained at 185° C. for 1 h. The reaction mixture was cooled to room temperature and water (200 mL) was added to quench the reaction. Saturated NaHCO₃ solution was added to the mixture until pH>8. The mixture was extracted with EA (3*50 mL). The combined organic layers were washed with brine, dried over dried over Na₂SO₄, filtered and concentrated in vacuo. The residue was dried to afford the 6-(aminomethyl)-N-(3,4-dichloro-2-fluorophenyl)-6,7-dihydrofuro[3,2-g]quinazolin-4-amine (2 g, 87% yield) as a white solid. LC/MS (ESI, m/z): [M+1]⁺=379.1.

Step B: A mixture of 6-(aminomethyl)-N-(3,4-dichloro-2-fluorophenyl)-6,7-dihydrofuro[3,2-g]quinazolin-4-amine (2 g, 4.46 mmol), Boc₂O (1.44 g, 6.71 mmol), Et₃N (1.23 g, 12.2 mmol) in DCM (20 mL) was stirred at room temperature for 2 h. The mixture concentrated in vacuum. The residue was purified by flash chromatography eluting with EA/PE=60% to afford tert-butyl ((4-((3,4-dichloro-2-fluorophenyl)amino)-6,7-dihydrofuro[3,2-g]quinazolin-6-yl)methyl)carbamate (1.5 g, 70% yield) as a white solid. LC/MS (ESI, m/z): [M+1]⁺=479.1. ¹H NMR (400 MHz, DMSO-d₆) δ 9.80 (s, 1H), 8.39 (s, 1H), 8.35 (s, 1H), 7.50-7.62 (m, 2H), 7.24 (t, J=5.75 Hz, 1H), 7.01 (s, 1H), 4.75 (t, J=8.88 Hz, 1H), 4.54 (dd, J=5.25, 9.13 Hz, 1H), 3.68-3.80 (m, 1H), 3.40-3.53 (m, 1H), 3.05-3.15 (m, 1H), 1.38 (s, 9 H).

Step C: To a mixture of tert-butyl ((4-((3,4-dichloro-2-fluorophenyl)amino)-6,7-dihydrofuro[3,2-g]quinazolin-6-yl)methyl)carbamate (100 mg, 0.21 mmol), K₂CO₃ (58 mg, 0.42 mmol) in DMF (5 mL) was added CD₃I (33.4 mg, 0.23 mmol) dropwise at RT. The mixture was stirred at room temperature overnight. Then the reaction mixture was poured into water, extracted with EA (3×20 mL). The combined organic layers were concentrated in vacuum. The residue was purified by flash chromatography eluting with EA/PE=60% to afford (Z)-tert-butyl ((4-((3,4-dichloro-2-fluorophenyl)imino)-1-trideuteriomethyl-1,4,6,7-tetrahydrofuro[3,2-g]quinazolin-6-yl)methyl)carbamate (29 mg, 23% yield) as a yellow solid. LC/MS (ESI, m/z): [M+H]⁺=496.1. ¹H NMR (400 MHz, DMSO-d₆) δ 8.15 (s, 1H), 7.97 (s, 1H), 7.32 (dd, J=1.69, 8.69 Hz, 1H), 7.19 (t, J=5.63 Hz, 1H), 6.98 (t, J=8.44 Hz, 1H), 6.87 (s, 1H), 4.70 (t, J=9.01 Hz, 1H), 4.46 (dd, J=5.19, 9.07 Hz, 1H), 3.62-3.72 (m, 1H), 3.21-3.29 (m, 1H), 3.08-3.18 (m, 1H), 1.36 (s, 9H).

Step D: To a solution of (Z)-tert-butyl ((4-((3,4-dichloro-2-fluorophenyl)imino)-1-trideuteriomethyl-1,4,6,7-tetrahydrofuro[3,2-g]quinazolin-6-yl)methyl)carbamate (128 mg, 0.258 mmol) in DCM (3 mL) was added TFA (1 mL). The mixture was stirred at room temperature for 2 h. The reaction mixture was concentrated in vacuum. The residue was dried to afford (Z)—N-(6-(aminomethyl)-1-trideuteriomethyl-6,7-dihydrofuro[3,2-g]quinazolin-4(1H)-ylidene)-3,4-dichloro-2-fluoroaniline 2,2,2-trifluoroacetate (130 mg, 100% yield) as a yellow solid. LC/MS (ESI, m/z): [M+H]⁺=396.1.

Step E: To a mixture of (Z)—N-(6-(aminomethyl)-1-trideuteriomethyl-6,7-dihydrofuro[3,2-g]quinazolin-4(1H)-ylidene)-3,4-dichloro-2-fluoroaniline 2,2,2-trifluoroacetate (130 mg, 0.258 mmol) and DIPEA (66.5 mg, 0.516 mmol) in DCM (5 mL) was added acryloyl chloride (23 mg, 0.258 mmol). The mixture was stirred at RT for 1 hour. The reaction mixture was quenched by addition of MeOH and concentrated in vacuum. The residue was purified by prep-HPLC to afford (Z)—N-((4-((3,4-dichloro-2-fluorophenyl)imino)-1-trideuteriomethyl-1,4,6,7-tetrahydrofuro[3,2-g]quinazolin-6-yl)methyl)acrylamide (Compound 35) (38.3 mg, 33.1% yield) as a white solid. ¹H NMR (400 MHz, DMSO-d₆) δ 8.39 (t, J=5.8 Hz, 1H), 8.22-8.15 (m, 1H), 7.98 (s, 1H), 7.32 (dd, J=1.7, 8.7 Hz, 1H), 6.99 (t, J=8.5 Hz, 1H), 6.88 (s, 1H), 6.24 (dd, J=10.1, 17.1 Hz, 1H), 6.10 (dd, J=2.3, 17.1 Hz, 1H), 5.60 (dd, J=2.3, 10.0 Hz, 1 H), 4.72 (t, J=9.0 Hz, 1H), 4.48 (dd, J=5.4, 9.1 Hz, 1H), 3.82-3.72 (m, 1H), 3.61-3.52 (m, 1H), 3.40-3.33 (m, 1H). LC/MS (ESI, m/z): [M+H]⁺=449.9, 452.0, 454.0.

### Example 36: Preparation of N-(((Z)-4-((3,4-dichloro-2-fluorophenyl)imino)-1-((S)-tetrahydrofuran-3-yl)-1,4,6,7-tetrahydrofuro[3,2-2]quinazolin-6-yl)methyl)acrylamide (Compound 36)

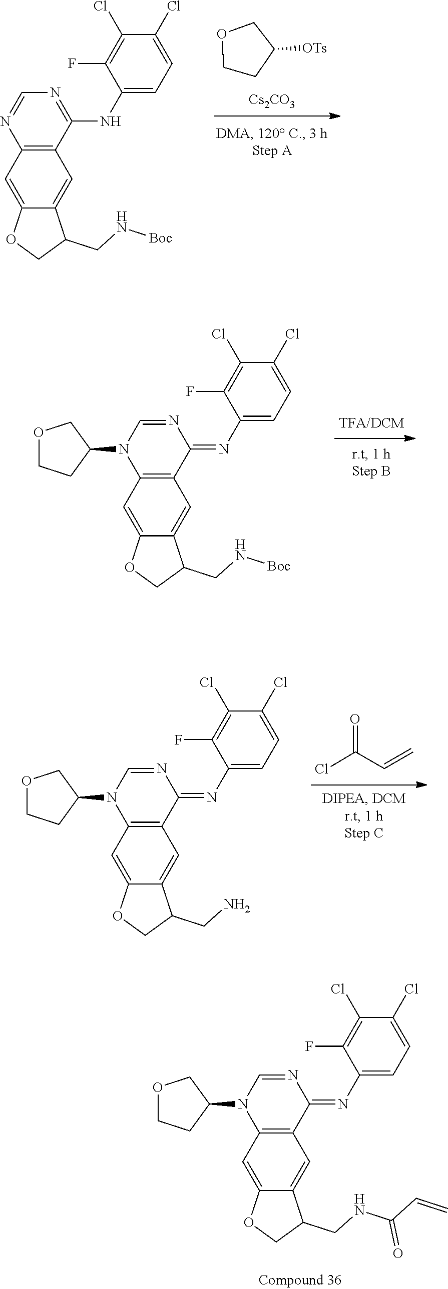

Step A: A mixture of tert-butyl ((4-((3,4-dichloro-2-fluorophenyl)amino)-6,7-dihydrofuro[3,2-g]quinazolin-6-yl)methyl)carbamate (100 mg, 0.21 mmol), (R)-tetrahydrofuran-3-yl 4-methylbenzenesulfonate (66 mg, 0.273 mmol) and Cs₂CO₃ (137 mg, 0.42 mmol) in DMA (5 mL) was warmed to 120° C. and stirred for 3 h. The reaction mixture was cooled to room temperature, then poured into water, and extracted with EA (3×20 mL). The combined organic layers were washed with water (30 mL), brine (30 mL), dried over Na₂SO₄, filtered and concentrated in vacuum. The residue was purified by silica gel column chromatography eluting with EA/PE=1/1 to afford tert-butyl (((Z)-4-((3,4-dichloro-2-fluorophenyl)imino)-1-((S)-tetrahydrofuran-3-yl)-1,4,6,7-tetrahydrofuro[3,2-g]quinazolin-6-yl)methyl)carbamate (35 mg, 30.4 yield) as a yellow solid. LC/MS (ESI, m/z): [M+1]⁺=549.1.

Step B: To a solution of tert-butyl (((Z)-4-((3,4-dichloro-2-fluorophenyl)imino)-1-((S)-tetrahydrofuran-3-yl)-1,4,6,7-tetrahydrofuro[3,2-g]quinazolin-6-yl)methyl)carbamate (35 mg, 0.064 mmol) in DCM (3 mL) was added TFA (1 mL). The mixture was stirred at room temperature for 2 h. The reaction mixture was concentrated in vacuo. The residue was dried to afford (Z)—N-(6-(aminomethyl)-1-((S)-tetrahydrofuran-3-yl)-6,7-dihydrofuro[3,2-g]quinazolin-4(1H)-ylidene)-3,4-dichloro-2-fluoroaniline 2,2,2-trifluoroacetate (35.9 mg, 100% yield) as a yellow solid. LC/MS (ESI, m/z): [M+1]⁺=449.1.

Step C: To a mixture of (Z)—N-(6-(aminomethyl)-1-((S)-tetrahydrofuran-3-yl)-6,7-dihydrofuro[3,2-g]quinazolin-4(1H)-ylidene)-3,4-dichloro-2-fluoroaniline 2,2,2-trifluoroacetate (35.9 mg, 0.064 mmol) and DIPEA(16.5 mg, 0.128 mmol) in DCM (5 mL) was added acryloyl chloride (5.8 mg, 0.064 mmol). The mixture was stirred at RT for 1 hour. The reaction mixture was quenched by MeOH and concentrated in vacuo. The residue was purified by prep HPLC to afford N-(((Z)-4-((3,4-dichloro-2-fluorophenyl)imino)-1-((S)-tetrahydrofuran-3-yl)-1,4,6,7-tetrahydrofuro[3,2-g]quinazolin-6-yl)methyl)acrylamide (Compound 36) (8 mg, 25% yield) as a white solid. ¹H NMR (400 MHz, DMSO-d₆) δ 8.39 (t, J=5.9 Hz, 1H), 8.22 (s, 1H), 7.95 (d, J=0.9 Hz, 1H), 7.33 (dd, J=1.7, 8.7 Hz, 1H), 7.12 (s, 1H), 6.99 (t, J=8.5 Hz, 1H), 6.24 (dd, J=10.0, 17.1 Hz, 1H), 6.10 (dd, J=2.2, 17.1 Hz, 1H), 5.60 (dd, J=10.0, 2.0 Hz, 1H), 5.16-5.06 (m, 1H), 4.72 (t, J=9.1 Hz, 1H), 4.48 (dd, J=5.4, 9.3 Hz, 1H), 4.12-4.00 (m, 2H), 3.89-3.71 (m, 3H), 3.60-3.50 (m, 1H), 2.48-2.43 (m, 1H), 2.24-2.11 (m, 1H). LC/MS (ESI, m/z): [M+H]⁺=503.1, 505.0, 507.1.

### Example 37: Preparation of (S)-1-(3-(4-((3,4-dichloro-2-fluorophenyl)amino)-7-((tetrahydrofuran-3-yl)oxy)quinazolin-6-yl)azetidin-1-yl)-2-fluoroprop-2-en-1-one (Compound 37)

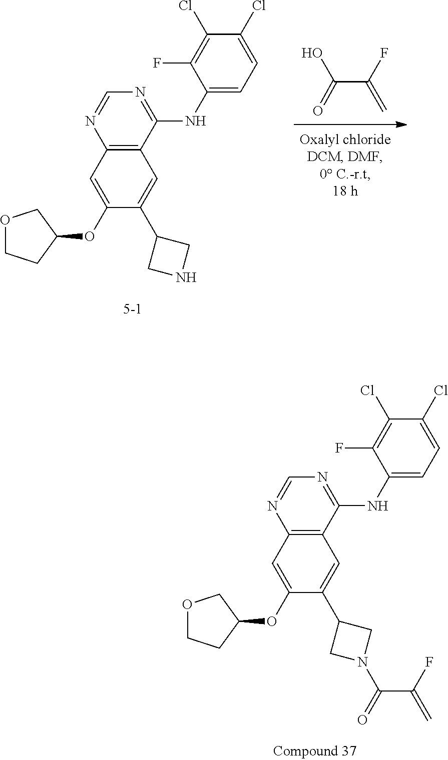

Oxalyl chloride (16 mg, 0.17 mmol) was added to the solution of the 2-fluoroacrylic acid (8 mg, 0.087 mmol) in DCM (5 mL) and DMF (1 drop) at 0° C. under N₂. The resulting mixture was stirred at room temperature for 3 h. A solution of (S)-6-(azetidin-3-yl)-N-(3,4-dichloro-2-fluorophenyl)-7-((tetrahydrofuran-3-yl)oxy)quinazolin-4-amine (30 mg, 0.067 mmol) in DCM was added at 0° C. The mixture was warmed to room temperature and stirred overnight. The reaction mixture was poured into water and extracted with EA (3×10 mL). The combined organic layers were washed with water, brine, dried over anhydrous sodium sulfate, filtered and concentrated in vacuum. The residue was purified by prep-TLC eluting with MeOH/DCM=1:10 to afford (S)-1-(3-(4-((3,4-dichloro-2-fluorophenyl)amino)-7-((tetrahydrofuran-3-yl)oxy)quinazolin-6-yl)azetidin-1-yl)-2-fluoroprop-2-en-1-one (Compound 37) (4.4 mg, 13% yield). LC/MS (ESI, m/z): [M+H]⁺=521.1, 523.1, 525.2. ¹H NMR (400 MHz, DMSO-d₆) δ 9.93 (s, 1H), 8.46 (s, 1H), 8.34 (s, 1H), 7.66-7.50 (m, 2H), 7.22 (s, 1H), 5.50 (dd, J=3.5, 48.4 Hz, 1H), 5.36-5.25 (m, 2H), 4.79 (dt, J=4.8, 8.7 Hz, 1H), 4.54 (t, J=8.1 Hz, 1H), 4.40 (t, J=9.8 Hz, 1H), 4.31 (t, J=6.8 Hz, 1H), 4.21-4.15 (m, 1H), 3.99 (td, J=3.8, 9.9 Hz, 1H), 3.90-3.78 (m, 3H), 2.37-2.24 (m, 1H), 2.10-2.02 (m, 1H).

Examples 38-64 were prepared using similar procedures as described in the preceding Examples.

| EX. | | Compound | | |
|---|---|---|---|---|
| NO. | IUPAC Name | structure | salt | Compound characterization |
| 38 | 1-(3-(4-((3,4- dichloro-2- fluorophenyl)amino)- 7- methoxyquinazolin- 6-yl)azetidin-1-yl)- 2-fluoroprop-2-en- 1-one | 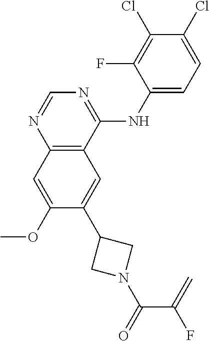 [SMILES: C=C(F)C(=O)N1CC(c2cc3c(Nc4ccc(Cl)c(Cl)c4F)ncnc3cc2OC)C1] | None | LC/MS (ESI, m/z): [M + 1]⁺ = 465.0, 467.0 ¹H NMR (400 MHz, DMSO-d₆) δ 9.94 (s, 1 H), 8.45 (s, 1 H), 8.36 (s, 1 H), 7.59 (s, 2 H), 7.23 (s, 1 H), 5.50 (dd, J = 48.5, 3.5 Hz, 1 H), 5.31 (dd, J = 16.6, 3.5 Hz, 1 H), 4.89-4.76 (m, 1 H), 4.56-4.45 (m, 1 H), 4.41 (t, J = 9.7 Hz, 1 H), 4.37- 4.27 (m, 1 H), 4.27-4.14 (m, 1 H), 3.96 (s, 3 H). |
| 39 | 1-(3-(4-((3,4- dichloro-2- fluorophenyl)amino)- 7-(methoxy- d3)quinazolin-6- yl)azetidin-1-yl)-2- fluoroprop-2-en-1- one | 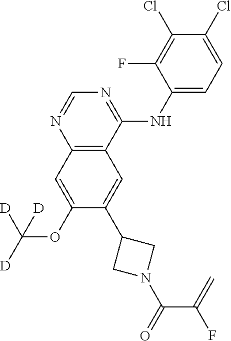 [SMILES: [2H]C([2H])([2H])Oc1cc2ncnc(Nc3ccc(Cl)c(Cl)c3F)c2cc1C1CN(C(=O)C(=C)F)C1] | None | LC/MS (ESI, m/z): [M + 1]⁺ = 468.2, 470.2, 472.2. ¹H NMR (400 MHz, CDCl₃) δ 8.64 (s, 1 H), 8.21 (t, J = 8.4 Hz, 1 H), 7.76 (s, 2 H), 7.26 (dd, J = 1.9, 9.1 Hz, 1 H), 7.21 (s, 1H), 5.60 (dd, J = 3.2, 46.8 Hz, 1 H), 5.02 (dd, J = 3.1, 15.8 Hz, 1 H), 4.80 (dt, J = 4.3, 9.3 Hz, 1 H), 4.52-4.32 (m, 3 H), 4.25-4.12 (m, 1 H). |
| 40 | N-((1s,3s)-3-((4- ((3,4-dichloro-2- fluorophenyl)amino)- 7- methoxyquinazolin- 6- yl)oxy)cyclobutyl)- 2-fluoroacrylamide | 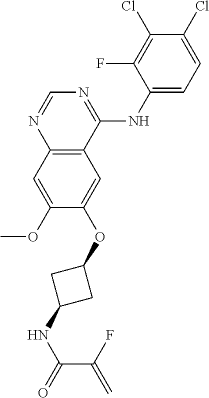 [SMILES: C=C(F)C(=O)N[C@H]1C[C@@H](Oc2cc3c(Nc4ccc(Cl)c(Cl)c4F)ncnc3cc2OC)C1] | TFA | LC/MS (ESI, m/z): [M + H]⁺ = 495.0. ¹H NMR (400 MHz, DMSO-d₆) δ: 10.60 (br s, 1 H), 8.87 (d, J = 7.6 Hz, 1 H), 8.65 (s, 1 H), 7.61-7.71 (m, 3 H), 7.29 (s, 1 H), 5.53 (dd, J = 48.0, 3.2 Hz, 1 H), 5.27 (dd, J = 16.0, 3.2 Hz, 1 H), 4.58-4.67 (m, 1 H), 4.07-4.13 (m, 1 H), 3.99 (s, 3 H), 3.03-3.06 (m, 2 H), 2.25-2.28 (m, 2 H). |
| 41 | 1-(3-(4-((4- (benzyloxy)-3- chloro-2- fluorophenyl)amino)- 7- methoxyquinazolin- 6-yl)azetidin-1- yl)prop-2-yn-1-one | 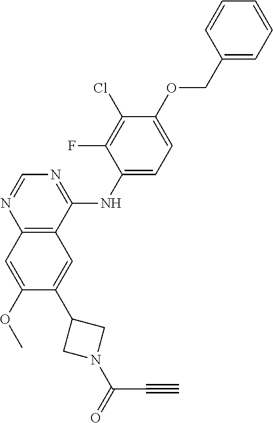 [SMILES: C#CC(=O)N1CC(c2cc3c(Nc4ccc(OCc5ccccc5)c(Cl)c4F)ncnc3cc2OC)C1] | None | LC/MS (ESI, m/z): [M + 1]⁺ = 517.1. ¹H NMR (400 MHz, DMSO-d₆) δ 9.69 (s, 1 H), 8.39 (s, 1 H), 8.35 (s, 1 H), 7.60 (dd, J = 9.0, 6.0 Hz, 1H), 7.32 (t, J = 8.9 Hz, 1 H), 7.23-7.01 (m, 6 H), 4.91 (s, 2 H), 4.61 (t, J = 8.5 Hz, 1 H), 4.50 (s, 1 H), 4.36-4.33 (m, 2 H), 4.22-4.18 (m, 2 H), 3.95 (s, 3 H). |
| 42 | (E)-N-(3-((4-((3,4- dichloro-2- fluorophenyl)amino)- 7- methoxyquinazolin- 6- yl)oxy)cyclobutyl)- 4- (dimethylamino)but- 2-enamide | 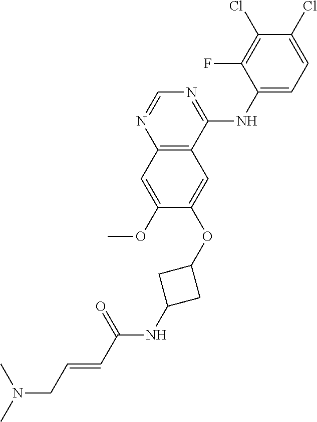 [SMILES: COc1cc2ncnc(Nc3ccc(Cl)c(Cl)c3F)c2cc1OC1CC(NC(=O)/C=C/CN(C)C)C1] | None | LC/MS (ESI, m/z): [M + 1]⁺ = 534.1, 536.1. ¹H NMR (400 MHz, DMSO-d₆, a = 0.5 cis, b = 0.5 trans) δ 9.73-9.63 (m, 1 H), 8.49-8.35 (m, 2 H), 7.62-7.54 (m, 2 H), 7.50-7.46 (m, 1 H), 7.25-7.21 (m, 1 H), 6.61-6.51 (m, 1 H), 6.02 (dd, J = 20.2, 15.6 Hz, 1H), 5.04-4.96 (m, 0.5 H, trans), 4.651-4.55 (m, 0.5 H, cis), 4.45-4.35 (m, 0.5 H, trans), 4.11-4.01 (m, 0.5 H, cis), 3.96 (s, 3 H), 3.08-2.96 (m, 2 H), 2.62-2.54 (m, 2 H), 2.16 (s, 6 H), 2.08-1.94 (m, 2 H). |
| 43 | N-(3-((4-((3,4- dichloro-2- fluorophenyl)amino)- 7- methoxyquinazolin- 6- yl)oxy)cyclobutyl)but- 2-ynamide | 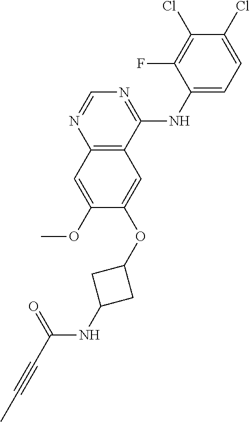 [SMILES: CC#CC(=O)NC1CC(Oc2cc3c(Nc4ccc(Cl)c(Cl)c4F)ncnc3cc2OC)C1] | None | LC/MS (ESI, m/z): [M + 1]⁺ = 489.1, 491.1. ¹H NMR (400 MHz, DMSO-d₆, a cis = 0.7, b trans = 0.3) δ 9.75-9.61 (m, 1 H), 8.97 (d, J = 6.8 Hz, 0.3 H, trans), 8.87 (d, J = 7.6 Hz, 0.7 H, cis), 8.38 (s, 1 H), 7.68-7.43 (m, 3 H), 7.23 (s, 1 H), 5.00-4.92 (m, 0.3 H, trans), 4.61-4.51 (m, 0.7 H, cis), 4.39-4.29 (m, 0.3 H, trans), 4.01-3.95 (m, 0.7 H, cis), 3.98 (s, 3 H), 3.04-2.98 (m, 2 H), 2.17-2.02 (m, 2 H), 1.98 (s, 3 H). |
| 44 | N-((1r,3r)-3-((4- ((2,3-dihydro-1H- inden-4-yl)amino)- 7- methoxyquinazolin- 6- yl)oxy)cyclobutyl)pro- piolamide | 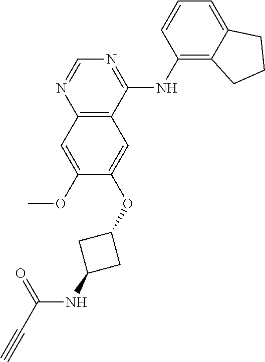 [SMILES: C#CC(=O)N[C@H]1C[C@H](Oc2cc3c(Nc4cccc5c4CCC5)ncnc3cc2OC)C1] | None | LC/MS (ESI, m/z): [M + 1]⁺ = 429.2. ¹H NMR (400 MHz, DMSO-d₆) δ 10.94 (br s, 1 H), 9.22 (d, J = 6.6 Hz, 1 H), 8.72 (s, 1 H), 7.68 (s, 1 H), 7.30-7.25 (m, 3 H), 7.19- 7.17 (m, 1 H), 5.02-4.95 (m, 1 H), 4.40- 4.33 (m, 1 H), 4.18 (s, 1H), 4.01 (s, 3 H), 2.97 (t, J = 7.4 Hz, 2 H), 2.74 (t, J = 7.2 Hz, 2 H), 2.67-2.60 (m, 2 H), 2.74-2.42 (m, 2 H), 2.05-1.97 (m, 2 H). |
| 45 | N-((1s,3s)-3-((4- ((2,3-dihydro-1H- inden-4-yl)amino)- 7- methoxyquinazolin- 6- yl)oxy)cyclobutyl)pro- piolamide | 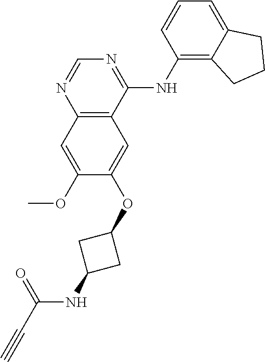 [SMILES: C#CC(=O)N[C@H]1C[C@@H](Oc2cc3c(Nc4cccc5c4CCC5)ncnc3cc2OC)C1] | None | LC/MS (ESI, m/z): [M + 1]⁺ = 429.2. ¹H NMR (400 MHz, DMSO-d₆) δ 9.41 (s, 1 H), 9.11 (d, J = 7.5 Hz, 1 H), 8.30 (s, 1 H), 7.56 (s, 1 H), 7.18-7.13 (m, 4 H), 4.56 (dd, J = 15.2, 7.5 Hz, 1 H), 4.18 (s, 1 H), 3.98 (dd, J = 15.8, 8.3 Hz, 1 H), 3.93 (s, 3 H), 3.08-2.97 (m, 2 H), 2.93 (t, J = 7.3 Hz, 2 H), 2.73 (t, J = 7.4 Hz, 2 H), 2.09 (dd, J = 19.5, 9.0 Hz, 2 H), 1.97 (dd, J = 14.7, 7.4 Hz, 2 H). |
| 46 | N-(3-((7-methoxy- 4-(naphthalen-1- ylamino)quinazolin- 6- yl)oxy)cyclobutyl)pro- piolamide | 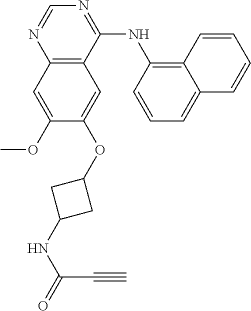 [SMILES: C#CC(=O)NC1CC(Oc2cc3c(Nc4cccc5ccccc45)ncnc3cc2OC)C1] | None | LC/MS (ESI, m/z): [M + 1]⁺ = 439.1. ¹H NMR (400 MHz, DMSO-d₆, a cis = 0.3, b trans = 0.7) δ 9.81 (s, 1 H), 9.20 (d, J = 6.8 Hz, 0.7 H, trans), 9.11 (d, J = 7.2 Hz, 0.3 H, cis), 8.18 (s, 1 H), 8.00 (d, J = 8.0 Hz, 1 H), 7.91 (dd, J = 7.0, 1.8 Hz, 1 H), 7.88-7.84 (m, 1 H), 7.67-7.45 (m, 5 H), 7.20 (s, (s, 1 H), 5.04-4.96 (m, 0.7 H, trans), 4.62-4.56 (m, 0.3 H, cis), 4.43- 4.33 (m, 0.7 H, trans), 4.17 (s, 0.3 H, cis), 4.14 (s, 0.7 H, trans), 4.01-3.95 (m, 0.3 H, cis), 3.96 (s, 3 H), 3.12-3.00 (m, 0.5 H), 2.68-2.56 (m, 2 H), 2.54-2.44 (m, 1 H), 2.17-2.05 (m, 0.5 H). |
| 47 | N-((1s,3s)-3-((7- methoxy-4- (naphthalen-1- ylamino)quinazolin- 6- yl)oxy)cyclobutyl)pro- piolamide | 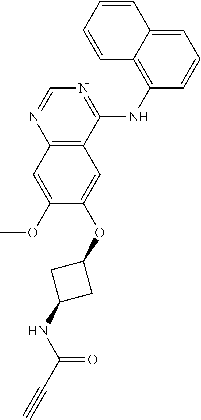 [SMILES: C#CC(=O)N[C@H]1C[C@@H](Oc2cc3c(Nc4cccc5ccccc45)ncnc3cc2OC)C1] | None | LC/MS (ESI, m/z): [m + 1]⁺ = 439.2. ¹H NMR (400 MHz, DMSO-d₆) δ 9.80 (s, 1 H), 9.11 (d, J = 7.5 Hz, 1 H), 8.18 (s, 1 H), 8.00 (d, J = 7.5 Hz, 1 H), 7.91 (d, J = 7.2 Hz, 1 H), 7.85 (d, J = 8.3 Hz, 1 H), 7.72 (s, 1 H), 7.61-7.47 (m, 4 H), 7.20 (s, 1 H), 4.63-4.56 (m, 1 H), 4.17 (s, 1H), 4.02-3.96 (m, 1 H), 3.95 (s, 3 H), 3.09-3.01 (m, 2 H), 2.16-2.08 (m, 2 H). |
| 48 | N-((1r,3r)-3-((7- methoxy-4- (naphthalen-2- ylamino)quinazolin- 6- yl)oxy)cyclobutyl)pro- piolamide | 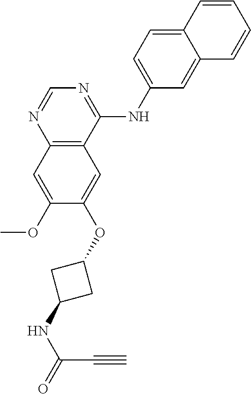 [SMILES: C#CC(=O)N[C@H]1C[C@H](Oc2cc3c(Nc4ccc5ccccc5c4)ncnc3cc2OC)C1] | None | LC/MS (ESI, m/z): [M + H]⁺ = 439.2. ¹H NMR (400 MHz, DMSO-d₆) δ 11.06 (s, 1 H), 9.24 (d, J = 6.8 Hz, 1 H), 8.82 (s, 1 H) 8.16 (d, J = 1.8 Hz, 1 H), 8.03 (d, J = 8.9 Hz, 1 H), 8.00-7.93 (m, 2 H), 7.81- 7.74 (m, 2 H) 7.61-7.48 (m, 2 H), 7.31 (s, 1 H), 5.12-4.96 (m, 1 H), 4.40 (dt, J = 14.7, 7.4 Hz, 1 H), 4.20 (s, 1 H), 4.02 (s, 3 H), 2.70-2.60 (m, 2 H), 2.55-2.45 (m, 2 H). |
| 49 | N-(3-((4-((3,4- dichloro-2- fluorophenyl)amino)- 7- methoxyquinazolin- 6- yl)oxy)cyclobutyl)pro- piolamide | 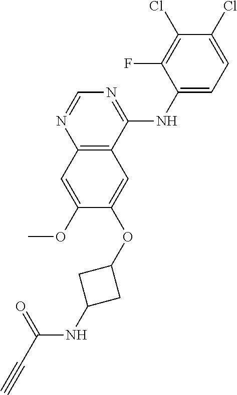 [SMILES: C#CC(=O)NC1CC(Oc2cc3c(Nc4ccc(Cl)c(Cl)c4F)ncnc3cc2OC)C1] | None | LC/MS (ESI, m/z): [M + H]⁺ = 475.1. ¹H NMR (400 MHz, DMSO-d₆, cis/trans = 1/1) δ 9.67 (d, J = 4.8 Hz, 1 H), 9.17 (dd, J = 40.1, 7.1 Hz, 1 H), 8.38 (s, 1 H), 7.66- 7.55 (m, 2 H), 7.50 (d, J = 27.1 Hz, 1 H), 7.23 (s, 1 H), 5.02-4.94 (m, 0.5 H trans), 4.60-4.54 (m, 0.5 H, cis), 4.40- 4.32 (m, 0.5 H, trans), 4.03-3.97 (m, 0.5 H, cis), 4.17 (s, 1 H), 3.95 (s, 3 H), 3.06- 2.98 (m, 1 H), 2.54-2.50 (m, 1 H), 2.50- 2.44 (m, 1 H), 2.12-2.00 (m, 1 H). |
| 50 | 1-(4-((4-((3,4- dichloro-2 fluorophenyl)amino)- 7- methoxyquinazolin- 6-yl)oxy)piperidin- 1-yl)-2-fluoroprop- 2-en-1-one | 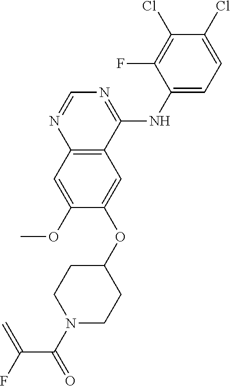 [SMILES: C=C(F)C(=O)N1CCC(Oc2cc3c(Nc4ccc(Cl)c(Cl)c4F)ncnc3cc2OC)CC1] | TFA | LC/MS (ESI, m/z): [M + H]⁺ = 509.1. ¹H NMR (400 MHz, DMSO-d₆) δ: 10.88 (br s, 1 H), 8.75 (s, 1 H), 8.06 (s, 1 H), 7.59- 7.70 (m, 2 H), 7.32 (s, 1 H), 5.30 (dd, J = 14.8, 4.0 Hz, 1 H), 5.22 (dd, J = 46.4, 4.0 Hz, 1H), 4.88-4.84 (m, 1 H), 4.01 (s, 3 H), 3.75-3.83 (m, 2 H), 3.48-3.58 (m, 2 H), 2.05-2.15 (m, 2 H), 1.74- 1.85 (m, 2 H). |
| 51 | 1-(4-((4-(3,4- dichloro-2- fluorobenzyl)-7- methoxyquinazolin- 6-yl)oxy)piperidin- 1-yl)prop-2-yn-1- one | 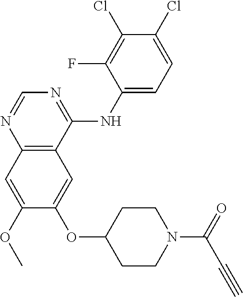 | None | LC/MS (ESI, m/z): [M + H]⁺ = 502.0. ¹H NMR (400 MHz, DMSO-d₆) δ 9.20 (s, 1 H), 7.93-7.71 (m, 3 H), 7.55 (s, 1 H), 4.93-4.81 (m, 1 H), 4.56 (s, 1 H), 4.05 (s, 3 H), 3.99-3.90 (m, 1 H), 3.83- 3.74 (m, 1 H), 3.73-3.62 (m, 1 H), 3.49-3.41 (m, 1 H), 2.15-1.93 (m, 2 H), 1.86-1.60 (m, 2 H). |
| 52 | 1-(4-((4-((3,4- dichloro-2- fluorophenyl)amino)- 7- methoxyquinazolin- 6-yl)oxy)piperidin-1- 1-yl)prop-2-yn-1- one | 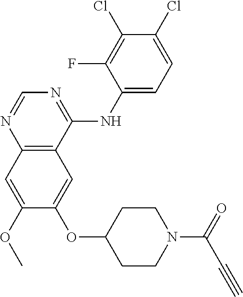 [SMILES: C#CC(=O)N1CCC(Oc2cc3c(Nc4ccc(Cl)c(Cl)c4F)ncnc3cc2OC)CC1] | None | LC/MS (ESI, m/z): [M + H]⁺ = 489.1, 491.1. ¹H NMR (400 MHz, DMSO-d₆) δ 9.65 (s, 1 H), 8.40 (s, 1 H), 7.89 (s, 1 H), 7.67-7.51 (m, 2 H), 7.25 (s, 1 H), 4.87- 4.74 (m, 1 H), 4.57 (s, 1 H), 4.00-3.94 (m, 4 H), 3.83-3.66 (m, 2 H), 3.57- 3.47 (m, 1 H), 2.12-1.98 (m, 2 H), 1.83- 1.68 (m, 2 H). |
| 53 | 1-(4-((7-methoxy- 4-(naphthalen-2- ylamino)quinazolin- 6-yl)oxy)piperidin- 1-yl)prop-2-yn-1- one | 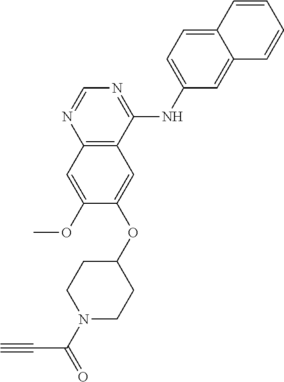 [SMILES: C#CC(=O)N1CCC(Oc2cc3c(Nc4ccc5ccccc5c4)ncnc3cc2OC)CC1] | None | LC/MS (ESI, m/z): [M + H]⁺ = 453.1. ¹H NMR (400 MHz, DMSO-d6) δ 11.21 (s, 1 H), 8.87 (s, 1 H), 8.21 (s, 1 H), 8.17 (d, J = 1.2 Hz, 1 H), 8.06 (d, J = 8.8 Hz, 1 H), 8.00-7.97 (m, 2 H), 7.80 (dd, J = 8.8 Hz, 1 H), 7.61-7.54 (m, 2 H), 7.28 (s, 1 H), 4.89 (m, 1 H), 4.58 (s, 1 H), 4.03 (s, 3 H), 3.99-3.92 (m, 1 H), 3.78-3.73 (m, 2 H), 3.58-3.52 (m, 1 H), 2.17-1.99 (m, 2 H), 1.88-1.72 (m, 2 H). |
| 54 | 1-(4-((7-methoxy- 4-(naphthalen-1- ylamino)quinazolin- 6-yl)oxy)piperidin- 1-yl)prop-2-yn-1- one | 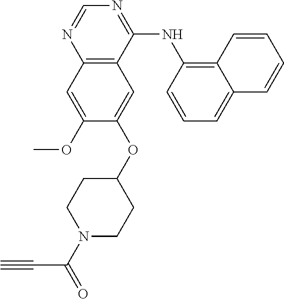 [SMILES: C#CC(=O)N1CCC(Oc2cc3c(Nc4cccc5ccccc45)ncnc3cc2OC)CC1] | None | LC/MS (ESI, m/z): [M + H]⁺ = 453.1. ¹H NMR (400 MHz, DMSO-d₆) δ 9.79 (s, 1 H), 8.19 (s, 1 H), 8.06 (s, 1 H), 8.00 (d, J = 8 Hz, 1 H), 7.91 (dd, J = 7.2 Hz, 1 H), 7.86 (d, J = 8.4 Hz, 1 H), 7.62-7.47 (m, 4 H), 7.22 (s, 1 H), 4.83-4.78 (m, 1 H), 4.55 (s, 1 H), 4.03-4.00 (m, 1 H), 3.99 (s, 3 H), 3.85-3.67 (m, 2 H), 3.51-3.46 (m, 1 H), 2.14-1.97 (m, 2 H), 1.88-1.71 (m, 2 H). |
| 55 | 1-(3-((4-(2,3- dihydro-1H-inden- 4-yl)amino)-7- methoxyquinazolin- 6-yl)oxy)-9-aza- bicyclo[3.3.1]nonan- 9-yl)prop-2-yn- 1-one | 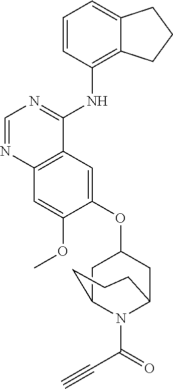 [SMILES: C#CC(=O)N1C2CCCC1CC(Oc1cc3c(Nc4cccc5c4CCC5)ncnc3cc1OC)C2] | None | LC/MS (ESI, m/z): [M + H]⁺ = 483.2. 1H NMR (400 MHz, DMSO) δ 10.94 (br s, 1 H), 8.73 (s, 1 H), 8.01 (s, 1 H), 7.30- 7.22 (m, 4 H), 5.43-5.35 (m, 1 H), 4.73-4.69 (m, 1 H), 4.67-4.63 (m, 1 H), 4.56 (s, 1 H), 3.99 (s, 3 H), 2.98 (t, J = 7.4 Hz, 2 H), 2.77 (t, J = 7.4 Hz, 2 H), 2.47-2.40 (m, 2 H), 2.06-1.96 (m, 2 H), 1.91-1.60 (m, 8 H). |
| 56 | 1-(3-((4-((2,3- dihydro-1H-inden- 4-yl)amino)-7- methoxyquinazolin- 6-yl)oxy)-8- azabicyclo[3.2.1]oc- tan-8-yl)prop-2-yn- 1-one | 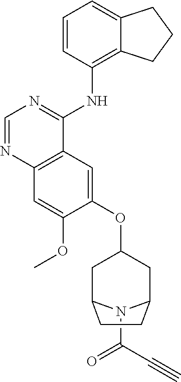 [SMILES: C#CC(=O)N1C2CCC1CC(Oc1cc3c(Nc4cccc5c4CCC5)ncnc3cc1OC)C2] | None | LC/MS (ESI, m/z): [M + H]⁺ = 469.2. ¹H NMR (400 MHz, DMSO-d₆) δ 9.32 (s, 1 H), 8.32 (s, 1 H), 7.79 (s, 1 H), 7.26- 7.11 (m, 4 H), 5.03-4.91 (m, 1 H), 4.59- 4.51 (m, 2 H), 4.48 (s, 1 H), 3.91 (s, 3 H), 2.94 (t, J = 7.2 Hz, 2 H), 2.74 (t, J = 7.2 Hz, 2 H), 2.46-2.38 (m, 1 H), 2.34- 2.25 (m, 1 H), 2.09-1.88 (m, 6 H), 1.68- 1.53 (m, 2 H). |
| 57 | 1-(3-((7-methoxy- 4-(naphthalen-1- ylamino)quinazolin- 6-yl)oxy)-9-aza- bicyclo[3.3.1]nonan- 9-yl)prop-2-yn- 1-one | 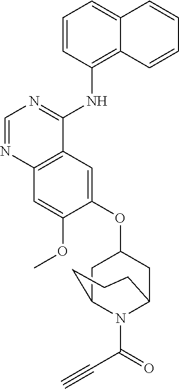 [SMILES: C#CC(=O)N1C2CCCC1CC(Oc1cc3c(Nc4cccc5ccccc45)ncnc3cc1OC)C2] | None | LC/MS (ESI, m/z): [M + 1]⁺ = 493.2. ¹H NMR (400 MHz, DMSO-d₆) δ 11.25 (br s, 1 H), 8.60 (s, 1 H), 8.16 (s, 1 H), 8.11-8.02 (m, 2 H), 7.89 (d, J = 8.2 Hz, 1 H), 7.72-7.52 (m, 4 H), 7.30 (s, 1 H), 5.54-5.37 (m, 1 H), 4.83-4.61 (m, 2 H), 4.56 (s, 1 H), 4.01 (s, 3 H), 2.45-2.30 (m, 2 H), 1.95-1.59 (m, 8 H). |
| 58 | 1-(3-((7-methoxy- 4-(naphthalen-2- ylamino)quinazolin- 6-yl)oxy)-9-aza- bicyclo[3.3.1]nonan- 9-yl)prop-2-yn- 1-one | 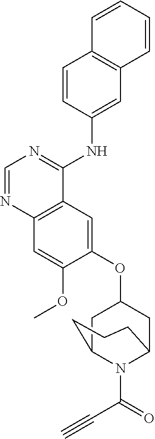 [SMILES: C#CC(=O)N1C2CCCC1CC(Oc1cc3c(Nc4ccc5ccccc5c4)ncnc3cc1OC)C2] | None | LC/MS (ESI, m/z): [M + 1]⁺ = 493.2. ¹H NMR (400 MHz, DMSO-d₆₎ δ 10.97 (br s, 1 H), 8.82 (s, 1 H), 8.24 (d, 1 H), 8.18 (s, 1 H), 8.04 (d, J = 8.8 Hz, 1 H), 8.01-7.93 (m, 2 H), 7.82 (dd, J = 8.8, 2.0 Hz, 1 H), 7.61-7.51 (m, 2 H), 7.31 (s, 1 H), 5.51-5.31 (m, 1 H), 4.82- 4.60 (m, 2 H), 4.56 (s, 1 H), 4.01 (s, 3 H), 2.33-2.51 (m, 2 H), 1.97-1.58 (m, 8 H). |
| 59 | 1-(3-((7-methoxy- 4-(naphthalen-2- ylamino)quinazolin- 6-yl)oxy)-8- azabicyclo[3.2.1]oc- tan-8-yl)prop-2-yn- 1-one | 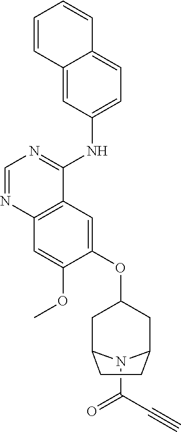 [SMILES: C#CC(=O)N1C2CCC1CC(Oc1cc3c(Nc4ccc5ccccc5c4)ncnc3cc1OC)C2] | None | LC/MS (ESI, m/z): [M + 1]⁺ = 479.2. ¹H NMR (400 MHz, DMSO-d₆) δ 9.65 (s, 1 H), 8.53 (s, 1 H), 8.40 (s, 1 H), 8.02- 7.85 (m, 5 H), 7.57-7.40 (m, 2 H), 7.23 (s, 1 H), 5.13-4.94 (m, 1 H), 4.62- 4.52 (m, 2 H), 4.49 (s, 1 H), 3.94 (s, 3 H), 2.45-2.26 (m, 2 H), 2.00-1.94 (m, 2 H), 1.72-1.54 (m, 2 H). |
| 60 | 1-(3-((7-methoxy- 4-(naphthalen-1- ylamino)quinazolin- 6-yl)oxy)-8- azabicyclo[3.2.1]oc- tan-8-yl)prop-2-yn- 1-one | 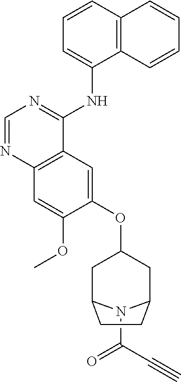 [SMILES: C#CC(=O)N1C2CCC1CC(Oc1cc3c(Nc4cccc5ccccc45)ncnc3cc1OC)C2] | None | LC/MS (ESI, m/z): [M + 1]⁺ = 479.2. ¹H NMR (400 MHz, DMSO-d₆) δ 11.29 (s, 1 H), 8.62 (s, 1 H), 8.18-7.99 (m, 3 H), 7.89 (d, J = 8.3 Hz, 1 H), 7.76- 7.51 (m, 4 H), 7.32 (s, 1 H), 5.18-5.01 (m, 1 H), 4.66-4.55 (m, 2 H), 4.51 (s, 1 H), 4.01 (s, 3 H), 2.41-2.27 (m, 2 H), 2.17-1.86 (m, 4 H), 1.76-1.55 (m, 2 H). |
| 61 | 1-(3-(4-((3,4- dichloro-2- fluorophenyl)amino) quinazolin-6- yl)piperidin-1- yl)prop-2-yn-1-one | 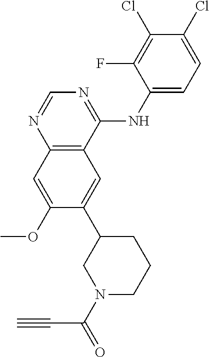 | None | LC/MS (ESI, m/z): [M + H]⁺ = 443.0, 445.0. ¹H NMR (400 MHz, DMSO-d₆) δ 8.79 (d, J = 6.5 Hz, 1 H), 8.51 (s, 1 H), 8.03 (dd, J = 8.6, 1.0 Hz, 1H), 7.87 (dd, J = 8.6, 3.2 Hz, 1 H), 7.69-7.59 (m, 2 H), 4.61 (s, 0.5 H), 4.55 (s, 0.5 H), 4.52-4.34 (m, 2 H), 3.24 (dt, J = 2.2, 13.0 Hz, 1 H), 3.01-2.80 (m, 2 H), 2.10- 2.07 (m, 1 H), 1.95-1.86 (m, 2 H), 1.67- 1.50 (m, 1 H). |
| 62 | 1-(5-(4-((3,4- dichloro-2- fluorophenyl)amino) quinazolin-6-yl)- 3,6-dihydropyridin- 1(2H)-yl)prop-2- yn-1-one | 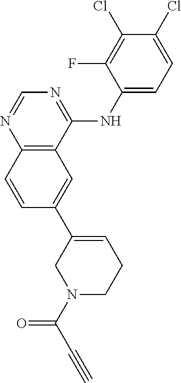 [SMILES: C#CC(=O)N1CCC=C(c2ccc3ncnc(Nc4ccc(Cl)c(Cl)c4F)c3c2)C1] | None | LC/MS (ESI, m/z): [M + 1]⁺ = 441.0 It showed two sets of peaks. ¹H NMR (a = 0.3, b = 0.7, 400 MHz, CDCl₃) δ 8.82 (s, 0.3 Ha), 8.78 (s, 0.7 Hb), 8.58 (t, J = 7.6 Hz, 0.3 Ha), 8.33 (t, J = 8.4 Hz, 0.7 Hb), 7.99-7.64 (m, 4 H), 7.38-7.34 (m, 1 H), 6.44-6.39 (m, 1 H), 4.73 (dd, J = 4.4, 2.2 Hz, 0.6 Ha), 4.61 (dd, J = 4.2, 2.0 Hz, 1.4 Hz), 3.99 (t, J = 5.8 Hz, 1.4 Hb), 3.86 (t, J = 5.8 Hz, 0.6 Ha), 3.25 (s, 0.3 Ha), 3.17 (s, 0.7 Ha), 2.57-2.51 (m, 1.4 Hb), 2.49-2.44 (m, 0.6 Ha). |
| 63 | 1-(5-(4-((3,4- dichloro-2- fluorophenyl)amino)- 7- methoxyquinazolin- 6-yl)-3,6- dihydropyridin- 1(2H)-yl)prop-2- yn-1-one | 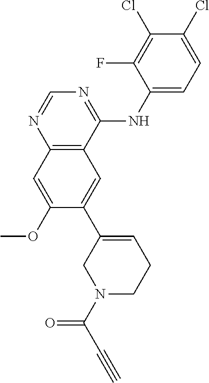 [SMILES: C#CC(=O)N1CCC=C(c2cc3c(Nc4ccc(Cl)c(Cl)c4F)ncnc3cc2OC)C1] | None | LC/MS (ESI, m/z): [M + H]⁺ = 471.1, 473.1. ¹H NMR (400 MHz, DMSO-d₆) δ 9.95 (s, 1 H), 8.46 (d, J = 2.5 Hz, 1 H), 8.27 (d, J = 8.6 Hz, 1 H), 7.59-7.54 (m, 2 H), 7.25 (d, J = 5.9 Hz, 1 H), 6.07 (d, J = 19.5 Hz, 1 H), 4.62 (d, J = 14.7 Hz, 2 H), 4.38 (s, 1H), 4.05-384 (m, 4 H), 3.71 (t, J = 6.2 Hz, 1 H), 2.33-2.31 (m, 2 H). |
| 64 | 1-(4-((7-methoxy- 4-(naphthalen-2- ylamino)quinazolin- 6-yl)oxy)piperidin- 1-yl)prop-2-yn-1- one | 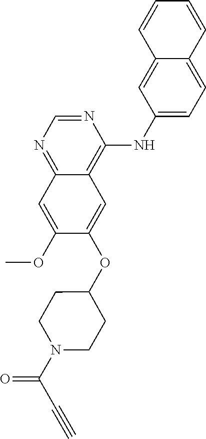 [SMILES: C#CC(=O)N1CCC(Oc2cc3c(Nc4ccc5ccccc5c4)ncnc3cc2OC)CC1] | None | LC/MS (ESI, m/z): [M + H]⁺ = 453.1. ¹H NMR (400 MHz, DMSO-d6) δ 11.21 (s, 1 H), 8.87 (s, 1 H), 8.21 (s, 1 H), 8.17 (d, J = 1.2 Hz, 1 H), 8.06 (d, J = 8.8 Hz, 1 H), 8.00-7.97 (m, 2 H), 7.80 (dd, J = 8.8 Hz, 1 H), 7.61-7.54 (m, 2 H), 7.28 (s, 1 H), 4.89 (m, 1 H), 4.58 (s, 1 H), 4.03 (s, 3H), 3.99-3.92 (m, 1 H), 3.78-3.73 (m, 2 H), 3.58-3.52 (m, 1 H), 2.17-1.99 (m, 2 H), 1.88-1.72 (m, 2 H). |

### Example 65: Preparation of 3-(4-((4-((3,4-dichloro-2-fluorophenyl)amino)-7-methoxyquinazolin-6-yl)oxy)piperidin-1-yl)-3-oxopropanenitrile (Compound 65)

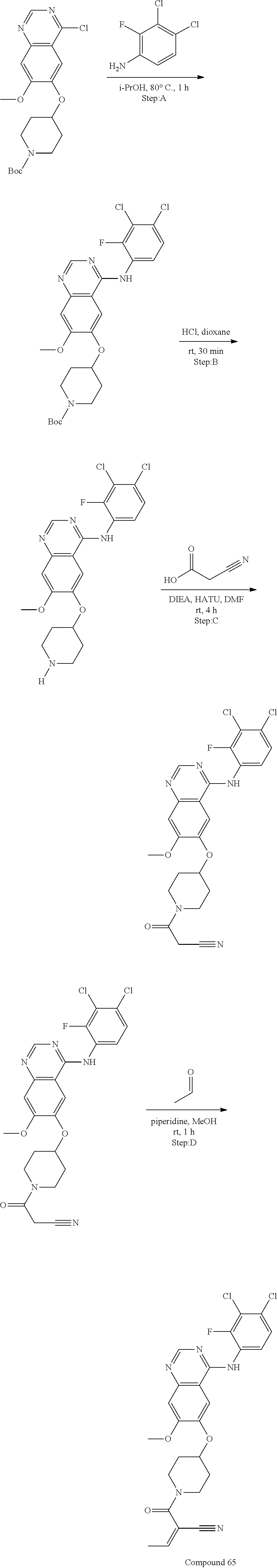

Step A: To a mixture of tert-butyl 4-((4-chloro-7-methoxyquinazolin-6-yl)oxy)piperidine-1-carboxylate (226 mg, 0.57 mmol) and 3,4-dichloro-2-fluoroaniline (103 mg, 0.57 mmol) in isopropanol was degassed with nitrogen, heated to 80° C. and stirred for 1 hour under nitrogen atmosphere. The reaction was cooled to r.t, filtered and concentrated in vacuo and purified by SGC to give 4-((7-fluoro-1-oxo-2,3-dihydro-1H-inden-4-yl)amino)-7-methoxyquinazolin-6-yl acetate (308 mg, 100% yield). LC/MS (ESI, m/z): [M+H]⁺=537.2.

Step B: A solution of tert-butyl 4-((4-((3,4-dichloro-2-fluorophenyl)amino)-7-methoxyquinazolin-6-yl)oxy)piperidine-1-carboxylate (308 mg, 0.57 mmol) in 4M HCl(g)-dioxane (5 mL) was stirred at rt for 30 min. The reaction mixture was concentrated in vacuo to afford N-(3,4-dichloro-2-fluorophenyl)-7-methoxy-6-(piperidin-4-yloxy)quinazolin-4-amine (251 mg, crude, 100% yield). LC/MS (ESI, m/z): [M+H]⁺=437.4.

Step C: A mixture of N-(3,4-dichloro-2-fluorophenyl)-7-methoxy-6-(piperidin-4-yloxy)quinazolin-4-amine (251 mg, 0.57 mmol), 2-cyanoacetic acid (48 mg, 0.57 mmol), HATU (542 mg, 1.43 mmol) and DIPEA (296 mg, 2.28 mmol) in DMF (15 mL) was degassed with nitrogen, stirred at RT for 1 hour under a nitrogen atmosphere. Water (30 mL) was added and the mixture was extracted with EA. The organic layer was washed with brine, dried over anhydrous sodium sulfate, filtered and concentrated in vacuo. The residue was purified by prep-HPLC to give 3-(4-((4-((3,4-dichloro-2-fluorophenyl)amino)-7-methoxyquinazolin-6-yl)oxy)piperidin-1-yl)-3-oxopropanenitrile (176 mg, 61% yield). LC/MS (ESI, m/z): [M+H]⁺=505.2. ¹H NMR (400 MHz, DMSO-d₆) δ 9.64 (s, 1H), 8.40 (s, 1H), 7.87 (s, 1H), 7.67-7.48 (m, 2H), 7.25 (s, 1H), 4.77 (s, 1H), 4.09 (s, 2H), 3.95 (s, 3H), 3.79 (s, 1H), 3.59 (s, 1H), 3.43 (s, 1H), 2.67 (s, 1H), 2.04 (s, 2H), 1.74 (d, J=30.3 Hz, 2H).

Step D: A solution of 3-(4-((4-((3,4-dichloro-2-fluorophenyl)amino)-7-methoxyquinazolin-6-yl)oxy)piperidin-1-yl)-3-oxopropanenitrile (30 mg, 0.06 mmol), acetaldehyde (5.2 mg, 0.12 mmol) and piperidine (10.2 mg, 0.12 mmol) in MeOH (5 mL) was stirred at RT for 1 h. The reaction mixture was concentrated in vacuo and purified by pre-HPLC to afford (Z)-2-(4-((4-((3,4-dichloro-2-fluorophenyl)amino)-7-methoxyquinazolin-6-yl)oxy)piperidine-1-carbonyl)but-2-enenitrile (10 mg, 32% yield). LC/MS (ESI, m/z): [M+H]⁺=530.0, 532.2. ¹H NMR (400 MHz, DMSO-d₆) δ 9.65 (br s, 1H), 8.39 (s, 1H), 7.89 (s, 1H), 7.58 (s, 2H), 7.24 (s, 1H), 6.95 (t, J=8.8 Hz, 0.5 H), 6.54 (q, J=5.2 Hz, 0.5 H), 4.80 (s, 1H), 3.95 (s, 3H), 3.88-3.79 (m, 2H), 3.61-3.43 (m, 2H), 2.60 (s, 3H), 2.11-2.00 (m, 2H), 1.85-1.71 (m, 2H).

### Example 66: Preparation of N-((1s,3s)-3-((4-((3,4-dichloro-2-fluorophenyl)amino)-7-methoxyquinazolin-6-yl)oxy)cyclobutyl)acrylamide (Compound 66)

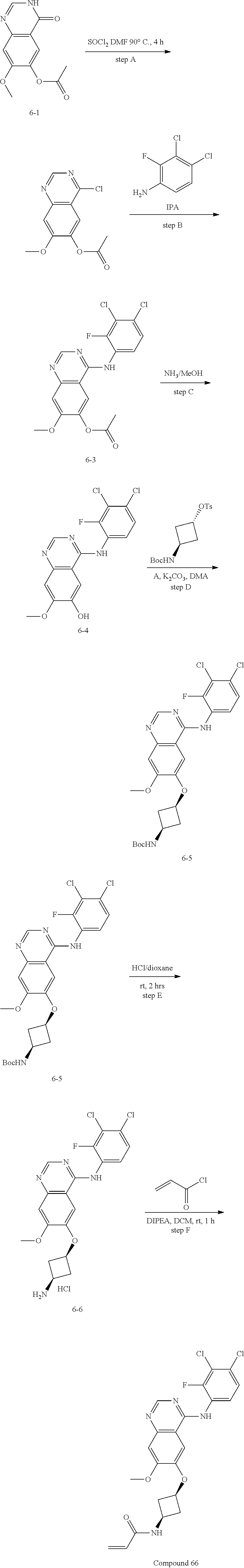

Step A: To a solution of 7-methoxy-4-oxo-3,4-dihydroquinazolin-6-yl acetate (500 mg, 2.13 mmol) in SOCl₂ (5 mL) was added DMF (20 mg). The mixture was stirred at 90° C. for 2 hours. The mixture was concentrated in vacuum to afford 4-chloro-7-methoxyquinazolin-6-yl acetate (539 mg, 100% yield) as a yellow solid. LC/MS (ESI, m/z): [M+H]⁺=253.0.

Step B: A mixture of 4-chloro-7-methoxyquinazolin-6-ylacetate (300 mg, 1.19 mmol) and 3,4-dichloro-2-fluoroaniline (192.37 mg, 1.19 mmol) in isopropanol (10 mL) was degassed with nitrogen, heated to 80° C. and stirred for 1 hour under nitrogen atmosphere. The reaction was cooled to r.t., filtered and concentrated in vacuum to give 4-((3,4-dichloro-2-fluorophenyl)amino)-7-methoxyquinazolin-6-yl acetate (344 mg, 77% yield) as a brown solid.

LC/MS (ESI, m/z): [M+H]⁺=396.0.

Step C: To a solution of 4-((3,4-dichloro-2-fluorophenyl)amino)-7-methoxyquinazolin-6-yl acetate in MeOH (4 mL) was slowly added ammonium hydroxide (2 mL) at RT. After addition, the reaction mixture was stirred at RT for 2 h. The reaction was then filtered and the cake was dried to give 4-((3,4-dichloro-2-fluorophenyl)amino)-7-methoxyquinazolin-6-ol (260 mg, 85% yield) as a white solid. LC/MS (ESI, m/z): [M+H]⁺=354.0.

Step D: A mixture of 4-((3,4-dichloro-2-fluorophenyl)amino)-7-methoxyquinazolin-6-ol (60 mg, 178.48 umol), (1r,3r)-3-((tert-butoxycarbonyl)amino)cyclobutyl 4-methylbenzenesulfonate (91.41 mg, 267.72 umol), and K₂CO₃ (49.3 mg, 356.96 umol) in DMA (2 mL) was degassed with nitrogen, heated to 115° C. and stirred for 2 hours under nitrogen atmosphere. Then the mixture was quenched with water (30 mL) and extracted with EtOAc (5 mL×3). The combined organic layers were washed with brine, dried over anhydrous sodium sulfate, filtered and concentrated in vacuum. The residue was purified by column chromatography (PE/EA=1/1) to give tert-butyl ((1s,3s)-3-((4-((3,4-dichloro-2-fluorophenyl)amino)-7-methoxyquinazolin-6-yl)oxy)cyclobutyl)carbamate (67 mg, 74% yield) as a white solid. LC/MS (ESI, m/z): [M+H]⁺=523.1.

Step E: tert-butyl ((1s,3s)-3-((4-((3,4-dichloro-2-fluorophenyl)amino)-7-methoxyquinazolin-6-yl)oxy)cyclobutyl)carbamate (356 mg, 680 umol) in 4M HCl dioxane(5 mL) was stirred for 1 hour at room temperature. The reaction was then concentrated in vacuum to give 6-((1s,3s)-3-aminocyclobutoxy)-N-(3,4-dichloro-2-fluorophenyl)-7-methoxyquinazolin-4-amine hydrochloride (296 mg, 94% yield) as a yellow solid. LC/MS (ESI, m/z): [M+H]⁺=423.1.

Step F: To a mixture of 6-((1s,3s)-3-aminocyclobutoxy)-N-(3,4-dichloro-2-fluorophenyl)-7-methoxyquinazolin-4-amine hydrochloride (300 mg, 649 umol), N-isopropyl-N-methylpropan-2-amine (252 mg, 194.7 umol) in DCM (5 mL) was added acryloyl chloride (59 mg, 649 umol) in DCM (1 mL) at 0° C. The reaction mixture was stirred for 0.5 h at room temperature, quenched by water, and then extracted with DCM (5 mL×3). The combined organic layers were washed with brine, dried over anhydrous sodium sulfate, filtered and concentrated in vacuum. The residue was purified by column chromatography (DCM:CH₃OH=20:1) to afford N-((1s,3s)-3-((4-((3,4-dichloro-2-fluorophenyl)amino)-7-methoxyquinazolin-6-yl)oxy)cyclobutyl)acrylamide (160 mg, 52% yield). LC/MS (ESI, m/z): [M+H]⁺=477.2, 479.2. ¹H NMR (400 MHz, DMSO-d₆) δ 9.67 (s, 1H), 8.47 (d, J=7.6 Hz, 1 H), 8.39 (s, 1H), 7.64-7.41 (m, 3H), 7.23 (s, 1H), 6.17 (dd, J=17.1, 9.7 Hz, 1H), 6.09 (dd, J=17.1, 2.7 Hz, 1H), 5.61 (dd, J=9.7, 2.7 Hz, 1H), 4.69-4.53 (m, 1H), 4.12-4.05 (m, 1H), 3.95 (s, 3H), 3.12-2.98 (m, 2H), 2.07 (dd, J=19.4, 9.0 Hz, 2H).

### Example 67: Preparation of N-(3-((7-methoxy-4-((7-methyl-2,3-dihydro-1H-inden-4-yl)amino)quinazolin-6-yl)oxy)cyclobutyl)acrylamide (Compound 67)

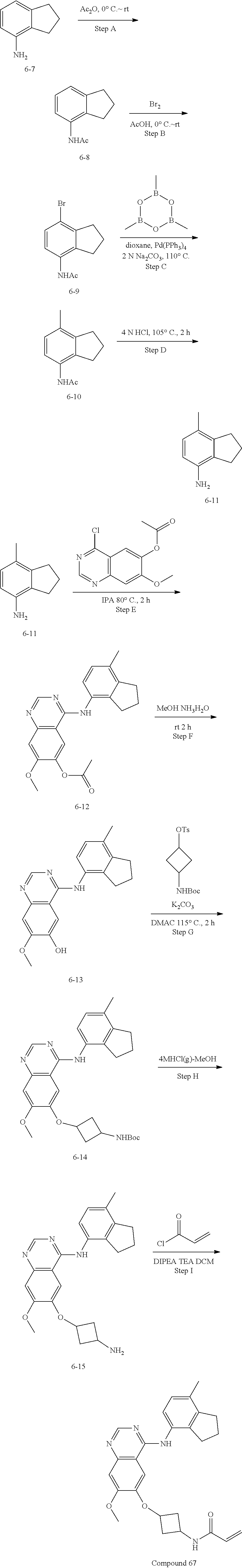

Step A: A mixture of 2,3-dihydro-1H-inden-4-amine (500 mg, 3.75 mmol) and acetic anhydride (5 mL) was stirred from 0° C. to RT for 1 h. The mixture was filtered. The filter cake was washed with water and dried to give the N-(2,3-dihydro-1H-inden-4-yl)acetamide (509 mg, 77% yield) as a white solid. LC/MS (ESI, m/z): [2M+H]⁺=351.4.

Step B: A solution of N-(2,3-dihydro-1H-inden-4-yl)acetamide (500 mg, 0.23 mmol) in AcOH was added Br₂ (912 mg, 5.7 mmol) dropwise at 0° C. The mixture was stirred from 0° C.˜RT for 1 h, and then the mixture was then quenched by Na₂CO₃ (aq) and Na₂S203 (aq), and then extracted with EA. The organic solution was concentrated in vacuum to give N-(7-bromo-2,3-dihydro-1H-inden-4-yl)acetamide as a white solid (700 mg, 97% yield) as a white solid. LC/MS (ESI, m/z): [M+H]⁺=254.1, 256.1.

Step C: A solution of N-(7-bromo-2,3-dihydro-1H-inden-4-yl)acetamide (254 mg, 1 mmol) in dioxane was added 2 N Na₂CO₃ (5 mL), tetrakis(triphenylphosphine)palladium (115 mg, 0.1 mmol), and 2,4,6-trimethyl-1,3,5,2,4,6-trioxatriborinane (188 mg, 1.5 mmol) at RT. The mixture was warmed to 110° C. and stirred for 4 h, The mixture was cooled, quenched by water, extracted with EA. The organic solution was washed with brine, dried by Na₂SO₄, filtered, and concentrated in vacuo to give N-(7-methyl-2,3-dihydro-1H-inden-4-yl)acetamide as a yellow solid (150 mg, 39% yield) as a yellow solid. LC/MS (ESI, m/z): [M+H]⁺=190.1.

Step D: A solution of N-(7-methyl-2,3-dihydro-1H-inden-4-yl)acetamide (150 mg, 0.8 mmol) in 4 N HCl (10 mL) was warmed up to 105° C. and stirred for 2 h. The mixture was cooled to RT, balanced pH with Na₂CO₃ (aq), and extracted with EA. The organic solution was washed with brine, dried by Na₂SO₄, filtered, and concentrated in vacuo. The residue was purified by column chromatography to give the N-(7-methyl-2,3-dihydro-1H-inden-4-yl)acetamide as a white solid (30 mg, 25% yield). LC/MS (ESI, m/z): [M+H]⁺=148.1.

Steps E, F, G, H, and I were completed in a similar manner as described in Example 66 steps B, C, D, E, and F to afford N-(3-((7-methoxy-4-((7-methyl-2,3-dihydro-1H-inden-4-yl)amino)quinazolin-6-yl)oxy)cyclobutyl)acrylamide (Compound 67). LC/MS (ESI, m/z): [M+H]⁺=445.2. ¹H NMR (400 MHz, DMSO-d₆) δ 10.99 (s, 1H), 8.69 (s, 1H), 8.56-8.54 (m, 1H), 7.81-7.73 (m, 1H), 7.27 (s, 1H), 7.13-7.03 (m, 2H), 6.27-6.03 (m, 2H), 5.61 (dd, J=10.0, 2.0 Hz, 1H), 5.02-5.01 (m, 1H), 4.47-4.35 (m, 1H), 4.01 (d, J=6.4 Hz, 3H), 2.88 (t, J=7.6 Hz, 2H), 2.75 (t, J=7.2 Hz, 2H), 2.68-2.54 (m, 4H), 2.28 (s, 3H), 2.09-1.93 (m, 2H).

### Example 68: Preparation of N-(3-((4-((7-fluoro-1-oxo-2,3-dihydro-1H-inden-4-yl)amino)-7-methoxyquinazolin-6-yl)oxy)cyclobutyl)acrylamide (Compound 68)

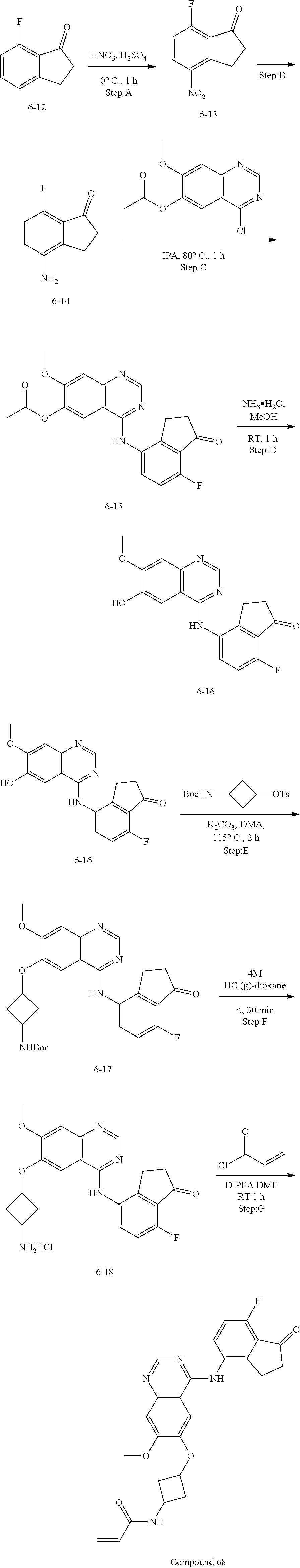

Step A: A solution of 7-fluoro-2,3-dihydro-1H-inden-1-one (500 mg, 3.33 mmol) in HNO₃ and H₂SO₄ (5 mL) was stirred at 0° C. for 60 min. H₂O (30 mL) was added and the mixture was extracted with EA. The organic phase was concentrated in vacuo and purified by SGC to afford 7-fluoro-4-nitro-2,3-dihydro-1H-inden-1-one (180 mg, 28% yield). ¹H NMR (400 MHz, CDCl₃-d) δ 8.50 (dd, J=9.0, 4.4 Hz, 1H), 7.23-7.19 (m, 1H), 3.68-3.65 (m, 2H), 2.84-2.81 (m, 2H).

Step B: A solution of 7-fluoro-4-nitro-2,3-dihydro-1H-inden-1-one (180 mg, 0.92 mmol) in MeOH (5 mL) was added Raney Ni (20 mg). The reaction mixture was purged with H₂ three times. The reaction mixture was stirred at RT for 4 h under H₂ balloon. The mixture was concentrated in vacuum to afford 4-amino-7-fluoro-2,3-dihydro-1H-inden-1-one (65 mg, 42% yield). LC/MS (ESI, m/z): [M+H]⁺=166.2.

Steps C, D, E, F and G were completed in a similar manner as described in Example 66 steps B, C, D, E, and F to afford N-(3-((4-((7-fluoro-1-oxo-2,3-dihydro-1H-inden-4-yl)amino)-7-methoxyquinazolin-6-yl)oxy)cyclobutyl)acrylamide (Compound 68). LC/MS (ESI, m/z): [M+H]⁺=463.2. ¹H NMR showed two pair of isomers, the ratio is 1/4 (a=1, b=4. cis/trans=1/4). ¹H NMR (400 MHz, DMSO-d₆) δ 10.85 (br s, 1H), 8.71 (s, 1H), 8.57 (d, J=6.8 Hz, 0.2 Ha), 8.49 (d, J=7.8 Hz, 0.8 Hb), 7.81-7.78 (m, 2H), 7.38-7.32 (m, 1H), 7.30 (s, 1H), 6.25-6.06 (m, 2H), 5.63-5.60 (m, 1H), 5.06-5.01 (m, 0.2 Ha, cis), 4.66-4.61 (m, 0.8 Hb, trans), 4.45-4.39 (m, 0.2 Ha, cis), 4.12-4.02 (m, 0.8 Hb, trans), 4.02 (s, 0.6 Ha, cis), 4.01 (s, 2.4 Hb, trans), 3.12-3.06 (m, 2H), 3.03-2.99 (m, 2H), 2.72-2.68 (m, 2H), 2.12-2.05 (m, 2H).

### Example 69: Preparation of N-(3-((4-((7-fluoro-1-hydroxy-2,3-dihydro-1H-inden-4-yl)amino)-7-methoxyquinazolin-6-yl)oxy)cyclobutyl)acrylamide (Compound 69)

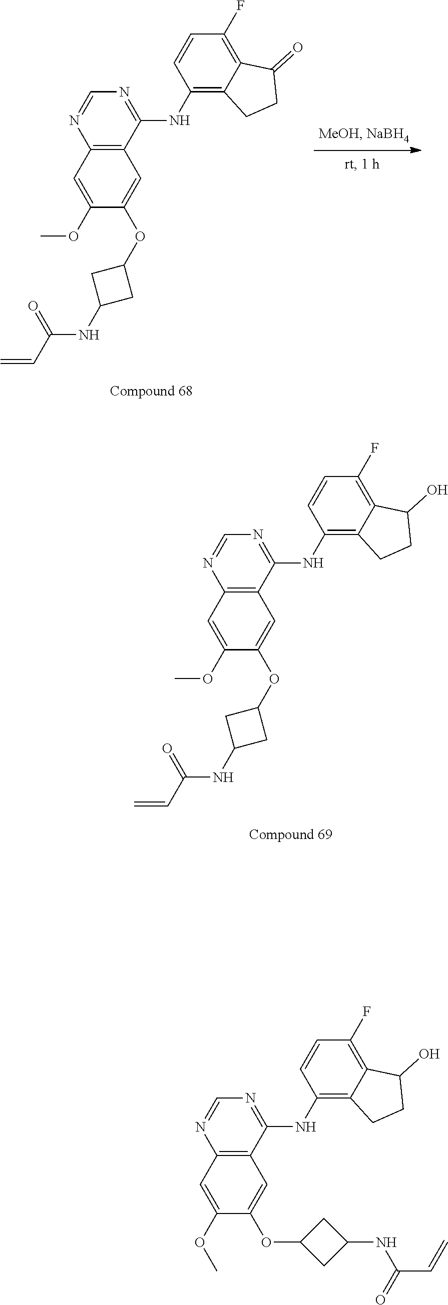 [SMILES: C=CC(=O)NC1CC(Oc2cc3c(Nc4ccc(F)c5c4CCC5=O)ncnc3cc2OC)C1 ; C=CC(=O)NC1CC(Oc2cc3c(Nc4ccc(F)c5c4CCC5O)ncnc3cc2OC)C1 ; C=CC(=O)NC1CC(Oc2cc3c(Nc4ccc(F)c5c4CCC5O)ncnc3cc2OC)C1]

A solution of N-(3-((4-((7-fluoro-1-oxo-2,3-dihydro-1H-inden-4-yl)amino)-7-methoxyquinazolin-6-yl)oxy)cyclobutyl)acrylamide (Compound 68) (10 mg, 0.03 mmol) in MeOH (5 mL) was added NaBH₄ (3 mg, 0.04 mmol) in one portion. The reaction was stirred at rt for 60 min. The mixture was concentrated in vacuo and purified by pre-HPLC to afford N-(3-((4-((7-fluoro-1-hydroxy-2,3-dihydro-1H-inden-4-yl)amino)-7-methoxyquinazolin-6-yl)oxy)cyclobutyl)acrylamide (Compound 69) (6 mg, 43% yield). LC/MS (ESI, m/z): [M+H]⁺=465.1. ¹H NMR showed two pair of isomers, the ratio is 1/4 (a=1, b=4. cis/trans=1/4). ¹H NMR (400 MHz, DMSO-d₆) δ 10.76 (br s, 1H), 8.68 (s, 1H), 8.56 (d, J=6.1 Hz, 0.2 Ha), 8.49 (d, J=7.5 Hz, 0.8 Hb), 7.74 (s, 0.8 Hb), 7.67 (s, 0.2 Ha), 7.36-7.32 (m, 1H), 7.28-7.25 (m, 1H), 7.16-7.11 (m, 1H), 6.25-6.06 (m, 2H), 5.63-5.60 (m, 1H), 5.35-5.33 (m, 1H), 5.04-4.99 (m, 0.2 Ha), 4.66-4.59 (m, 0.8 Hb), 4.44-4.39 (m, 0.2 Ha), 4.13-4.01 (m, 0.8 Hb), 4.01 (s, 0.6 Ha), 4.00 (s, 2.4 Hb), 3.11-3.04 (m, 2H), 3.01-2.88 (m, 1H), 2.68-2.61 (m, 1H), 2.34-2.23 (m, 1H), 2.10-2.00 (m, 2H), 1.94-1.88 (m, 1H).

### Example 70: Preparation of N-(3-((7-methoxy-4-((5-oxo-5,6,7,8-tetrahydronaphthalen-2-yl)amino)quinazolin-6-yl)oxy)cyclobutyl)acrylamide (Compound 70)

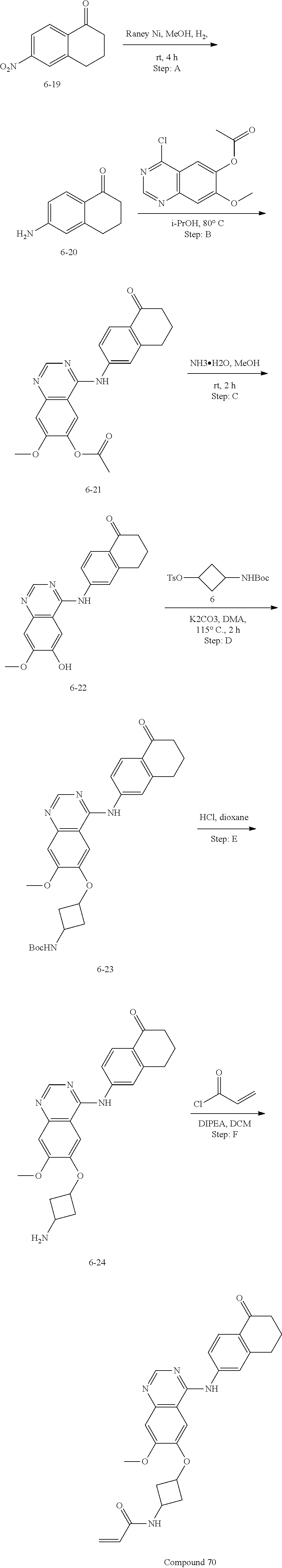

Step A: To a solution of 6-nitro-3,4-dihydronaphthalen-1(2H)-one (200 mg, 1.05 mmol) in MeOH (5 mL) was added Raney Ni (20 mg). The mixture was purged with H₂ for three times. The reaction was stirred at RT for 4 h under H₂ balloon. The mixture was concentrated in vacuum to afford 6-amino-3,4-dihydronaphthalen-1(2H)-one (168.6 mg, crude, 100% yield). LC/MS (ESI, m/z): [M+1]⁺=162.2.

Steps B, C, D, E, and F were completed in a similar manner as described in Example 66 steps B, C, D, E, and F to afford N-(3-((7-methoxy-4-((5-oxo-5,6,7,8-tetrahydronaphthalen-2-yl)amino)quinazolin-6-yl)oxy)cyclobutyl)acrylamide (Compound 70). LC/MS (ESI, m/z): [M+H]⁺=459.1. ¹H NMR showed two pair of isomers, the ratio is 3/7 (a=3, b=7. cis/trans=3/7). ¹H NMR (400 MHz, DMSO-d₆) δ 10.89 (br s, 1H), 8.79 (s, 1H), 8.57 (d, J=4.8 Hz, 0.3 Ha), 8.50 (d, J=7.4 Hz, 0.7 Hb), 8.10 (d, J=2.3 Hz, 0.7 Hb), 8.08 (d, J=2.2 Hz, 0.3 Ha), 7.90 (dd, J=8.2, 2.0 Hz, 1H), 7.81 (s, 0.7 Hb), 7.74 (s, 0.3 Ha), 7.50-7.47 (m, 1H), 7.30-7.26 (m, 1H), 6.26-6.05 (m, 2H), 5.63-5.57 (m, 1H), 5.08-5.03 (m, 0.3 Ha), 4.71-4.63 (m, 0.7 Hb), 4.46-4.39 (m, 0.3 Ha), 4.19-4.05 (m, 0.7 Hb), 4.02 (s, 0.9 Ha), 4.00 (s, 2.1 Hb), 3.12-3.05 (m, 2H), 3.00 (t, J=5.8 Hz, 2H), 2.68-2.60 (m, 3H), 2.12-2.05 (m, 3H).

### Example 71: Preparation of N-(3-((4-((5-hydroxy-5,6,7,8-tetrahydronaphthalen-2-yl)amino)-7-methoxyuinazolin-6-yl)oxy)cyclobutyl)acrylamide (Compound 71)

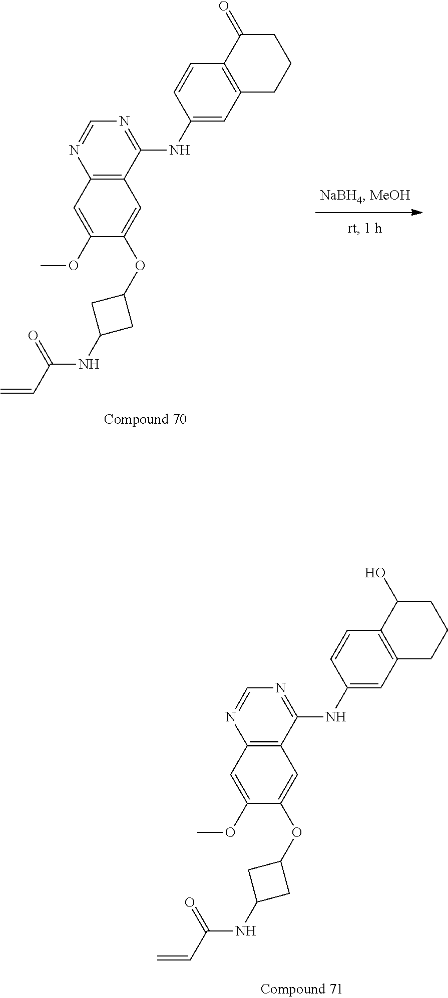 [SMILES: C=CC(=O)NC1CC(Oc2cc3c(Nc4ccc5c(c4)CCCC5=O)ncnc3cc2OC)C1 ; C=CC(=O)NC1CC(Oc2cc3c(Nc4ccc5c(c4)CCCC5O)ncnc3cc2OC)C1]

N-(3-((4-((5-hydroxy-5,6,7,8-tetrahydronaphthalen-2-yl)amino)-7-methoxyquinazolin-6-yl)oxy)cyclobutyl)acrylamide (Compound 71) was prepared from N-(3-((7-methoxy-4-((5-oxo-5,6,7,8-tetrahydronaphthalen-2-yl)amino)quinazolin-6-yl)oxy)cyclobutyl)acrylamide (Compound 70) in a similar manner as described in Example 69. LC/MS (ESI, m/z): [M+H]⁺=461.4. ¹H NMR showed two pair of isomers, the ratio is 3/7 (a=3, b=7. cis/trans=3/7). ¹H NMR (400 MHz, DMSO-d₆) δ 10.79 (br s, 1H), 8.73 (s, 1H), 8.57-8.55 (m, 0.3 Ha), 8.49 (d, J=7.6 Hz, 0.7 Hb), 7.81 (s, 0.7 Hb), 7.74 (s, 0.3 Ha), 7.59 (d, J=2.0 Hz, 0.7 Hb), 7.57 (d, J=2.0 Hz, 0.3 Ha), 7.48-7.44 (m, 1H), 7.27-7.26 (m, 1H), 7.18-7.16 (m, 1H), 6.26-6.06 (m, 2H), 5.61 (dd, J=9.6, 2.8 Hz, 1H), 5.26 (br s, 1H), 5.07-5.02 (m, 0.3 Ha), 4.69-4.65 (m, 0.7 Hb), 4.61-4.57 (m, 1H), 4.47-4.40 (m, 0.3 Ha), 4.12-4.06 (m, 0.7 Hb), 4.01 (s, 0.9 Ha), 4.00 (s, 2.1 Hb), 3.13-3.05 (m, 2H), 2.79-2.65 (m, 2H), 2.11-2.02 (m, 2H), 1.99-1.88 (m, 2H), 1.74-1.68 (m, 2H).

### Example 72: Preparation of N-(3-((4-((3,4-dichloro-2-fluorophenyl)amino)-7-methoxyuinazolin-6-yl)oxy)cyclobutyl)-N-methylacrylamide (Compound 72)

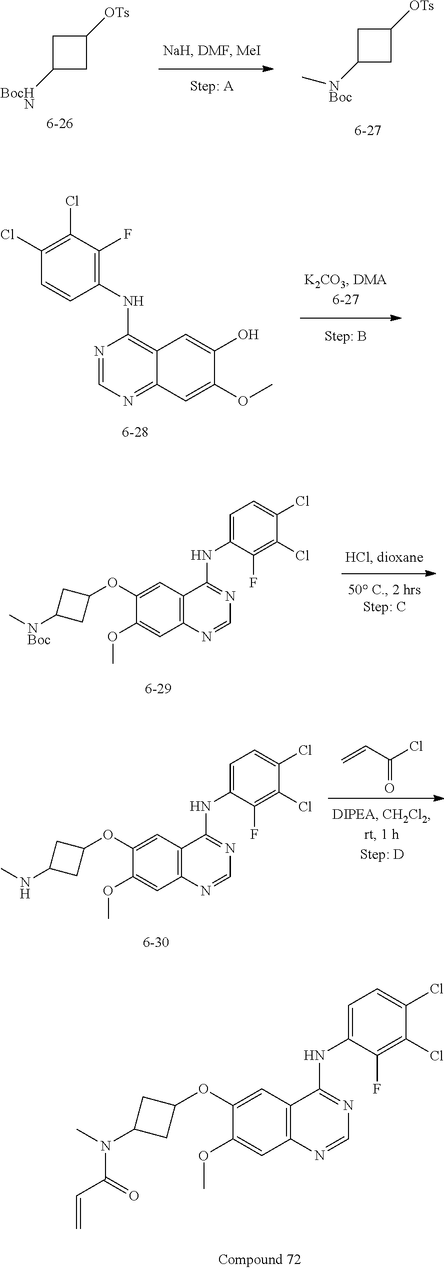

Step A: To a solution of 3-((tert-butoxycarbonyl)amino)cyclobutyl 4-methylbenzenesulfonate (350 mg, 1.03 mmol) in dry DMF (5 mL) was added NaH (62 mg, 1.55 mmol) (60% in mineral oil) at 0° C. under N₂. The reaction mixture was stirred at 0° C. for 20 min. Then Mel (176 mg, 1.236 mmol) was added to the reaction mixture. The reaction mixture was stirred at room temperature of 2 h. The reaction mixture was poured into ice water, extracted with EtOAc (10 mL×3). The combined organic layers were washed with brine, dried over anhydrous Na₂SO₄. The solid was filtered and the filtrate was concentrated to give a crude product which was purified by column chromatography on silica gel (Pe/EA=10/1 to 5/1) to give the product 3-((tert-butoxycarbonyl)(methyl)amino)cyclobutyl 4-methylbenzenesulfonate as a white solid (200 mg, 55% yield). LC/MS (ESI, m/z): [M+H]⁺=356.2.

Steps B, C, and D were completed in a similar manner as described in Example 66 steps D, E, and F to afford N-(3-((4-((3,4-dichloro-2-fluorophenyl)amino)-7-methoxyquinazolin-6-yl)oxy)cyclobutyl)-N-methylacrylamide (Compound 72). LC/MS (ESI, m/z): [M+1]⁺=491.1, 493.1. ¹H NMR (400 MHz, DMSO-d₆) δ 9.72 (s, 1H), 8.39 (s, 1H), 7.69-7.41 (m, 3H), 7.25 (s, 1H), 6.74 (dd, J=16.3, 10.7 Hz, 1H), 6.12-6.03 (m, 1H), 5.67 (s, 1H), 5.23-4.74 (m, 2 H), 3.97 (s, 3H), 3.05-2.95 (m, 3H), 2.84-2.80 (m, 2H), 2.43-2.37 (m, 2H).

Examples 73-91 were prepared using similar procedures as described in the preceding Examples.

| EX. | | Compound | | |
|---|---|---|---|---|
| NO. | IUPAC Name | structure | salt | Compound characterization |
| 73 | N-(3-((4-((3,4- dichlorophenyl) amino)-7- methoxyquinazo- lin-6- yl)oxy)cyclobu- tyl)acrylamide | 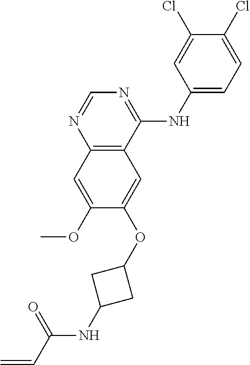 [SMILES: C=CC(=O)NC1CC(Oc2cc3c(Nc4ccc(Cl)c(Cl)c4)ncnc3cc2OC)C1] | None | LC/MS (ESI, m/z): [M + 1]+ = 459.0, 461.0. H NMR showed cis/trans = 2/8. ¹H NMR (400 MHz, DMSO-d₆) δ 10.70 (br s, 1 H), 8.81 (s, 1 H), 8.56 (d, J = 3.5 Hz, 0.8 H, trans), 8.49 (d, J = 7.6 Hz, 0.2 H, cis), 8.09-8.05 (m, 1 H), 7.80- 7.61 (m, 3 H), 7.31 (s, 1 H), 6.30-6.01 (m, 2 H), 5.63-5.60 (m, 1 H), 5.10-4.96 (m, 0.8 H, trans), 4.71-4.63 (m, 0.2 H, cis), 4.48-4.38 (m, 0.8 H, trans), 4.14-4.08 (m, 0.2 H, cis), 4.02 (s, 2.4 H, trans), 4.00 (s, 0.6 H, cis), 2.69-2.59 (m, 2 H), 2.49-2.41 (m, 2 H). |
| 74 | N-(3-((4-((3- chloro-2- fluorophenyl)a- mino)-7- methoxyquinazo- lin-6- yl)oxy)cyclobu- tyl)acrylamide | 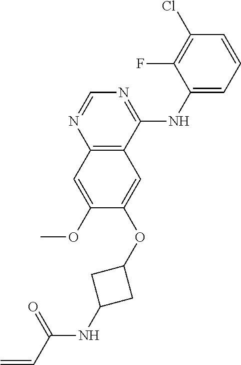 [SMILES: C=CC(=O)NC1CC(Oc2cc3c(Nc4cccc(Cl)c4F)ncnc3cc2OC)C1] | | LC/MS (ESI, m/z): [M + H]⁺ = 443.2, 446.2. ¹H NMR showed cis/trans = 4/6. ¹H NMR (400 MHz, DMSO-d₆) δ 8.63 (s, 1 H), 8.58 (d, J = 4.8 Hz, 0.6 H, trans), 8.49 (d, J = 7.6 Hz, 0.4 H, cis), 7.73-7.53 (m, 3 H), 7.41-7.33 (m, 1 H), 7.32- 7.28 (m, 1H), 6.25-6.06 (m, 2 H), 5.61 (dd, J = 9.6, 2.7 Hz, 1 H), 5.06-5.03 (m, 0.6 H, trans), 4.66-4.62 (m, 0.4 H, cis), 4.45-4.39 (m, 0.6 H, trans), 4.12-4.06 (m, 0.4 H, cis), 4.02 (s, 1.8 H, trans, 4.00 (s, 1.2 H, cis), 3.12-3.05 (m, 1 H), 2.67-2.60 (m, 1 H), 2.48-2.44 (m, 1 H), 2.12- 2.05 (m, 1 H). |
| 75 | N-(3-((4-((2,4- dichloro-3- fluorophenyl)a- mino)-7- methoxyquinazo- lin-6- yl)oxy)cyclobu- tyl)acrylamide | 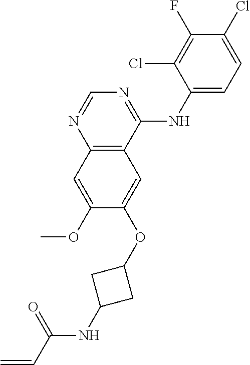 [SMILES: C=CC(=O)NC1CC(Oc2cc3c(Nc4ccc(Cl)c(F)c4Cl)ncnc3cc2OC)C1] | None | LC/MS (ESI, m/z): [M + H]⁺ = 477.0, 479.0 ¹H NMR showed cis/trans = (7/3). ¹H NMR (400 MHz, DMSO-d₆) δ 8.67-8.60 (m, 1 H), 8.57-8.46 (m, 1 H), 7.77-7.73 (m, 1 H), 7.66- 7.62 (m, 0.3 H, trans), 7.55-7.51 (m, 0.7 H, cis), 7.30-7.25 (m, 1 H), 6.25-6.05 (m, 2 H), 5.61 (dd, J = 9.6, 2.7 Hz, 1 H), 5.06-5.00 (m, 0.3 H, trans), 4.67-4.59 (m, 0.7 H, cis), 4.44-4.36 (m, 0.3 H, trans), 4.11-4.09 (m, 0.7 H, cis), 4.01 (s, 0.9 H, trans), 3.99 (s, 2.1 H, cis), 3.13-3.03 (m, 1 H), 2.65-2.57 (m, 1 H), 2.46-2.40 (m, 1 H), 2.12- 2.03 (m, 1 H). |
| 76 | N-(3-((4-((2,3- dichloro-4- fluorophenyl)a- mino)-7- methoxyquinazo- lin-6- yl)oxy)cyclobu- tyl)acrylamide | 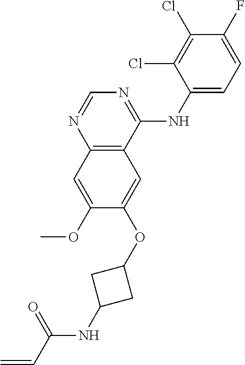 [SMILES: C=CC(=O)NC1CC(Oc2cc3c(Nc4ccc(F)c(Cl)c4Cl)ncnc3cc2OC)C1] | TFA | LC/MS (ESI, m/z): [M + H]⁺ = 477.0. H NMR showed cis/trans = (1/4). ¹H NMR (400 MHz, DMSO-d₆) δ 10.96 (br s, 1 H), 8.69-8.68 (m, 1 H), 8.58 (d, J = 5.6 Hz, 0.8 H, trans), 8.49 (d, J = 7.6 Hz, 0.2 H, cis), 7.74-7.59 (m, 3 H), 7.30- 7.29 (m, 1 H), 6.25-6.06 (m, 2 H), 5.63-5.60 (m, 1 H), 5.06-5.00 (m, 0.8 H, trans), 4.65-4.62 (m, 0.2 H, cis), 4.45-4.37 (m, 0.8 H, trans), 4.12- 4.06 (m, 0.2 H, cis), 4.02 (s, 2.4 H, trans), 4.00 (s, 0.6 H, cis), 2.69-2.60 (m, 2 H), 2.49-2.44 (m, 2 H). |
| 77 | N-(3-((4-((2- fluorophenyl)a- mino)-7- methoxyquinazo- lin-6- yl)oxy)cyclobu- tyl)acrylamide | 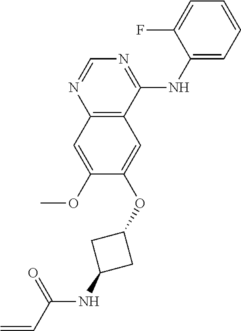 [SMILES: C=CC(=O)N[C@H]1C[C@H](Oc2cc3c(Nc4ccccc4F)ncnc3cc2OC)C1] | None | LC/MS (ESI, m/z): [M + H]⁺ = 409.2. ¹H NMR (400 MHz, DMSO-d₆) δ 9.48 (s, 1 H), 8.54 (d, J = 6.8 Hz, 1 H), 8.32 (s, 1 H), 7.55-7.46 (m, 2 H), 7.33-7.28 (m, 2 H), 7.28-7.22 (m, 1 H), 7.20 (s, 1 H), 6.22 (dd, J = 17.2, 10.0 Hz, 1 H), 6.10 (dd, J = 17.2, 2.4 Hz, 1 H), 5.60 (dd, J = 10.0, 2.0 Hz, 1 H), 5.01-4.97 (m, 1 H), 4.44- 4.38 (m, 1 H), 3.95 (s, 3 H), 2.68-2.56 (m, 2 H), 2.51-2.32 (m, 2 H). |
| 78 | N-(3-((4-((4- chloro-2- fluorophenyl)a- mino)-7- methoxyquinazo- lin-6- yl)oxy)cyclobu- tyl)acrylamide | 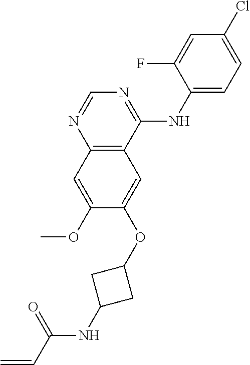 [SMILES: C=CC(=O)NC1CC(Oc2cc3c(Nc4ccc(Cl)cc4F)ncnc3cc2OC)C1] | None | LC/MS (ESI, m/z): [M +1]⁺ = 443.1, 445.1 ¹H NMR showed two pair of isomers, the ratio is cis/trans = 1/1. ¹H NMR (400 MHz, DMSO-d₆) δ 10.70 (br, 1 H), 8.69 (s, 1 H), 8.58-8.45 (m, 1 H), 7.80-7.54 (m, 3 H), 7.46-7.34 (m, 1 H), 7.31-7.19 (m, 1 H), 6.27-6.05 (m, 2 H), 5.61 (dd, J = 9.8, 2.5 Hz, 1 H), 5.07-4.97 (m, 0.5 H, trans), 4.68-4.58 (m, 0.5 H, cis), 4.46-4.36 (m, 0.5 H, trans), 4.15-4.04 (m, 0.5 H, cis), 4.01 (d, J = 6.7 Hz, 3 H), 3.13-3.02 (m, 1 H), 2.69-2.58 (m, 1 H), 2.48-2.38 (m, 1 H), 2.14- 2.02 (m, 1 H). |
| 79 | N-((1r,3r)-3- ((4-((3,4- dichloro-2- fluorophenyl)a- mino)-7- methoxyquinazo- lin-6- yl)oxy)cyclobu- tyl)acrylamide | 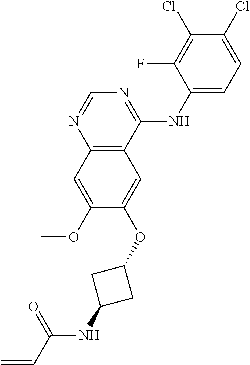 [SMILES: C=CC(=O)N[C@H]1C[C@H](Oc2cc3c(Nc4ccc(Cl)c(Cl)c4F)ncnc3cc2OC)C1] | None | LC/MS (ESI, m/z): [M + H]⁺ = 477.1, 479.1. 1H NMR (400 MHz, DMSO-d₆) δ 9.72 (br s, 1 H), 8.57 (br s, 1 H), 8.38 (s, 1 H), 7.61-7.55 (m, 2 H), 7.49 (s, 1 H), 7.23 (s, 1 H), 6.22 (dd, J = 16.8, 9.6 Hz, 1 H), 6.10 (dd, J = 16.8, 2.4 Hz, 1 H), 5.60 (dd, J = 10.0, 2.4 Hz, 1 H), 5.65-5.57 (m, 1 H), 4.45-4.34 (m, 1 H), 3.96 (s, 3 H), 2.67- 2.46 (m, 4 H). |
| 80 | N-(3-((7- methoxy-4- (naphthalen-2- ylamino)quinazo- lin-6- yl)oxy)cyclobu- tyl)acrylamide | 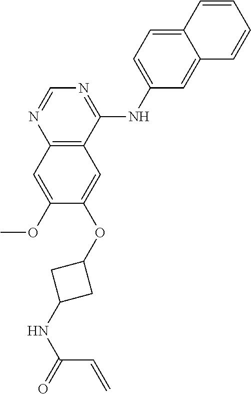 [SMILES: C=CC(=O)NC1CC(Oc2cc3c(Nc4ccc5ccccc5c4)ncnc3cc2OC)C1] | None | LC/MS (ESI, m/z): [M + H]⁺ = 441.1. H NMR showed cis/trans = (3/7) ¹H NMR (400 MHz, DMSO-d₆) δ 11.03 (br s, 1 H), 8.79 (s, 1 H), 8.57 (d, J = 6.8 Hz, 0.7 H, trans), 8.50 (d, J = 7.6 Hz, 0.3 H, cis), 8.05-8.02 (m, 1 H), 7.98- 7.95 (m, 2 H), 7.76-7.76 (m, 2 H), 7.59-7.52 (m, 2 H), 7.30-7.29 (m, 1 H), 6.26-6.07 (m, 2 H), 5.62 (dd, J = 9.9, 2.4 Hz, 1H), 5.10-5.06- 4.96 (m, 0.7 H, trans), 4.72-4.67 (m, 0.3 H, cis), 4.49-4.40 (m, 0.7 H, trans), 4.15-4.08 (m, 0.3 H, cis), 4.03 (s, 2.1 H, trans), 4.01 (s, 0.9 H, cis), 3.14-3.08 (m, 1 H), 2.73-2.57 (m, 2 H), 2.13-2.06 (m, 1 H). |
| 81 | N-(3-((7- methoxy-4- (naphthalen-1- ylamino)quinazo- lin-6- yl)oxy)cyclobu- tyl)acrylamide | 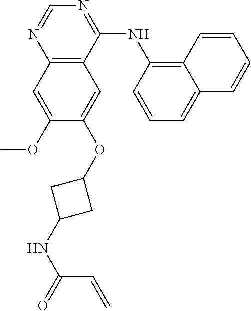 [SMILES: C=CC(=O)NC1CC(Oc2cc3c(Nc4cccc5ccccc45)ncnc3cc2OC)C1] | None | LC/MS (ESI, m/z): [M + 1]⁺ = 441.2. ¹H NMR (400 MHz, DMSO-d₆) δ 10.01 (br s, 1 H), 8.55 (d, J = 6.7 Hz, 1 H), 8.22 (s, 1 H), 8.01 (d, J = 7.9 Hz, 1 H), 7.92 (d, J = 7.3 Hz, 1 H), 7.84 (d, J = 8.4 Hz, 1 H), 7.71 (s, 1 H), 7.63-7.44 (m, 4 H), 7.22 (s, 1 H), 6.19 (dd, J = 22.0, 11.0 Hz, 1 H), 6.08 (dd, J = 17.1, 2.3 Hz, 1 H), 5.59 (dd, J = 9.7, 2.4 Hz, 1 H), 5.07-5.03 (m, 1 H), 4.45- 4.41 (m, 1 H), 3.97 (s, 3 H), 2.63-2.53 (m, 2 H), 1.28 (m, 2 H). |
| 82 | N-((1s,3s)-3- ((4-((3,4- dichlorophenyl) amino)-7- methoxyquinazo- lin-6- yl)oxy)cyclobu- tyl)acrylamide | 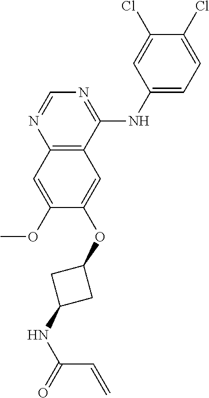 [SMILES: C=CC(=O)N[C@H]1C[C@@H](Oc2cc3c(Nc4ccc(Cl)c(Cl)c4)ncnc3cc2OC)C1] | TFA | LC/MS (ESI, m/z): [M + H]⁺ = 459.0, 461.0. ¹H NMR (400 MHz, DMSO-d₆) δ 10.61 (br s, 1 H), 8.79 (s, 1 H), 8.49 (d, J = 7.4 Hz, 1 H), 8.10 (s, 1 H), 7.74 (s, 3 H), 7.30 (s, 1 H), 6.18- 6.05 (m, 2 H), 5.61 (dd, J = 9.6, 2.7 Hz, 1 H), 4.72-4.59 (m, 1 H), 4.15-4.05 (m, 1 H), 4.00 (s, 3 H), 3.11-3.02 (m, 2 H), 2.11-2.04 (m, 2 H). |
| 83 | N-((1s,3s)-3- ((7-methoxy-4- (naphthalen-1- ylamino)quinazo- lin-6- yl)oxy)cyclobu- tyl)acrylamide | 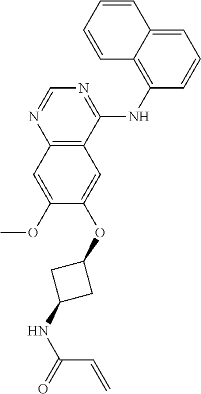 [SMILES: C=CC(=O)N[C@H]1C[C@@H](Oc2cc3c(Nc4cccc5ccccc45)ncnc3cc2OC)C1] | None | LC/MS (ESI, m/z): [M + H]⁺ = 441.0.¹H NMR (400 MHz, DMSO-d₆) δ 9.81 (s, 1 H), 8.47 (d, J = 7.6 Hz, 1 H), 8.18 (s, 1 H), 8.01 (d, J = 8.1 Hz, 1 H), 7.91 (d, J = 7.3 Hz, 1 H), 7.86 (d, J = 8.4 Hz, 1 H), 7.75 (s, 1 H), 7.62-7.48 (m, 4 H), 7.21 (s, 1 H), 6.17 (dd, J = 17.1, 9.8 Hz, 1 H), 6.08 (dd, J = 17.1, 2.5 Hz, 1 H), 5.60 (dd, J = 9.7, 2.5 Hz, 1 H), 4.66-4.62 (m, 1 H), 4.07- 4.05 (m, 1 H), 3.95 (s, 3H), 3.15-3.02 (m, 2 H), 2.09-2.04 (m, 2 H). |
| 84 | N-(3-((4-((4- fluorobenzyl)a- mino)-7- methoxyquinazo- lin-6- yl)oxy)cyclobu- tyl)acrylamide | 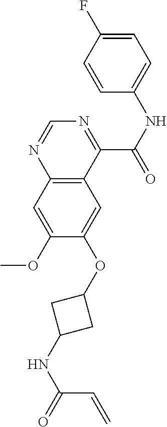 | None | LC/MS (ESI, m/z): [M + H]⁺ = 423.2. H NMR showed the ratio of cis/trans is about 1/1. ¹H NMR (400 MHz, DMSO-d6, a = trans, b = cis) δ 10.06 (s, 1 H), 8.79 (s, 1 H), 8.54-8.46 (m, 1 H), 7.62 (s, 0.5 H), 7.55 (s, 0.5 H), 7.45-7.40 (m, 2 H), 7.22-7.17 (m, 2 H), 6.24-6.02 (m, 2 H), 5.62-5.60 (m, 1 H), 5.00-4.96 (m, 0.5 Ha), 4.93 (s, 2 H), 4.65-4.55 (m, 0.5 Hb), 4.41-4.35 (m, 0.5 Ha), 4.09-4.04 (m, 0.5 Hb), 3.99 (s, 1.5 H), 3.97 (s, 1.5 H), 3.10-3.00 (m, 1 H), 2.47-2.42 (m, 2 H), 2.07-2.01 (m, 1 H). |
| 85 | N-(3-((4-((3- chloro-4- fluorophenyl)a- mino)-7- methoxyquinazo- lin-6- yl)oxy)cyclobu- tyl)acrylamide | 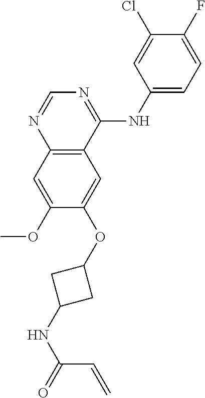 [SMILES: C=CC(=O)NC1CC(Oc2cc3c(Nc4ccc(F)c(Cl)c4)ncnc3cc2OC)C1] | None | LC/MS (ESI, m/z): [M + H]⁺ = 443.1, 445.1. H NMR showed the ratio of cis/trans is about 3/7. 1H NMR (400 MHz, DMSO-d₆) δ 10.67 (br s, 1 H), 8.76 (s, 1 H), 8.58 (d, J = 6.6 Hz, 0.7 H, trans), 8.49 (d, J = 8.0 Hz, 0.3 H, cis), 8.14- 7.84 (m, 1 H), 7.83-7.61 (m, 2 H), 7.54 (td, J = 9.1, 3.1 Hz, 1 H), 7.29 (s, 1 H), 6.37-5.90 (m, 2 H), 5.62 (dd, J = 10.1, 2.1 Hz, 1 H), 5.07- 5.02 (m, 0.7 H, trans), 4.68-4.64 (m, 0.3 H, cis), 4.47-4.38 (m, 0.7 H, trans), 4.13-4.07 (m, 0.3 H, cis), 4.01 (s, 2.1 H, trans), 3.99 (s, 0.9 H, cis), 3.12-3.02 (m, 1 H), 2.66-2.56 (m, 2 H), 2.12- 2.04 (m, 1 H). |
| 86 | N-(3-((4-((3- chloro-4- cyanophenyl)a- mino)-7- methoxyquinazo- lin-6- yl)oxy)cyclobu- tyl)acrylamide | 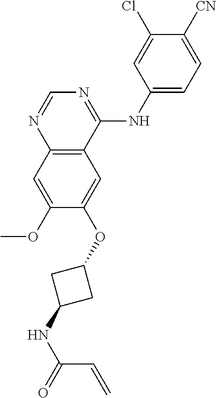 [SMILES: C=CC(=O)N[C@H]1C[C@H](Oc2cc3c(Nc4ccc(C#N)c(Cl)c4)ncnc3cc2OC)C1] | TFA | LC/MS (ESI, m/z): [M + H]⁺ = 450.2. ¹H NMR (400 MHz, DMSO-d₆) δ 10.48 (br s, 1 H), 8.82 (s, 1 H), 8.55 (d, J = 6.6 Hz, 1 H), 8.28 (s, 1 H), 8.02-7.96 (m, 2 H), 7.65 (s, 1 H), 7.32 (s, 1 H), 6.23 (dd, J = 17.1, 10.0 Hz, 1 H), 6.11 (dd, J = 17.1, 2.3 Hz, 1 H), 5.62 (dd, J = 10.0, 2.3 Hz, 1 H), 5.10 - 5.01 (m, 1 H), 4.47 - 4.41 (m, 1 H), 4.02 (s, 3 H), 2.64-2.58 (m, 2 H), 2.50-2.46 (m, 2 H). |
| 87 | N-(3-((4-((3- cyano-4- fluorophenyl)a- mino)-7- methoxyquinazo- lin-6- yl)oxy)cyclobu- tyl)acrylamide | 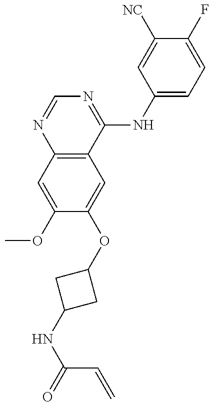 [SMILES: C=CC(=O)NC1CC(Oc2cc3c(Nc4ccc(F)c(C#N)c4)ncnc3cc2OC)C1] | | LC/MS (ESI, m/z): [M + H]⁺ = 434.2. ¹H NMR (400 MHz, DMSO-d₆) δ 9.69 (s, 1 H), 8.62- 8.45 (m, 2 H), 8.40-8.26 (m, 1 H), 8.17- 8.03 (m, 1 H), 7.59-7.54 (m, 2 H), 7.25 (s, 1 H), 6.23 (dd, J = 17.1, 10.0 Hz, 1 H), 6.10 (dd, J = 17.1, 2.3 Hz, 1 H), 5.62 (dd, J = 10.0, 2.3 Hz, 1 H), 5.08 - 4.98 (m, 1 H), 4.45-4.41 (m, 1), 3.97 (s, 3H), 2.59-2.51 (m, 2 H), 2.50-2.46 (m, 2 H). |
| 88 | N-(3-((4-((2,3- dihydro-1H- inden-4- yl)amino)-7- methoxyquinazo- lin-6- yl)oxy)cyclobu- tyl)acrylamide | 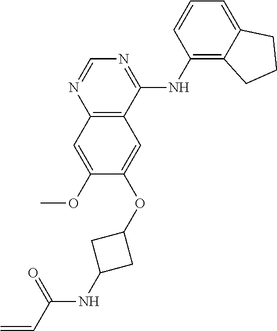 [SMILES: C=CC(=O)NC1CC(Oc2cc3c(Nc4cccc5c4CCC5)ncnc3cc2OC)C1] | None | LC/MS (ESI, m/z): [M + H]⁺ = 431.2. H NMR showed mainly trans. ¹H NMR (400 MHz, DMSO-d₆) δ 11.08 (br s, 1 H), 8.73 (s, 1 H), 8.57 (d, J = 6.5 Hz, 1 H), 7.70 (s, 1 H), 7.33- 7.24 (m, 3 H), 7.18 (dd, J = 6.1, 2.7 Hz, 1H), 6.22 (dd, J = 10.0, 7.0 Hz, 1 H), 6.10 (dd, J = 17.2, 2.2 Hz, 1H), 5.61 (dd, J = 10.0, 2.3 Hz, 1 H), 5.05-4.99 (m, 1 H), 4.46-4.37 (m, 1 H), 4.02 (s, 3 H), 2.97 (t, J = 7.3 Hz, 2 H), 2.74 (t, J = 7.2 Hz, 2 H), 2.66-2.59 (m, 2 H), 2.04-1.97 (m, 2 H). |
| 89 | N-(3-((7- methoxy-4- ((5,6,7,8- tetrahydronaph- thalen-1- yl)amino)quina- zolin-6- yl)oxy)cyclobu- tyl)acrylamide | 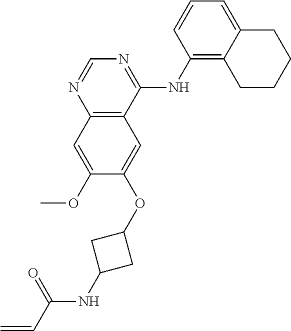 [SMILES: C=CC(=O)NC1CC(Oc2cc3c(Nc4cccc5c4CCCC5)ncnc3cc2OC)C1] | None | LC/MS (ESI, m/z): [M + H]⁺ = 445.2. ¹H NMR (400 MHz, DMSO-d₆) δ 9.22 (s, 1 H), 8.52 (d, J = 6.6 Hz, 1 H), 8.23 (s, 1 H), 7.47 (s, 1 H), 7.22- 7.12 (m, 2 H), 7.06 (dd, J = 23.2, 7.2 Hz, 2 H), 6.22 (dd, J = 17.1, 10.0 Hz, 1 H), 6.09 (dd, J = 17.1, 2.3 Hz, 1 H), 5.60 (dd, J = 10.0, 2.3 Hz, 1 H), 5.03-4.90 (m, 1 H), 4.48-4.32 (m, 1 H), 3.94 (s, 3 H), 2.83-2.71 (m, 2 H), 2.67-2.54 (m, 4 H), 2.47-2.38 (m, 2 H), 1.77-1.55 (m, 4 H). |
| 90 | N-((1s,4s)-4- ((4-((3,4- dichloro-2- fluorophenyl)a- mino)-7- methoxyquinazo- lin-6- yl)oxy)cyclohex- yl)acrylamide | 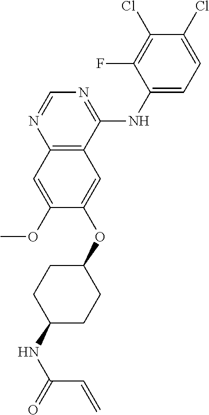 [SMILES: C=CC(=O)N[C@H]1CC[C@@H](Oc2cc3c(Nc4ccc(Cl)c(Cl)c4F)ncnc3cc2OC)CC1] | None | LC/MS (ESI, m/z): [M + 1]⁺ = 505.1, 507.1. ¹H NMR (400 MHz, DMSO-d₆) δ 9.71 (br s, 1 H), 8.39 (s, 1 H), 8.12 (d, J = 7.7 Hz, 1 H), 7.83 (s, 1 H), 7.58 (s, 2 H), 7.24 (s, 1 H), 6.29 (dd, J = 17.1, 10.1 Hz, 1 H), 6.09 (dd, J = 17.1, 2.2 Hz, 1 H), 5.57 (dd, J = 10.1, 2.3 Hz, 1 H), 4.72- 4.70 (m, 1 H), 3.95 (s, 3 H), 3.87-3.79 (m, 1 H), 2.07-1.95 (m, 2 H), 1.84-1.76 (m, 2 H), 1.72-1.64 (m, 4 H). |
| 91 | N-((1r,4r)-4- ((4-((3,4- dichloro-2- fluorophenyl)a- mino)-7- methoxyquinazo- lin-6- yl)oxy)cyclohex- yl)acrylamide | 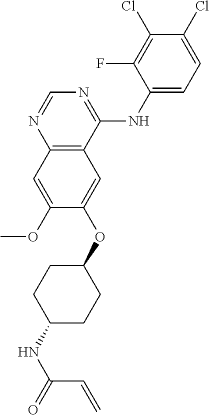 [SMILES: C=CC(=O)N[C@H]1CC[C@H](Oc2cc3c(Nc4ccc(Cl)c(Cl)c4F)ncnc3cc2OC)CC1] | None | LC/MS (ESI, m/z): [M +1]⁺ = 505.1, 507.1. ¹H NMR (400 MHz, DMSO-d₆) δ 9.79 (s, 1 H), 8.43 (s, 1 H), 8.05 (d, J = 7.4 Hz, 1 H), 7.82 (s, 1 H), 7.70-7.54 (m, 2 H), 7.23 (s, 1 H), 6.24 (dd, J = 17.1, 10.1 Hz, 1 H), 6.08 (dd, J = 17.1, 2.1 Hz, 1 H), 5.58 (dd, J = 10.1, 2.1 Hz, 1 H), 4.59-4.42 (m, 1 H), 3.94 (s, 3 H), 3.79-3.64 (m, 1 H), 2.26-2.11 (m, 2 H), 2.01-1.88 (m, 2 H), 1.63-1.48 (m, 2 H), 1.49-1.34 (m, 2 H). |

### Example 92: Preparation of N-(3-((4-((3,4-dichloro-2-fluorophenyl)amino)-7-methoxyquinazolin-6-yl)amino)cyclobutyl)acrylamide (Compound 92)

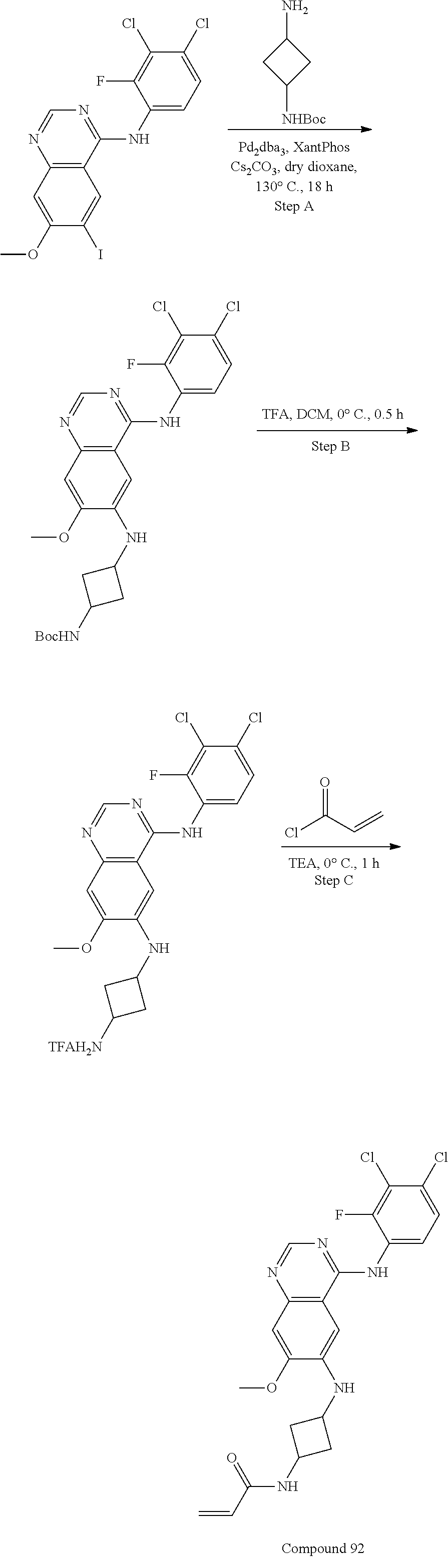

Step A: A mixture of N-(3,4-dichloro-2-fluorophenyl)-6-iodo-7-methoxyquinazolin-4-amine (231 mg, 0.50 mmol), tert-butyl (3-aminocyclobutyl)carbamate (140 mg, 0.75 mmol), Pd₂dba₃ (46 mg, 0.05 mmol), XantPhos (58 mg, 0.10 mmol) and Cs₂CO₃ (325 mg, 1.00 mmol) in dry 1,4-dioxane (10 mL) was stirred at 130° C. in a sealed tube. After being cooled to room temperature, the mixture was concentrated and purified by column chromatography (EA) to obtained tert-butyl (3-((4-((3,4-dichloro-2-fluorophenyl)amino)-7-methoxyquinazolin-6-yl)amino)cyclobutyl)carbamate (28 mg, 10.7% yield) as a yellow solid. LC/MS (ESI, m/z): [M+H]⁺=522.1.

Step B: To a solution of tert-butyl (3-((4-((3,4-dichloro-2-fluorophenyl)amino)-7-methoxyquinazolin-6-yl)amino)cyclobutyl)carbamate (113 mg, 022 mmol) in DCM (2 mL) was added TFA (1 mL) at 0° C. The mixture was stirred at room temperature for 0.5 hour. The mixture was diluted with DCM (40 mL) and concentrated to give N⁶-(3-aminocyclobutyl)-N⁴-(3,4-dichloro-2-fluorophenyl)-7-methoxyquinazoline-4,6-diamine TFA salt (135 mg, 100% yield) as a yellow solid. LC/MS (ESI, m/z): [M+H]⁺=422.0.

Step C: To a solution of N⁶-(3-aminocyclobutyl)-N⁴-(3,4-dichloro-2-fluorophenyl)-7-methoxyquinazoline-4,6-diamine TFA salt (105 mg, 0.20 mmol) and TEA (60 mg, 0.60 mmol) in dry DCM (4 mL) was dropwise added a solution of acryloyl chloride (20 mg, 0.22 mmol) in DCM (1 mL) over 2 min at 0° C. The reaction was stirred at 0° C. for 30 min. The mixture was directly purified by Prep-HPLC (NH₄OH/MeCN). The product was purified again by Prep-TLC (DCM/MeOH=20:1) to give N-(3-((4-((3,4-dichloro-2-fluorophenyl)amino)-7-methoxyquinazolin-6-yl)amino)cyclobutyl)acrylamide (Compound 92) (24.6 mg, 25% yield) as a pale-yellow solid. LC/MS (ESI, m/z): [M+H]⁺=476.0, 478.0. ¹H NMR (400 MHz, DMSO-d₆) δ 9.49 (d, J=57.2 Hz, 1H), 8.58-8.09 (m, 2H), 7.76-7.47 (m, 2H), 7.38-6.81 (m, 2H), 6.38-6.01 (m, 2H), 5.84 (dd, J=48.9, 6.4 Hz, 1H), 5.59 (dd, J=10.0, 2.3 Hz, 1H), 4.48-3.67 (m, 5H), 2.93 (s, 1H), 2.45-2.22 (m, 2H), 1.91 (m, 1H).

### Example 93: Preparation of N-((1r,3r)-3-((4-((3-chlorobenzyl)amino)-7-methoxyuinazolin-6-yl)oxy)cyclobutyl)acrylamide 2,2,2-trifluoroacetate (Compound 93)

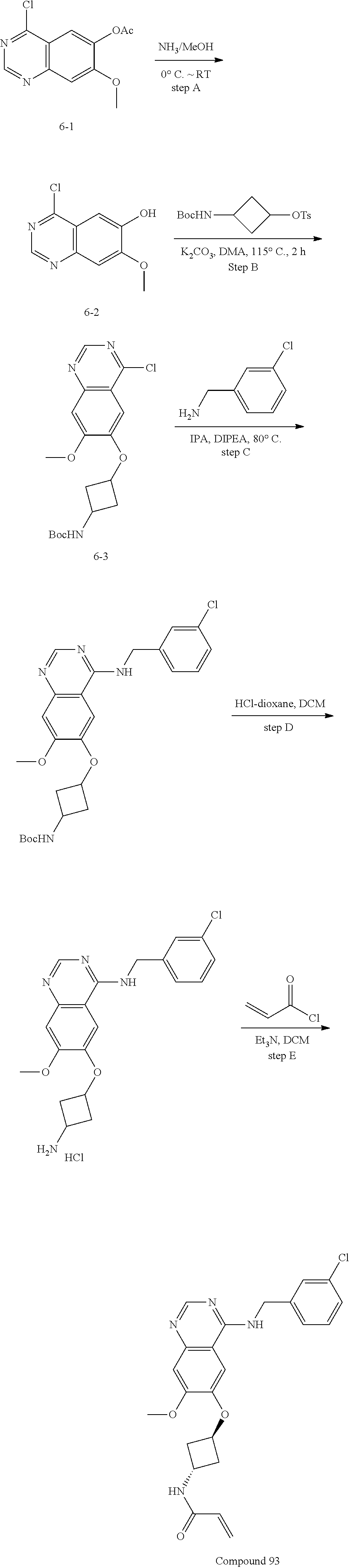

Step A: To a solution of NH₃ in MeOH (7 mol/L, 100 mL, 700 mmol) was slowly added 4-chloro-7-methoxyquinazolin-6-yl acetate (5 g, 19.8 mmol) at 0° C. After the addition, the reaction mixture was stirred at room temperature for 2 hours. The reaction mixture was concentrated in vacuo and the residue was purified by chromatography on silica gel (eluted with DCM/MeOH=100/3˜100/8) to give 4-chloro-7-methoxyquinazolin-6-ol as a white solid (4.0 g, 96% yield). LC/MS (ESI, m/z): [M+H]⁺=211.0.

Step B: A mixture of 4-chloro-7-methoxyquinazolin-6-ol (1.45 g, 6.9 mmol), 3-((tert-butoxycarbonyl)amino)cyclobutyl 4-methylbenzenesulfonate (2.8 g, 8.3 mmol) and K₂CO₃ (1.9 g, 13.8 mmol) in DMA (10 mL) was degassed with nitrogen, heated to 120° C. and stirred for 2 hours under nitrogen atmosphere. Then the mixture was quenched with water (50 mL) and extracted with EtOAc (40 mL×3). The combined organic layers were washed with brine, dried over anhydrous sodium sulfate and concentrated in vacuo. The residue was purified by column chromatography (eluted with DCM/MeOH=100/1) to give tert-butyl (3-((4-chloro-7-methoxyquinazolin-6-yl)oxy)cyclobutyl)carbamate as a yellow solid (150 mg, 6% yield). LC/MS (ESI, m/z): [M+H]⁺=380.1.

Step C: A mixture of tert-butyl (3-((4-chloro-7-methoxyquinazolin-6-yl)oxy)cyclobutyl)carbamate (150 mg, 0.39 mmol), (3-chlorophenyl)methanamine (112 mg, 0.79 mmol) in isopropanol was degassed with nitrogen, heated to 80° C. and stirred for 10 hours under nitrogen atmosphere. The reaction was cooled to room temperature and concentrated in vacuo. The residue was purified by column chromatography (eluted with DCM/MeOH=40/1) to give tert-butyl (3-((4-((3-chlorobenzyl)amino)-7-methoxyquinazolin-6-yl)oxy)cyclobutyl)carbamate as a white solid (110 mg, 57% yield). LC/MS (ESI, m/z): [M+H]⁺=485.2.

Step D: To a solution of tert-butyl (3-((4-((3-chlorobenzyl)amino)-7-methoxyquinazolin-6-yl)oxy)cyclobutyl)carbamate (110 mg, 0.23 mmol) in DCM (5 mL) was added HCl (4 mol/L in 1,4-dioxane, 1 mL, 4 mmol, 17.2 equiv) at 0° C. The mixture was stirred for 2 hours at room temperature. The reaction mixture was then concentrated in vacuo to give crude 6-(3-aminocyclobutoxy)-N-(3-chlorobenzyl)-7-methoxyquinazolin-4-amine hydrochloride as a white solid (120 mg crude). LC/MS (ESI, m/z): [M+H]⁺=385.0.

Step E: To a solution of 6-(3-aminocyclobutoxy)-N-(3-chlorobenzyl)-7-methoxyquinazolin-4-amine hydrochloride (120 mg, 0.28 mmol) and triethylamine (84 mg, 0.84 mmol) in DCM (5 mL) was added acryloyl chloride (28 mg, 0.31 mmol) at 0° C. The reaction solution was stirred at the room temperature for 1 hour. Then the reaction solution was quenched with water (5 mL) and extracted with EtOAc (15 mL×3). The combined organic layer was washed with brine, dried over Na₂SO₄ and concentrated in vacuo. The residue was purified by column chromatography (eluted with DCM/MeOH=16/1) and further purified by reverse phase prep-HPLC (eluted with CH₃CN/H₂O=5%˜90% with 0.1% TFA) to give N-((1r,3r)-3-((4-((3-chlorobenzyl)amino)-7-methoxyquinazolin-6-yl)oxy)cyclobutyl)acrylamide 2,2,2-trifluoroacetate as a white solid (Compound 93) (30 mg, 24% yield). H NMR showed only trans isomer. LC/MS (ESI, m/z): [M+H]⁺=439.1. ¹H NMR (400 MHz, DMSO-d₆) δ: 10.13 (t, J=5.4 Hz, 1H), 8.80 (s, 1H), 8.58 (d, J=6.7 Hz, 1H), 7.58 (s, 1H), 7.45 (s, 1H), 7.35-7.40 (m, 3H), 7.26 (s, 1H), 6.22 (dd, J=17.1, 10.0 Hz, 1H), 6.10 (dd, J=17.1, 2.3 Hz, 1 H), 5.61 (dd, J=10.0, 2.3 Hz, 1H), 4.97-5.04 (m, 1H), 4.95 (d, J=5.8 Hz, 2H), 4.41 (m, 1H), 4.00 (s, 3H), 2.57-2.66 (m, 2H), 2.39-2.49 (m, 2H).

### Example 94: Preparation of 6-((1-acryloylpiperidin-4-yl)oxy)-N-(4-fluorobenzyl)-7-methoxyquinazoline-4-carboxamide (Compound 94)

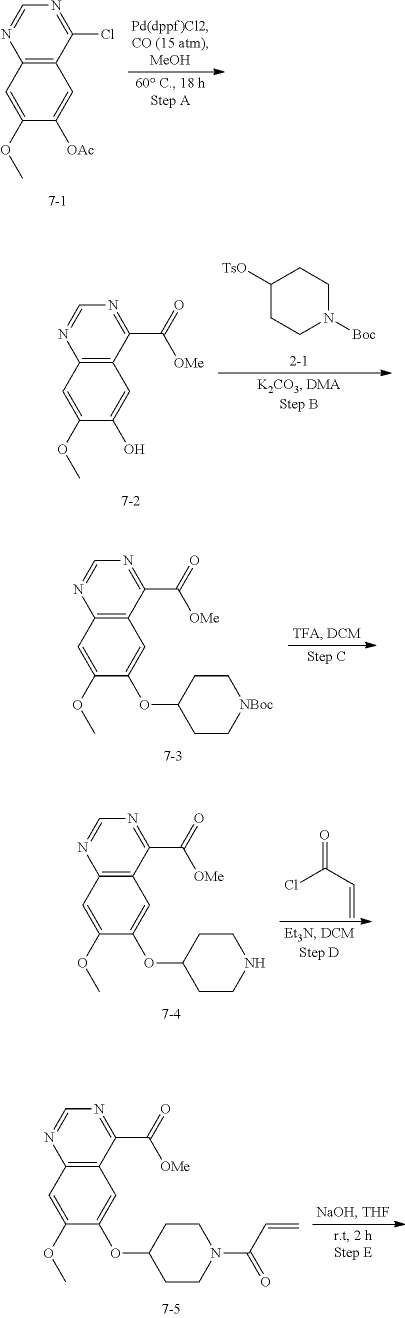

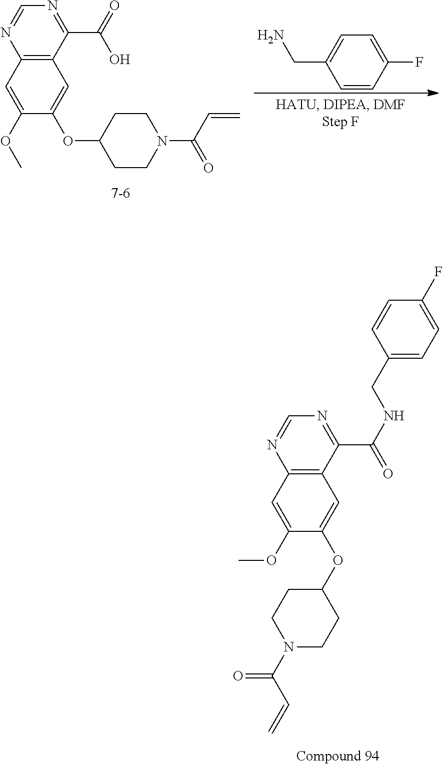 [SMILES: C=CC(=O)N1CCC(Oc2cc3c(C(=O)O)ncnc3cc2OC)CC1 ; NCc1ccc(F)cc1 ; C=CC(=O)N1CCC(Oc2cc3c(C(=O)NCc4ccc(F)cc4)ncnc3cc2OC)CC1]

Step A: To 4-chloro-7-methoxyquinazolin-6-yl acetate (2 g, 7.94 mmol) and PdCl₂(dppf) (580 mg, 0.794 mmol) was added a mixture of Et₃N (2.4 g, 23.82 mmol) and MeOH (50 mL). The mixture was bubbled with CO (gas) to 20 atm. The mixture was warmed to 60° C. at 20 atm and stirred for 10 h. The mixture was cooled to room temperature, and the resulting mixture was concentrated in vacuum. The residue was purified by flash chromatography eluting with MeOH/DCM=5% to afford methyl 6-hydroxy-7-methoxyquinazoline-4-carboxylate (1 g, 54% yield). LC/MS (ESI, m/z): [M+H]⁺=235.1.

Step B: A mixture of methyl 6-hydroxy-7-methoxyquinazoline-4-carboxylate (500 mg, 2.14 mmol), tert-butyl 4-(tosyloxy)piperidine-1-carboxylate (1.52 g, 4.27 mmol), and K₂CO₃ (885 mg, 6.41 mmol) in DMA (5 mL) was warmed to 120° C. and stirred for 2 h. The reaction mixture was cooled to room temperature, then poured into water and extracted with EA (3×20 mL). The combined organic layers were washed with water (30 mL), brine (30 mL), dried over Na₂SO₄, filtered and concentrated in vacuum. The residue was purified by flash chromatography eluting with EA/DCM=50% to afford methyl 6-((1-(tert-butoxycarbonyl)piperidin-4-yl)oxy)-7-methoxyquinazoline-4-carboxylate (500 mg, 56% yield) as a yellow solid. LC/MS (ESI, m/z): [M+H]⁺=418.0.

Step C: To a solution of methyl 6-((1-(tert-butoxycarbonyl)piperidin-4-yl)oxy)-7-methoxyquinazoline-4-carboxylate (404 mg, 0.969 mmol) in DCM (3 mL) was slowly added trifluoroacetic acid (1 mL). The mixture was stirred at RT for 1 h. The reaction mixture was then concentrated in vacuum. The residue was dried to give methyl 7-methoxy-6-(piperidin-4-yloxy)quinazoline-4-carboxylate 2,2,2-trifluoroacetate (418 mg, 100%) which was used directly in the next step without further purification. LC/MS (ESI, m/z): [M+1]⁺=318.1.

Step D: To a mixture of methyl 7-methoxy-6-(piperidin-4-yloxy)quinazoline-4-carboxylate 2,2,2-trifluoroacetate (418 mg, 0.969 mmol) and DIPEA (375 mg, 2.91 mmol) in DCM (5 mL) was added acryloyl chloride (114 mg, 1.26 mmol), The mixture was stirred at RT for 1 hour. The reaction mixture was quenched by MeOH and concentrated in vacuum. The residue was purified by flash chromatography eluting with EA/DCM=50% to afford methyl 6-((1-acryloylpiperidin-4-yl)oxy)-7-methoxyquinazoline-4-carboxylate (235 mg, 65.4% yield) as a yellow solid. LC/MS (ESI, m/z): [M+H]⁺=372.3.

Step E: To a solution of methyl 6-((1-acryloylpiperidin-4-yl)oxy)-7-methoxyquinazoline-4-carboxylate (237 mg, 0.637 mmol) in THF(5 mL) was added NaOH (51.1 mg, 1.27 mmol) in H₂O (1 mL). The mixture was stirred at RT for 2 hour. The reaction mixture was quenched by H₂O (10 mL) and the aqueous solution was washed with Et₂O (2×10 mL). To the aqueous solution was added 1M HCl solution until pH<5. The solution was extracted with DCM/iPrOH=3:1 (3×20 mL). The combined organic layers were washed with brine, dried over Na₂SO₄, filtered and concentrated in vacuum. The residue was dried to afford 6-((1-acryloylpiperidin-4-yl)oxy)-7-methoxyquinazoline-4-carboxylic acid (163 mg, 72% yield) as a yellow solid. LC/MS (ESI, m/z): [M+1]⁺=358.1.

Step F: To a mixture of 6-((1-acryloylpiperidin-4-yl)oxy)-7-methoxyquinazoline-4-carboxylic acid (40 mg, 0.112 mmol), 4-fluorophenyl)methanamine (17 mg, 0.134 mmol), HATU (46.8 mg, 0.123 mmol) in DCM (5 mL) was added DIPEA (43 mg, 0.336 mmol). The mixture was stirred at RT for 2 hours. The reaction mixture was poured into water and extracted with EA (3×20 mL). The combined organic layers were washed with brine, dried over Na₂SO₄, filtered and concentrated in vacuum. The residue was purified by prep-HPLC to give 6-((1-acryloylpiperidin-4-yl)oxy)-N-(4-fluorobenzyl)-7-methoxyquinazoline-4-carboxamide (Compound 94) (6.3 mg, 12% yield) as a yellow solid. LC/MS (ESI, m/z): [M+H]⁺=465.1. ¹H NMR (400 MHz, DMSO-d₆) δ 9.68 (t, J=6.3 Hz, 1H), 9.17 (s, 1H), 8.43 (s, 1H), 7.46 (s, 1 H), 7.45-7.38 (m, 2H), 7.22-7.13 (m, 2H), 6.84 (dd, J=10.5, 16.8 Hz, 1H), 6.12 (dd, J=2.4, 16.8 Hz, 1H), 5.69 (dd, J=2.4, 10.4 Hz, 1H), 4.77-4.72 (m, 1H), 4.53 (d, J=6.1 Hz, 2H), 4.02 (s, 3H), 3.90-3.78 (m, 2H), 3.45-3.42 (m, 2H), 2.08-1.96 (m, 2H), 1.76-1.62 (m, 2H).

Examples 95-97 were prepared using similar procedures as described in the preceding Examples.

| EX. | | Compound | | |
|---|---|---|---|---|
| NO. | IUPAC Name | structure | salt | Compound characterization |
| 95 | (R)-6-((1- acryloylpiperidin-4- yl)oxy)-N-(1-(4- fluorophenyl)ethyl)- 7- methoxyquinazoline- 4-carboxamide | 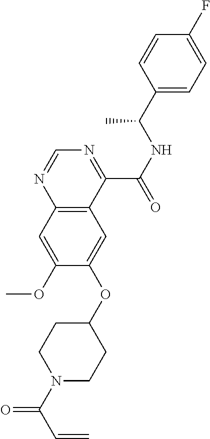 [SMILES: C=CC(=O)N1CCC(Oc2cc3c(C(=O)N[C@H](C)c4ccc(F)cc4)ncnc3cc2OC)CC1] | None | LC/MS (ESI, m/z): [M + H]⁺ = 479.1. ¹H NMR (400 MHz, DMSO-d₆) δ 9.46 (d, J = 8.4 Hz, 1 H), 9.17 (s, 1 H), 8.15 (s, 1 H), 7.53-7.47 (m, 2 H), 7.45 (s, 1 H), 7.23- 7.14 (m, 2 H), 6.83 (dd, J = 10.5, 16.8 Hz, 1 H), 6.11 (dd, J = 2.4, 16.8 Hz, 1 H), 5.69 (dd, J = 2.4, 10.5 Hz, 1 H), 5.27-5.21 (m, 1 H), 4.73-4.62 (m, 1 H), 4.01 (s, 3 H), 3.83-3.76 (m, 3 H), 3.54-3.47 (m, 1 H), 2.04-1.93 (m, 2 H), 1.73-1.61 (m, 2 H), 1.52 (d, J = 7.0 Hz, 3 H). |
| 96 | 6-(3- acrylamidocyclobutoxy)- N-(4- fluorobenzyl)-7- methoxyquinazoline- 4-carboxamide | 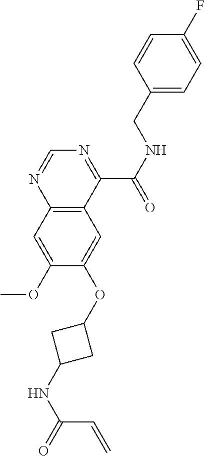 [SMILES: C=CC(=O)NC1CC(Oc2cc3c(C(=O)NCc4ccc(F)cc4)ncnc3cc2OC)C1] | None | LC/MS (ESI, m/z): [M + 1]⁺ = 451.2. ¹H NMR (400 MHz, DMSO-d₆) δ 9.68 (s, 1 H), 9.16 (s, 1 H), 8.50 (s, 1 H), 8.25 (s, 1 H), 7.48-7.42 (m, 3 H), 7.21-7.13 (m, 2 H), 6.30-6.00 (m, 2 H), 5.64-5.60 (m, 1 H), 4.58-4.50 (m, 3 H), 4.14-4.10 (m, 1 H), 4.02 (s, 3 H), 2.94-2.86 (m, 2 H), 2.14-2.06 (m, 2 H). |
| 97 | 6-((1s,3S)-3- acrylamidocyclobutoxy)- N-((R)-1-(4- fluorophenyl)ethyl)- 7- methoxyquinazoline- 4-carboxamide | 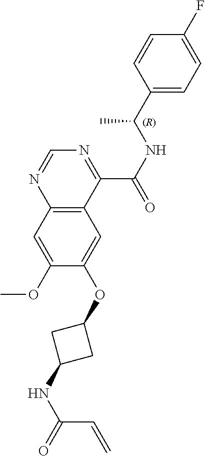 | None | LC/MS (ESI, m/z): [M + 1]⁺ = 465.2. ¹H NMR (400 MHz, DMSO-d₆) δ 9.46 (d, J = 8.3 Hz, 1 H), 9.17 (s, 1 H), 8.48 (d, J = 7.6 Hz, 1 H), 7.98 (s, 1 H), 7.59-7.48 (m, 2 H), 7.44 (s, 1 H), 7.19 (t, J = 8.8 Hz, 2 H), 6.23-6.00 (m, 2 H), 5.61 (dd, J = 9.6, 2.6 Hz, 1 H), 5.41-5.14 (m, 1 H), 4.64- 4.44 (m, 1 H), 4.18-4.06 (m, 1 H), 4.01 (s, 3 H), 2.86-2.83 (m, 2 H), 2.10-2.06 (m, 2 H), 1.52 (d, J = 7.0 Hz, 3 H). |

### Example 98: Preparation of N-(6-((l-acryloylpiperidin-4-yl)oxy)-7-methoxyquinazolin-4-yl)-4-fluorobenzamide (Compound 98)

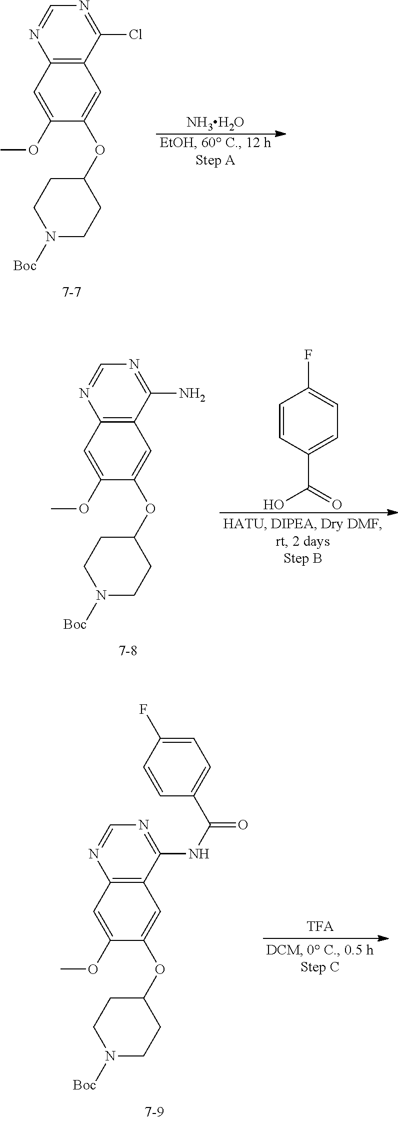 [SMILES: COc1cc2ncnc(Cl)c2cc1OC1CCN(C(=O)OC(C)(C)C)CC1 ; "EtOH, 60° C., 12 hStep A" ; "NH3·H2O" ; COc1cc2ncnc(N)c2cc1OC1CCN(C(=O)OC(C)(C)C)CC1 ; O=C(O)c1ccc(F)cc1 ; COc1cc2ncnc(NC(=O)c3ccc(F)cc3)c2cc1OC1CCN(C(=O)OC(C)(C)C)CC1]

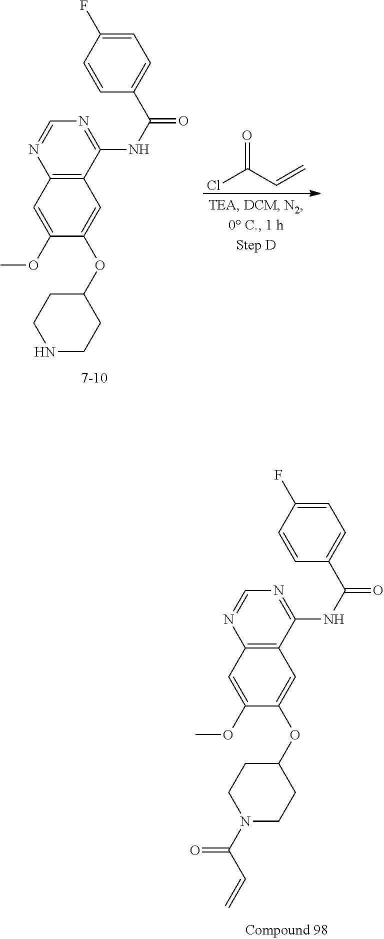

Step A: To a solution of tert-butyl 4-((4-chloro-7-methoxyquinazolin-6-yl)oxy)piperidine-1-carboxylate (250 mg, 0.63 mmol) in EtOH (6 mL) was added NH4OH (6 mL), the mixture was stirred at 60° C. under N₂ atmosphere overnight. After being cooled to RT, the solution was concentrated in vacuo and the solid was placed in ice water for dissociation. Filtered to give tert-butyl 4-((4-amino-7-methoxy quinazolin-6-yl)oxy)piperidine-1I-carboxylate (245 mg, 93% yield) as a white solid. LC/MS (ESI, m/z): [M+H]⁺=375.1.

Step B: To a solution of tert-butyl 4-((4-amino-7-methoxyquinazolin-6-yl)oxy)piperidine-1-carboxylate (195 mg, 0.44 mmol), 4-fluorobenzoic acid (93 mg, 0.66 mmol) and HATU (252 mg, 0.66 mmol) in dry DMF was added DIPEA (171 mg, 1.33 mmol) at 0° C. The mixture was stirred at RT overnight. After evaporated, the solid was purified by SGC (EA) to give tert-butyl 4-((4-(4-fluorobenzamido)-7-methoxyquinazolin-6-yl)oxy)piperidine-1-carboxylate (88 mg, 34% yield) as a white solid. LC/MS (ESI, m/z): [M+1]⁺=497.1.

Step C: To an ice-cooled solution of tert-butyl 4-((4-(4-fluorobenzamido)-7-methoxyquinazolin-6-yl)oxy)piperidine-1-carboxylate (100 mg, 0.20 mmol) in DCM (1.5 mL) was added TFA (1.5 mL). The reaction solution was stirred at RT for 0.5 h. The solution was concentrated in vacuo to give 4-fluoro-N-(7-methoxy-6-(piperidin-4-yloxy)quinazolin-4-yl)benzamide (80 mg, 100% yield) as a yellow solid. LC/MS (ESI, m/z): [M+1]⁺=397.1.

Step D: A mixture of 4-fluoro-N-(7-methoxy-6-(piperidin-4-yloxy)quinazolin-4-yl)benzamide (80 mg, 0.20 mmol), acryloyl chloride (18 mg, 0.20 mmol),Et₃N(61 mg, 0.61 mmol) in dry DCM (3 mL) was stirred for 1 h at 0° C. under N₂ atmosphere. The reaction was quenched by MeOH and concentrated in vacuum. The residue was purified by SGC (DCM:MeOH=20:1) then purified by reversed HPLC (CH₃CN and H₂O with 0.01% TFA as mobile phase) to give N-(6-((1-acryloylpiperidin-4-yl)oxy)-7-methoxyquinazolin-4-yl)-4-fluorobenzamide TFA salt (Compound 98) (12 mg, 12% yield) as a white solid. LC/MS (ESI, m/z): [M+H]⁺=451.1. ¹H NMR (400 MHz, DMSO-d₆) δ 8.68 (br s, 1H), 8.22 (s, 2H), 7.74 (br s, 1H), 7.39-7.35 (m, 3H), 6.84 (dd, J=16.7, 10.5 Hz, 1H), 6.11 (dd, J=16.7, 2.4 Hz, 1 H), 5.68 (dd, J=10.5, 2.4 Hz, 1H), 4.83 (s, 1H), 4.00 (s, 3H), 3.96-3.86 (m, 2H), 3.49-3.39 (m, 2H), 2.10-1.98 (m, 2H), 1.75-1.69 (m, 2H).

### Example 99: Preparation of N-((1R,3s)-3-((4-((3,4-dichloro-2-fluorophenyl)amino)-7-(((S)-tetrahydrofuran-3-yl)oxy)quinazolin-6-yl)oxy)cyclobutyl)acrylamide (Compound 99)

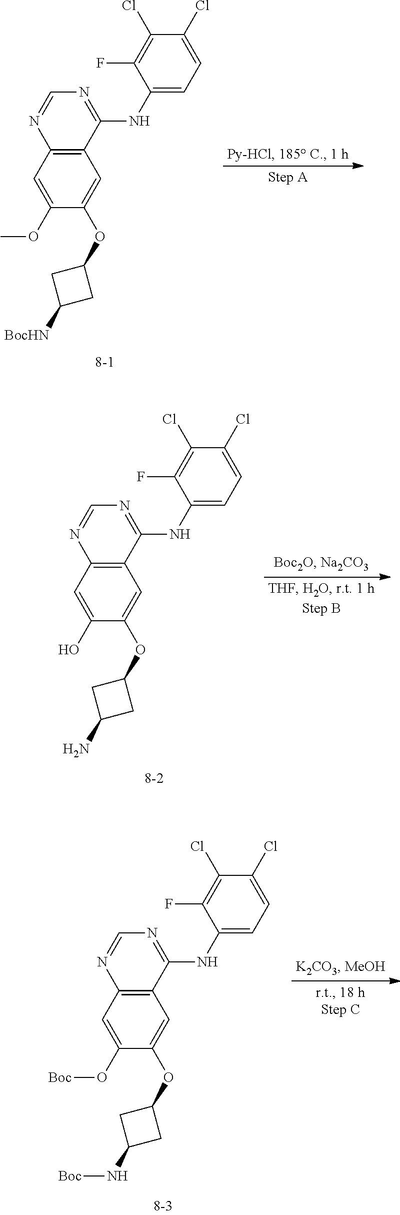 [SMILES: COc1cc2ncnc(Nc3ccc(Cl)c(Cl)c3F)c2cc1O[C@H]1C[C@@H](NC(=O)OC(C)(C)C)C1 ; N[C@H]1C[C@@H](Oc2cc3c(Nc4ccc(Cl)c(Cl)c4F)ncnc3cc2O)C1 ; CC(C)(C)OC(=O)N[C@H]1C[C@@H](Oc2cc3c(Nc4ccc(Cl)c(Cl)c4F)ncnc3cc2OC(=O)OC(C)(C)C)C1]

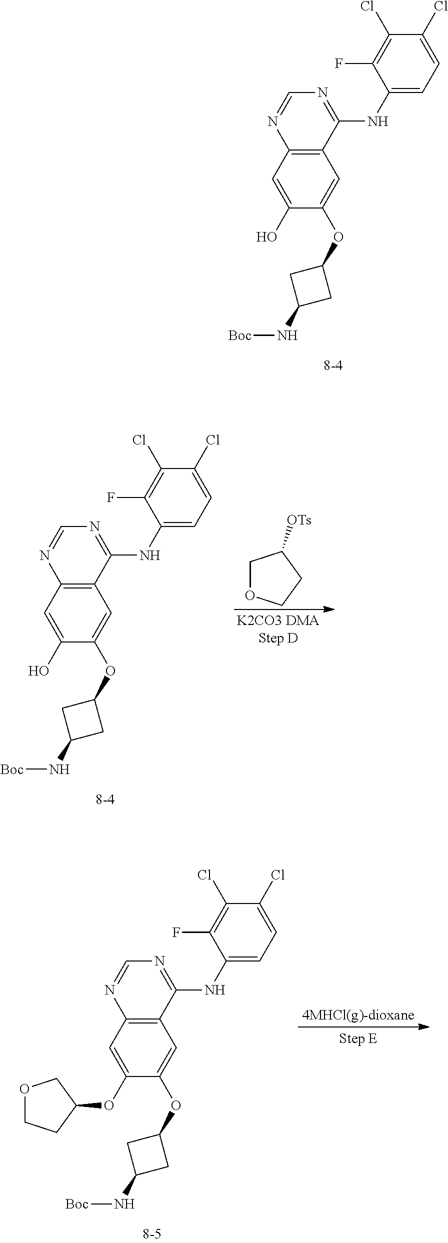 [SMILES: CC(C)(C)OC(=O)N[C@H]1C[C@@H](Oc2cc3c(Nc4ccc(Cl)c(Cl)c4F)ncnc3cc2O)C1 ; CC(C)(C)OC(=O)N[C@H]1C[C@@H](Oc2cc3c(Nc4ccc(Cl)c(Cl)c4F)ncnc3cc2O)C1 ; Cc1ccc(S(=O)(=O)O[C@@H]2CCOC2)cc1 ; CC(C)(C)OC(=O)N[C@H]1C[C@@H](Oc2cc3c(Nc4ccc(Cl)c(Cl)c4F)ncnc3cc2O[C@H]2CCOC2)C1]

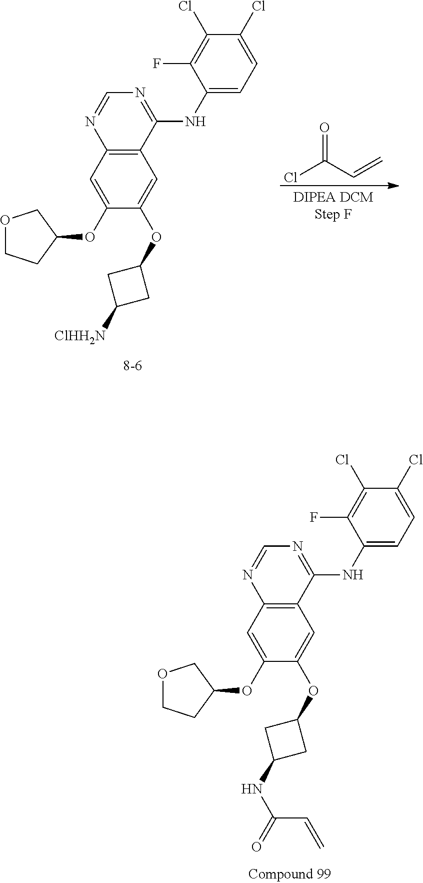

Step A: tert-butyl ((1s,3s)-3-((4-((3,4-dichloro-2-fluorophenyl)amino)-7-methoxyquinazolin-6-yl)oxy)cyclobutyl)carbamate (180 mg, 344 umol) and pyridine hydrochloride (1.96 g, 17 mmol) mixture was placed in a flask. The mixture was stirred at 180° C. for 1 hour, then cooled to room temperature and poured into water. The mixture was filtered and the cake was dried to give 6-((1s,3s)-3-aminocyclobutoxy)-4-((3,4-dichloro-2-fluorophenyl)amino)quinazolin-7-ol hydrochloride (140 mg, 91% yield) as a yellow solid. LC/MS (ESI, m/z): [M+H]⁺=409.1.

Step B: To a solution of 6-((1s,3s)-3-aminocyclobutoxy)-4-((3,4-dichloro-2-fluorophenyl)amino)quinazolin-7-ol hydrochloride (140 mg, 342 umol) in H₂O (10 mL) was added THF (5 mL) and (Boc)₂O (224 mg, 1.02 mmol). The mixture was stirred at room temperature for 1 hour. The mixture was quenched with water (30 mL) and extracted with EtOAc (10 mL×3). The combined organic layers were washed with brine, dried over anhydrous sodium sulfate, filtered and concentrated in vacuum to afford tert-butyl ((1s,3s)-3-((7-((tert-butoxycarbonyl)oxy)-4-((3,4-dichloro-2-fluorophenyl)amino)quinazolin-6-yl)oxy)cyclobutyl)carbamate (208 mg) as a brown solid. LC/MS (ESI, m/z): [M+H]⁺=609.1.

Step C: A mixture of tert-butyl ((1s,3s)-3-((7-((tert-butoxycarbonyl)oxy)-4-((3,4-dichloro-2-fluorophenyl)amino)quinazolin-6-yl)oxy)cyclobutyl)carbamate (208 mg) and K₂CO₃ (141 mg, 1.02 mmol) in MeOH (5 mL) was stirred at RT for 18 h. The mixture was concentrated and the residue was dissolved in EA and H₂O. The organic layer was washed with brine, dried over Na₂SO₄, filtered and concentrated in vacuum. The residue was purified by column chromatography (DCM:CH₃OH=20:1) to give tert-butyl ((1s,3s)-3-((4-((3,4-dichloro-2-fluorophenyl)amino)-7-hydroxyquinazolin-6-yl)oxy)cyclobutyl)carbamate (70 mg, 40% yield) as a brown solid. LC/MS (ESI, m/z): [M+H]⁺=509.1.

Step D: To a solution of tert-butyl ((1s,3s)-3-((4-((3,4-dichloro-2-fluorophenyl)amino)-7-hydroxyquinazolin-6-yl)oxy)cyclobutyl)carbamate (70 mg, 137.43 umol) in dimethylacetamide (2.5 mL) were added (R)-tetrahydrofuran-3-yl 4-methylbenzenesulfonate (49.95 mg, 206.14 umol) and K₂CO₃ (37.99 mg, 274.86 umol). The mixture was heated to 115° C. and stirred for 1 hour under nitrogen atmosphere. The mixture was cooled, quenched by water (30 mL) and extracted with EA. The organic layer was washed with brine, dried over anhydrous sodium sulfate, filtered and concentrated in vacuum. The residue was purified by column chromatography (PE/EA) to give tert-butyl ((1R,3s)-3-((4-((3,4-dichloro-2-fluorophenyl)amino)-7-(((S)-tetrahydrofuran-3-yl)oxy)quinazolin-6-yl)oxy)cyclobutyl)carbamate (55 mg, 69% yield) as a brown solid. LC/MS (ESI, m/z): [M+H]⁺=579.1.

Step E: A solution of tert-butyl ((1R,3s)-3-((4-((3,4-dichloro-2-fluorophenyl)amino)-7-(((S)-tetrahydrofuran-3-yl)oxy)quinazolin-6-yl)oxy)cyclobutyl)carbamate (55 mg, 94.9 umol) in 4M HCl(g)-dioxane (3 mL) was stirred at RT for 1 h. The reaction solution was concentrated to give 6-((1s,3R)-3-aminocyclobutoxy)-N-(3,4-dichloro-2-fluorophenyl)-7-(((S)-tetrahydrofuran-3-yl)oxy)quinazolin-4-aminehydrochloride (45 mg, 92% yield) which was used in the next step without any purification. LC/MS (ESI, m/z): [M+H]⁺=479.1.

Step F: To a solution of 6-((1s,3R)-3-aminocyclobutoxy)-N-(3,4-dichloro-2-fluorophenyl)-7-(((S)-tetrahydrofuran-3-yl)oxy)quinazolin-4-aminehydrochloride was added N-isopropyl-N-methylpropan-2-amine (45.1 mg, 348.98 umol) in DCM (1 mL). Acryloyl chloride (7.9 mg, 87.24 umol) was then added. The reaction was stirred for 0.5 h at r.t., quenched by water, and extracted with DCM. The organic layer was washed with brine, dried over anhydrous sodium sulfate, filtered and concentrated in vacuum. The residue was purified via reverse phase column chromatography (CH₃CN/H₂O) to give N-((1R,3s)-3-((4-((3,4-dichloro-2-fluorophenyl)amino)-7-(((S)-tetrahydrofuran-3-yl)oxy)quinazolin-6-yl)oxy)cyclobutyl)acrylamide (Compound 99) (12 mg, 26% yield) as a white solid. LC/MS (ESI, m/z): [M+H]⁺=533.0, 535.0. ¹H NMR (400 MHz, DMSO) δ 10.65 (br s, 1H), 8.67 (s, 1 H), 8.50 (d, J=7.8 Hz, 1H), 7.75-7.56 (m, 3H), 7.25 (s, 1H), 6.16 (dd, J=17.1, 9.5 Hz, 1 H), 6.09 (dd, J=17.1, 2.8 Hz, 1H)., 5.61 (dd, J=9.5, 2.8 Hz, 1H), 5.25 (m, 1H), 4.69-4.51 (m, 1H), 4.09 (m, 1H),3.99-3.90 (m, 3H), 3.84-3.78 (m, 1H), 3.12-3.05 (m, 2H), 2.42-2.31 (m,2 H) 2.12-2.02 (m, 2H).

### Example 100: Preparation of N-((1s,3s)-3-((4-((3,4-dichloro-2-fluorophenyl)amino)-7-(2-hydroxyethoxy)quinazolin-6-yl)oxy)cyclobutyl)acrylamide (Compound 100)

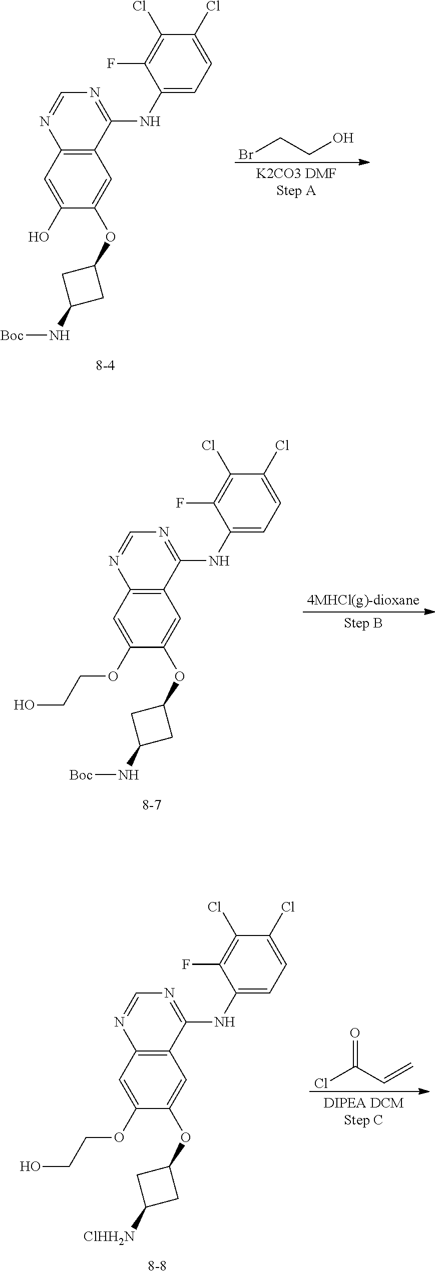

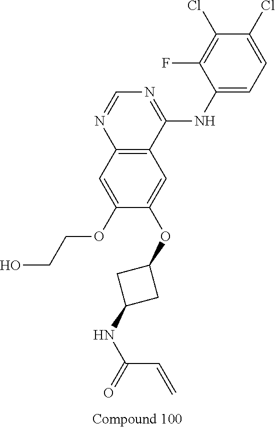 [SMILES: C=CC(=O)N[C@H]1C[C@@H](Oc2cc3c(Nc4ccc(Cl)c(Cl)c4F)ncnc3cc2OCCO)C1]

Step A: To a solution of tert-butyl ((1s,3s)-3-((4-((3,4-dichloro-2-fluorophenyl)amino)-7-hydroxyquinazolin-6-yl)oxy)cyclobutyl)carbamate (150 mg, 294 umol) in DMF (5 mL). was added K₂CO₃ (122 mg, 883 umol) and then 2-bromoethan-1-ol (55 mg, 442 umol). The reaction was stirred at 40° C. for 2 hours. The mixture was quenched by water (30 mL) and extracted with EtOAc. The combined organic layers were washed with brine, dried over anhydrous sodium sulfate, filtered and concentrated in vacuum. The residue was purified by column chromatography (PE/EA) to give tert-butyl ((1s,3s)-3-((4-((3,4-dichloro-2-fluorophenyl)amino)-7-(2-hydroxyethoxy)quinazolin-6-yl)oxy)cyclobutyl)carbamate (59 mg, 36% yield) as a white solid. LC/MS (ESI, m/z): [M+H]⁺=553.1.

Steps B and C were completed in a similar manner as described in Example 99 steps E and F to afford N-((1s,3s)-3-((4-((3,4-dichloro-2-fluorophenyl)amino)-7-(2-hydroxyethoxy)quinazolin-6-yl)oxy)cyclobutyl)acrylamide (Compound 100). LC/MS (ESI, m/z): [M+H]⁺=507.2, 509.2. ¹H NMR (400 MHz, DMSO-d₆) δ 10.65(br s, 1H), 8.68 (s, 1H), 8.50 (d, J=7.6 Hz, 1H), 7.75-7.56 (m, 3H), 7.31 (s, 1H), 6.17 (dd, J=17.1, 9.7 Hz, 1H), 6.09 (dd, J=17.0, 2.6 Hz, 1H), 5.61 (dd, J=9.6, 2.7 Hz, 1H), 4.73-4.56 (m, 1H), 4.27-4.16 (m, 2H), 4.14-4.05 (m, 1H), 3.88-3.79 (m, 2H), 3.13-3.04 (m, 2H), 2.10-2.02 (m, 2H).

### Example 101: Preparation of N-((1s,3s)-3-((4-((3,4-dichloro-2-fluorophenyl)amino)-7-(2-(dimethylamino)ethoxy)quinazolin-6-yl)oxy)cyclobutyl)acrylamide (Compound 101)

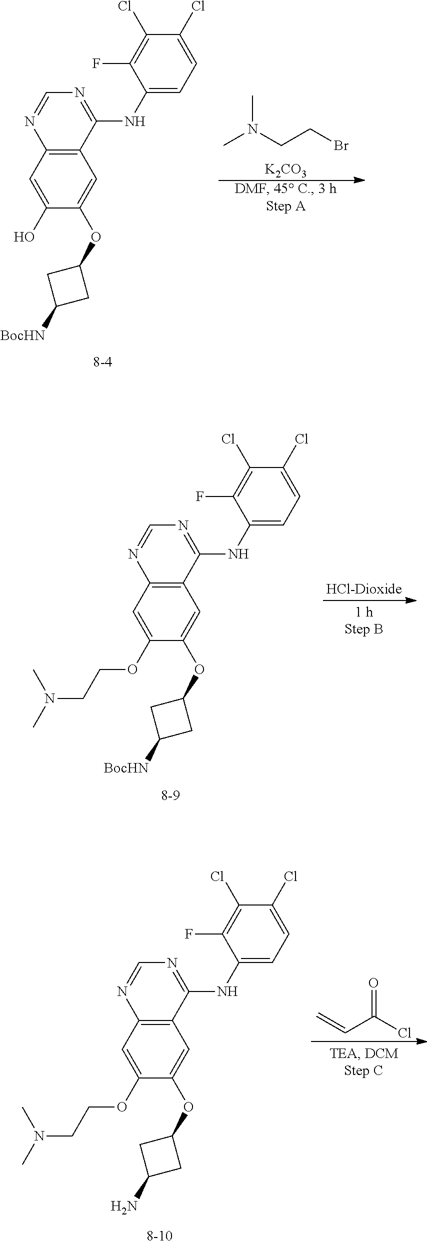

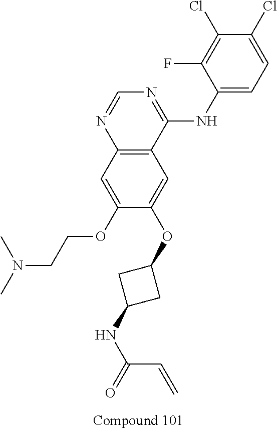 [SMILES: C=CC(=O)N[C@H]1C[C@@H](Oc2cc3c(Nc4ccc(Cl)c(Cl)c4F)ncnc3cc2OCCN(C)C)C1]

Step A: A mixture of tert-butyl ((1s,3s)-3-((4-((3,4-dichloro-2-fluorophenyl)amino)-7-hydroxyquinazolin-6-yl)oxy)cyclobutyl)carbamate (100 mg, 0.196 mmol), 2-bromo-N,N-dimethylethanamine hydrobromide (46 mg, 0.196 mmol) and K₂CO₃ (135 mg, 0.98 mmol) in DMF (5 mL) was degassed with nitrogen, heated to 40° C. and stirred for 2 hours under nitrogen atmosphere. Then the mixture was quenched with water (15 mL), extracted with EtOAc (15 mL×3). The combined organic layer was washed with brine, dried over anhydrous sodium sulfate and concentrated in vacuo. The residue was purified by column chromatography on silica gel (eluted with DCM/MeOH=10/1) to give tert-butyl ((1s,3s)-3-((4-((3,4-dichloro-2-fluorophenyl)amino)-7-(2-(dimethylamino)ethoxy)quinazolin-6-yl)oxy)cyclobutyl)carbamate as a yellow solid (75 mg, 66% yield). LC/MS (ESI, m/z): [M+H]⁺=580.2.

Steps B and C were completed in a similar manner as described in Example 99 steps E and F to afford N-((1s,3s)-3-((4-((3,4-dichloro-2-fluorophenyl)amino)-7-(2-(dimethylamino)ethoxy)quinazolin-6-yl)oxy)cyclobutyl)acrylamide (Compound 101). LC/MS (ESI, m/z): [M+H]⁺=534.1. ¹H NMR (400 MHz, DMSO-d₆) δ: 10.54 (br s, 1H), 10.01 (br s, 1 H), 8.63 (s, 1H), 8.50 (d, J=7.2 Hz, 1H), 7.73 (s, 1H), 7.56-7.68 (m, 2H), 7.43 (s, 1H), 6.19 (dd, J=17.2, 10.0 Hz, 1H), 6.09 (dd, J=17.2, 2.4 Hz, 1H), 5.62 (dd, J=10.0, 2.4 Hz, 1 H), 4.67-4.62 (m, 1H), 4.58 (t, J=5.2 Hz, 2H), 4.01-4.08 (m, 1H), 3.61-3.70 (m, 2H), 3.03-3.13 (m, 2H), 2.97 (s, 6H), 2.02-2.12 (m, 2H).

### Example 102: Preparation of N-((1s,3s)-3-((4-((3,4-dichloro-2-fluorophenyl)amino)-7-(methoxy-d3)quinazolin-6-yl)oxy)cyclobutyl)acrylamide (Compound 102)

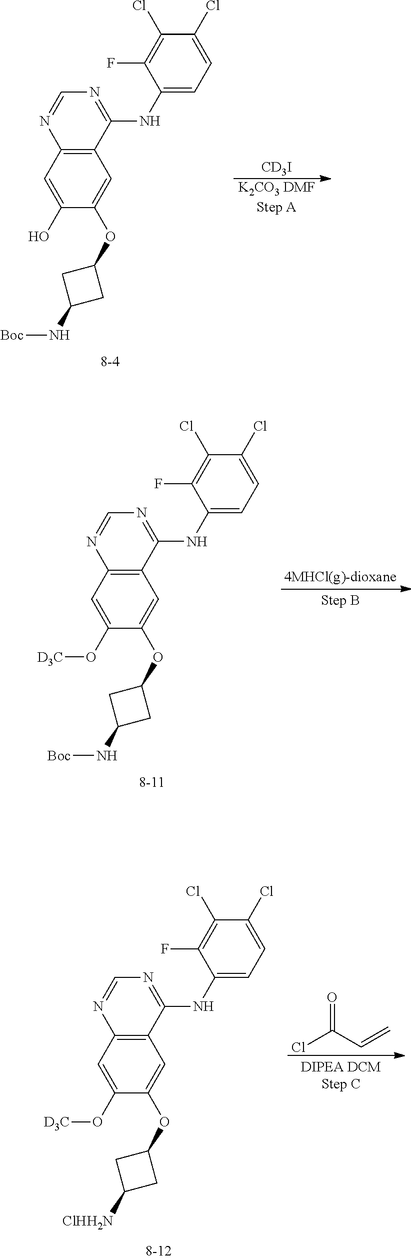

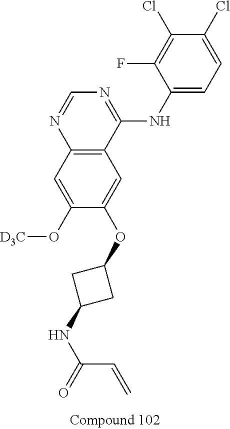 [SMILES: [2H]C([2H])([2H])Oc1cc2ncnc(Nc3ccc(Cl)c(Cl)c3F)c2cc1O[C@H]1C[C@@H](NC(=O)C=C)C1]

Step A: To a solution of tert-butyl ((1s,3s)-3-((4-((3,4-dichloro-2-fluorophenyl)amino)-7-hydroxyquinazolin-6-yl)oxy)cyclobutyl)carbamate (300 mg, 589 umol) in DMF (10 mL) was added K₂CO₃ (161.7 mg, 1.17 mmol) and then iodomethane-d₃ (85.4 mg, 589 umol). The reaction was stirred for 1 hour. The mixture was quenched by water (30 mL) and extracted with EtOAc (50 mL×3). The combined organic layers were washed with brine, dried over anhydrous sodium sulfate, filtered and concentrated in vacuum. The residue was purified by column chromatography (PE/EA) to give tert-butyl((1s,3s)-3-((4-((3,4-dichloro-2-fluorophenyl)amino)-7-(methoxy-d3)quinazolin-6-yl)oxy)cyclobutyl)carbamate (212 mg, 68.4 yield) as a white solid. ¹H NMR (400 MHz, DMSO) δ 9.64 (s, 1H), 8.38 (s, 1H), 7.63-7.57 (m, 2H), 7.54 (s, 1H), 7.25 (d, J=7.8 Hz, 1H), 7.22 (s, 1H), 4.55-4.45 (m, 1H), 3.78-3.70 (m, 1H), 2.99-2.93 (m, 2 H), 2.10-2.00 (m, 2H).

Steps B and C were completed in a similar manner as described in Example 99 steps E and F to afford N-((1s,3s)-3-((4-((3,4-dichloro-2-fluorophenyl)amino)-7-(methoxy-d3)quinazolin-6-yl)oxy)cyclobutyl)acrylamide (Compound 102). LC/MS (ESI, m/z): [M+H]⁺=480.0, 482.0. ¹H NMR (400 MHz, DMSO-d₆) δ 9.68 (s, 1H), 8.73 (s, 1H), 8.49 (d, J=7.5 Hz, 1H), 7.65-7.50 (m, 3H), 7.29 (d, J=6.5 Hz, 1H), 6.17 (dd, J=17.1, 9.7 Hz, 1H), 6.09 (dd, J=17.0, 2.6 Hz, 1H), 5.61 (dd, J=9.6, 2.6 Hz, 1H), 4.71-4.53 (m, 1H), 4.13-4.05 (m, 1H), 3.12-3.03 (m, 2H), 2.12-2.03 (m, 2H).

### Example 103: Preparation of N-((1s,3s)-3-((4-((3,4-dichloro-2-fluorophenyl)amino)-7-(methylamino)quinazolin-6-yl)oxy)cyclobutyl)acrylamide (Compound 103)

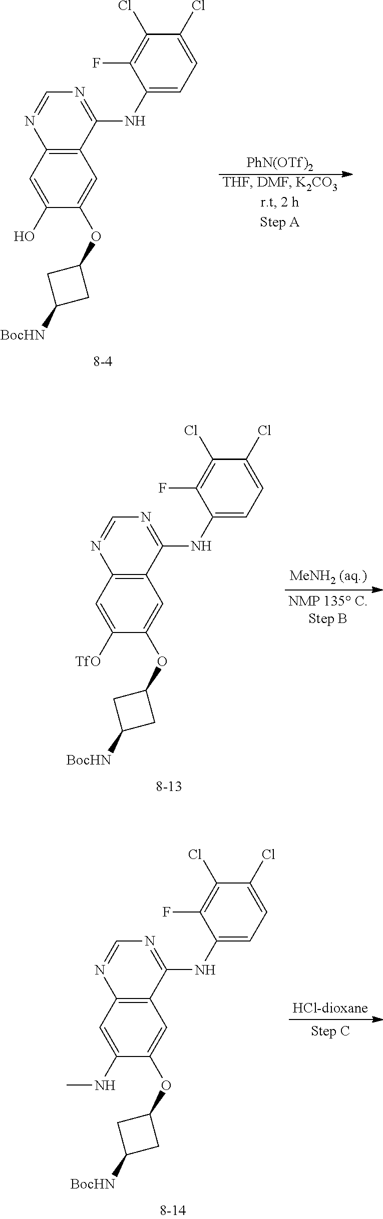 [SMILES: "PhN(OTf)2" ; CC(C)(C)OC(=O)N[C@H]1C[C@@H](Oc2cc3c(Nc4ccc(Cl)c(Cl)c4F)ncnc3cc2O)C1 ; "THF, DMF, K2CO3r.t, 2 hStep A" ; CC(C)(C)OC(=O)N[C@H]1C[C@@H](Oc2cc3c(Nc4ccc(Cl)c(Cl)c4F)ncnc3cc2OS(=O)(=O)C(F)(F)F)C1 ; CNc1cc2ncnc(Nc3ccc(Cl)c(Cl)c3F)c2cc1O[C@H]1C[C@@H](NC(=O)OC(C)(C)C)C1]

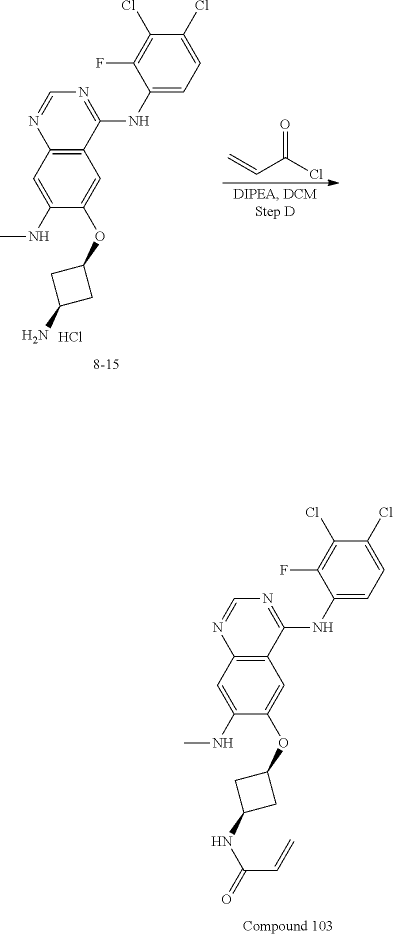

Step A: To a solution of tert-butyl ((1s,3s)-3-((4-((3,4-dichloro-2-fluorophenyl)amino)-7-hydroxyquinazolin-6-yl)oxy)cyclobutyl)carbamate (260 mg, 0.512 mmol) in dry DMF (5 mL) was added K₂CO₃ (106 mg, 0.767 mmol) and N-phenyl-O-((trifluoromethyl)sulfonyl)-N-(((trifluoromethyl)sulfonyl)oxy)hydroxylamine (219 mg, 0.563 mmol) at room temperature. The reaction mixture was stirred at room temperature for 2 h. H₂O (30 mL) was added, the mixture was extracted with EtOAc (20 mL×3), washed with brine, dried over Na₂SO₄. The solid was filtered and the filtrate was concentrated to give the crude product (300 mg) which used directly to the next step. LC/MS (ESI, m/z): [M+H]⁺=641.0, 643.0.

Step B: To a solution of 6-((1s,3s)-3-((tert-butoxycarbonyl)amino)cyclobutoxy)-4-((3,4-dichloro-2-fluorophenyl)amino)quinazolin-7-yl trifluoromethanesulfonate (300 mg) in NMP (3 mL) was added MeNH₂ (aq.) (1 mL) in a seal tube and the reaction mixture was stirred at 135° C. for 5 h. H₂O (15 mL) was added, the mixture was extracted with EtOAc (20 mL×3), washed with brine, dried over Na₂SO₄. The solid was filtered and the filtrate was concentrated to give the crude product which purified by column chromatography on silica gel to give the product tert-butyl ((1s,3s)-3-((4-((3,4-dichloro-2-fluorophenyl)amino)-7-(methylamino)quinazolin-6-yl)oxy)cyclobutyl)carbamate (35 mg, yield 11% for two steps).

Steps C and D were completed in a similar manner as described in Example 99 steps E and F to afford N-((1s,3s)-3-((4-((3,4-dichloro-2-fluorophenyl)amino)-7-(methylamino)quinazolin-6-yl)oxy)cyclobutyl)acrylamide (Compound 103). LC/MS (ESI, m/z): [M+H]⁺=476.0, 478.0. ¹H NMR (400 MHz, DMSO-d₆) δ 14.40 (br s, 1H), 10.87 (s, 1H), 8.69 (s, 1H), 8.58-8.45 (m, 1H), 7.68 (dd, J=8.8, 1.4 Hz, 1H), 7.64-7.57 (m, 1H), 7.54 (s, 1H), 7.30 (d, J=5.0 Hz, 1H), 6.61 (s, 1H), 6.20 (dd, J=17.1, 9.9 Hz, 1H), 6.10 (dd, J=17.1, 2.4 Hz, 1H), 5.61 (dd, J=9.9, 2.4 Hz, 1H), 4.66 (p, J=6.9 Hz, 1H), 4.14-4.01 (m, 1H), 3.10 (dd, J=10.5, 5.9 Hz, 2H), 2.90 (d, J=4.7 Hz, 3H), 2.14 (dd, J=19.2, 9.1 Hz, 2H).

Examples 104-105 were prepared using similar procedures as described in the preceding Examples.

| EX. | | Compound | | |
|---|---|---|---|---|
| NO. | IUPAC Name | structure | salt | Compound characterization |
| 104 | N-((1s,3s)-3-((4-((3,4- dichloro-2- fluorophenyl)amino)-7- (oxetan-3- yloxy)quinazolin-6- yl)oxy)cyclobutyl) acrylamide | 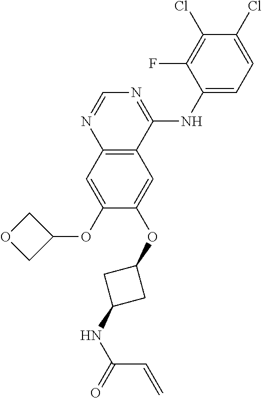 [SMILES: C=CC(=O)N[C@H]1C[C@@H](Oc2cc3c(Nc4ccc(Cl)c(Cl)c4F)ncnc3cc2OC2COC2)C1] | None | LC/MS (ESI, m/z): [M + 1]⁺ = 519.0, 521.0. ¹H NMR (400 MHz, DMSO- d₆) δ 9.72 (s, 1 H), 8.50 (d, J = 7.7 Hz, 1 H), 8.38 (s, 1 H), 7.71-7.51 (m, 3 H), 6.86 (s, 1 H), 6.25-6.01 (m, 2 H), 5.61 (dd, J = 9.6, 2.7 Hz, 1 H), 5.55- 5.41 (m, 1 H), 5.03 (t, J = 6.7 Hz, 2 H), 4.73-4.53 (m, 3 H), 4.17-4.02 (m, 1 H), 3.15-3.03 (m, 2 H), 2.21- 2.00 (m, 2 H). |
| 105 | N-((1s,3s)-3-((4-((3,4- dichloro-2- fluorophenyl)amino)-7- ethoxyquinazolin-6- yl)oxy)cyclobutyl) acrylamide | 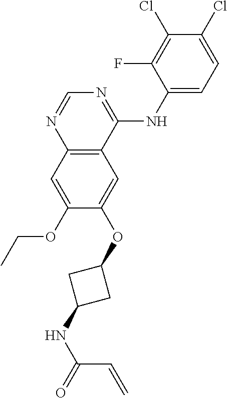 [SMILES: C=CC(=O)N[C@H]1C[C@@H](Oc2cc3c(Nc4ccc(Cl)c(Cl)c4F)ncnc3cc2OCC)C1] | None | LC/MS (ESI, m/z): [M + 1]⁺ = 491.0, 492.0. ¹H NMR (400 MHz, DMSO- d₆) δ 9.66 (s, 1 H), 8.48 (d, J = 7.7 Hz, 1 H), 8.38 (s, 1 H), 7.67-7.46 (m, 3 H), 7.21 (s, 1 H), 6.13 (dd, J = 14.2, 6.1 Hz, 2 H), 5.61 (dd, J = 9.6, 2.7 Hz, 1 H), 4.69-4.54 (m, 1 H), 4.22 (q, J = 7.0 Hz, 2 H), 4.15-4.00 (m, 1 H), 3.13-2.98 (m, 2 H), 2.16-2.01 (m, 2 H), 1.42 (t, J = 6.9 Hz, 3 H). |

### Example 106: Preparation of 1-(4-((4-((3-chlorobenzyl)amino)-7-methoxyquinazolin-6-yl)oxy)piperidin-1-yl)prop-2-en-1-one (Compound 106)

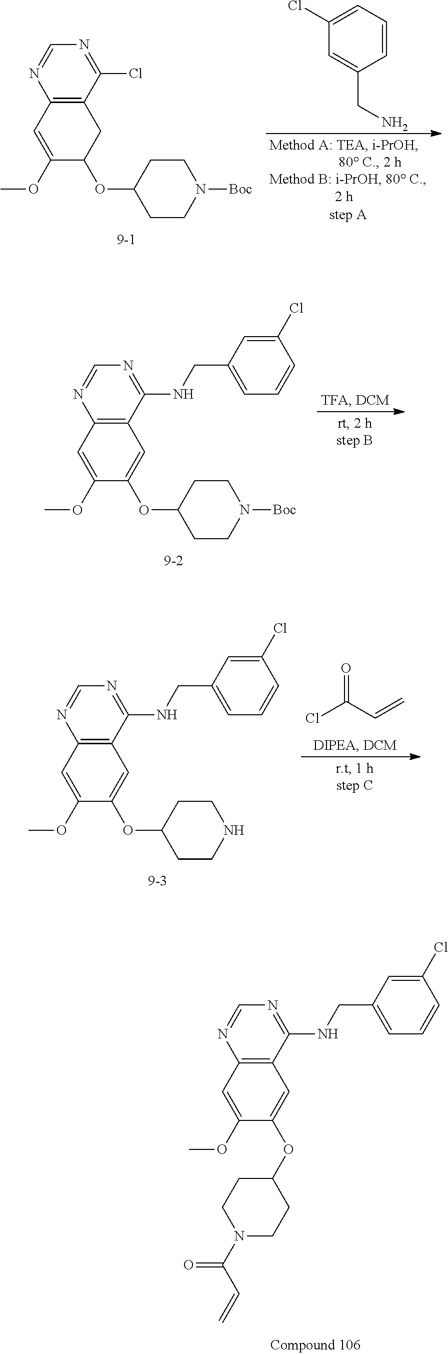

Step A (method A): To a mixture of tert-butyl 4-((4-chloro-7-methoxy-5,6-dihydroquinazolin-6-yl)oxy)piperidine-1I-carboxylate (109 mg, 0.277 mmol) in isopropanol (8 mL) was added Et₃N (42 mg, 0.416 mmol) and (3-chlorophenyl)methanamine (78 mg, 0.544 mmol). The reaction mixture was stirred at 80° C. for 5 h. The mixture was concentrated in vacuum. The residue was purified by column chromatography on silica gel eluting with DCM:MeOH=20:1 to afford tert-butyl 4-((4-((3-chlorobenzyl)amino)-7-methoxyquinazolin-6-yl)oxy)piperidine-1-carboxylate (131 mg). LC/MS (ESI, m/z): [M+H]⁺=499.1.

Step A (method B): A mixture of tert-butyl 4-((4-chloro-7-methoxy-5,6-dihydroquinazolin-6-yl)oxy)piperidine-1-carboxylate (0.3 mmol) in isopropanol (8 mL) was added (3-chlorophenyl)methanamine (0.6 mmol). The reaction mixture was stirred at 80° C. for 5 h. The mixture was concentrated in vacuum. The residue was purified by column chromatography on silica gel eluting with DCM:MeOH=20:1 to give tert-butyl 4-((4-((3-chlorobenzyl)amino)-7-methoxyquinazolin-6-yl)oxy)piperidine-1-carboxylate.

Step B: A mixture of tert-butyl 4-((4-((3-chlorobenzyl)amino)-7-methoxyquinazolin-6-yl)oxy)piperidine-1-carboxylate (131 mg, 0.263 mmol) in DCM (3 mL) was added TFA (1 mL). The reaction mixture was stirred at rt for 2 h. The mixture was concentrated in vacuum. The residue was washed by 1,2-dichloroethane (2×10 mL) and then concentrated in vacuum to give N-(3-chlorobenzyl)-7-methoxy-6-(piperidin-4-yloxy)quinazolin-4-amine. LC/MS (ESI, m/z): [M+H]⁺=399.2.

Step C: To a mixture of N-(3-chlorobenzyl)-7-methoxy-6-(piperidin-4-yloxy)quinazolin-4-amine (135 mg, 0.263 mmol) in DCM (8 mL) was added DIPEA (102 mg, 0.789 mmol) and acryloyl chloride (23.7 mg, 0.263 mmol). The reaction mixture was stirred at rt for 4 h. The mixture was concentrated in vacuum. The residue was washed with water (1×30 mL) and extracted with EtOAc (2×25 mL). The residue was purified by prep-TLC eluting with DCM:MeOH=20:1 and prep-HPLC to give 1-(4-((4-((3-chlorobenzyl)amino)-7-methoxyquinazolin-6-yl)oxy)piperidin-1-yl)prop-2-en-1-one (7.8 mg, 7% yield) as a white solid. LC/MS (ESI, m/z): [M+H]⁺=453.1. ¹H NMR (400 MHz, DMSO-d₆) δ 8.48 (t, J=6.0 Hz, 1 H), 8.33 (s, 1H), 7.76 (s, 1H), 7.39-7.29 (m, 4H), 7.15 (s, 1H), 6.84 (dd, J=16.6, 10.4 Hz, 1 H), 6.11 (dd, J=16.6, 2.4 Hz, 1H), 5.68 (dd, J=10.4, 2.4 Hz, 1H), 4.79 (d, J=5.5 Hz, 2H), 4.75-4.70 (m, 1H), 3.91 (s, 3H), 3.87-3.83 (m, 2H), 3.53-3.43 (m, 2H), 2.03-1.96 (m, 2H), 1.70-1.65 (m, 2H).

### Example 107: Preparation of 1-(4-((4-((3-chlorobenzyl)oxy)-7-methoxyquinazolin-6-yl)oxy)piperidin-1-yl)prop-2-en-1-one (Compound 107)

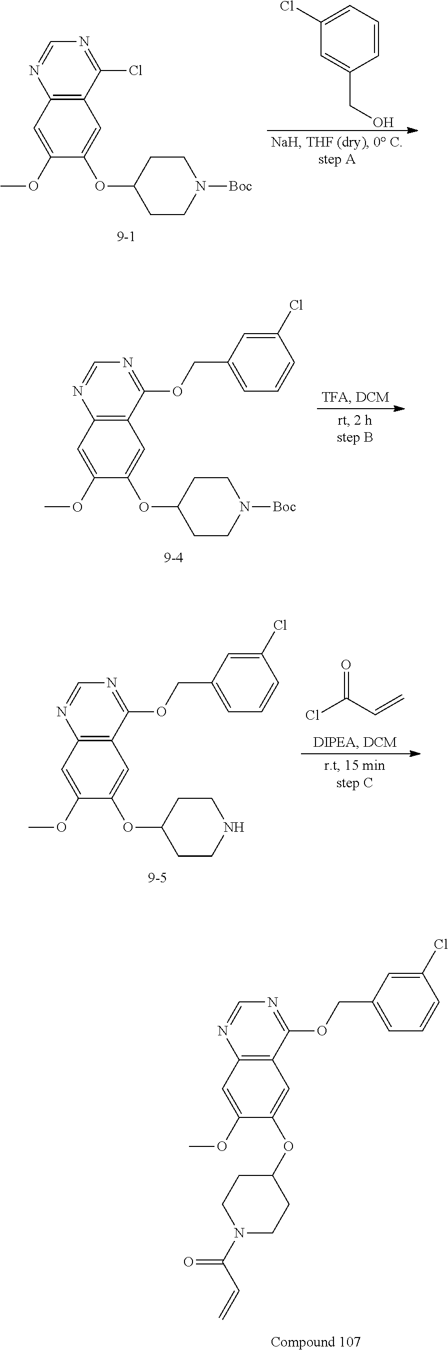

To a solution of (3-chlorophenyl)methanol (43 mg, 0.305 mmol) in dry THE (6 mL) was added NaH (19 mg, 0.458 mmol) (60% in mineral oil) at 0° C. The reaction mixture was stirred at 0° C. for 20 min. Tert-butyl 4-((4-chloro-7-methoxyquinazolin-6-yl)oxy)piperidine-1-carboxylate (100 mg, 0.25 mmol) was added at 0° C. The reaction mixture was stirred at room temperature for 3 h. Water (10 mL) was added. The reaction mixture was extracted with EtOAc (20 mL×3), washed with brine, dried over anhydrous Na₂SO₄, filtered and concentrated under reduced pressure to give a residue. The residue was purified by column chromatography on silica gel eluting with DCM:MeOH=20:1 to afford tert-butyl 4-((4-((3-chlorobenzyl)oxy)-7-methoxyquinazolin-6-yl)oxy)piperidine-1-carboxylate (72 mg, yield 58%). LC/MS (ESI, m/z): [M+H]⁺=500.1.

Steps B and C were completed in a similar manner as described in Example 106 steps B and C to afford 1-(4-((4-((3-chlorobenzyl)oxy)-7-methoxyquinazolin-6-yl)oxy)piperidin-1-yl)prop-2-en-1-one (Compound 107). LC/MS (ESI, m/z): [M+1]⁺=454.2. ¹H NMR (400 MHz, DMSO-d₆) δ 8.75 (s, 1H), 7.76-7.28 (m, 6H), 6.83 (dd, J=16.7, 10.5 Hz, 1H), 6.11 (dd, J=16.7, 2.3 Hz, 1H), 5.69 (s, 2H), 5.67 (d, J=8.4 Hz, 1H), 4.90-4.84 (m, 1H), 3.98 (s, 3H), 3.94-3.79 (m, 2H), 3.58-3.35 (m, 2H), 2.10-1.92 (m, 2H), 1.76-1.60 (m, 2H).

Examples 108-125 were prepared using similar procedures as described in the preceding Examples.

| EX. | IUPAC | Step A | Compound | | |
|---|---|---|---|---|---|
| NO. | Name | method | structure | salt | Compound characterization |
| 108 | 1-(4-((4-((4- chlorobenzyl) amino)-7- methoxy- quinazolin-6- yl)oxy) piperidin-1- yl)prop-2- en-1-one | Method A | 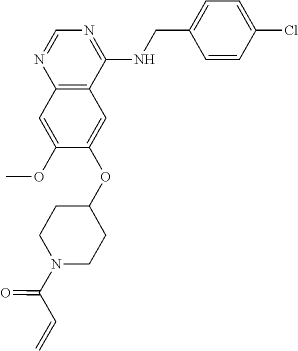 [SMILES: C=CC(=O)N1CCC(Oc2cc3c(NCc4ccc(Cl)cc4)ncnc3cc2OC)CC1] | None | LC/MS (ESI, m/z): [M + H]⁺ = 453.1. ¹H NMR (400 MHz, DMSO-d₆) δ 10.02 (br s, 1 H), 8.79 (s, 1 H), 7.95 (s, 1 H), 7.43 (d, J = 9.4 Hz, 4 H), 7.22 (s, 1 H), 6.84 (dd, J = 16.7, 10.4 Hz, 1 H), 6.12 (dd, J = 16.7, 2.4 Hz, 1 H), 5.69 (dd, J = 10.5, 2.3 Hz, 1 H), 4.94 (d, J = 4.8 Hz, 2 H), 4.78-4.75 (m, 1 H), 3.98 (s, 3 H), 3.82-3.81 (m, 2 H), 3.59-3.50 (m, 2 H), 2.04-2.01 (m, 2 H), 1.70-1.64 (m, 2 H). |
| 109 | 1-(4-((4- ((2,4- dichlorobenzyl) amino)-7- methoxy- quinazolin-6- yl)oxy) piperidin-1- yl)prop-2- en-1-one | Method A | 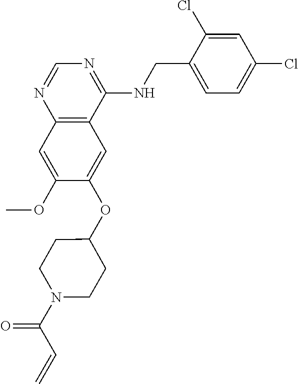 [SMILES: C=CC(=O)N1CCC(Oc2cc3c(NCc4ccc(Cl)cc4Cl)ncnc3cc2OC)CC1] | None | LC/MS (ESI, m/z): [M + H]+ = 487.0, 489.0. ¹H NMR (400 MHz, CDCl₃) δ 8.56 (s, 1 H), 7.42 (t, J = 5.6 Hz, 2 H), 7.24 (s, 1 H), 7.21 (dd, J = 8.2, 2.1 Hz, 1 H), 7.13 (s, 1 H), 6.60 (dd, J = 16.8, 10.6 Hz, 1 H), 6.28 (dd, J = 16.8, 1.9 Hz, 1 H), 6.02 (s, 1 H), 5.70 (dd, J = 10.6, 1.9 Hz, 1 H), 4.92 (d, J = 5.8 Hz, 2 H), 4.71-4.50 (m, 1 H), 3.97 (s, 3 H), 3.87-3.70 (m, 2 H), 3.61-3.50 (m, 2 H), 1.98-1.85 (m, 4 H). |
| 110 | 1-(4-((4-((2- chlorobenzyl) amino)-7- methoxy- quinazolin-6- yl)oxy) piperidin-1- yl)prop-2- en-1-one | Method A | 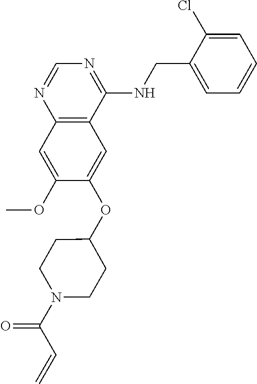 [SMILES: C=CC(=O)N1CCC(Oc2cc3c(NCc4ccccc4Cl)ncnc3cc2OC)CC1] | None | LC/MS (ESI, m/z): [M + H]⁺ = 453.0, 455.0. ¹H NMR (400 MHz, CDCl₃) δ 8.57 (s, 1 H), 7.51-7.45 (m, 1 H), 7.43-7.37 (m, 1 H), 7.29 (d, J = 2.5 Hz, 1 H), 7.25 (s, 1 H), 7.24-7.21 (m, 1H), 7.16 (s, 1 H), 6.60 (dd, J = 16.8, 10.6 Hz, 1 H), 6.29 (dd, J = 16.8, 1.9 Hz, 1 H), 5.70 (dd, J = 10.6, 1.9 Hz, 1 H), 4.97 (d, J = 5.7 Hz, 2 H), 4.67-4.65 (m, 1 H), 3.98 (s, 3 H), 3.87-3.83 (m, 2 H), 3.71-3.67 (m, 1 H), 3.54-3.52 (m, 1 H), 2.01-1.87 (m, 4 H). |
| 111 | 1-(4-((4-((3- fluorobenzyl) amino)-7- methoxy- quinazolin-6- yl)oxy) piperidin-1- yl)prop-2- en-1-one | Method A | 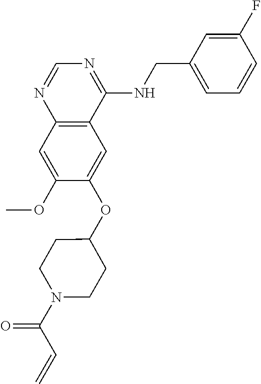 [SMILES: C=CC(=O)N1CCC(Oc2cc3c(NCc4cccc(F)c4)ncnc3cc2OC)CC1] | TFA | LC/MS (ESI, m/z): [M + H]⁺ = 437.2. ¹H NMR (400 MHz, DMSO-d₆) δ: 10.11 (s, 1 H), 8.82 (s, 1 H), 7.98 (s, 1 H), 7.41 (td, J = 8.0, 6.1 Hz, 1 H), 7.21- 7.26 (m, 3 H), 7.10-7.16 (m, 1 H), 6.84 (dd, J = 16.7, 10.5 Hz, 1 H), 6.12 (dd, J = 16.7, 2.4 Hz, 1 H), 5.69 (dd, J = 10.4, 2.4 Hz, 1 H), 4.98 (d, J = 5.9 Hz, 2 H), 4.77-4.83 (m, 1 H), 3.98 (s, 3 H), 3.86-3.79 (m, 2 H), 3.70-3.59 (m, 2 H), 1.96-2.04 (m, 2H), 1.75-1.64 (m, 2 H). |
| 112 | 1-(4-((4-((2- fluorobenzyl) amino)-7- methoxy- quinazolin-6- yl)oxy) piperidin-1- yl)prop-2- en-1-one | Method A | 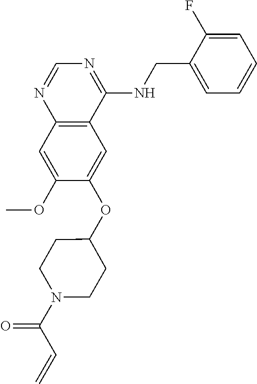 [SMILES: C=CC(=O)N1CCC(Oc2cc3c(NCc4ccccc4F)ncnc3cc2OC)CC1] | TFA | LC/MS (ESI, m/z): [M + H]⁺ = 437.2. ¹H NMR (400 MHz, DMSO-d₆) δ: 10.10 (t, J = 6.2 Hz, 1 H), 8.84 (s, 1 H), 8.01 (s, 1 H), 7.34-7.46 (m, 2 H), 7.22-7.28 (m, 2 H), 7.19 (td, J = 7.5, 1.2 Hz, 1 H), 6.84 (dd, J = 16.7, 10.5 Hz, 1 H), 6.12 (dd, J = 16.7, 2.4 Hz, 1 H), 5.69 (dd, J = 10.5, 2.4 Hz, 1 H), 5.00 (d, J = 5.6 Hz, 2 H), 4.84-4.78 (m, 1 H), 3.98 (s, 3 H), 3.84-3.77 (m, 2 H), 3.58-3.47 (m, 2 H), 2.07-1.96 (m, 2 H), 1.777-1.62 (m, 2 H). |
| 113 | 1-(4-((4-((4- chloro-3- fluorobenzyl) amino)-7- methoxy- quinazolin-6- yl)oxy) piperidin-1- yl)prop-2- en-1-one | Method A | 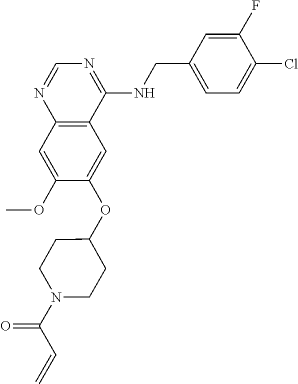 [SMILES: C=CC(=O)N1CCC(Oc2cc3c(NCc4ccc(Cl)c(F)c4)ncnc3cc2OC)CC1] | None | LC/MS (ESI, m/z): [M + H]⁺ = 471.1. ¹H NMR (400 MHz, DMSO-d₆) δ: 8.56 (t, J = 5.8 Hz, 1 H), 8.32 (s, 1 H), 7.78 (s, 1 H), 7.53 (t, J = 8.0 Hz, 1 H), 7.38 (dd, J = 10.4, 1.8 Hz, 1 H), 7.21 (dd, J = 8.3, 1.4 Hz, 1 H), 7.15 (s, 1 H), 6.84 (dd, J = 16.7, 10.5 Hz, 1 H), 6.12 (dd, J = 16.7, 2.4 Hz, 1 H), 5.68 (dd, J = 10.5, 2.4 Hz, 1 H), 4.78 (d, 2 H), 4.71-4.74 (m, 1 H), 3.91 (s, 3 H), 3.80-3.89 (m, 2 H), 3.56-3.43 (m, 2 H), 1.95-2.06 (m, 2 H), 1.59-1.77 (m, 2 H). |
| 114 | 1-(4-((4-((3- chloro-4- fluorobenzyl) amino)-7- methoxy- quinazolin-6- yl)oxy) piperidin-1- yl)prop-2- en-1-one | Method A | 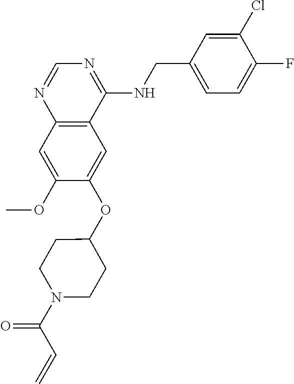 [SMILES: C=CC(=O)N1CCC(Oc2cc3c(NCc4ccc(F)c(Cl)c4)ncnc3cc2OC)CC1] | None | LC/MS (ESI, m/z): [M + H]⁺ = 471.1. ¹H NMR (400 MHz, DMSO-d₆) δ: 8.50 (t, J = 5.9 Hz, 1 H), 8.34 (s, 1 H), 7.77 (s, 1 H), 7.55 (d, J = 7.5 Hz, 1 H), 7.36 (d, J = 8.1 Hz, 2 H), 7.15 (s, 1 H), 6.84 (dd, J = 16.7, 10.5 Hz, 1 H), 6.12 (dd, J = 16.7, 2.4 Hz, 1 H), 5.68 (dd, J = 10.5, 2.4 Hz, 1 H), 4.76 (d, J = 5.6 Hz, 2 H), 4.71-4.76 (m, 1 H), 3.91 (s, 3 H), 3.80-3.89 (m, 2 H), 3.41-3.57 (m, 2 H), 1.94-2.07 (m, 2 H), 1.60-1.75 (m, 2 H). |
| 115 | 1-(4-((4- ((3,4- dichlorobenzyl) amino)-7- methoxy- quinazolin-6- yl)oxy) piperidin-1- yl)prop-2- en-1-one | Method A | 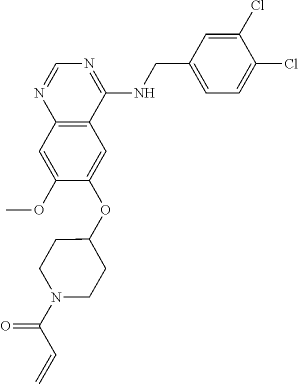 [SMILES: C=CC(=O)N1CCC(Oc2cc3c(NCc4ccc(Cl)c(Cl)c4)ncnc3cc2OC)CC1] | TFA | LC/MS (ESI, m/z): [M + H]⁺ = 487.1. ¹H NMR (400 MHz, DMSO-d₆) δ: 10.01 (br s, 1 H), 8.80 (s, 1 H), 7.95 (s, 1 H), 7.68 (d, J = 2.0 Hz, 1 H), 7.63 (d, J = 8.4 Hz, 1 H), 7.38 (dd, J = 8.4, 2.0 Hz, 1 H), 7.22 (s, 1 H), 6.84 (dd, J = 16.8, 10.4 Hz, 1 H), 6.12 (dd, J = 16.8, 2.4 Hz, 1 H), 5.69 (dd, J = 10.4, 2.4 Hz, 1 H), 4.94 (d, J = 5.6 Hz, 2 H), 4.75-4.83 (m, 1 H), 3.98 (s, 3 H), 3.78- 3.86 (m, 2 H), 3.48-3.54 (m, 2 H), 1.96-2.08 (m, 2 H), 1.63-1.77 (m, 2 H). |
| 116 | 1-(4-((4-((3- chloro-2- fluorobenzyl) amino)-7- methoxy- quinazolin-6- yl)oxy) piperidin-1- yl)prop-2- en-1-one | Method A | 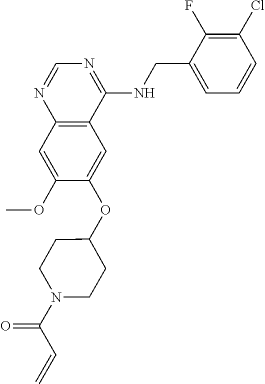 [SMILES: C=CC(=O)N1CCC(Oc2cc3c(NCc4cccc(Cl)c4F)ncnc3cc2OC)CC1] | TFA | LC/MS (ESI, m/z): [M + H]⁺ = 471.2. ¹H NMR (400 MHz, DMSO-d₆) δ: 10.04 (br s, 1 H), 8.81 (s, 1 H), 7.99 (s, 1 H), 7.51-7.57 (m, 1 H), 7.37-7.43 (m, 1 H), 7.23 (s, 1 H), 7.18-7.24 (m, 1 H), 6.84 (dd, J = 16.4, 10.4 Hz, 1 H), 6.12 (dd, J = 16.4, 2.4 Hz, 1 H), 5.69 (dd, J = 10.4, 2.4 Hz, 1 H), 5.00 (d, J = 5.6 Hz, 2 H), 4.85-4.73 (m, 1 H), 3.98 (s, 3 H), 3.84-3.78 (m, 2 H), 3.48-3.52 (m, 2 H), 1.96-2.06 (m, 2 H), 1.63-1.77 (m, 2 H). |
| 117 | 1-(4-((4- ((3,5- dichlorobenzyl) amino)-7- methoxy- quinazolin-6- yl)oxy) piperidin-1- yl)prop-2- en-1-one | Method A | 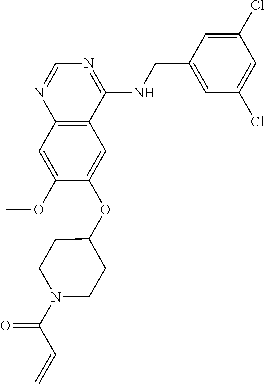 [SMILES: C=CC(=O)N1CCC(Oc2cc3c(NCc4cc(Cl)cc(Cl)c4)ncnc3cc2OC)CC1] | None | LC/MS (ESI, m/z): [M + 1]⁺ = 487.2, 489.2 ¹H NMR (400 MHz, DMSO-d₆) δ 8.53 (t, J = 5.8 Hz, 1 H), 8.34 (s, 1 H), 7.77 (s, 1 H), 7.48 (t, J = 1.9 Hz, 1 H), 7.39 (d, J = 1.8 Hz, 2 H), 7.16 (s, 1 H), 6.85 (dd, J = 16.8, 10.4 Hz, 1 H), 6.11 (dd, J = 16.7, 2.4 Hz, 1 H), 5.68 (dd, J = 10.5, 2.4 Hz, 1 H), 4.84-4.63 (m, 3 H), 3.91 (s, 3 H), 3.89-3.73 (m, 2 H), 3.60-3.38 (m, 2 H), 2.08-1.92 (m, 2 H), 1.78-1.60 (m, 2 H). |
| 118 | 1-(4-((4-((5- chloro-2- fluorobenzyl) amino)-7- methoxy- quinazolin-6- yl)oxy) piperidin-1- yl)prop-2- en-1-one | Method A | 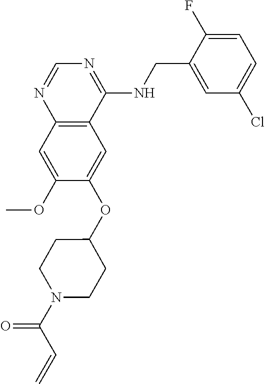 [SMILES: C=CC(=O)N1CCC(Oc2cc3c(NCc4cc(Cl)ccc4F)ncnc3cc2OC)CC1] | TFA | LC/MS (ESI, m/z): [M + H]⁺ = 471.2, 473.2. ¹H NMR (400 MHz, DMSO-d₆) δ 10.17 (s, 1 H), 8.84 (s, 1 H), 8.04 (s, 1 H), 7.50 (dd, J = 6.4, 2.6 Hz, 1 H), 7.47- 7.40 (m, 1 H), 7.36-7.21 (m, 2 H), 6.85 (dd, J = 16.7, 10.5 Hz, 1 H), 6.12 (dd, J = 16.7, 2.4 Hz, 1 H), 5.69 (dd, J = 10.5, 2.4 Hz, 1 H), 4.96 (d, J = 5.5 Hz, 2 H), 4.89-4.75 (m, 1 H), 3.98 (s, 3 H), 3.86-3.79 (m, 2 H), 3.58-3.45 (m, 2 H), 2.09-1.96 (m, 2 H), 1.78-1.62 (m, 2 H). |
| 119 | 1-(4-((4- ((2,5- dichlorobenzyl) amino)-7- methoxy- quinazolin-6- yl)oxy) piperidin-1- yl)prop-2- en-1-one | Method A | 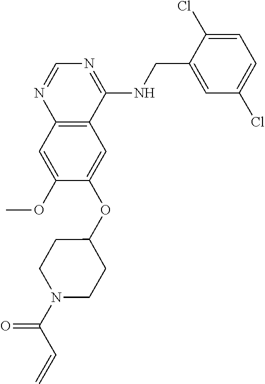 [SMILES: C=CC(=O)N1CCC(Oc2cc3c(NCc4cc(Cl)ccc4Cl)ncnc3cc2OC)CC1] | TFA | LC/MS (ESI, m/z): [M + H]⁺ = 487.1. ¹H NMR (400 MHz, DMSO-d₆) δ 9.99 (br s, 1 H), 8.82 (s, 1 H), 8.00 (s, 1 H), 7.57 (d, J = 8.4 Hz, 1 H), 7.47 (d, J = 2.0 Hz, 1 H), 7.44 (dd, J = 8.4, 2.8 Hz, 1 H), 7.24 (d, J = 9.6 Hz, 1H), 6.85 (dd, J = 16.8, 10.4 Hz, 1 H), 6.12 (dd, J = 16.8, 2.4 Hz, 1 H), 5.69 (dd, J = 10.4, 2.4 Hz, 1 H), 4.98 (d, J = 4.8 Hz, 2 H), 4.78-4.84 (m, 1 H), 3.99 (s, 3 H), 3.77- 3.85 (m, 2 H), 3.48-3.54 (m, 2 H), 1.95- 2.09 (m, 2 H), 1.63-1.79 (m, 2 H). |
| 120 | 1-(4-((4-((3- bromobenzyl) amino)-7- methoxy- quinazolin-6- yl)oxy) piperidin-1- yl)prop-2- en-1-one | Method B | 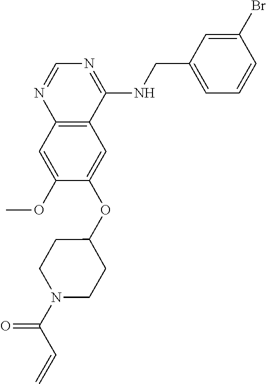 [SMILES: C=CC(=O)N1CCC(Oc2cc3c(NCc4cccc(Br)c4)ncnc3cc2OC)CC1] | None | LC/MS (ESI, m/z): [M + H]⁺ = 497.0, 499.0. ¹H NMR (400 MHz, DMSO-d₆) δ 8.47 (t, J = 6.0 Hz, 1 H), 8.33 (s, 1 H), 7.76 (s, 1 H), 7.54 (s, 1 H), 7.44 (d, J = 7.8 Hz, 1 H), 7.36 (d, J = 7.7 Hz, 1 H), 7.29 (t, J = 7.7 Hz, 1 H), 7.15 (s, 1 H), 6.84 (dd, J = 16.7, 10.5 Hz, 1 H), 6.11 (dd, J = 16.7, 2.4 Hz, 1 H), 5.68 (dd, J = 10.5, 2.4 Hz, 1 H), 4.79 (d, J = 5.3 Hz, 2 H), 4.74-4.70 (m, 1 H), 3.91 (s, 3 H), 3.90-3.86 (m, 2 H), 3.50-3.43 (m, 2 H), 2.05-1.96 (m, 2 H), 1.74-1.62 (m, 2 H). |
| 121 | 1-(4-((7- methoxy-4- ((3- methylbenzyl) amino) quinazolin-6- yl)oxy) piperidin-1- yl)prop-2- en-1-one | Method B | 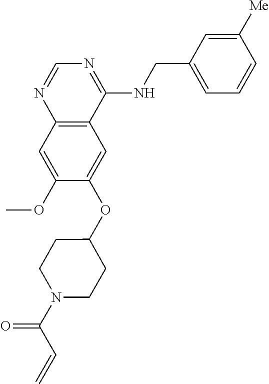 [SMILES: C=CC(=O)N1CCC(Oc2cc3c(NCc4cccc(C)c4)ncnc3cc2OC)CC1] | None | LC/MS (ESI, m/z): [M + H]⁺ = 433.2. ¹H NMR (400 MHz, DMSO-d₆) δ 8.40 (t, J = 5.7 Hz, 1 H), 8.32 (s, 1 H), 7.78 (s, 1 H), 7.22-7.12 (m, 4 H), 7.05 (d, J = 7.4 Hz, 1 H), 6.84 (dd, J = 16.7, 10.5 Hz, 1 H), 6.11 (dd, J = 16.7, 2.2 Hz, 1 H), 5.68 (dd, J = 10.5, 2.2 Hz, 1 H), 4.76-4.69 (m, 3 H), 3.90 (s, 3 H), 3.86-3.80 (m, 2 H), 3.54-3.40 (m, 2 H), 2.27 (s, 3 H), 2.03-1.93 (m, 2 H), 1.73- 1.60 (m, 2 H). |
| 122 | 1-(4-((4-((3- chloro-5- fluorobenzyl) amino)-7- methoxy- quinazolin-6- yl)oxy) piperidin-1- yl)prop-2- en-1-one | Method B | 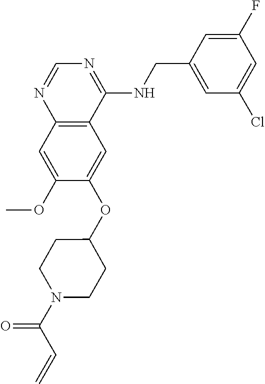 [SMILES: C=CC(=O)N1CCC(Oc2cc3c(NCc4cc(F)cc(Cl)c4)ncnc3cc2OC)CC1] | None | LC/MS (ESI, m/z): [M + H]⁺ = 471.0, 473.0. ¹H NMR (400 MHz, DMSO-d₆) δ 8.49 (t, J = 5.9 Hz, 1 H), 8.33 (s, 1 H), 7.75 (s, 1 H), 7.33-7.24 (m, 2 H), 7.18- 7.16 (m, 2 H), 6.84 (dd, J = 16.7, 10.5 Hz, 1 H), 6.11 (dd, J = 16.7, 2.4 Hz, 1 H), 5.68 (dd, J = 10.5, 2.4 Hz, 1 H), 4.79 (d, J = 5.8 Hz, 2 H), 4.76-4.68 (m, 1 H), 3.91 (s, 3 H), 3.87-3.85 (m, 2 H), 3.48-3.45 (m, 2 H), 2.04-1.98 (m, 2H), 1.78-1.60 (m, 2 H). |
| 123 | 1-(4-((4- (benzylamino)- 7- methoxy- quinazolin-6- yl)oxy) piperidin-1- yl)prop-2- en-1-one | Method B | 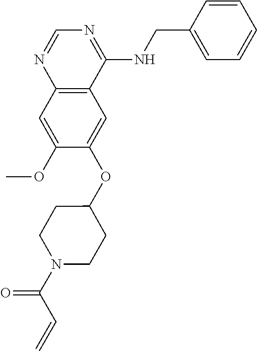 [SMILES: C=CC(=O)N1CCC(Oc2cc3c(NCc4ccccc4)ncnc3cc2OC)CC1] | TFA | LC/MS (ESI, m/z): [M + H]⁺ = 419.2. ¹H NMR (400 MHz, DMSO-d₆) δ 10.14 (br s, 1 H), 8.83 (s, 1 H), 7.98 (s, 1 H), 7.33-7.40 (m, 5 H), 7.22 (s, 1 H), 6.84 (dd, J = 16.7, 10.5 Hz, 1 H), 6.11 (dd, J = 16.7, 2.4 Hz, 1 H), 5.69 (dd, J = 10.4, 2.4 Hz, 1 H), 4.98 (d, J = 5.9 Hz, 2 H), 4.82-4.76 (m, 1 H), 3.98 (s, 3 H), 3.84-3.78 (m, 2 H), 3.74-3.66 (m, 2 H), 2.08-1.95 (m, 2 H), 1.76-1.63 (m, 2 H). |
| 124 | 3-(((6-((1- acryloyl- piperidin-4- yl)oxy)-7- methoxy- quinazolin-4- yl)amino) methyl) benzonitrile | Method B | 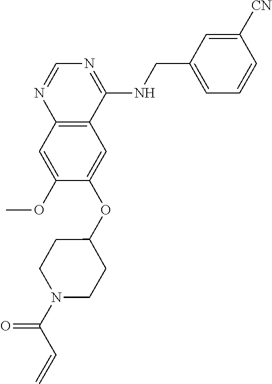 [SMILES: C=CC(=O)N1CCC(Oc2cc3c(NCc4cccc(C#N)c4)ncnc3cc2OC)CC1] | TFA | LC/MS (ESI, m/z): [M + H]⁺ = 444.2. ¹H NMR (400 MHz, DMSO-d₆) δ 10.32 (br s, 1 H), 8.83 (s, 1 H), 7.96-8.07 (m, 1 H), 7.87 (s, 1 H), 7.71-7.82 (m, 2 H), 7.58 (t, J = 7.7 Hz, 1 H), 7.24-7.32 (m, 1 H), 6.84 (dd, J = 16.8, 10.8 Hz, 1 H), 6.12 (dd, J = 16.8, 2.4 Hz, 1 H), 5.69 (dd, J = 10.8, 2.4 Hz, 1 H), 5.01 (d, J = 6.0 Hz, 2 H), 4.78-4.86 (m, 1 H), 3.99 (s, 3 H), 3.79-3.90 (m, 2 H), 3.51- 3.56 (m, 2 H), 1.97-2.09 (m, 2 H), 1.61- 1.76 (m, 2 H). |
| 125 | 1-(4-((4-((3- iodobenzyl) amino)-7- methoxy- quinazolin-6- yl)oxy) piperidin-1- yl)prop-2- en-1-one | Method B | 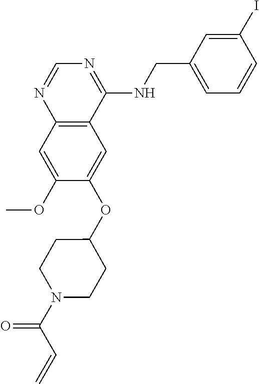 [SMILES: C=CC(=O)N1CCC(Oc2cc3c(NCc4cccc(I)c4)ncnc3cc2OC)CC1] | TFA | LC/MS (ESI, m/z): [M + H]⁺ = 545.0. ¹H NMR (400 MHz, DMSO-d₆) δ 10.16 (s, 1 H), 8.84 (s, 1 H), 7.98 (s, 1 H), 7.79 (s, 1 H), 7.67 (d, J = 7.9 Hz, 1 H), 7.41 (d, J = 7.8 Hz, 1 H), 7.28 (s, 1 H), 7.17 (t, J = 7.8 Hz, 1 H), 6.84 (dd, J = 16.7, 10.5 Hz, 1 H), 6.12 (dd, J = 16.7, 2.4 Hz, 1 H), 5.69 (dd, J = 10.5, 2.4 Hz, 1 H), 4.93 (d, J = 5.8 Hz, 2 H), 4.81-4.79 (m, 1 H), 3.98 (s, 3 H), 3.82-3.80 (m, 2 H), 3.56-3.50 (m, 2 H), 2.03-2.01 (m, 2 H), 1.73-1.65 (m, 2 H). |

### Example 126: Preparation of 2-((6-((1-acryloylpiperidin-4-yl)oxy)-7-methoxyquinazolin-4-yl)amino)-2-(3-chlorophenyl)acetic acid (Compound 126)

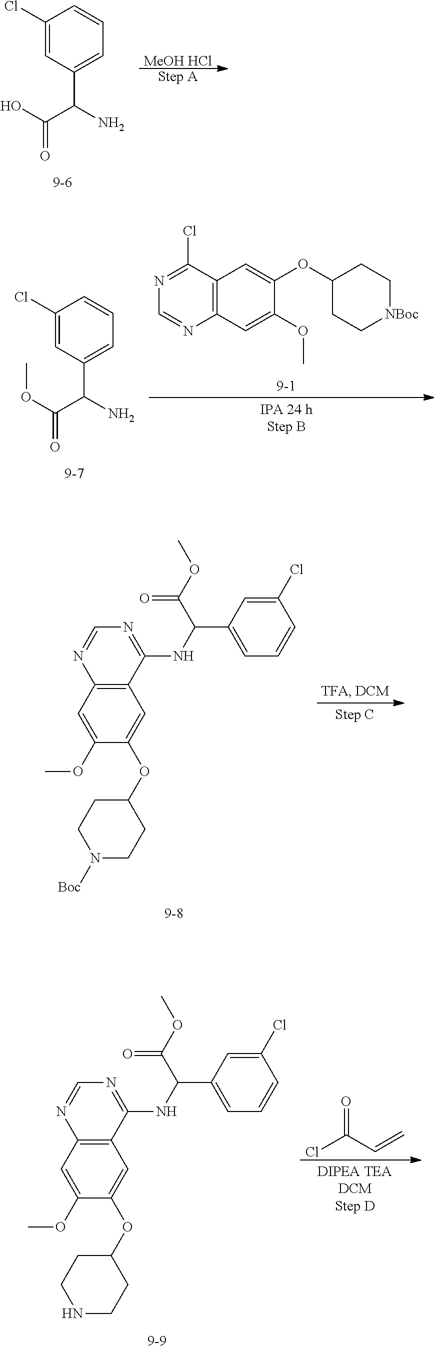

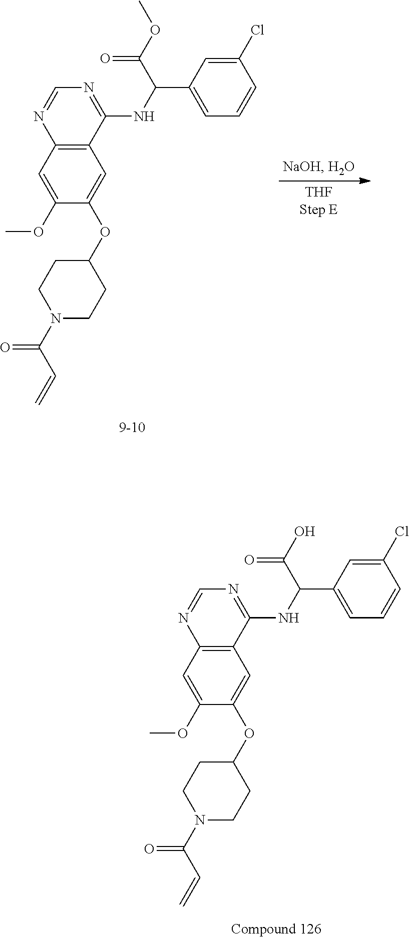 [SMILES: C=CC(=O)N1CCC(Oc2cc3c(NC(C(=O)OC)c4cccc(Cl)c4)ncnc3cc2OC)CC1 ; C=CC(=O)N1CCC(Oc2cc3c(NC(C(=O)O)c4cccc(Cl)c4)ncnc3cc2OC)CC1]

Step A: A mixture of 2-amino-2-(3-chlorophenyl)acetic acid (500 mg, 2.69 mmol) and HCl (8 mL, 4M in MeOH), the mixture was stirred at r.t. for 3 h. The mixture was concentrated in vacuo. The residue was purified by column chromatography to give the methyl 2-amino-2-(3-chlorophenyl)acetate (250 mg, 46% yield) as a white solid. LC/MS (ESI, m/z): [M+1]⁺=200.1.

Step B: A mixture of methyl 2-amino-2-(3-chlorophenyl)acetate (200 mg, 1.00 mmol), tert-butyl 4-((4-chloro-7-methoxyquinazolin-6-yl)oxy)piperidine-1-carboxylate (393 mg, 1.00 mmol), and Et₃N (303 mg, 3.00 mmol) in isopropanol (8 mL) was degassed with nitrogen. The reaction mixture was heated to 90° C. for 24 hour under nitrogen atmosphere. The reaction was cooled to r.t. and concentrated in vacuum. The residue was purified by column chromatography to give the tert-butyl 4-((4-((1-(3-chlorophenyl)-2-methoxy-2-oxoethyl)amino)-7-methoxyquinazolin-6-yl)oxy)piperidine-1-carboxylate (140 mg, 25% yield) as an oil. LC/MS (ESI, m/z): [M+1]⁺=557.3

Step C: A solution of tert-butyl 4-((4-((1-(3-chlorophenyl)-2-methoxy-2-oxoethyl)amino)-7-methoxyquinazolin-6-yl)oxy)piperidine-1-carboxylate (140 mg, 0.25 mmol) in DCM (10 mL) was slowly added TFA (5 mL) and stirred at r.t. for 1 h. The reaction was then concentrated in vacuo to give the methyl 2-(3-chlorophenyl)-2-((7-methoxy-6-(piperidin-4-yloxy)quinazolin-4-yl)amino)acetate (113 mg, 99% yield) as a yellow oil. LC/MS (ESI, m/z): [M+1]⁺=457.2

Step D: To a solution of methyl 2-(3-chlorophenyl)-2-((7-methoxy-6-(piperidin-4-yloxy)quinazolin-4-yl) amino)acetate (113 mg, 0.24 mmol) in DCM (5 mL) was added acryloyl chloride (23 mg, 0.24 mmol) and DIPEA (96 mg, 0.74 mmol). The mixture was stirred at r.t. for 1 hour. Then, the reaction mixture was quenched by water, and extracted with DCM. The organic layer was washed with brine, dried over anhydrous sodium sulfate, filtered and concentrated in vacuo. The residue was purified by column chromatography to give the methyl 2-((6-((1-acryloylpiperidin-4-yl)oxy)-7-methoxyquinazolin-4-yl)amino)-2-(3-chlorophenyl)acetate (60 mg, 45% yield) as an oil. LC/MS (ESI, m/z): [M+1]⁺=511.3

Step E: A solution of methyl 2-((6-((1-acryloylpiperidin-4-yl)oxy)-7-methoxyquinazolin-4-yl)amino)-2-(3-chlorophenyl)acetate (60 mg, 0.11 mmol) in THF (10 mL) was slowly added NaOH (aq) (9 mg, 0.24 mmol) at r.t. The mixture was stirred at r.t. for 2 h. The pH was adjusted to 5 with 1 N HCl(aq) and the mixture was then extracted with EA. The organic layer was washed with brine, dried over anhydrous sodium sulfate, filtered and concentrated in vacuo. The residue was purified by prep-HPLC to give the 2-((6-((1-acryloylpiperidin-4-yl)oxy)-7-methoxyquinazolin-4-yl)amino)-2-(3-chlorophenyl)acetic acid (2.4 mg, 4% yield) as a white solid. LC/MS (ESI, m/z): [M+1]⁺=497.2, 500.2. ¹H NMR (400 MHz, DMSO-d₆) δ 13.32 (br, 1H), 9.39-8.78 (m, 1H), 8.62 (s, 1H), 8.07 (s, 1H), 7.63 (s, 1 H), 7.59-7.40 (m, 3H), 7.23 (s, 1H), 6.84 (dd, J=16.7, 10.5 Hz, 1H), 6.16-6.02 (m, 2H), 5.68 (dd, J=10.5, 2.4 Hz, 1H), 4.89-4.74 (m, 1H), 3.95 (s, 3H), 3.86-3.69 (m, 2H), 3.63-3.56 (m, 2H), 2.05-1.86 (m, 2H), 1.83-1.64 (m, 2H).

### Example 127: Preparation of 2-((6-((1-acryloylpiperidin-4-yl)oxy)-7-methoxyquinazolin-4-yl)amino)-2-(3-chlorophenyl)acetamide (Compound 127)

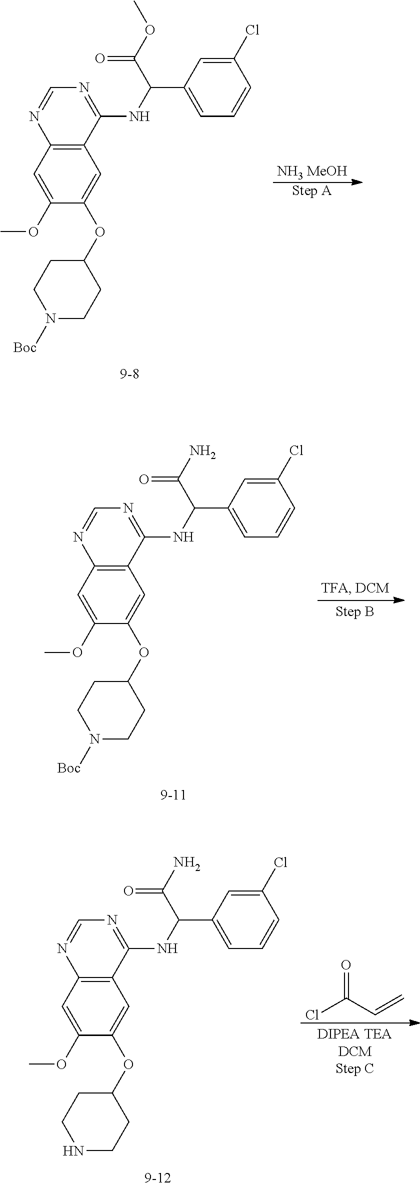

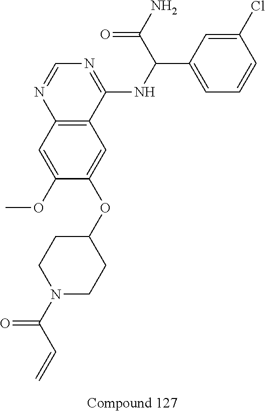 [SMILES: C=CC(=O)N1CCC(Oc2cc3c(NC(C(N)=O)c4cccc(Cl)c4)ncnc3cc2OC)CC1]

Step A: A solution of tert-butyl 4-((4-((1-(3-chlorophenyl)-2-methoxy-2-oxoethyl)amino)-7-methoxy quinazolin-6-yl)oxy)piperidine-1-carboxylate (70 mg, 0.12 mmol) was added Ammonia solution in methanol(5 mL, 7M) and stirred at r.t. overnight. The reaction was then concentrated and purified by column chromatography to give the tert-butyl 4-((4-((2-amino-1-(3-chlorophenyl)-2-oxoethyl)amino)-7-methoxyquinazolin-6-yl)oxy)piperidine-1-carboxylate (60 mg, 88% yield) as a yellow oil. LC/MS (ESI, m/z): [M+1]⁺=542.2.

Steps B and C were completed in a similar manner as described in Example 126 steps C and D to afford 2-((6-((1-acryloylpiperidin-4-yl)oxy)-7-methoxyquinazolin-4-yl)amino)-2-(3-chlorophenyl)acetamide (Compound 127). LC/MS (ESI, m/z): [M+1]⁺=496.2, 498.2. ¹H NMR (400 MHz, DMSO-d₆) δ 9.69-9.51 (m, 1H), 8.81 (s, 1H), 8.23 (s, 1H), 7.98 (s, 1H), 7.65 (s, 1H), 7.61-7.39 (m, 4H), 7.25 (s, 1H), 6.84 (dd, J=16.7, 10.5 Hz, 1H), 6.19 (d, J=7.4 Hz, 1H), 6.12 (dd, J=16.7, 2.3 Hz, 1H), 5.69 (dd, J=10.5, 2.4 Hz, 1H), 4.98-4.81 (m, 1 H), 3.98 (s, 3H), 3.77-3.58 (m, 4H), 2.06-1.86 (m, 2H), 1.84-1.65 (m, 2H).

### Example 128: Preparation of 1-(4-((4-((1-(3-chlorophenyl)-2-hydroxyethyl)amino)-7-methoxyuinazolin-6-yl) oxy)piperidin-1-yl)prop-2-en-1-one (Compound 128)

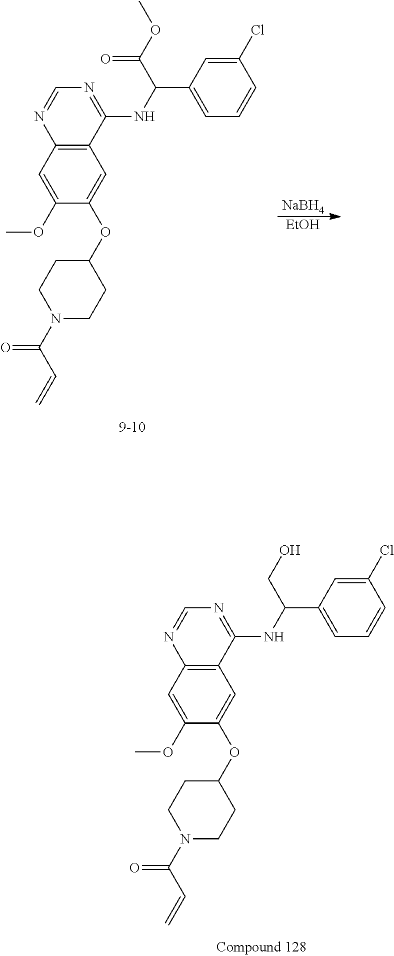 [SMILES: "EtOH" ; C=CC(=O)N1CCC(Oc2cc3c(NC(C(=O)OC)c4cccc(Cl)c4)ncnc3cc2OC)CC1 ; "NaBH4" ; C=CC(=O)N1CCC(Oc2cc3c(NC(CO)c4cccc(Cl)c4)ncnc3cc2OC)CC1]

A solution of methyl 2-((6-((1-acryloylpiperidin-4-yl)oxy)-7-methoxyquinazolin-4-yl)amino)-2-(3-chlorophenyl)acetate (68 mg, 0.13 mmol) in EtOH (10 mL) was slowly added NaBH₄ (22 mg, 0.58 mmol) at r.t., then the mixture was stirred at RT for 3 h. The reaction was then quenched by water, extracted with EA. The combined organic layer was washed with brine, dried over anhydrous sodium sulfate, filtered and concentrated in vacuo. The residue was purified by prep-HPLC to give the 1-(4-((4-((1-(3-chlorophenyl)-2-hydroxyethyl)amino)-7-methoxyquinazolin-6-yl) oxy)piperidin-1-yl)prop-2-en-1-one (Compound 128) (18.7 mg, 29% yield) as a white solid. LC/MS (ESI, m/z): [M+1]⁺=483.0, 485.0. ¹H NMR (400 MHz, DMSO-d₆) δ 8.27 (s, 1H), 7.99 (d, J=7.8 Hz, 1H), 7.93 (s, 1H), 7.52-7.45 (m, 1H), 7.42-7.31 (m, 3H), 7.30-7.25 (m, 1H), 7.13 (s, 1H), 6.84 (dd, J=16.7, 10.5 Hz, 1H), 6.11 (dd, J=16.7, 2.4 Hz, 1H), 5.68 (dd, J=10.5, 2.4 Hz, 1H), 5.49 (dd, J=13.4, 7.4 Hz, 1H), 5.09 (s, 1 H),4.91-4.66 (m, 1H), 3.90 (s, 3H), 3.86-3.67 (m, 4H), 3.63-3.51 (m, 2H), 2.10-1.90 (m, 2H), 1.81-1.64 (m, 2H).

### Example 129: Preparation of 3-((6-((1-acryloylpiperidin-4-yl)oxy)-7-methoxyquinazolin-4-yl)amino)-3-(3-chlorophenyl)propanoic acid (Compound 129)

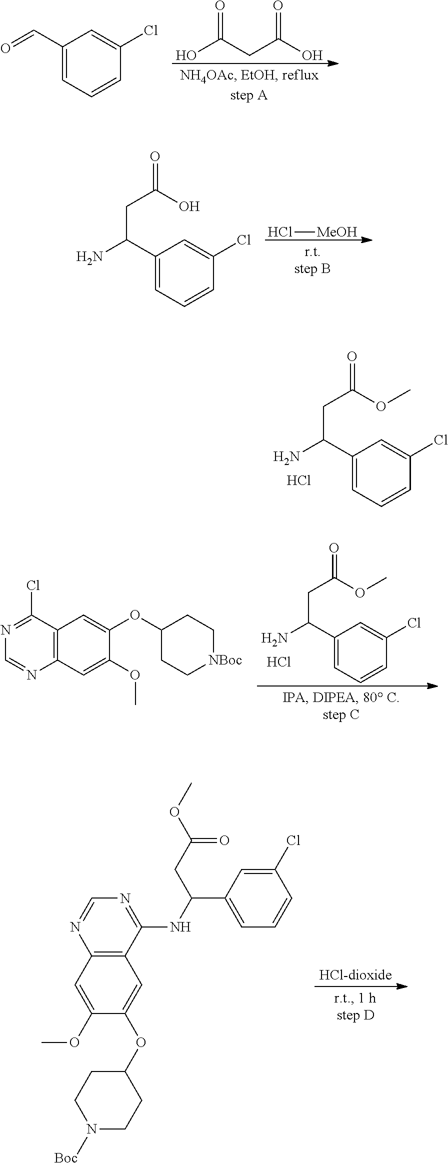

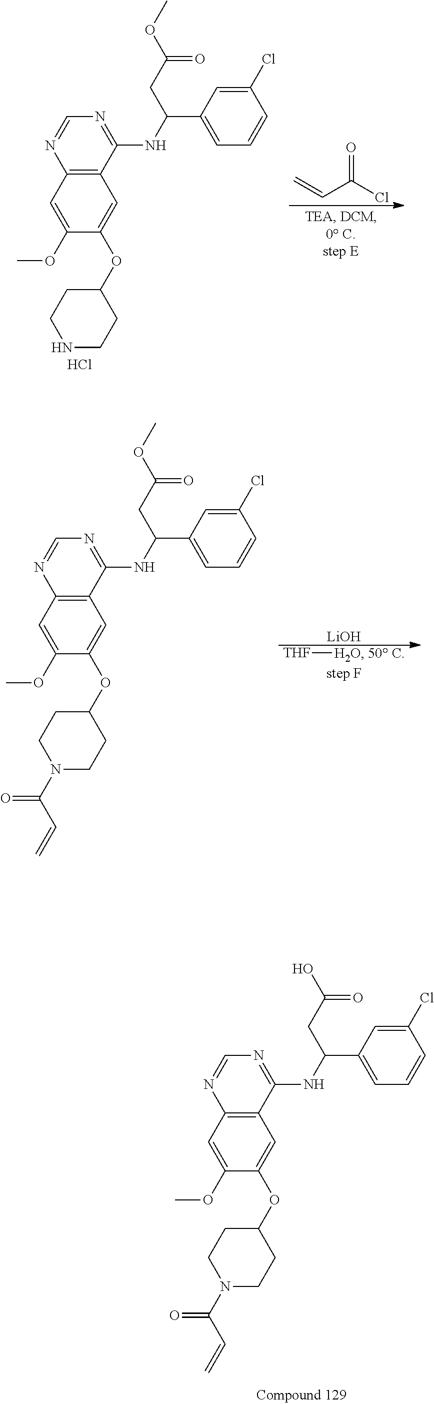

Step A: A solution of 3-chlorobenzaldehyde (4.22 g, 30 mmol, 1 equiv), malonic acid (3.12 g, 30 mmol, 1 equiv) and ammonium acetate (6.94 g, 90 mmol, 3 equiv) in EtOH (100 mL) was refluxed for 6 hours. The reaction mixture was allowed to cool to room temperature and the white precipitate was collected by filtration. The precipitate was washed with MeOH and dried in vacuo to afford the title compound 3-amino-3-(3-chlorophenyl)propanoic acid as a white solid (2.7 g, 48% yield).

Step B: To a suspension of 3-amino-3-(3-chlorophenyl)propanoic acid (2.7 g, 12.6 mmol, 1 equiv) in MeOH (10 mL) was added HCl-MeOH solution (4 mol/L, 31.5 mL, 126 mmol, 10 equiv). The resulting mixture was stirred at room temperature for 12 hours. The volatile was evaporated to afford the title compound 3-amino-3-(3-chlorophenyl)propanoic acid as a white solid (3.1 g, 100% yield). LC/MS (ESI, m/z): [M+H]⁺=214.1.

Step C: To a solution of tert-butyl 4-((4-chloro-7-methoxyquinazolin-6-yl)oxy)piperidine-1-carboxylate (300 mg, 0.76 mmol, 1 equiv) in isopropanol (10 mL) was added methyl 3-amino-3-(3-chlorophenyl)propanoate hydrochloride (568 mg, 2.28 mmol, 3 equiv) and DIPEA (588 mg, 4.56 mmol, 6 equiv). The resulted mixture was stirred at 80° C. for 24 hours. The reaction mixture was concentrated in vacuo. To the residue was added water (30 mL) and the mixture was extracted with EtOAc (30 mL×2). The combined organic layers were dried over Na₂SO₄ and concentrated in vacuo. The residue was purified by silica gel chromatography (eluted with DCM/MeOH=10/1) to afford the title compound tert-butyl 4-((4-((1-(3-chlorophenyl)-3-methoxy-3-oxopropyl)amino)-7-methoxyquinazolin-6-yl)oxy) piperidine-1-carboxylate as a white solid (320 mg, 74% yield). LC/MS (ESI, m/z): [M+H]⁺=571.2.

Step D: To a solution of compound tert-butyl 4-((4-((1-(3-chlorophenyl)-3-methoxy-3-oxopropyl)amino)-7-methoxyquinazolin-6-yl)oxy) piperidine-1-carboxylate (80 mg, 0.14 mmol, 1 equiv) in DCM (2 mL) was added HCl (4M in 1,4-dioxane, 3.5 mL, 100 equiv), the reaction solution was stirred at room temperature for 1 hour, and then concentrated in vacuo to give crude compound methyl 3-(3-chlorophenyl)-3-((7-methoxy-6-(piperidin-4-yloxy) quinazolin-4-yl) amino) propanoate hydrochloride as a white solid (71 mg crude). LC/MS (ESI, m/z): [M+H]⁺=471.2.

Step E: To a solution of methyl 3-(3-chlorophenyl)-3-((7-methoxy-6-(piperidin-4-yloxy) quinazolin-4-yl)amino) propanoate hydrochloride (71 mg, 0.14 mmol, 1 equiv) and triethylamine (56 mg, 0.56 mmol, 4 equiv) in DCM (5 mL) was added acryloyl chloride (14 mg, 0.15 mmol, 1.1 equiv) at 0° C. The reaction solution was stirred at room temperature for 1 hour. Then the reaction solution was quenched with water (5 mL) and extracted with EtOAc (5 mL×3). The combined organic layers were washed with brine, dried over Na₂SO₄ and concentrated in vacuo. The residue was purified by silica gel chromatography (eluted with DCM/MEOH=10/1) to give the title compound methyl 3-((6-((1-acryloylpiperidin-4-yl)oxy)-7-methoxyquinazolin-4-yl)amino)-3-(3-chlorophenyl) propanoate as a white solid (70 mg, 93% yield). LC/MS (ESI, m/z): [M+H]⁺=525.1.

Step F: To a solution of methyl 3-((6-((1-acryloylpiperidin-4-yl)oxy)-7-methoxyquinazolin-4-yl) amino)-3-(3-chlorophenyl) propanoate (60 mg, 0.11 mmol, 1 equiv) in THF/H₂O (5 mL/0.25 mL) was added lithium hydroxide (9 mg, 0.22 mmol, 2 equiv). The mixture was stirred at 50° C. for 4 hours. The reaction mixture was concentrated in vacuo. To the residue was added EtOAc (10 mL) and the mixture was washed by 1N HCl solution (10 mL×2). The organic layer was separated, dried over Na₂SO₄ and concentrated in vacuo. The residue was purified by silica gel chromatography (eluted with DCM/MeOH=10/1) and further purified by reverse phase prep-HPLC (eluted with CH₃CN/H₂O=5%˜90% with 0.1% TFA) to afford the title compound 3-((6-((1-acryloylpiperidin-4-yl)oxy)-7-methoxyquinazolin-4-yl)amino)-3-(3-chlorophenyl)propanoic acid (Compound 129) as a white solid (8 mg, 14% yield). LC/MS (ESI, m/z): [M+H]⁺=511.2. ¹H NMR (600 MHz, DMSO-d₆) δ 12.53 (br s, 1H), 9.45 (br s, 1 H), 8.75 (s, 1H), 8.03 (s, 1H), 7.54 (s, 1H), 7.45 (d, J=7.8 Hz, 1H), 7.40 (t, J=7.8 Hz, 1H), 7.36 (d, J=8.0 Hz, 1H), 7.21 (s, 1H), 6.85 (dd, J=16.7, 10.5 Hz, 1H), 6.12 (dd, J=16.7, 2.4 Hz, 1H), 5.99 (dd, J=14.6, 8.0 Hz, 1H), 5.69 (dd, J=10.5, 2.3 Hz, 1H), 4.84 (m, 1H), 3.97 (s, 3H), 3.79-3.67 (m, 2H), 3.67-3.57 (m, 2H), 3.10 (dd, J=16.5, 9.1 Hz, 1H), 3.03 (dd, J=16.5, 5.9 Hz, 1H), 2.03-1.93 (m, 2H), 1.79-1.69 (m, 2H). LC/MS (ESI, m/z): [M+H]⁺=511.2.

### Example 130: Preparation of 3-((6-((1-acryloylpiperidin-4-yl)oxy)-7-methoxyquinazolin-4-yl)amino)-3-(3-chlorophenyl)propanamide (Compound 130)

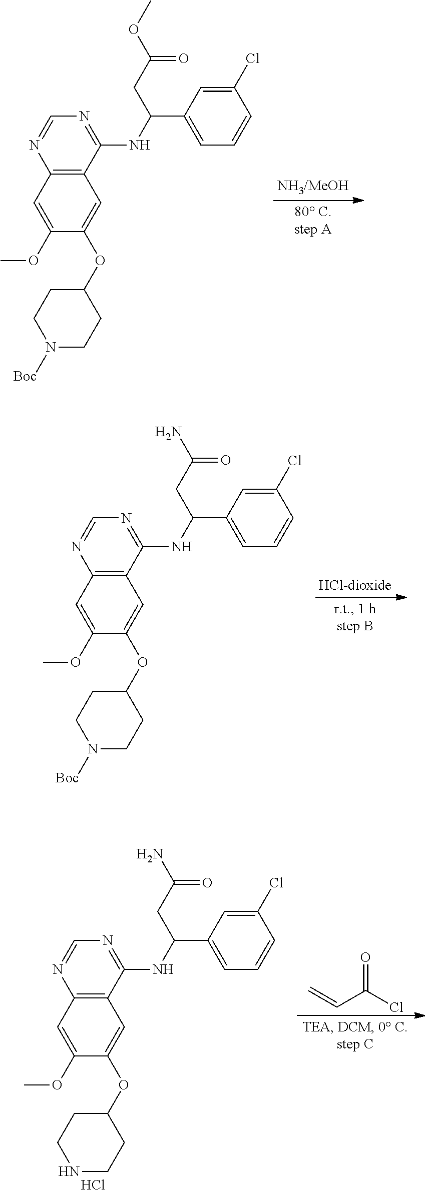

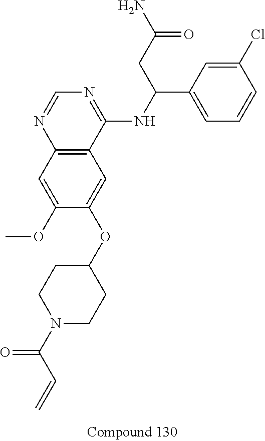 [SMILES: C=CC(=O)N1CCC(Oc2cc3c(NC(CC(N)=O)c4cccc(Cl)c4)ncnc3cc2OC)CC1]

Step A: To a solution of tert-butyl 4-((4-((1-(3-chlorophenyl)-3-methoxy-3-oxopropyl) amino)-7-methoxyquinazolin-6-yl)oxy)piperidine-1-carboxylate (100 mg, 0.19 mmol, 1 equiv) in MeOH (1 mL) was added NH₃ (7 mol/L in MeOH, 5 mL, 35 mmol, 184 equiv). The reaction solution was stirred at 80° C. for 24 hours and then concentrated in vacuo. The residue was purified by silica gel chromatography (eluted with DCM/MeOH=10/1) to afford the title compound tert-butyl 4-((4-((3-amino-1-(3-chlorophenyl)-3-oxopropyl)amino)-7-methoxyquinazolin-6-yl)oxy)piperidine-1-carboxylate as a white solid (90 mg, 85% yield). LC/MS (ESI, m/z): [M+H]⁺=556.2.

Step B: To a solution of tert-butyl 4-((4-((3-amino-1-(3-chlorophenyl)-3-oxopropyl) amino)-7-methoxyquinazolin-6-yl)oxy)piperidine-1-carboxylate (80 mg, 0.14 mmol, 1 equiv) in DCM (2 mL) was added HCl (4 mol/L in 1,4-dioxane, 3.5 mL, 100 equiv). The reaction solution was stirred at room temperature for 1 hour and then concentrated to give crude compound 3-(3-chlorophenyl)-3-((7-methoxy-6-(piperidin-4-yloxy)quinazolin-4-yl)amino)propanamide hydrochloride as a white solid (80 mg, 100% yield). LC/MS (ESI, m/z): [M+H]⁺=456.2.

Step C: To a solution of compound 3-(3-chlorophenyl)-3-((7-methoxy-6-(piperidin-4-yloxy) quinazolin-4-yl)amino)propanamide hydrochloride (80 mg, 0.14 mmol, 1.0 equiv) and triethylamine (58 mg, 0.56 mmol, 4.0 equiv) in DCM (5 mL) was added acryloyl chloride (14 mg, 0.15 mmol, 1.1 equiv) at 0° C. The reaction solution was stirred at the room temperature for 1 hour. Then the reaction solution was quenched with water (5 mL) and extracted with EtOAc (5 mL×3). The combined organic layers were washed with brine, dried over Na₂SO₄, and concentrated. The residue was pre-purified by silica gel chromatography (eluted with DCM/MeOH=10/1) and further purified by reverse phase prep-HPLC (eluted with CH₃CN/H₂O=5%˜90% with 0.1% TFA) to give the compound 3-((6-((1-acryloylpiperidin-4-yl)oxy)-7-methoxyquinazolin-4-yl)amino)-3-(3-chlorophenyl)propanamide (Compound 130) as TFA salt as a white solid (32 mg, 45% yield). LC/MS (ESI, m/z): [M+H]⁺=510.2. ¹H NMR (400 MHz, DMSO-d₆) δ 9.72 (d, J=6.8 Hz, 1H), 8.81 (s, 1H), 8.05 (s, 1H), 7.55-7.51 (m, 2 H), 7.44-7.34 (m,3 H), 7.23 (s, 1H), 6.99 (s, 1H), 6.85 (dd, J=16.7, 10.5 Hz, 1H), 6.12 (dd, J=16.7, 2.4 Hz, 1H), 6.03 (dd, J=6.8, 7.6 Hz, 1H), 5.70 (dd, J=10.5, 2.4 Hz, 1H), 4.88-4.79 (m, 1H), 3.98 (s, 3H), 3.81-3.53 (m, 4H), 2.88 (d, J=7.6 Hz, 2H), 2.06-1.92 (m, 2H), 1.82-1.66 (m, 2H).

### Example 131: Preparation of 1-(4-((4-((1-(3-chlorophenyl)-3-hydroxypropyl)amino)-7-methoxyuinazolin-6-yl)oxy)piperidin-1-yl)prop-2-en-1-one (Compound 131)

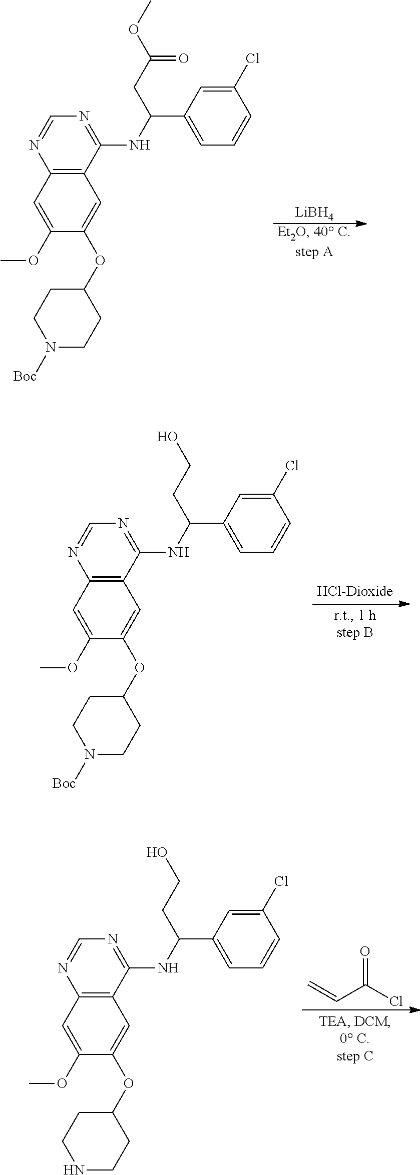

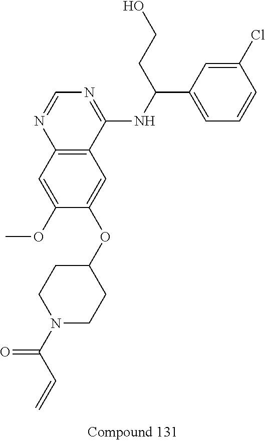 [SMILES: C=CC(=O)N1CCC(Oc2cc3c(NC(CCO)c4cccc(Cl)c4)ncnc3cc2OC)CC1]

Step A: To a solution of tert-butyl 4-((4-((1-(3-chlorophenyl)-3-methoxy-3-oxopropyl)amino)-7-methoxyquinazolin-6-yl)oxy)piperidine-1-carboxylate (150 mg, 0.26 mmol, 1 equiv) in Et₂O (10 mL) was added lithium borohydride (8.6 mg, 0.39 mmol, 1.5 equiv). The resulting mixture was stirred at 40° C. for 6 hours. The reaction mixture was quenched with water (30 mL) and extracted with EtOAc (30 mL×3). The combined organic layers were dried over Na₂SO₄ and concentrated in vacuo. The residue was purified by chromatography on silica gel (eluted with DCM/MeOH=10/1) to afford the title compound tert-butyl 4-((4-((1-(3-chlorophenyl)-3-hydroxypropyl) amino)-7-methoxyquinazolin-6-yl)oxy)piperidine-1-carboxylate as a white solid (84 mg, 59% yield). LC/MS (ESI, m/z): [M+H]⁺=543.1.

Step B: To a solution of tert-butyl 4-((4-((1-(3-chlorophenyl)-3-hydroxypropyl)amino)-7-methoxyquinazolin-6-yl)oxy)piperidine-1-carboxylate (84 mg, 0.15 mmol, 1 equiv) in DCM (2 mL) was added HCl (4 mol/L in 1,4-dioxane, 5 mL, 131 equiv). The reaction solution was stirred at room temperature for 1 hour, then concentrated in vacuo to give crude 3-(3-chlorophenyl)-3-((7-methoxy-6-(piperidin-4-yloxy)quinazolin-4-yl) amino) propan-1-ol hydrochloride as a white solid without further purification (90 mg crude). LC/MS (ESI, m/z): [M+H]⁺=443.1.

Step C: To a solution of 3-(3-chlorophenyl)-3-((7-methoxy-6-(piperidin-4-yloxy) quinazolin-4-yl)amino) propan-1-ol hydrochloride (72 mg, 0.15 mmol, 1.0 equiv) and triethylamine (46 mg, 0.45 mmol, 3.0 equiv) in DCM (5 mL) was added acryloyl chloride (13 mg, 0.15 mmol, 1.0 equiv) at 0° C. The reaction solution was stirred at the room temperature for 1 hour. Then the reaction solution was quenched with water (5 mL) and extracted with EtOAc (5 mL×3). The combined organic layers were washed with brine, dried over Na₂SO₄ and concentrated in vacuo. The residue was pre-purified by silica gel chromatography (eluted with DCM/MeOH=10/1) and further purified by reverse phase prep-HPLC (eluted with CH₃CN/H₂O=5%˜90% with 0.1% TFA) to give the compound 1-(4-((4-((1-(3-chlorophenyl)-3-hydroxypropyl)amino)-7-methoxyquinazolin-6-yl)oxy)piperidin-1-yl)prop-2-en-1-one (Compound 131) as a white solid (11 mg, 15% yield). LC/MS (ESI, m/z): [M+H]⁺=497.2. ¹H NMR (400 MHz, DMSO-d₆) δ 8.30 (s, 1H), 8.14 (d, J=7.8 Hz, 1H), 7.91 (s, 1H), 7.47 (s, 1 H), 7.40-7.33 (m, 2H), 7.27 (d, J=7.6 Hz, 1H), 7.14 (s, 1H), 6.85 (dd, J=16.7, 10.5 Hz, 1H), 6.12 (dd, J=16.7, 2.3 Hz, 1H), 5.69 (dd, J=10.5, 2.3 Hz, 1H), 5.57 (dd, J=14.3, 8.1 Hz, 1 H), 4.83-4.75 (m, 1H), 4.66 (s, 1H), 3.91 (s, 3H), 3.85-3.72 (m, 2H), 3.63-3.54 (m, 2H), 3.54-3.43 (m, 2H), 2.20-2.10 (m, 1H), 2.06-1.92 (m, 3H), 1.80-1.68 (m, 2H).

### Example 132: Preparation of 1-(3-((4-((3,4-dichloro-2-fluorophenyl)amino)-7-methoxyuinazolin-6-yl)oxy)piperidin-1-yl)prop-2-en-1-one (Compound 132)

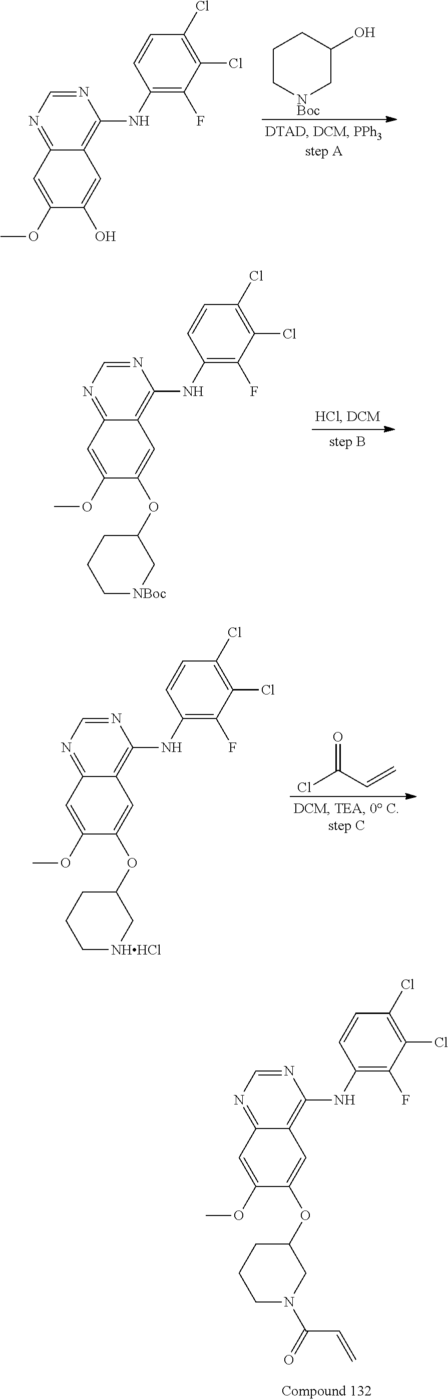

Step A: To a solution of 4-((3,4-dichloro-2-fluorophenyl)amino)-7-methoxyquinazolin-6-ol (353 mg, 1.0 mmol), PPh₃ (786 mg, 3.0 mmol) and tert-butyl 3-hydroxypiperidine-1-carboxylate (301 mg, 1.5 mmol) in anhydrous DCM (8 mL) was added dropwise a solution of DTAD (460 mg, 2.0 mmol) in anhydrous DCM (2 mL) under N2, the reaction mixture was stirred at the room temperature for 16 hr. then the mixture was concentrated and purified to give tert-butyl 3-((4-((3,4-dichloro-2-fluorophenyl) amino)-7-methoxyquinazolin-6-yl)oxy)piperidine-1-carboxylate as a yellow solid (200 mg, 0.372 mmol, 37.2% yield). LC/MS (ESI, m/z): [M+H]⁺=537.1.

Step B: To a solution of tert-butyl 3-((4-((3,4-dichloro-2-fluorophenyl)amino)-7-methoxyquinazolin-6-yl) oxy)piperidine-1-carboxylate (200 mg, 0.372 mmol) in MeOH (1 mL) was added HCl (4M in 1.4-dioxane, 2 ml), the reaction solution was stirred at the room temperature for 1 hour, then concentrated to give crude N-(3,4-dichloro-2-fluorophenyl)-7-methoxy-6-(piperidin-3-yloxy) quinazolin-4-amine hydrochloride as a yellow solid (162 mg, 0.372 mmol, 100% yield). LC/MS (ESI, m/z): [M+H]⁺=437.1.

Step C: To a solution of N-(3,4-dichloro-2-fluorophenyl)-7-methoxy-6-(piperidin-3-yloxy)quinazolin-4-amine hydrochloride (50 mg, 0.11 mmol) and DIPEA (30 mg, 0.22 mmol) in DCM (1 mL) was added acryloyl chloride (10 mg, 0.11 mmol) at 0° C., the reaction solution was stirred at the room temperature for 1 h. Then the reaction solution was quenched with water (5 mL). Then extracted with EA (5 mL*3). The combined organic layers were washed with brine, dried over Na2SO4, filtered and concentrated and purified by prep-HPLC to give 1-(3-((4-((3,4-dichloro-2-fluorophenyl)amino)-7-methoxyquinazolin-6-yl)oxy)piperidin-1-yl)prop-2-en-1-one (Compound 132) as a pale-yellow solid (5 mg, 0.01 mmol, 9.6% yield). LC/MS (ESI, m/z): [M+H]⁺=491.2. ¹H NMR (400 MHz, CDCl₃) δ 8.58 (s, 1H), 8.06-8.00 (m, 2H), 7.24-7.19 (m, 2H), 6.57 (dd, J=16.8 Hz, 1H), 6.37 (d, J=16.4 Hz, 1H), 5.73 (d, J=10 Hz, 1H), 5.27 (s, 1 H), 4.53 (d, J=12.4 Hz, 1H), 4.28-4.18 (m, 1H), 3.94 (s, 3H), 3.77 (d, J=13.6, 1H), 3.39-3.33 (m, 1H), 3.13-3.06 (m, 1H), 2.21-1.94 (m, 4H).

### Example 133: Preparation of 1-(4-((4-((3,4-dichloro-2-fluorophenyl)amino)-7-methoxyuinazolin-6-yl)oxy)piperidin-1-yl)prop-2-en-1-one (Compound 133)

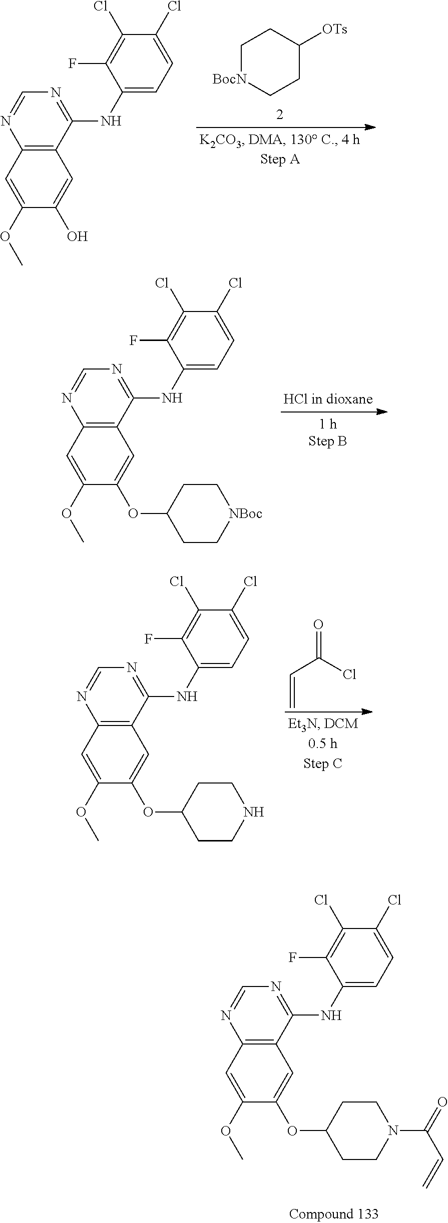

Step A: A mixture of 4-((3,4-dichloro-2-fluorophenyl)amino)-7-methoxyquinazolin-6-ol (100 mg, 0.28 mmol), tert-butyl 4-(tosyloxy)piperidine-1-carboxylate (201 mg, 0.56 mmol), K₂CO₃ (78 mg, 0.56 mmol) in DMA was degassed with nitrogen, heated to 130° C. and stirred for 2 hours under nitrogen atmosphere. The reaction was cooled to r.t and quenched by water, extracted with DCM. The combined organic layer was washed with brine, dried over anhydrous sodium sulfate, filtered and concentrated in vacuo. The residue was purified by column chromatography (DCM/MeOH) to give tert-butyl 4-((4-((3,4-dichloro-2-fluorophenyl)amino)-7-methoxyquinazolin-6-yl)oxy)piperidine-1-carboxylate (1.24 g, 66% yield) as a yellow oil. LC/MS (ESI, m/z): [M+H]⁺=537.0.

Step B: tert-butyl 4-((4-((3,4-dichloro-2-fluorophenyl)amino)-7-methoxyquinazolin-6-yl)oxy)piperidine-1-carboxylate (100 mg, 0.186 mmol) in HCl dioxane (5 mL) was stirred for 1 hour at r.t. The reaction was then quenched by water, extracted with DCM. The combined organic layer was washed with brine, dried over anhydrous sodium sulfate, filtered and concentrated in vacuo. The residue was purified by column chromatography (DCM/MeOH) to give N-(3,4-dichloro-2-fluorophenyl)-7-methoxy-6-(piperidin-4-yloxy)quinazolin-4-amine (V1668-016) (80 mg, 99% yield) as a yellow solid. LC/MS (ESI, m/z): [M+H]⁺=437.0.

Step C: A mixture of N-(3,4-dichloro-2-fluorophenyl)-7-methoxy-6-(piperidin-4-yloxy)quinazolin-4-amine (80 mg, 0.18 mmol), acryloyl chloride (17 mg, 0.18 mmol), Et₃N (37 mg, 0.37 mmol) in DCM (10 mL) was stirred for 0.5 h at r.t. The reaction was cooled to r.t and quenched by water, extracted with DCM. The combined organic layer was washed with brine, dried over anhydrous sodium sulfate, filtered and concentrated in vacuo. The residue was purified by column chromatography (DCM/MeOH) to give 1-(4-((4-((3,4-dichloro-2-fluorophenyl)amino)-7-methoxyquinazolin-6-yl)oxy)piperidin-1-yl)prop-2-en-1-one (Compound 133) (24 mg, 27% yield) as a white solid. LC/MS (ESI, m/z): [M+H]⁺=491.2, 4936.2. ¹H NMR (400 MHz, DMSO-d₆) δ 9.65 (s, 1H), 8.40 (s, 1H), 7.88 (s, 1H), 7.63-7.57 (m, 2H), 7.25 (s, 1H), 6.85 (dd, J=16.8, 10.4 Hz, 1H), 6.12 (dd, J=16.8, 2.4 Hz, 1H), 5.69 (dd, J=10.4, 2.4 Hz, 1H), 4.84-4.73 (m, 1H), 3.95 (s, 3H), 3.92-3.84 (m, 2H), 3.51-3.42 (m, 2H), 2.08-2.03 (m, 2H), 1.70-1.67 (m, 2H).

### Example 134: Preparation of 1-(4-((7-methoxy-4-((1-(methylsulfonyl)indolin-5-yl)amino)quinazolin-6-yl)oxy)piperidin-1-yl)prop-2-en-1-one (Compound 134)

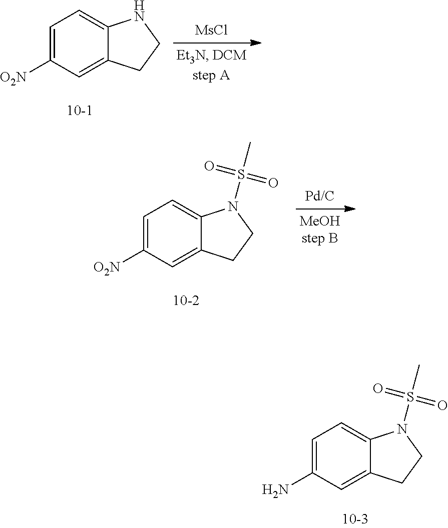

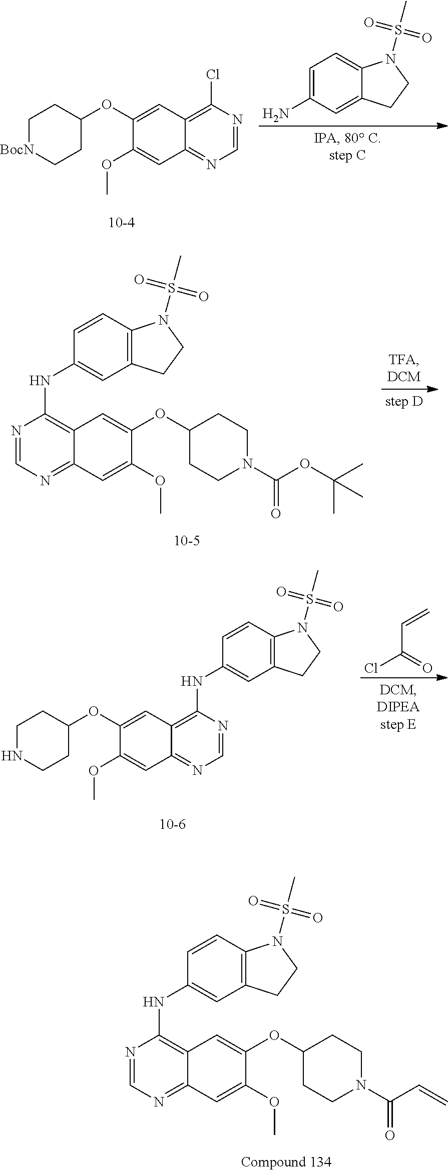

Step A: To a solution of 5-nitroindoline(600 mg, 3.66 mmol), Et₃N (444.4 mg, 4.40 mmol) in DCM (21 mL) at 0° C. was slowly added methanesulfonyl chloride (542.6 mg, 4.76 mmol). The mixture was warmed up and stirred at rt for 1.5 h. The reaction was quenched with water (30 mL) and extracted with DCM (25 mL×4). The organic layers were combined and concentrated in vacuum. The residue was purified by flash chromatography eluting with PE:EA=2:1 to give 1-(methylsulfonyl)-5-nitroindoline (774 mg, yield 90%) as a yellow solid.

Step B: A mixture of 1-(methylsulfonyl)-5-nitroindoline (100 mg, 0.41 mmol) and 10% Pd/C (43.5 mg, 0.041 mmol) in methanol (15 mL) was stirred at RT under H₂ atmosphere for 18 h. The reaction mixture was filtered and washed with methanol (10 mL). Solvent was removed in vacuum to afford 1-(methylsulfonyl)indolin-5-amine (68.8 mg, yield 79%) as a brown solid.

Step C: To a mixture of tert-butyl 4-((4-chloro-7-methoxyquinazolin-6-yl)oxy)piperidine-1-carboxylate (125.7 mg, 0.32 mmol) in IPA (15 mL) was added 1-(methylsulfonyl)indolin-5-amine (68.8 mg, 0.32 mmol). The reaction mixture was stirred at 80° C. for 3 h. The mixture was concentrated in vacuum. The residue was purified by column chromatography on silica gel eluting with DCM:MeOH=20:1 to give tert-butyl 4-((7-methoxy-4-((1-(methylsulfonyl)indolin-5-yl)amino)quinazolin-6-yl)oxy)piperidine-1-carboxylate(79.7 mg) as a yellow solid. LC/MS (ESI, m/z): [M+H]⁺=570.2.

Step D: To a mixture of tert-butyl 4-((7-methoxy-4-((1-(methylsulfonyl)indolin-5-yl)amino)quinazolin-6-yl)oxy)piperidine-1-carboxylate (79.2 mg, 0.14 mmol) in DCM (3 mL) was added TFA (1 mL). The reaction mixture was stirred at rt for 2 h. The mixture was concentrated in vacuum. The residue was washed by 1,2-dichloroethane (2×10 mL) and then concentrated in vacuum to give 7-methoxy-N-(1-(methylsulfonyl)indolin-5-yl)-6-(piperidin-4-yloxy)quinazolin-4-amine (95.6 mg) as a yellow solid. LC/MS (ESI, m/z): [M+H]⁺=470.2.

Step E: To a mixture of 7-methoxy-N-(1-(methylsulfonyl)indolin-5-yl)-6-(piperidin-4-yloxy)quinazolin-4-amine (81.6 mg, 0.14 mmol) in DCM (10 mL) was added DIPEA (54.2 mg, 0.42 mmol) and acryloyl chloride (12.6 mg, 0.14 mmol). The reaction mixture was stirred at RT for 1 h. The reaction mixture was concentrated in vacuum. The residue was washed with water (15 mL) and extracted with EtOAc (3×15 mL). The combined layers was concentrated in vacuum. The residue was purified by column chromatography on silica gel eluting with DCM:MeOH=10:1 and dried to afford 1-(4-((7-methoxy-4-((1-(methylsulfonyl)indolin-5-yl)amino)quinazolin-6-yl)oxy)piperidin-1-yl)prop-2-en-1-one (Compound 134) (50.2 mg, 69% yield) as a yellow solid. LC/MS (ESI, m/z): [M+H]⁺=524.2. ¹H NMR (400 MHz, DMSO-d₆) δ 9.60 (s, 1H), 8.41 (s, 1H), 8.09 (s, 1H), 7.78 (d, J=1.1 Hz, 1H), 7.55 (d, J=8.5 Hz, 1H), 7.26 (d, J=8.6 Hz, 1H), 7.19 (s, 1H), 6.85 (dd, J=16.7, 10.3 Hz, 1H), 6.12 (dd, J=16.8, 2.4 Hz, 1 H), 5.68 (dd, J=10.4, 2.4 Hz, 1H), 4.91-4.88 (m, 1H), 3.97 (t, J=8.4 Hz, 2H), 3.93 (s, 3H), 3.92-3.82 (m, 2H), 3.56-3.43 (m, 2H), 3.16 (t, J=8.4 Hz, 2H), 2.99 (s, 3H), 2.10-1.96 (m, 2H), 1.76-1.58 (m, 2H).

Examples 135-165 were prepared using similar procedures as described in the preceding Examples.

| EX. | | Compound | | |
|---|---|---|---|---|
| NO. | IUPAC Name | structure | salt | Compound characterization |
| 135 | 1-(4-((7-methoxy-4- (quinolin-3- ylamino)quinazolin- 6-yl)oxy)piperidin- 1-yl)prop-2-en-1- one | 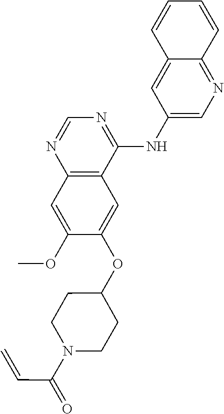 [SMILES: C=CC(=O)N1CCC(Oc2cc3c(Nc4cnc5ccccc5c4)ncnc3cc2OC)CC1] | None | LC/MS (ESI, m/z): [M + 1]⁺ = 456.2. ¹H NMR (400 MHz, DMSO-d₆) δ 11.14 (br s, 1 H), 9.20 (d, J = 2.5 Hz, 1 H), 8.86 (s, 1 H), 8.66 (d, J = 2.3 Hz, 1 H), 8.19 (s, 1 H), 8.07 (t, J = 7.7 Hz, 2 H), 7.80 (ddd, J = 8.4, 6.9, 1.5 Hz, 1 H), 7.73-7.60 (m, 1 H), 7.35 (s, 1 H), 6.86 (dd, J = 16.7, 10.5 Hz, 1 H), 6.13 (dd, J = 16.7, 2.4 Hz, 1 H), 5.70 (dd, J = 10.5, 2.4 Hz, 1 H), 4.99-4.80 (m, 1 H), 4.05 (s, 3 H), 3.91-3.78 (m, 2 H), 3.63-3.38 (m, 2 H), 2.27-1.91 (m, 2 H), 1.90-1.54 (m, 2 H). |
| 136 | 1-(4-((4-((5- chlorobenzo[d]oxazol- 2-yl)amino)-7- methoxyquinazolin- 6-yl)oxy)piperidin- 1-yl)prop-2-en-1- one | 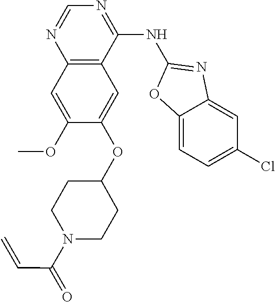 [SMILES: C=CC(=O)N1CCC(Oc2cc3c(Nc4nc5cc(Cl)ccc5o4)ncnc3cc2OC)CC1] | None | LC/MS (ESI, m/z): [M + 1]⁺ = 480.1. ¹H NMR (400 MHz, DMSO-d₆) δ 8.54 (s, 1 H), 7.87 (s, 1 H), 7.61-7.57 (m, 2 H), 7.30-7.27 (m, 2 H), 6.88-6.81 (m, 1 H), 6.12 (dd, J = 2 Hz, J = 16.4 Hz, 1 H), 5.69 (dd, J = 2 Hz, J = 10 Hz, 1 H), 4.87-4.82 (m, 1 H), 3.97 (s, 3 H), 3.94-3.85 (m, 2 H), 3.57-3.43 (m, 2 H), 2.08-1.97 (m, 2 H), 1.76-1.63 (m, 2 H). |
| 137 | 1-(4-((4-((1H- indazol-6-yl)amino)- 7- methoxyquinazolin- 6-yl)oxy)piperidin- 1-yl)prop-2-en-1- one | 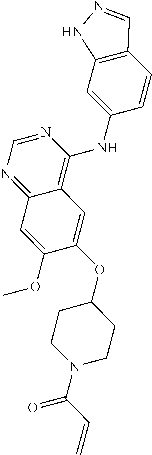 [SMILES: C=CC(=O)N1CCC(Oc2cc3c(Nc4ccc5cn[nH]c5c4)ncnc3cc2OC)CC1] | None | LC/MS (ESI, m/z): [M + 1]⁺ = 445.1. ¹H NMR (400 MHz, DMSO-d₆) δ 12.96 (s, 1 H), 9.54 (s, 1 H), 8.51 (s, 1 H), 8.27 (s, 1 H), 8.04 (s, 1 H), 8.03 (s, 1 H), 7.74 (d, J = 8.6 Hz, 1 H), 7.44 (dd, J = 8.7, 1.7 Hz, 1 H), 7.24 (s, 1 H), 6.85 (dd, J = 16.7, 10.5 Hz, 1 H), 6.12 (dd, J = 16.7, 2.4 Hz, 1 H), 5.69 (dd, J = 10.5, 2.4 Hz, 1 H), 4.98-4.73 (m, 1 H), 3.95 (s, 3 H), 3.89-3.85 (m, 2 H), 3.53- 3.49 (m, 2 H), 2.06-2.02 (m, 2 H), 1.73-1.69 (m, 2 H). |
| 138 | 1-(4-((7-methoxy-4- ((1-methyl-1H- indazol-6- yl)amino)quinazolin- 6-yl)oxy)piperidin- 1-yl)prop-2-en-1- one | 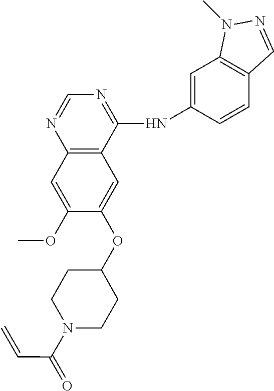 [SMILES: C=CC(=O)N1CCC(Oc2cc3c(Nc4ccc5cnn(C)c5c4)ncnc3cc2OC)CC1] | None | LC/MS (ESI, m/z): [M + H]⁺ = 459.2. ¹H NMR (400 MHz, DMSO-d₆) δ 9.60 (s, 1 H), 8.50 (s, 1 H), 8.14 (s, 1 H), 8.03 (s, 1 H), 7.99 (s, 1 H), 7.74 (d, J = 8.7 Hz, 1 H), 7.48 (dd, J = 8.7, 1.7 Hz, 1 H), 7.24 (s, 1 H), 6.85 (dd, J = 16.7, 10.5 Hz, 1 H), 6.12 (dd, J = 16.7, 2.4 Hz, 1 H), 5.69 (dd, J = 10.5, 2.4 Hz, 1 H), 4.85-4.83 (m, 1 H), 4.03 (s, 3 H), 3.95 (s, 3 H), 3.90-3.84 (m, 2 H), 3.55-3.47 (m, 2 H), 2.08-2.00 (m, 2 H), 1.75-1.67 (m, 2 H). |
| 139 | 1-(4-((4-((1H- indazol-7-yl)amino)- 7- methoxyquinazolin- 6-yl)oxy)piperidin- 1-yl)prop-2-en-1- one | 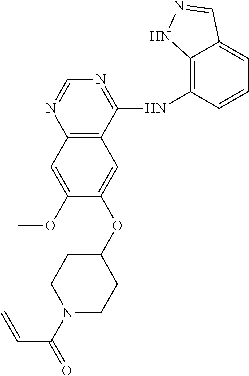 [SMILES: C=CC(=O)N1CCC(Oc2cc3c(Nc4cccc5cn[nH]c45)ncnc3cc2OC)CC1] | None | LC/MS (ESI, m/z): [M + H]⁺ = 445.2. ¹H NMR (400 MHz, DMSO-d₆) δ 12.82 (s, 1 H), 9.69 (s, 1 H), 8.30 (s, 1 H), 8.10 (d, J = 1.4 Hz, 1 H), 7.99 (s, 1 H), 7.67 (d, J = 8.0 Hz, 1 H), 7.39 (d, J = 7.1 Hz, 1 H), 7.20 (d, J = 24.1 Hz, 1 H), 7.15 (d, J = 7.6 Hz, 1 H), 6.85 (dd, J = 16.7, 10.5 Hz, 1 H), 6.12 (dd, J = 16.7, 2.4 Hz, 1 H), 5.69 (dd, J = 10.5, 2.4 Hz, 1 H), 4.90-4.68 (m, 1 H), 3.97-3.94 (m, 5 H), 3.51-3.43 (m, 2 H), 2.08-2.04 (m, 2 H), 1.73-1.69 (m, 2 H). |
| 140 | 1-(4-((4- (isoquinolin-6- ylamino)-7- methoxyquinazolin- 6-yl)oxy)piperidin- 1-yl)prop-2-en-1- one | 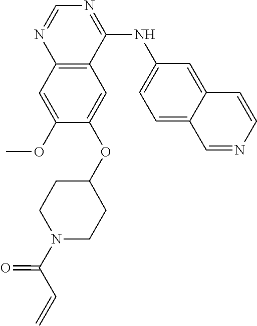 [SMILES: C=CC(=O)N1CCC(Oc2cc3c(Nc4ccc5cnccc5c4)ncnc3cc2OC)CC1] | None | LC/MS (ESI, m/z): [M + H]⁺ = 456.2. ¹H NMR (400 MHz, DMSO-d₆) δ 9.72 (s, 1 H), 9.24 (s, 1 H), 8.61 (dd, J = 7.1, 1.5 Hz, 1 H), 8.20 (d, J = 9.2 Hz, 1 H), 7.98 (d, J = 7.1 Hz, 2 H), 7.68 (s, 1 H), 7.41-7.34 (m, 2 H), 7.04 (d, J = 1.7 Hz, 1 H), 6.81 (dd, J = 16.7, 10.5 Hz, 1 H), 6.10 (dd, J = 16.7, 2.4 Hz, 1 H), 5.68 (dd, J = 10.5, 2.4 Hz, 1 H), 4.84- 4.82 (m, 1 H), 4.08 (s, 3 H), 3.81-3.78 (m, 2 H), 3.28-3.23 (m, 2 H), 2.03-1.98 (m, 2 H), 1.73-1.65 (m, 2 H). |
| 141 | 1-(4-((7-methoxy-4- (quinolin-7- ylamino)quinazolin- 6-yl)oxy)piperidin- 1-yl)prop-2-en-1- one | 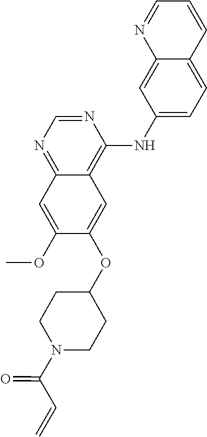 [SMILES: C=CC(=O)N1CCC(Oc2cc3c(Nc4ccc5cccnc5c4)ncnc3cc2OC)CC1] | None | ¹H NMR (400 MHz, DMSO-d₆) δ 9.78 (s, 1 H), 8.86 (d, J = 1.2 Hz, 1 H), 8.60-8.58 (m, 2 H), 8.32-8.28 (m, 1 H), 8.10-8.07 (m, 2 H), 7.98 (d, J = 8.8 Hz, 1 H), 7.45-7.42 (m, 1 H), 7.27 (s, 1 H), 6.88-6.82 (m, 1 H), 6.12 (dd, J = 16.4 Hz, 1 H), 5.69 (dd, J = 10.4 Hz, 1 H), 4.88-4.86 (m, 1 H), 3.96 (s, 3 H), 3.88-3.87 (m, 2 H), 3.65-3.57 (m, 2 H), 2.10-1.99 (m, 2 H), 1.79-1.66 (m, 2 H). |
| 142 | 1-(4-((4- (benzo[d]thiazol-5- ylamino)-7- methoxyquinazolin- 6-yl)oxy)piperidin- 1-yl)prop-2-en-1- one | 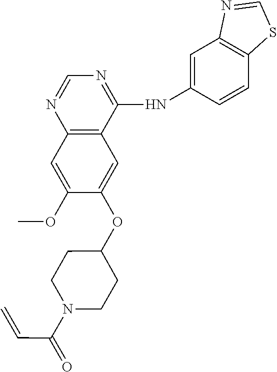 [SMILES: C=CC(=O)N1CCC(Oc2cc3c(Nc4ccc5scnc5c4)ncnc3cc2OC)CC1] | None | LC/MS (ESI, m/z): [M + 1]⁺ = 462.1. ¹H NMR (400 MHz, DMSO-d₆) δ 9.65 (s, 1 H), 9.41 (s, 1 H), 8.64 (d, J = 1.9 Hz, 1 H), 8.51 (s, 1 H), 8.16 (d, J = 8.7 Hz, 1 H), 8.03 (s, 1 H), 7.89 (dd, J = 8.7, 2.0 Hz, 1 H), 7.25 (s, 1 H), 6.85 (dd, J = 16.7, 10.5 Hz, 1 H), 6.12 (dd, J = 16.7, 2.4 Hz, 1 H), 5.69 (dd, J = 10.5, 2.4 Hz, 1 H), 4.89-4.74 (m, 1 H), 3.96 (s, 3 H), 3.89-3.85 (m, 2 H), 3.54-3.50 (m, 2 H), 2.06-2.02 (m, 2 H), 1.74-1.70 (m, 2 H). |
| 143 | 1-(4-((7-methoxy-4- (quinolin-6- ylamino)quinazolin- 6-yl)oxy)piperidin- 1-yl)prop-2-en-1- one | 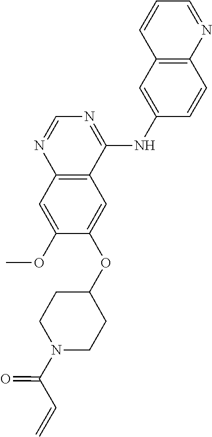 [SMILES: C=CC(=O)N1CCC(Oc2cc3c(Nc4ccc5ncccc5c4)ncnc3cc2OC)CC1] | None | LC/MS (ESI, m/z): [M + H]⁺ = 456.2. ¹H NMR (400 MHz, DMSO-d₆) δ 11.24 (s, 1 H), 8.99 (dd, J = 4.3, 1.4 Hz, 1 H), 8.89 (s, 1H), 8.54 (d, J = 8.3 Hz, 1H), 8.33 (d, J = 2.0 Hz, 1H), 8.25-8.10 (m, 3 H), 7.67 (dd, J = 8.3, 4.3 Hz, 1H), 7.37 (s, 1 H), 6.86 (dd, J = 16.7, 10.5 Hz, 1 H), 6.13 (dd, J = 16.7, 2.4 Hz, 1 H), 5.70 (dd, J = 10.5, 2.4 Hz, 1H), 4.92- 4.87 (m, 1 H), 4.03 (s, 3 H), 3.92-3.82 (m, 2 H), 3.58-3.47 (m, 2 H), 2.15-2.02 (m, 2H), 1.82-1.67 (m, 2 H). |
| 144 | 1-(4-((7-methoxy-4- (quinoxalin-6- ylamino)quinazolin- 6-yl)oxy)piperidin- 1-yl)prop-2-en-1- one | 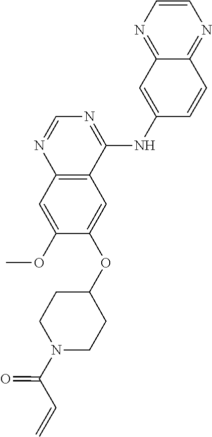 [SMILES: C=CC(=O)N1CCC(Oc2cc3c(Nc4ccc5nccnc5c4)ncnc3cc2OC)CC1] | None | LC/MS (ESI, m/z): [M + H]⁺ = 457.0. ¹H NMR (400 MHz, DMSO-d₆) δ 10.68 (s, 1 H), 8.95 (s, 1 H), 8.91 (s, 1 H), 8.81 (s, 1 H), 8.63 (s, 1 H), 8.30 (dd, J = 9.2, 1.8 Hz, 1H), 8.21 (s, 1 H), 8.17 (d, J = 5.0 Hz, 1 H), 7.33 (s, 1 H), 6.86 (dd, J = 16.7, 10.4 Hz, 1 H), 6.13 (dd, J = 16.6, 2.2 Hz, 1 H), 5.70 (dd, J = 10.4, 2.2 Hz, 1 H), 4.96-4.89 (m, 1 H), 4.01 (s, 3 H), 3.92-3.82 (m, 2 H), 3.68-3.61 (m, 2 H), 2.11-2.02 (m, 2 H), 1.80-1.67 (m, 2 H). |
| 145 | 1-(4-((4- (benzo[d]thiazol-6- ylamino)-7- methoxyquinazolin- 6-yl)oxy)piperidin- 1-yl)prop-2-en-1- one | 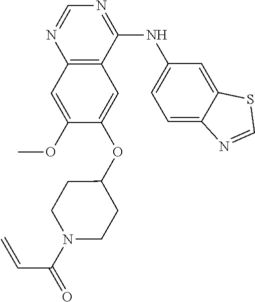 [SMILES: C=CC(=O)N1CCC(Oc2cc3c(Nc4ccc5ncsc5c4)ncnc3cc2OC)CC1] | None | LC/MS (ESI, m/z): [M + H]⁺ = 462.1. ¹H NMR (400 MHz, DMSO-d₆) δ 9.67 (s, 1 H), 9.31 (s, 1 H), 8.69 (d, J = 2 Hz, 1 H), 8.51 (s, 1 H), 8.11 (d, J = 8.8 Hz, 1 H), 8.02 (s, 1 H), 7.87 (dd, J = 2 Hz, J = 8.4 Hz, 1 H), 7.25 (s, 1 H), 6.89-6.82 (m, 1 H), 6.12 (dd, J = 2.4 Hz, J = 16.4 Hz, 1 H), 5.69 (dd, J = 2.4 Hz, J = 10.4 Hz, 1 H), 4.85-4.82 (m, 1 H), 3.96 (s, 3 H), 3.89-3.87 (m, 2 H), 3.57- 3.46 (m, 2 H), 2.08-2.00 (m, 2 H), 1.77-1.65 (m, 2 H). |
| 146 | 1-(4-((7-methoxy-4- (quinolin-8- ylamino)quinazolin- 6-y1)oxy)piperidin- 1-yl)prop-2-en-1- one | 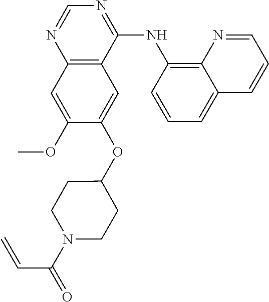 [SMILES: C=CC(=O)N1CCC(Oc2cc3c(Nc4cccc5cccnc45)ncnc3cc2OC)CC1] | None | LC/MS (ESI, m/z): [M + H]⁺ = 456.2. ¹H NMR (400 MHz, DMSO-d₆) δ 11.24 (s, 1 H), 8.96 (dd, J = 4.1, 1.6 Hz, 1 H), 8.68 (s, 1 H), 8.52 (dd, J = 8.3, 1.6 Hz, 1 H), 8.25-8.10 (m, 2 H), 8.01 (d, J = 7.9 Hz, 1 H), 7.76 (t, J = 7.9 Hz, 1 H), 7.67 (dd, J = 8.3, 4.2 Hz, 1 H), 7.31 (s, 1 H), 6.86 (dd, J = 16.7, 10.5 Hz, 1 H), 6.13 (dd, J = 16.7, 2.4 Hz, 1 H), 5.70 (dd, J = 10.5, 2.4 Hz, 1 H), 4.95-4.85 (m, 3 H), 3.84-3.76 (m, 4 H), 2.15-2.05 (m, 2 H), 1.78- 1.70 (m, 2 H). |
| 147 | 1-(4-((4-(4-(3- chlorophenyl) piperazin- 1-yl)-7- methoxyquinazolin- 6-yl)oxy)piperidin- 1-yl)prop-2-en-1- one | 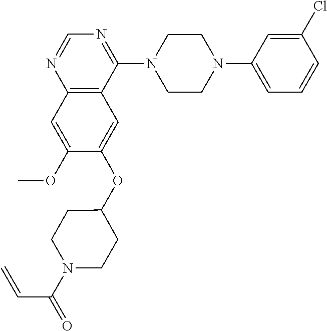 [SMILES: C=CC(=O)N1CCC(Oc2cc3c(N4CCN(c5cccc(Cl)c5)CC4)ncnc3cc2OC)CC1] | None | LC/MS (ESI, m/z): [M + H]⁺ = 508.2. ¹H NMR (400 MHz, DMSO-d6) δ 8.76 (s, 1 H), 7.52 (s, 1 H), 7.27-7.23 (m, 2 H), 6.96- 6.79 (m, 4 H), 6.10 (dd, J = 16.8 Hz, 1 H), 5.68 (dd, J = 10.4 Hz, 1 H), 4.82-4.79 (m, 1 H), 4.21-4.11 (m, 4 H), 3.98 (s, 3 H), 3.93-3.82 (m, 2 H), 3.52-3.50 (m, 6 H), 2.02-1.99 (m, 2 H), 1.75-1.61 (m, 2 H). |
| 148 | 1-(4-((7-methoxy-4- (naphthalen-2- ylamino)quinazolin- 6-yl)oxy)piperidin- 1-yl)prop-2-en-1- one | 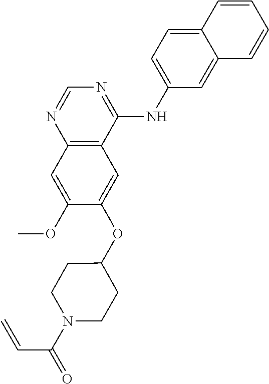 [SMILES: C=CC(=O)N1CCC(Oc2cc3c(Nc4ccc5ccccc5c4)ncnc3cc2OC)CC1] | None | LC/MS (ESI, m/z): [M + H]⁺ = 455.2. ¹H NMR (400 MHz, DMSO-d₆) δ 9.80 (s, 1 H), 8.55 (s, 1 H), 8.33 (s, 1 H), 8.07 (s, 1 H), 7.96- 7.89 (m, 4 H), 7.53-7.44 (m, 2 H), 7.25 (s, 1 H), 6.89-6.82 (m, 1 H), 6.12 (dd, J = 16.8 Hz, 1 H), 5.69 (dd, J = 10.4 Hz, 1 H), 4.88- 4.84 (m, 1 H), 3.96 (s, 3 H), 3.93-3.87 (m, 2 H), 3.62-3.50 (m, 2 H), 2.10-1.98 (m, 2 H), 1.78-1.68 (m, 2 H). |
| 149 | 1-(4-((7-methoxy-4- (naphthalen-1- ylamino)quinazolin- 6-yl)oxy)piperidin- 1-yl)prop-2-en-1- one | 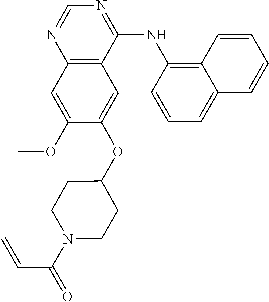 [SMILES: C=CC(=O)N1CCC(Oc2cc3c(Nc4cccc5ccccc45)ncnc3cc2OC)CC1] | None | LC/MS (ESI, m/z): [M + H]⁺ = 455.2. ¹H NMR (400 MHz, DMSO-d₆) δ 9.85 (s, 1 H), 8.20 (s, 1 H), 8.06 (s, 1 H), 8.00 (d, J = 8 Hz, 1 H), 7.91 (dd, J = 7.2 Hz, 1 H), 7.86 (d, J = 8 Hz, 1 H), 7.62-7.47 (m, 4 H), 7.22 (s, 1 H), 6.88-6.81 (m, 1H), 6.11 (dd, J = 16.8 Hz, 1H), 5.68 (dd, J = 10.4 Hz, 1 H), 4.82- 4.78 (m, 1 H), 3.95 (s, 3H), 3.92-3.88 (m, 2 H), 3.53-3.39 (m, 2 H), 2.13-2.02 (m, 2 H), 1.77-1.66 (m, 2 H). |
| 150 | 1-(4-((4-((1H- indazol-5-yl)amino)- 7- methoxyquinazolin- 6-yl)oxy)piperidin- 1-yl)prop-2-en-1- one | 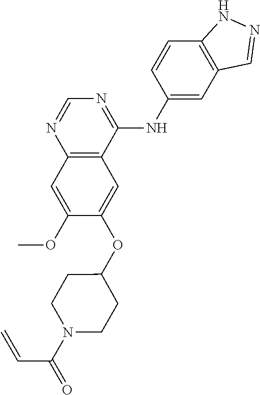 [SMILES: C=CC(=O)N1CCC(Oc2cc3c(Nc4ccc5[nH]ncc5c4)ncnc3cc2OC)CC1] | None | LC/MS (ESI, m/z): [M + H]⁺ = 445.2. ¹H NMR (400 MHz, DMSO-d₆) δ 13.05 (s, 1 H), 9.52 (s, 1 H), 8.40 (s, 1 H), 8.09 (d, J = 8 Hz, 2 H), 7.99 (s, 1 H), 7.62-7.55 (m, 2 H), 7.21 (s, 1 H), 6.89-6.82 (m, 1 H), 6.12 (dd, J = 2.4 Hz, J = 16.8 Hz, 1 H), 5.69 (dd, J = 2 Hz, J = 10.4 Hz, 1 H), 4.84-4.78 (m, 1 H), 3.94 (s, 3 H), 3.94-3.90 (m, 2 H), 3.52-3.38 (m, 2 H), 2.08-2.00 (m, 2 H), 1.76-1.63 (m, 2 H). |
| 151 | 1-(4-((7-methoxy-4- ((2- methoxydibenzo [b,d]furan-3- yl)amino)quinazolin- 6-yl)oxy)piperidin- 1-yl)prop-2-en-1- one | 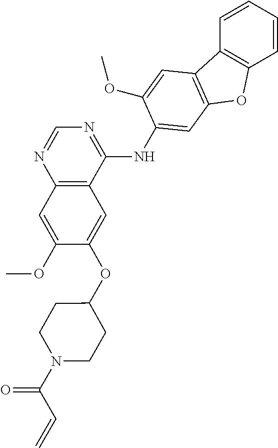 [SMILES: C=CC(=O)N1CCC(Oc2cc3c(Nc4cc5oc6ccccc6c5cc4OC)ncnc3cc2OC)CC1] | None | LC/MS (ESI, m/z): [M + H]⁺ = 525.2. ¹H NMR (400 MHz, DMSO-d₆) δ 10.87 (s, 1H), 8.74 (d, J = 16.2 Hz, 1H), 8.21 (d, J = 7.4 Hz, 1H), 8.15 (s, 1H), 8.03 (d, J = 16.0 Hz, 1H), 7.92 (s, 1H), 7.70 (t, J = 10.6 Hz, 1H), 7.60-7.50 (m, 1H), 7.44 (t, J = 7.5 Hz, 1H), 7.34 (s, 1H), 6.86 (dd, J = 16.7, 10.5 Hz, 1H), 6.13 (dd, J = 16.7, 2.3 Hz, 1H), 5.78-5.59 (m, 1H), 4.96-4.72 (m, 1H), 4.03 (d, J = 15.4 Hz, 3H), 3.94 (s, 3H), 3.92- 3.83 (m, 2H), 3.69-3.60 (m, 2H), 2.14- 2.01 (m, 2H), 1.80-1.64 (m, 2H). |
| 152 | 1-(4-((4- (benzo[d][1,3]dioxol- 5-ylamino)-7- methoxyquinazolin- 6-yl)oxy)piperidin- 1-yl)prop-2-en-1- one | 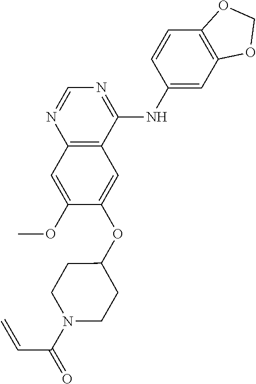 [SMILES: C=CC(=O)N1CCC(Oc2cc3c(Nc4ccc5c(c4)OCO5)ncnc3cc2OC)CC1] | None | LC/MS (ESI, m/z): [M + H]⁺ = 449.1. ¹H NMR (400 MHz, DMSO-d₆) δ 9.36 (s, 1 H), 8.40 (s, 1 H), 7.92 (s, 1 H), 7.39 (d, J = 2 Hz, 1 H), 7.19 (s, 1 H), 7.07 (dd, J = 8.2 Hz, 1 H), 6.94 (d, J = 8.4 Hz, 1 H), 6.88-6.81 (m, 1 H), 6.11 (dd, J = 16.4 Hz, 1 H), 6.04 (s, 2 H), 5.68 (dd, J = 10.4 Hz, 1 H), 4.80- 4.77 (m, 1 H), 3.93 (s, 3 H), 3.91-3.86 (m, 2 H), 3.50-3.43 (m, 2 H), 2.02-2.00 (m, 2 H), 1.71-1.67 (m, 2 H). |
| 153 | 1-(4-((4-((2,3- dihydro-1H-inden-5- yl)amino)-7- methoxyquinazolin- 6-yl)oxy)piperidin- 1-yl)prop-2-en-1- one | 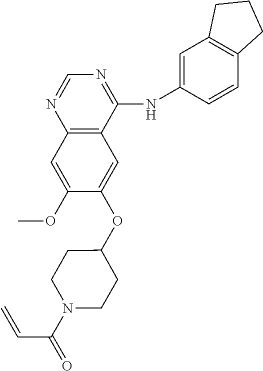 [SMILES: C=CC(=O)N1CCC(Oc2cc3c(Nc4ccc5c(c4)CCC5)ncnc3cc2OC)CC1] | None | LC/MS (ESI, m/z): [M + H]⁺ = 445.2. ¹H NMR (400 MHz, DMSO-d₆) δ 9.76 (s, 1 H), 8.40 (s, 1 H), 8.26 (s, 1 H), 7.73 (s, 1 H), 7.57 (dd, J = 8.0, 1.6 Hz, 1 H), 7.20 (d, J = 8.1 Hz, 1 H), 7.17 (s, 1 H), 6.85 (dd, J = 10.5, 6.7 Hz, 1 H), 6.11 (dd, J = 16.6, 2.6 Hz, 1 H), 5.68 (dd, J = 10.5, 2.4 Hz, 1 H), 5.06-5.01 (m, 1 H), 3.92 (s, 3 H), 3.92-3.82 (m, 2 H), 3.58-3.51 (m, 1 H), 3.47-3.41 (m, 1 H), 2.90-2.83 (m, 4 H), 2.08-2.00 (m, 4 H), 1.71-1.58 (m, 2 H). |
| 154 | 1-(4-((4-((5- chlorobenzo[d]oxazol- 2-yl)amino)-7- methoxyquinazolin- 6-yl)oxy)piperidin- 1-yl)prop-2-en-1- one | 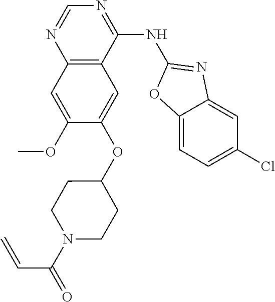 [SMILES: C=CC(=O)N1CCC(Oc2cc3c(Nc4nc5cc(Cl)ccc5o4)ncnc3cc2OC)CC1] | None | LC/MS (ESI, m/z): [M + H]⁺ = 480.1. ¹H NMR (400 MHz, DMSO-d₆) δ 8.54 (s, 1 H), 7.87 (s, 1 H), 7.61-7.57 (m, 2 H), 7.30-7.27 (m, 2 H), 6.88-6.81 (m, 1 H), 6.12 (dd, J = 2 Hz, J = 16.4 Hz, 1 H), 5.69 (dd, J = 2 Hz, J = 10 Hz, 1 H), 4.87-4.82 (m, 1 H), 3.97 (s, 3 H), 3.94-3.85 (m, 2 H), 3.57-3.43 (m, 2 H),2.08-1.97 (m, 2 H), 1.76-1.63 (m, 2 H). |
| 155 | 1-(4-((7-methoxy-4- ((2- methylbenzo[d] thiazol-5- yl)amino)quinazolin- 6-yl)oxy)piperidin- 1-yl)prop-2-en-1- one | 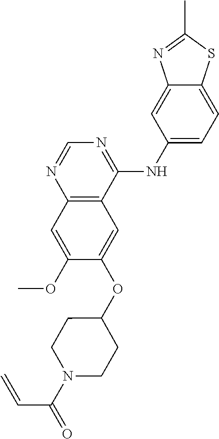 [SMILES: C=CC(=O)N1CCC(Oc2cc3c(Nc4ccc5sc(C)nc5c4)ncnc3cc2OC)CC1] | None | LC/MS (ESI, m/z): [M + H]⁺ = 476.1. ¹H NMR (400 MHz, DMSO-d₆) δ 8.61 (s, 1 H), 8.49 (s, 1 H), 8.43 (d, J = 1.9 Hz, 1 H), 8.02-8.00 (m, 2 H), 7.79 (dd, J = 8.6, 1.8 Hz, 1 H), 7.24 (s, 1 H), 6.85 (dd, J = 16.8, 10.6 Hz, 1 H), 6.12 (dd, J = 16.8, 2.4 Hz, 1 H), 5.69 (dd, J = 10.4, 2.4 Hz, 1 H), 4.85- 4.82 (m, 1 H), 3.95 (s, 3 H), 3.92-3.82 (m, 2 H), 3.59-3.43 (m, 2 H), 2.81 (s, 3 H), 2.08-1.98 (m, 2 H), 1.78-1.65 (m, 2 H). |
| 156 | 1-(4-((4-((2,3- dihydro-1H-inden-4- yl)amino)-7- methoxyquinazolin- 6-yl)oxy)piperidin- 1-yl)prop-2-en-1- one | 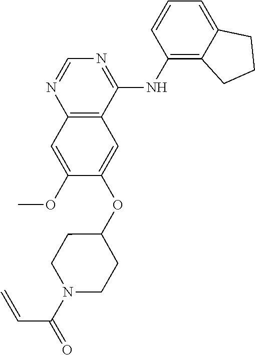 [SMILES: C=CC(=O)N1CCC(Oc2cc3c(Nc4cccc5c4CCC5)ncnc3cc2OC)CC1] | None | LC/MS (ESI, m/z): [M + H]⁺ = 445.2. ¹H NMR (400 MHz, CDCl₃) δ 8.62 (s, 1 H), 7.57 (d, J = 7.8 Hz, 1 H), 7.29 (s, 1 H), 7.22 (t, J = 9.8 Hz, 1 H), 7.18 (s, 1 H), 7.12 (d, J = 7.5 Hz, 1 H), 6.61 (dd, J = 16.8, 10.6 Hz, 1 H), 6.30 (dd, J = 16.9, 1.9 Hz, 1 H), 5.71 (dd, J = 10.7, 1.9 Hz, 1 H), 4.59- 4.54 (m, 1 H), 4.03 (s, 3 H), 3.96-3.83 (m, 2 H), 3.70-3.60 (m, 1 H), 3.55-3.45 (m, 1 H), 3.00 (t, J = 7.4 Hz, 1 H), 2.85 (t, J = 7.4 Hz, 1 H), 2.15-2.08 (m, 2 H), 2.01-1.86 (m, 4 H). |
| 157 | 1-(4-((4- (benzo[d]thiazol-5- ylamino)-7- methoxyquinazolin- 6-yl)oxy)piperidin- 1-yl)prop-2-en-1- one | 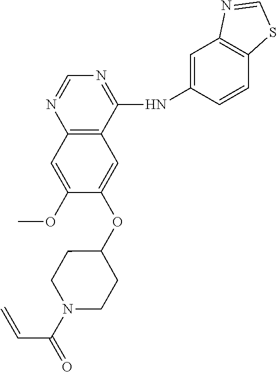 [SMILES: C=CC(=O)N1CCC(Oc2cc3c(Nc4ccc5scnc5c4)ncnc3cc2OC)CC1] | None | LC/MS (ESI, m/z): [M + 1]⁺ = 462.1. ¹H NMR (400 MHz, DMSO-d₆) δ 9.65 (s, 1 H), 9.41 (s, 1 H), 8.64 (d, J = 1.9 Hz, 1 H), 8.51 (s, 1 H), 8.16 (d, J = 8.7 Hz, 1 H), 8.03 (s, 1 H), 7.89 (dd, J = 8.7, 2.0 Hz, 1 H), 7.25 (s, 1 H), 6.85 (dd, J = 16.7, 10.5 Hz, 1 H), 6.12 (dd, J = 16.7, 2.4 Hz, 1 H), 5.69 (dd, J = 10.5, 2.4 Hz, 1 H), 4.89-4.74 (m, 1 H), 3.96 (s, 3 H), 3.89-3.85 (m, 2 H), 3.54-3.50 (m, 2 H), 2.06-2.02 (m, 2 H), 1.74-1.70 (m, 2 H). |
| 158 | 1-(4-((7-methoxy-4- (quinolin-2- ylamino)quinazolin- 6-yl)oxy)piperidin- 1-yl)prop-2-en-1- one | 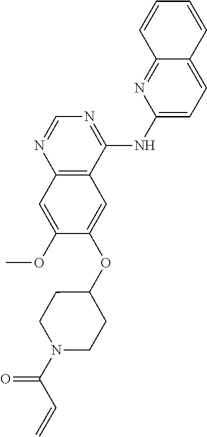 [SMILES: C=CC(=O)N1CCC(Oc2cc3c(Nc4ccc5ccccc5n4)ncnc3cc2OC)CC1] | None | LC/MS (ESI, m/z): [M + 1]⁺ = 456.1. ¹H NMR (400 MHz, DMSO-d₆) δ 8.82 (s, 1 H), 8.48 (d, J = 9.0 Hz, 1 H), 8.24 (s, 2 H), 8.00 (t, J = 8.6 Hz, 2 H), 7.79 (d, J = 7.3 Hz, 1 H), 7.59 (t, J = 7.5 Hz, 1 H), 7.32 (s, 1 H), 6.86 (dd, J = 16.7, 10.5 Hz, 1 H), 6.13 (dd, J = 16.7, 2.4 Hz, 1 H), 5.70 (dd, J = 10.5, 2.4 Hz, 1 H), 5.00-4.90 (m, 1 H), 4.01 (s, 3 H), 3.90-3.70 (m, 4 H), 2.04-1.92 (m, 2 H ), 1.71-1.68 (m, 2 H). |
| 159 | 1-(4-((4- (isoquinolin-3- ylamino)-7- methoxyquinazolin- 6-yl)oxy)piperidin- 1-yl)prop-2-en-1- one | 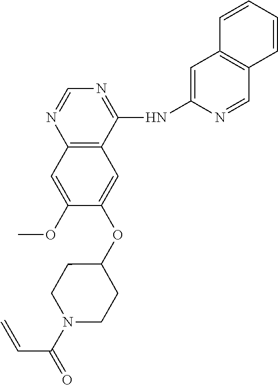 [SMILES: C=CC(=O)N1CCC(Oc2cc3c(Nc4cc5ccccc5cn4)ncnc3cc2OC)CC1] | None | LC/MS (ESI, m/z): [M + H]⁺ = 456.2. ¹H NMR (400 MHz, DMSO-d₆) δ 10.31 (s, 1 H), 9.24 (s, 1 H), 8.91 (s, 1 H), 8.67 (s, 1 H), 8.24 (s, 1 H), 8.10 (d, J = 8.1 Hz, 1 H), 7.95 (d, J = 8.3 Hz, 1 H), 7.73 (t, J = 7.6 Hz, 1 H), 7.55 (t, J = 7.5 Hz, 1 H), 7.27 (s, 1 H), 6.86 (dd, J = 16.7, 10.5 Hz, 1 H), 6.13 (dd, J = 16.7, 2.4 Hz, 1 H), 5.69 (dd, J = 10.5, 2.4 Hz, 1 H), 5.10-4.85 (m, 1 H), 3.96 (s, 3 H), 3.92-3.86 (m, 2 H), 3.54-3.48 (m, 2 H), 2.09-2.01 (m, 2 H), 1.73-1.67 (m, 2 H). |
| 160 | 1-(4-((4-((1- chloronaphthalen-2- yl)amino)-7- methoxyquinazolin- 6-yl)oxy)piperidin- 1-yl)prop-2-en-1- one | 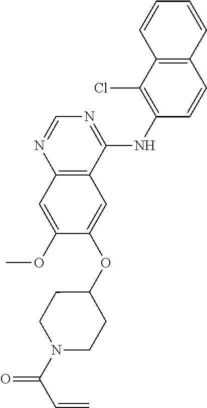 [SMILES: C=CC(=O)N1CCC(Oc2cc3c(Nc4ccc5ccccc5c4Cl)ncnc3cc2OC)CC1] | None | LC/MS (ESI, m/z): [M + H]⁺ = 489.1. ¹H NMR (400 MHz, DMSO-d₆) δ 11.18 (br s, 1 H), 8.70 (s, 1 H), 8.28 (d, J = 8.5 Hz, 1 H), 8.17 (s, 1 H), 8.13-8.09 (m, 2 H), 7.83- 7.67 (m, 3 H), 7.34 (s, 1 H), 6.86 (dd, J = 16.7, 10.5 Hz, 1 H), 6.13 (dd, J = 16.7, 2.4 Hz, 1 H), 5.70 (dd, J = 10.5, 2.4 Hz, 1 H), 4.88-4.84 (m, 1 H), 4.02 (s, 3 H), 3.96-3.83 (m, 2 H), 3.75-3.62 (m, 2 H), 2.15-2.03 (m, 2 H), 1.81-1.67 (m, 2 H). |
| 161 | 1-(4-((4- (isoquinolin-1- ylamino)-7- methoxyquinazolin- 6-yl)oxy)piperidin- 1-yl)prop-2-en-1- one | 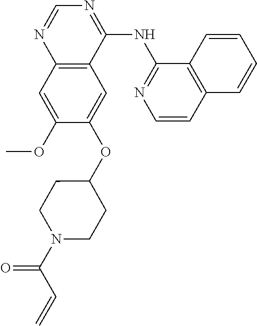 [SMILES: C=CC(=O)N1CCC(Oc2cc3c(Nc4nccc5ccccc45)ncnc3cc2OC)CC1] | None | LC/MS (ESI, m/z): [M + H]⁺ = 456.2. ¹H NMR (400 MHz, DMSO-d₆) δ 8.81 (br s, 1 H), 8.77 (s, 1 H), 8.13 (s, 1 H), 8.07-7.95 (m, 3 H), 7.81 (t, J = 7.7 Hz, 1 H), 7.58 (d, J = 5.6 Hz, 1 H), 7.30 (s, 1 H), 6.85 (dd, J = 16.7, 10.5 Hz, 1H), 6.13 (dd, J = 16.7, 2.4 Hz, 1H), 5.70 (dd, J = 10.5, 2.4 Hz, 1H), 4.98-4.91 (m, 1 H), 4.01 (s, 3 H), 3.97-3.85 (m, 2 H), 3.62-3.46 (m, 2 H), 2.13-2.00 (m, 2 H), 1.79-1.64 (m, 2 H). |
| 162 | 1-(4-((7-methoxy-4- (quinolin-4- ylamino)quinazolin- 6-y1)oxy)piperidin- 1-yl)prop-2-en-1- one | 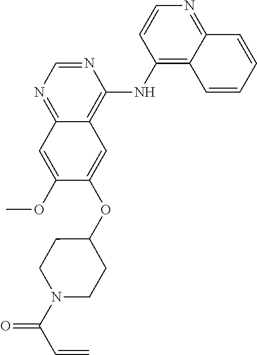 [SMILES: C=CC(=O)N1CCC(Oc2cc3c(Nc4ccnc5ccccc45)ncnc3cc2OC)CC1] | None | LC/MS (ESI, m/z): [M + H]⁺ = 456.1. ¹H NMR (400 MHz, CDCl₃) δ 8.51-8.45 (m, 2 H), 8.04 (d, J = 8 Hz, 1 H), 7.92 (d, J = 8.4 Hz, 1 H), 7.71-7.65 (m, 2 H), 7.60 (t, J = 8 Hz, 1 H), 7.48 (t, J = 8 Hz, 1 H), 7.24 (s, 1 H), 6.53-6.46 (m, 1 H), 6.19 (dd, J = 16.8 Hz, 1 H), 5.61 (dd, J = 10.8 Hz, 1 H), 4.72- 4.63 (m, 1 H), 3.95 (s, 3 H), 3.82-3.72 (m, 2 H), 3.55-3.43 (m, 2 H), 1.96-1.78 (m, 4 H). |
| 163 | 1-(4-((4-((4- fluoronaphthalen-1- yl)amino)-7- methoxyquinazolin- 6-yl)oxy)piperidin- 1-yl)prop-2-en-1- one | 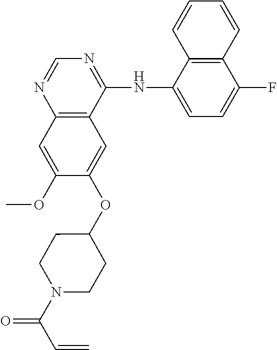 | None | LC/MS (ESI, m/z): [M + H]⁺ = 456.1. ¹H NMR (400 MHz, DMSO-d₆) δ 11.19 (s, 1 H), 8.61 (s, 1 H), 8.26-8.08 (m, 2 H), 7.93 (d, J = 8.3 Hz, 1 H), 7.78-7.69 (m, 1 H), 7.69- 7.61 (m, 2 H), 7.51 (dd, J = 10.3, 8.2 Hz, 1 H), 7.29 (s, 1 H), 6.86 (dd, J = 16.7, 10.5 Hz, 1 H), 6.12 (dd, J = 16.7, 2.4 Hz, 1 H), 5.70 (dd, J = 10.5, 2.4 Hz, 1 H), 4.88-4.80 (m, 1 H), 4.02 (s, 3 H), 3.94-3.82 (m, 2 H), 2.13- 2.03 (m, 2 H), 1.75-1.65 (m, 2 H). |
| 164 | 1-(4-((4-((4- chloronaphthalen-1- yl)amino)-7- methoxyquinazolin- 6-yl)oxy)piperidin- 1-yl)prop-2-en-1- one | 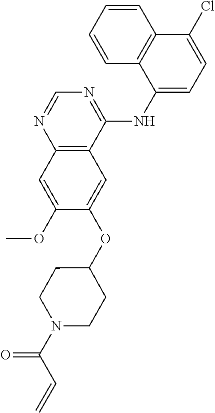 [SMILES: C=CC(=O)N1CCC(Oc2cc3c(Nc4ccc(Cl)c5ccccc45)ncnc3cc2OC)CC1] | None | LC/MS (ESI, m/z): [M + H]⁺ = 489.1. ¹H NMR (400 MHz, DMSO-d₆) δ 11.31 (s, 1 H), 8.59 (s, 1 H), 8.31-8.28 (m, 2 H), 8.00 (d, J = 8.4 Hz, 1 H), 7.87 (d, J = 8 Hz, 1 H), 7.81-7.77 (m, 1 H), 7.70-7.64 (m, 2 H), 7.34 (s, 1 H), 6.85 (dd, J = 10.4 Hz, J = 16.4 Hz, 1 H), 6.12 (dd, J = 2.4 Hz, J = 16.4 Hz, 1 H), 5.69 (dd, J = 2.4 Hz, J = 10.4 Hz, 1 H), 4.90-4.87 (m, 1 H), 4.02 (s, 3 H), 3.96-3.84 (m, 2 H), 3.58-3.51 (m, 2 H), 2.15-2.06 (m, 2 H), 1.79-1.68 (m, 2 H). |
| 165 | 1-(4-((4-((1H-indol- 4-yl)amino)-7- methoxyquinazolin- 6-yl)oxy)piperidin- 1-yl)prop-2-en-1- one | 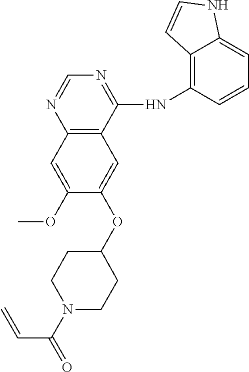 [SMILES: C=CC(=O)N1CCC(Oc2cc3c(Nc4cccc5[nH]ccc45)ncnc3cc2OC)CC1] | None | LC/MS (ESI, m/z): [M + 1]⁺ = 444.1. ¹H NMR (400 MHz, DMSO-d₆) δ 11.15 (s, 1 H), 9.56 (s, 1 H), 8.27 (s, 1 H), 8.02 (s, 1 H), 7.31-7.29 (m, 2 H), 7.23-7.06 (m, 3 H), 6.85 (dd, J = 16.6, 10.6 Hz, 1 H), 6.29 (s, 1 H), 6.11 (d, J = 16.6 Hz, 1 H), 5.68 (d, J = 9.4 Hz, 1 H), 4.82-4.78 (m, 1 H), 3.96-3.92 (m, 5 H), 3.52-3.34 (m, 2 H) , 2.06-1.98 (m, 2 H), 1.72-1.64 (m, 2 H). |

### Example 166: Preparation of 1-(4-((4-((1H-indol-7-yl)amino)-7-methoxyquinazolin-6-yl)oxy)piperidin-1-yl)prop-2-en-1-one (Compound 166)

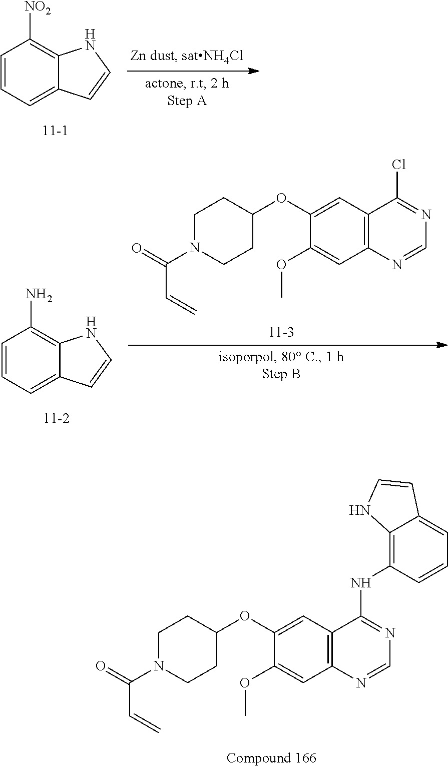 [SMILES: O=[N+]([O-])c1cccc2cc[nH]c12 ; Nc1cccc2cc[nH]c12 ; C=CC(=O)N1CCC(Oc2cc3c(Cl)ncnc3cc2OC)CC1 ; C=CC(=O)N1CCC(Oc2cc3c(Nc4cccc5cc[nH]c45)ncnc3cc2OC)CC1]

Step A: To a solution of 7-nitro-1H-indole (324 mg, 2 mmol) in acetone (20 mL) was added Zn dust (1.3 g, 20 mmol) and saturated ammonium chloride solution (1 mL). The mixture was stirred at room temperature for 2 h. The reaction mixture was filtered and concentrated in vacuum. The residue was dried to give 1H-indol-7-amine (250 mg) as a black solid, which was used directly in the next step without further purification. LC/MS (ESI, m/z): [M+H]⁺=133.1.

Step B: A mixture of 1-(4-((4-chloro-7-methoxyquinazolin-6-yl)oxy)piperidin-1-yl)prop-2-en-1-one (30 mg, 0.086 mmol) and 1H-indol-7-amine (11.4 mg, 0.086 mmol) in i-PrOH (5 mL) was warmed to 80° C. and stirred for 2 h. The reaction mixture was concentrated in vacuum and the residue was purified by prep-HPLC to afford 1-(4-((4-((1H-indol-7-yl)amino)-7-methoxyquinazolin-6-yl)oxy)piperidin-1-yl)prop-2-en-1-one (Compound 166) (17.3 mg, 45% yield). LC/MS (ESI, m/z): [M+H]⁺=444.2. ¹H NMR (400 MHz, DMSO-d₆) δ 11.25 (br. s., 1 H), 11.04 (br s, 1H), 8.70 (s, 1H), 8.18 (br. s., 1H), 7.62 (d, J=7.6 Hz, 1H), 7.37 (t, J=2.7 Hz, 1H), 7.33 (s, 1H), 7.19-7.09 (m, 2H), 6.85 (dd, J=10.5, 16.6 Hz, 1H), 6.54 (dd, J=1.9, 3.0 Hz, 1H), 6.12 (dd, J=2.4, 16.6 Hz, 1H), 5.70 (dd, J=2.4, 10.5 Hz, 1H), 4.83 (br. s., 1H), 4.03 (s, 3H), 3.96-3.82 (m, 2H), 3.50-3.36 (m, 2H), 2.15-2.04 (m, 2H), 1.79-1.64 (m, 2H).

### Example 167: Preparation of 1-(4-((7-methoxy-4-((4-methylnaphthalen-1-yl)amino)quinazolin-6-yl)oxy)piperidin-1-yl)prop-2-en-1-one (Compound 167)

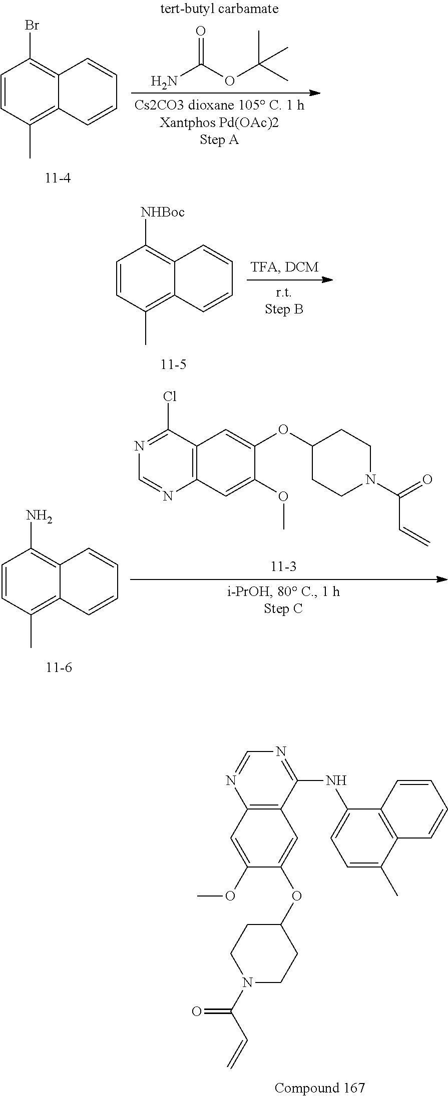

Step A: A solution of 1-bromo-4-methylnaphthalene (500 mg, 2.26 mmol) in dioxane was added tert-butyl carbamate (397 mg, 3.39 mmol), xantphos (262 mg, 0.45 mmol), palladium (II) acetate (51 mg, 0.23 mmol), Cs₂CO₃ (2.2 g, 6.78 mmol), the mixture was degassed with nitrogen, heated to 105° C. and stirred under microwave for 1 h, then the mixture then the mixture was then quenched by water, extracted with EA, washed by brine and dried by Na₂SO₄, filtered, the combined organic layers were concentrated in vacuo, the residue was purified by column chromatography to give the tert-butyl (4-methylnaphthalen-1-yl)carbamate (50 mg, 11% yield) as a yellow solid. LC/MS (ESI, m/z): [2M+1]⁺=202.2.

Step B was completed in a similar manner as described in Example 106 step B and step C was completed in a similar manner as described in Example 134 step C to afford 1-(4-((7-methoxy-4-((4-methylnaphthalen-1-yl)amino)quinazolin-6-yl)oxy)piperidin-1-yl)prop-2-en-1-one (Compound 167). LC/MS (ESI, m/z): [M+1]⁺=469.2. ¹H NMR (400 MHz, DMSO-d₆) δ 9.74 (s, 1H), 8.16 (s, 1H), 8.11-8.02 (m, 2H), 7.87 (d, J=8.4 Hz, 1H), 7.62-7.54 (m, 1H), 7.50 (dd, J=8.4, 6.8 Hz, 1H), 7.44 (s, 2H), 7.21 (s, 1H), 6.85 (dd, J=16.8, 10.4 Hz, 1H), 6.11 (dd, J=16.8, 2.4 Hz, 1H), 5.68 (dd, J=10.4, 2.4 Hz, 1H), 4.86-4.70 (m, 1H), 4.04-3.78 (m, 5H), 2.71 (s, 3H), 2.17-1.95 (m, 3H), 1.79-1.60 (m, 3H).

### Example 168: Preparation of 1-(4-((4-(indolin-5-ylamino)-7-methoxyquinazolin-6-yl)oxy)piperidin-1-yl)prop-2-en-1-one (Compound 168)

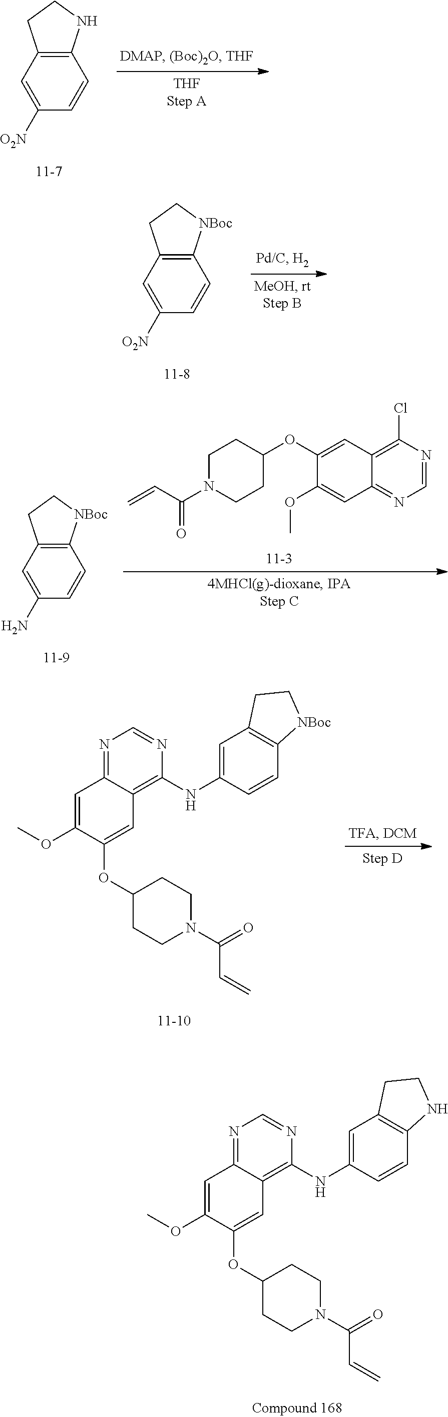 [SMILES: O=[N+]([O-])c1ccc2c(c1)CCN2 ; CC(C)(C)OC(=O)N1CCc2cc([N+](=O)[O-])ccc21 ; CC(C)(C)OC(=O)N1CCc2cc(N)ccc21 ; C=CC(=O)N1CCC(Oc2cc3c(Cl)ncnc3cc2OC)CC1 ; C=CC(=O)N1CCC(Oc2cc3c(Nc4ccc5c(c4)CCN5C(=O)OC(C)(C)C)ncnc3cc2OC)CC1 ; C=CC(=O)N1CCC(Oc2cc3c(Nc4ccc5c(c4)CCN5)ncnc3cc2OC)CC1]

Step A: To a solution of 5-nitroindoline (200 mg, 1.22 mmol) in THE (5 mL) were added (Boc)₂O (532 mg, 2.44 mmol) and 4-dimethylaminopyridine (14.9 mg, 0.122 mmol). The reaction was stirred at room temperature for 1 hour. The mixture was concentrated in vacuum and purified by column chromatography (PE/EA) to give tert-butyl 5-nitroindoline-1-carboxylate (288 mg, 89% yield). LC/MS (ESI, m/z): [M+H]⁺=265.1. ¹H NMR (400 MHz, DMSO-d₆) δ 8.17-7.95 (m, 2H), 7.74 (s, 1H), 4.02 (t, J=8.8 Hz, 2H), 3.16 (t, J=8.7 Hz, 2 H), 1.52 (s, 9H).

Step B: To a solution of tert-butyl 5-nitroindoline-1-carboxylate (288 mg, 1.09 mmol) in MeOH (10 mL) was added 10% Pd/C (20 mg). The reaction mixture was stirred at room temperature under H₂ for 18 hours. The mixture was concentrated in vacuum and purified by column chromatography (PE/EA) to give tert-butyl 5-aminoindoline-1-carboxylate (200 mg, 78% yield).

Step C: To a solution of tert-butyl 5-nitroindoline-1-carboxylate (33.68 mg, 144 umol) and 1-(4-((4-chloro-7-methoxyquinazolin-6-yl)oxy)piperidin-1-yl)prop-2-en-1-one (50 mg, 144 umol) in isopropanol (5 mL) was added 4 M HCl(g)-dioxane (cat. one drop). The reaction was stirred at 80° C. for 60 min. The mixture was concentrated in vacuum to give tert-butyl 5-((6-((1-acryloylpiperidin-4-yl)oxy)-7-methoxyquinazolin-4-yl)amino) indoline-1-carboxylate (60 mg, 77% yield). LC/MS (ESI, m/z): [M+H]⁺=546.2.

Step D: To a solution of tert-butyl 5-((6-((1-acryloylpiperidin-4-yl)oxy)-7-methoxyquinazolin-4-yl)amino)indoline-1-carboxylate (60 mg, 109.8 umol) in DCM (3 mL) and trifluoroacetic acid (2 mL). The mixture was stirred at RT for 1 hour. The mixture was concentrated in vacuum and the residue was purified via reverse phase column chromatography (CH₃CN/H₂O) to give 1-(4-((4-(indolin-5-ylamino)-7-methoxyquinazolin-6-yl)oxy)piperidin-1-yl)prop-2-en-1-one TFA salt (Compound 168) (17 mg, 35% yield) as a white solid. LC/MS (ESI, m/z): [M+H]⁺=446.2. ¹H NMR (400 MHz, DMSO-d₆) δ 10.94 (s, 1H), 8.75 (s, 1H), 8.15 (s, 1H), 7.38 (s, 1H), 7.28-7.08 (m, 2H), 6.95-6.68 (nM, 2H), 6.12 (dd, J=16.7, 2.3 Hz, 1 H), 5.70 (dd, J=10.5, 2.3 Hz, 1H), 4.90-4.81 (m, 1H), 4.00 (s, 3H), 3.60-3.51 (m, 6H), 3.04(t,J=8.2 Hz, 2H), 2.10-2.01 (m, 2H), 1.75-1.65 (m, 2H).

Examples 169-173 were prepared using similar procedures as described in the preceding Examples.

| EX. | | Compound | | |
|---|---|---|---|---|
| NO. | IUPAC Name | structure | salt | Compound characterization |
| 169 | 1-(4-((4-((1H-indol- 5-yl)amino)-7- methoxyquinazolin- 6-yl)oxy)piperidin-1- yl)prop-2-en-1-one | 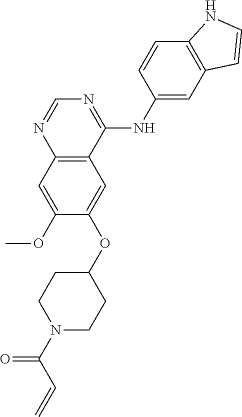 [SMILES: C=CC(=O)N1CCC(Oc2cc3c(Nc4ccc5[nH]ccc5c4)ncnc3cc2OC)CC1] | HCl | LC/MS (ESI, m/z): [M + H]⁺ = 444.2. ¹H NMR (400 MHz, DMSO-d₆) δ 14.58 (s, 1 H), 11.30 (d, J = 11.2 Hz, 2 H), 8.69 (s, 1 H), 8.40 (s, 1 H), 7.77 (d, J = 1.6 Hz, 1 H), 7.48 (d, J = 1.6 Hz, 1 ), 7.43 (t, J = 2.8 Hz, 1 H), 7.29 (dd, J = 2 Hz, J = 8.8 Hz, 1 H), 6.85 (dd, J = 10.4 Hz, J = 16.4 Hz, 1 H), 6.49 (s, 1 H), 6.12 (dd, J = 2.4 Hz, J = 16.8 Hz, 1 H), 5.69 (dd, J = 2.4 Hz, J = 10.4 Hz, 1 H), 2.01-4.94 (m, 1 H), 3.99 (s, 3 H), 3.95-3.87 (m, 2 H), 3.53-3.41 (m, 2 H), 2.13-2.03 (m, 2 H), 1.74-1.60 (m, 2 H). |
| 170 | 1-(4-((7-methoxy-4- ((1-methyl-1H-indol- 5- yl)amino)quinazolin- 6-yl)oxy)piperidin-1- yl)prop-2-en-1-one | 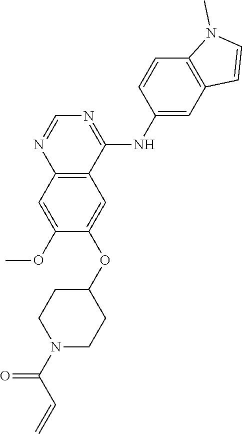 [SMILES: C=CC(=O)N1CCC(Oc2cc3c(Nc4ccc5c(ccn5C)c4)ncnc3cc2OC)CC1] | HCl | LC/MS (ESI, m/z): [M + H]⁺ = 458.2. ¹H NMR (400 MHz, DMSO-d₆) δ 14.53 (br s, 1 H), 11.35 (s, 1 H), 8.72 (s, 1 H), 8.37 (s, 1 H), 7.77 (d, J = 1.8 Hz, 1 H), 7.54 (d, J = 8.7 Hz, 1 H), 7.46-7.26 (m, 3 H), 6.85 (dd, J = 16.7, 10.5 Hz, 1 H), 6.49 (d, J = 2.5 Hz, 1 H), 6.12 (dd, J = 16.7, 2.4 Hz, 1 H), 5.69 (dd, J = 10.5, 2.4 Hz, 1 H), 5.01- 4.96 (m, 1 H), 4.06-3.78 (m, 8 H), 3.61- 3.41 (m, 2 H), 2.19-1.99 (m, 2 H), 1.81- 1.56 (m, 2 H). |
| 171 | 1-(4-((4-(indolin-5- ylamino)-7- methoxyquinazolin- 6-yl)oxy)piperidin-1- yl)prop-2-en-1-one | 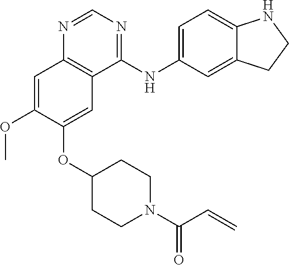 [SMILES: C=CC(=O)N1CCC(Oc2cc3c(Nc4ccc5c(c4)CCN5)ncnc3cc2OC)CC1] | TFA | LC/MS (ESI, m/z): [M + H]⁺ = 446.2. ¹H NMR (400 MHz, DMSO-d₆) δ10.94 (s, 1 H), 8.75 (s, 1 H), 8.15 (s, 1 H), 7.38 (s, 1 H), 7.28-7.08 (m, 2 H), 6.95-6.68 (m, 2 H), 6.12 (dd, J = 16.7, 2.3 Hz, 1 H), 5.70 (dd, J = 10.5, 2.3 Hz, 1 H), 4.90-4.81 (m, 1 H), 4.00 (s, 3 H), 3.60-3.51 (m, 6 H), 3.04 (t, J = 8.2 Hz, 2 H), 2.10-2.01 (m, 2 H), 1.75- 1.65 (m, 2 H) . |
| 172 | 1-(4-((4-((4- fluorobenzyl)amino)- 7- methoxyquinazolin- 6-yl)oxy)piperidin-1- yl)prop-2-en-1-one | 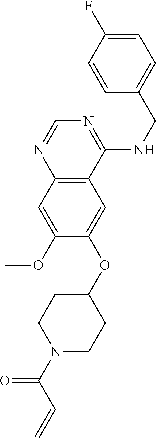 [SMILES: C=CC(=O)N1CCC(Oc2cc3c(NCc4ccc(F)cc4)ncnc3cc2OC)CC1] | None | LC/MS (ESI, m/z): [M + 1]+ = 437.2. ¹H NMR (400 MHz, DMSO) δ 8.44 (t, J = 6.0 Hz, 1 H), 8.33 (s, 1 H), 7.76 (s, 1 H), 7.39 (dd, J = 8.5, 5.7 Hz, 2 H), 7.22-6.98 (m, 3 H), 6.84 (dd, J = 16.7, 10.5 Hz, 1 H), 6.11 (dd, J = 16.7, 2.4 Hz, 1 H), 5.68 (dd, J = 10.5, 2.4 Hz, 1 H), 4.85-4.55 (m, 3 H), 3.90 (s, 3 H), 3.88-3.84 (m, 2 H), 3.47-3.37 (m, 2 H), 2.04-1.97 (m, 2 H), 1.74-1.58 (m, 2 H). |
| 173 | (R)-1-(4-((4-((1-(4- fluorophenyl)ethyl) amino)-7- methoxyquinazolin- 6-yl)oxy)piperidin-1- yl)prop-2-en-1-one | 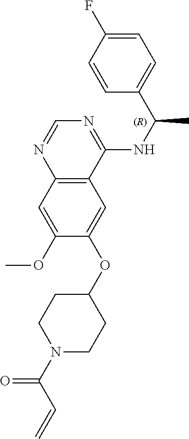 | None | LC/MS (ESI, m/z): [M + H]⁺ = 451.2. ¹H NMR (400 MHz, DMSO-d₆) δ 9.60 (d, 1 H), 8.75 (s, 1 H), 8.12 (s, 1 H), 7.49 (dd, J = 8.6, 5.5 Hz, 2 H), 7.30-7.12 (m, 3 H), 6.84 (dd, J = 16.7, 10.5 Hz, 1 H), 6.12 (dd, J = 16.7, 2.4 Hz, 1 H), 5.82-5.72 (m, 1 H), 5.69 (dd, J = 10.5, 2.4 Hz, 1 H), 4.91- 4.78 (m, 1 H), 3.99 (s, 3H ), 3.83-3.67 (m, 2 H), 3.67-3.55 (m, 2 H), 2.05-1.87 (m, 2 H), 1.81-1.69 (m, 2 H), 1.67 (d, J = 7.0 Hz, 3 H). |

### Example 174: Preparation of 1-(4-((7-methoxy-4-(4-methoxybenzoyl)quinazolin-6-yl)oxy)piperidin-1-yl)prop-2-en-1-one (Compound 174)

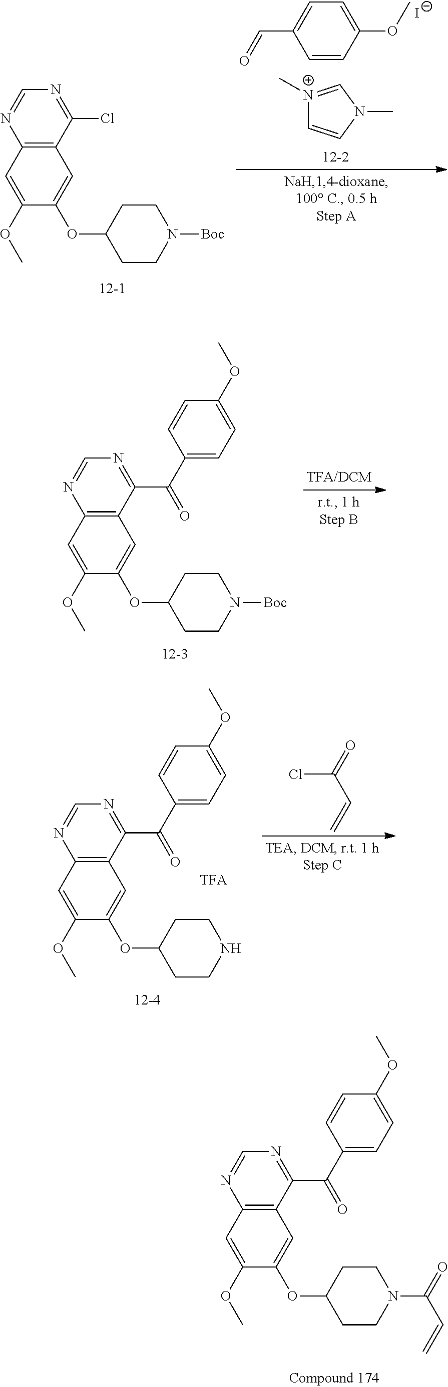

Step A: To a solution of tert-butyl 4-((4-chloro-7-methoxyquinazolin-6-yl)oxy)piperidine-1-carboxylate (100 mg, 0.25 mmol), 4-methoxybenzaldehyde (42 mg, 0.31 mmol) and 1,3-dimethyl-1H-imidazol-3-ium iodide (19 mg, 0.08 mmol) in dry 1,4-dioxane (5 mL) was added NaH (60% in oil, 12 mg, 0.31 mmol) under Ar at room temperature. The mixture was stirred at 100° C. for 0.5 h. After being cooled to room temperature, the mixture was quenched by sat. NH₄Cl aq (50 mL) and extracted with EtOAc (3×50 mL). The combined organic layer were washed with brine, dried over Na₂SO₄, filtered and concentrated. The residue was purified by column chromatography (PE:EA=1:1) to obtained tert-butyl 4-((7-methoxy-4-(4-methoxybenzoyl)quinazolin-6-yl)oxy)piperidine-1-carboxylate (77 mg, 62% yield) as a yellow oil. LC/MS (ESI, m/z): [M+H]⁺=494.2.

Step B: To a solution of tert-butyl 4-((7-methoxy-4-(4-methoxybenzoyl)quinazolin-6-yl)oxy)piperidine-1-carboxylate (75 mg, 0.15 mmol) in DCM (2 mL) was added TFA (1 mL) at 0° C. The mixture was stirred at room temperature for 1 hour. The mixture was diluted with DCM (40 mL) and concentrated to give (7-methoxy-6-(piperidin-4-yloxy)quinazolin-4-yl)(4-methoxyphenyl)methanone TFA salt (80 mg, 100% yield) as a yellow solid. LC/MS (ESI, m/z): [M+H]⁺=394.2.

Step C: To a solution of (7-methoxy-6-(piperidin-4-yloxy)quinazolin-4-yl)(4-methoxyphenyl)methanone TFA salt (80 mg, 0.15 mmol) and TEA (46 mg, 0.46 mmol) in dry DCM (4 mL) was dropwise added a solution of acryloyl chloride (28 mg, 0.30 mmol) in DCM (1 mL) over 2 min at 0° C. The reaction was stirred at 0° C. for 30 min. The mixture was directly purified by column chromatography (PE:EA=1:1) to obtained 1-(4-((7-methoxy-4-(4-methoxybenzoyl)quinazolin-6-yl)oxy)piperidin-1-yl)prop-2-en-1-one (Compound 174) (28.6 mg, 42.1% yield) as a pale-yellow solid. LC/MS (ESI, m/z): [M+H]⁺=448.2. ¹H NMR (400 MHz, DMSO-d₆) δ 9.19 (s, 1H), 7.93-7.82 (m, 2H), 7.53 (s, 1H), 7.29 (s, 1H), 7.14-7.04 (m, 2H), 6.80 (dd, J=16.7, 10.5 Hz, 1H), 6.09 (dd, J=16.7, 2.4 Hz, 1H), 5.67 (dd, J=10.5, 2.4 Hz, 1H), 4.82-4.68 (m, 1H), 4.03 (s, 3H), 3.87 (s, 3H), 3.84-3.76 (m, 2H), 3.49-3.38 (m, 2H), 1.96-1.83 (m, 2H), 1.67-1.53 (m, 2H).

### Example 175: Preparation of 1-(4-((4-(3,4-dichloro-2-fluorobenzoyl)-7-methoxyuinazolin-6-yl)oxy)piperidin-1-yl)prop-2-en-1-one (Compound 175)

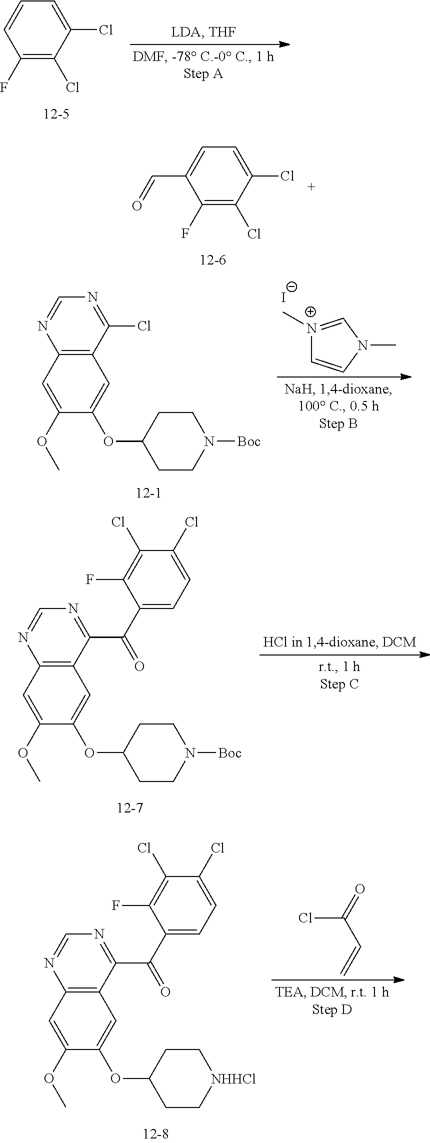

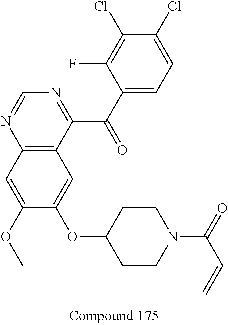 [SMILES: C=CC(=O)N1CCC(Oc2cc3c(C(=O)c4ccc(Cl)c(Cl)c4F)ncnc3cc2OC)CC1]

Step A: To a solution of 1, 2-dichloro-3-fluorobenzene (3.73 g, 22.74 mmol) in tetrahydrofuran (50 mL) at −78° C. was added lithium diisopropylamide (2 M in tetrahydrofuran-heptane, 17 mL, 34.15 mmol) over 17 min. The reaction was stirred for 2 hours. Then N N-dimethylformamide (5.2 mL, 68.22 mmol) was added over 10 min, and the reaction was stirred at −78° C. for another 1 hour. The mixture was slowly warmed to 0° C. over 30 min. The mixture was quenched by sat. NH₄Cl aq (100 mL) and extracted with EtOAc (3×100 mL). The combined organic layers were washed with brine, dried over Na₂SO₄, filtered and concentrated. The residue was purified by column chromatography (PE:EA=20:1) to obtained 3,4-dichloro-2-fluorobenzaldehyde (4.0 g, 92% yield) as a white solid. ¹H NMR (400 MHz, DMSO-d₆) δ 10.16 (s, 1H), 7.83 (dd, J=8.5, 7.2 Hz, 1H), 7.72 (dd, J=8.5, 1.1 Hz, 1H).

Steps B, C, and D were completed in a similar manner as described in Example 174 steps A, B, and C to afford 1-(4-((4-(3,4-dichloro-2-fluorobenzoyl)-7-methoxyquinazolin-6-yl)oxy)piperidin-1-yl)prop-2-en-1-one (Compound 175). LC/MS (ESI, m/z): [M+H]⁺=504.2, 506.2. ¹H NMR (400 MHz, DMSO-d₆) δ 9.19 (s, 1H), 7.94-7.70 (m, 3H), 7.55 (s, 1H), 6.83 (dd, J=16.7, 10.5 Hz, 1H), 6.11 (dd, J=16.7, 2.4 Hz, 1H), 5.68 (dd, J=10.5, 2.4 Hz, 1H), 4.88-4.84 (m, 1H), 4.05 (s, 3H), 3.94-3.80 (m, 2H), 3.53-3.36 (m, 2H), 2.09-1.98 (m, 2H), 1.76-1.64 (m, 2H).

### Example 176: Preparation of 1-(3-((4-((3,4-dichloro-2-fluorophenyl)amino)-7-methoxyquinazolin-6-yl)oxy)-9-azabicyclo[3.3.1]nonan-9-yl)prop-2-en-1-one (Compound 176)

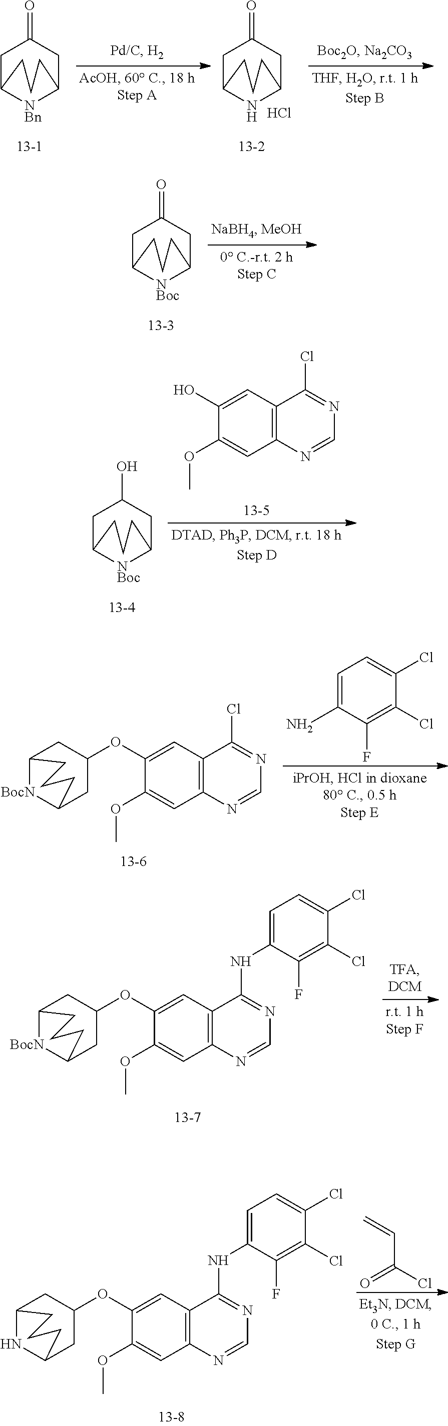

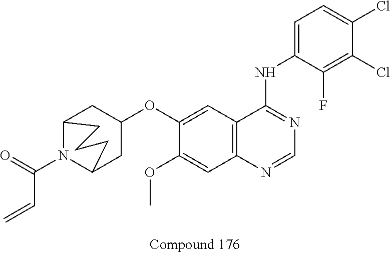 [SMILES: C=CC(=O)N1C2CCCC1CC(Oc1cc3c(Nc4ccc(Cl)c(Cl)c4F)ncnc3cc1OC)C2]

Step A: A suspension of 9-benzyl-9-azabicyclo[3.3.1]nonan-3-one (620 mg, 2.70 mmol) and 10 percent Pd/C (120 mg) in AcOH (15 mL) was stirred at 60° C. for 18 hours under H₂. The solid was filtered through a celite pad. HCl (4M in 1,4-dioxane, 0.7 mL) was added to the filtrate and the resulting mixture was evaporated to afford 9-azabicyclo[3.3.1]nonan-3-one HCl salt (1 g, crude) as a pale-yellow oil. LC/MS (ESI, m/z): [M+H]⁺=140.2.

Step B: To a suspension of 9-azabicyclo[3.3.1]nonan-3-one HCl salt (1 g, crude) and Boc₂O (883 mg, 4.05 mmol) in THF (5 mL) was added sat. Na₂CO₃ aq. (5 mL). The mixture was stirred at room temperature for 1 hour. The mixture was extracted with EtOAc (3×50 mL). The combined organic layers were washed with brine, dried over Na₂SO₄, filtered and concentrated. The residue was purified by column chromatography (PE:EA=4/1) to give tert-butyl 3-oxo-9-azabicyclo[3.3.1]nonane-9-carboxylate (509 mg, 79% yield 2 steps) as a white solid. LC/MS (ESI, m/z): [M−55]⁺=183.8.

Step C: To an ice-cooled solution of tert-butyl 3-oxo-9-azabicyclo[3.3.1]nonane-9-carboxylate (535 mg, 2.24 mmol) in MeOH (25 mL) was partwise added NaBH₄ (255 mg, 6.71 mmol). The mixture was stirred at room temperature for 1 hour. The mixture was quenched by Acetone (20 mL) and concentrated. The residue was purified by column chromatography (PE:EA=1:3) to give tert-butyl 3-hydroxy-9-azabicyclo[3.3.1]nonane-9-carboxylate (522 mg, 96.8% yield) as a white solid. LC/MS (ESI, m/z): [M−55]⁺=186.1.

Step D: DTAD (346 mg, 1.5 mmol) was added portionwise to an ice-cooled solution of 4-chloro-7-methoxyquinazolin-6-ol (210 mg, 1.0 mmol), tert-butyl 3-hydroxy-9-azabicyclo[3.3.1]nonane-9-carboxylate (360 mg, 1.5 mmol) and triphenylphosphine (394 mg, 1.5 mmol) in dry dichloromethane (10 mL). The mixture was stirred at room temperature for 18 hrs. After evaporation of the solvent under vacuum, the residue was purified by chromatography on silica gel (eluant:PE:EA:TEA=70:30:1) to give tert-butyl 3-((4-chloro-7-methoxyquinazolin-6-yl)oxy)-9-azabicyclo[3.3.1]nonane-9-carboxy late (304 mg, 52% yield). LC/MS (ESI, m/z): [M+H]⁺=434.2.

Step E: A mixture of tert-butyl 3-((4-chloro-7-methoxyquinazolin-6-yl)oxy)-9-azabicyclo[3.3.1]nonane-9-carboxylate (100 mg, 0.23 mmol), 3,4-dichloro-2-fluoroaniline (42 mg, 0.23 mmol) and HCl (4M in 1,4-dioxane, 0.06 mL) in i-PrOH (5 mL) was stirred at 80° C. for 1 hour. The mixture was concentrated and give tert-butyl 3-((4-((3,4-dichloro-2-fluorophenyl)amino)-7-methoxyquinazolin-6-yl)oxy)-9-azabicyclo[3.3.1]nonane-9-carboxylate (200 mg, crude) as a yellow solid, which used in next step without further purification. LC/MS (ESI, m/z): [M+H]⁺=577.2.

Step F: To a solution of tert-butyl 3-((4-((3,4-dichloro-2-fluorophenyl)amino)-7-methoxyquinazolin-6-yl)oxy)-9-azabicyclo[3.3.1]nonane-9-carboxylate (200 mg, crude) in DCM (1 mL) was added TFA (1 mL) at 0° C. The mixture was stirred at room temperature for 1 hour. The mixture was diluted with DCM (40 mL) and concentrated. The residue was freed by sat. Na₂CO₃ aq. to give 6-((9-azabicyclo[3.3.1]nonan-3-yl)oxy)-N-(3,4-dichloro-2-fluorophenyl)-7-methoxyquinazolin-4-amine (87 mg, 82% yield 2 steps) as a white solid. LC/MS (ESI, m/z): [M+H]⁺=477.2.

Step G: To a solution of 6-((9-azabicyclo[3.3.1]nonan-3-yl)oxy)-N-(3,4-dichloro-2-fluorophenyl)-7-methoxyquinazolin-4-amine (87 mg, 0.15 mmol) and TEA (46 mg, 0.46 mmol) in dry DCM (4 mL) was dropwise added a solution of acryloyl chloride (13 mg, 0.15 mmol) in DCM (1 mL) over 2 min at 0° C. The reaction was stirred at 0° C. for 30 min. The mixture was directly purified by column chromatography (EA) to obtained 1-(3-((4-((3,4-dichloro-2-fluorophenyl)amino)-7-methoxyquinazolin-6-yl)oxy)-9-azabicyclo[3.3.1]nonan-9-yl)prop-2-en-1-one (Compound 176) (15.7 mg, 19.7% yield) as a pale-yellow solid. LC/MS (ESI, m/z): [M+H]⁺=531.2, 533.2. ¹H NMR (400 MHz, DMSO-d₆) δ 9.64 (s, 1H), 8.40 (s, 1H), 7.85 (s, 1 H), 7.62 (dt, J=10.0, 8.8 Hz, 2H), 7.24 (s, 1H), 6.83 (dd, J=16.7, 10.5 Hz, 1H), 6.14 (dd, J=16.7, 2.4 Hz, 1H), 5.69 (dd, J=10.5, 2.4 Hz, 1H), 5.38-5.29 (m, 1H), 4.84-4.78 (m, 1H), 4.51-4.44 (m, 1H), 3.94 (s, 3H), 2.36-2.22 (m, 2H), 1.88-1.59 (m, 8H).

### Example 177: Preparation of 1-(3-((4-((3,4-dichloro-2-fluorophenyl)amino)-7-methoxyquinazolin-6-yl)oxy)-8-azabicyclo[3.2.1]octan-8-yl)prop-2-en-1-one (Compound 177)

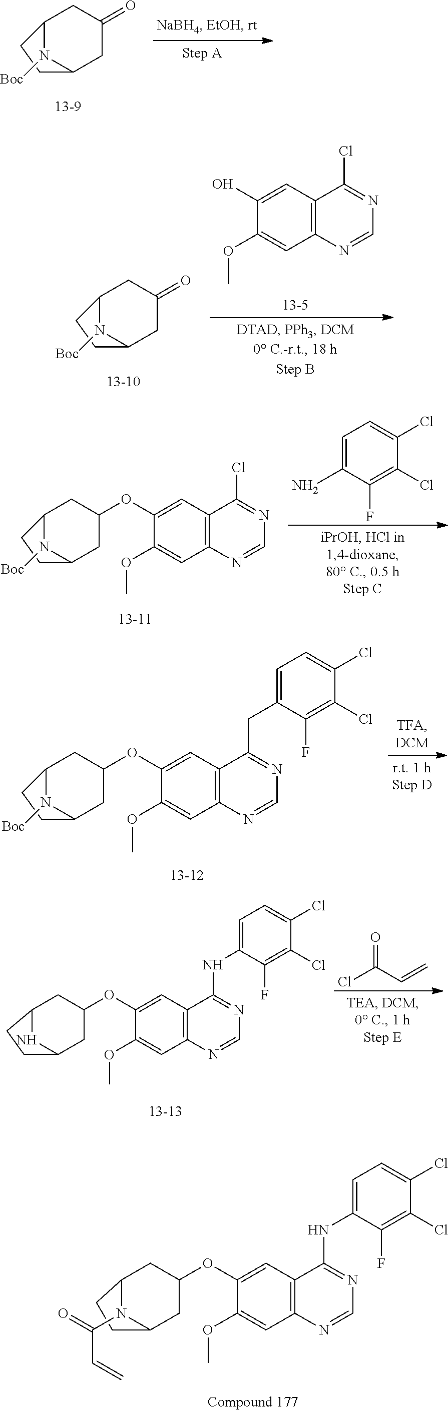

Step A: Sodium borohydride (178 mg, 4.7 mmol) was added to a solution of tert-butyl 3-oxo-8-azabicyclo[3.2.1]octane-8-carboxylate (0.50 g, 2.2 mmol) in ethanol (10 mL), and the resulting mixture was stirred at room temperature for one hour. The mixture was quenched with saturated ammonium chloride solution (30 mL), and extracted with ethyl acetate (3×20 mL). The combined organic layers were washed with water then brine, dried over sodium sulfate, filtered and concentrated to give tert-butyl 3-hydroxy-8-azabicyclo[3.2.1]octane-8-carboxylate (463 mg, 92% yield) as white solid. LC/MS (ESI, m/z): [M+H]⁺=228.2.

Steps B, C, D, and E were completed in a similar manner as described in Example 176 steps D, E, F, and G to afford 1-(3-((4-((3,4-dichloro-2-fluorophenyl)amino)-7-methoxyquinazolin-6-yl)oxy)-8-azabicyclo[3.2.1]octan-8-yl)prop-2-en-1-one (Compound 177). LC/MS (ESI, m/z): [M+1]⁺=517.2, 519.2. ¹H NMR (400 MHz, DMSO-d₆) δ 9.61 (s, 1H), 8.39 (s, 1H), 7.77 (s, 1H), 7.72-7.50 (m, 2H), 7.22 (s, 1H), 6.77 (dd, J=16.7, 10.3 Hz, 1H), 6.22 (dd, J=16.7, 2.3 Hz, 1H), 5.72 (dd, J=10.3, 2.3 Hz, 1H), 5.06-4.88 (m, 1H), 4.64-4.59 (m, 2H), 3.92 (s, 3H), 2.37-2.25 (m, 2H), 2.08-1.86 (m, 4H), 1.65 (t, J=10.2 Hz, 1H), 1.50 (t, J=10.3 Hz, 1H).

Examples 178-183 were prepared using similar procedures as described in the preceding Examples.

| EX. | | | | |
|---|---|---|---|---|
| NO. | IUPAC Name | Compound structure | salt | Compound characterization |
| 178 | 1-(3-((7-methoxy- 4-(naphthalen-1- ylamino)quinazolin- 6-yl)oxy)-9- azabicyclo[3.3.1] nonan-9-yl)prop-2-en- 1-one | 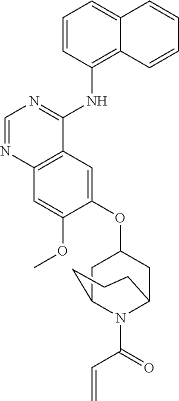 [SMILES: C=CC(=O)N1C2CCCC1CC(Oc1cc3c(Nc4cccc5ccccc45)ncnc3cc1OC)C2] | None | LC/MS (ESI, m/z): [M + H]⁺ = 495.2 ¹H NMR (400 MHz, DMSO-d₆) δ 11.29 (br s, 1 H), 8.61 (s, 1 H), 8.21- 8.02 (m, 3 H), 7.89 (d, J = 8.3 Hz, 1 H), 7.76-7.52 (m, 4 H), 7.30 (s, 1 H), 6.84 (dd, J = 16.7, 10.6 Hz, 1 H), 6.15 (dd, J = 16.7, 2.4 Hz, 1 H), 5.70 (dd, J = 10.5, 2.4 Hz, 1 H), 5.53-5.35 (m, 1 H), 4.90-4.77 (m, 1 H), 4.56-4.43 (m, 1 H), 4.00 (s, 3 H), 2.44-2.32 (m, 2 H), 2.05-1.60 (m, 8 H). |
| 179 | 1-(3-((7-methoxy- 4-(naphthalen-2- ylamino)quinazolin- 6-yl)oxy)-9- azabicyclo[3.3.1] nonan-9-yl)prop-2-en- 1-one | 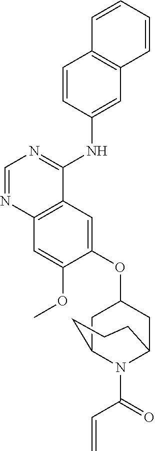 [SMILES: C=CC(=O)N1C2CCCC1CC(Oc1cc3c(Nc4ccc5ccccc5c4)ncnc3cc1OC)C2] | None | LC/MS (ESI, m/z): [M + 1]⁺ = 495.2. ¹H NMR (400 MHz, DMSO-d₆) δ 9.69 (s, 1 H), 8.53 (s, 1 H), 8.41 (s, 1 H), 8.05 (s, 1 H), 7.92 (dt, J = 14.4, 8.5 Hz, 4 H), 7.47 (dt, J = 14.9, 6.9 Hz, 2 H), 7.24 (s, 1 H), 6.84 (dd, J = 16.7, 10.5 Hz, 1 H), 6.14 (dd, J = 16.7, 2.4 Hz, 1 H), 5.69 (dd, J = 10.5, 2.4 Hz, 1 H), 5.44-5.27 (m, 1 H), 4.88-4.74 (m, 1 H), 4.55-4.40 (m, 1 H), 3.95 (s, 3 H), 2.30 (td, J = 13.8, 6.3 Hz, 2 H), 1.85-1.59 (m, 8 H). |
| 180 | 1-(3-((7-methoxy- 4-(naphthalen-2- ylamino)quinazolin- 6-yl)oxy)-8- azabicyclo[3.2.1] octan-8-yl)prop-2-en- 1-one | 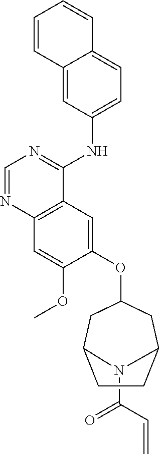 [SMILES: C=CC(=O)N1C2CCC1CC(Oc1cc3c(Nc4ccc5ccccc5c4)ncnc3cc1OC)C2] | None | LC/MS (ESI, m/z): [M + 1]⁺ = 481.2. ¹H NMR (400 MHz, DMSO-d₆) δ 10.95 (s, 1 H), 8.80 (s, 1 H), 8.24 (d, J = 1.8 Hz, 1 H), 8.15-7.93 (m, 4 H), 7.83 (dd, J = 8.8, 2.1 Hz, 1 H), 7.66- 7.48 (m, 2 H), 7.29 (s, 1 H), 6.77 (dd, J = 16.7, 10.3 Hz, 1 H), 6.22 (dd, J = 16.7, 2.4 Hz, 1 H), 5.73 (dd, J = 10.3, 2.4 Hz, 1 H), 5.29-5.00 (m, 1 H), 4.70-4.58 (m, 2 H), 3.98 (s, 3 H), 2.43-2.24 (m, 2 H), 1.98 (dt, J = 21.2, 11.2 Hz, 4 H), 1.67 (dt, J = 10.3 Hz, 1 H), 1.52 (dt, J = 10.1 Hz, 1 H). |
| 181 | 1-(3-((7-methoxy- 4-(naphthalen-1- ylamino)quinazolin- 6-yl)oxy)-8- azabicyclo[3.2.1] octan-8-yl)prop-2-en- 1-one | 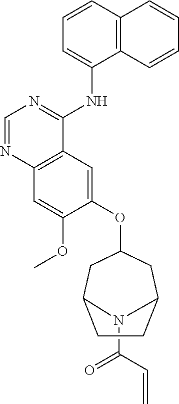 [SMILES: C=CC(=O)N1C2CCC1CC(Oc1cc3c(Nc4cccc5ccccc45)ncnc3cc1OC)C2] | None | LC/MS (ESI, m/z): [M + 1]⁺ = 481.2. ¹H NMR (400 MHz, DMSO-d₆) δ 9.77 (s, 1 H), 8.18 (s, 1 H), 8.01 (d, J = 7.8 Hz, 1 H), 7.98-7.89 (m, 2 H), 7.86 (d, J = 8.4 Hz, 1 H), 7.63-7.44 (m, 4 H), 7.20 (s, 1 H), 6.77 (dd, J = 16.7, 10.3 Hz, 1 H), 6.22 (dd, J = 16.7, 2.4 Hz, 1 H), 5.72 (dd, J = 10.3, 2.4 Hz, 1 H), 5.08-4.97 (m, 1 H), 4.70-4.54 (m, 2 H), 3.92 (s, 3 H), 2.43-2.23 (m, 2 H), 2.06-1.82 (m, 4 H), 1.67 (dt, J = 10.3 Hz, 1 H), 1.51 (dt, J = 10.2 Hz, 1 H). |
| 182 | 1-(3-((4-((2,3- dihydro-1H-inden- 4-yl)amino)-7- methoxyquinazolin- 6-yl)oxy)-9- azabicyclo[3.3.1] nonan-9-yl)prop-2-en- 1-one | 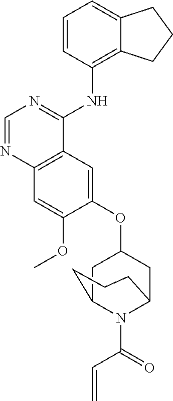 [SMILES: C=CC(=O)N1C2CCCC1CC(Oc1cc3c(Nc4cccc5c4CCC5)ncnc3cc1OC)C2] | None | LC/MS (ESI, m/z): [M + H]⁺ = 485.2. ¹H NMR (400 MHz, DMSO-d₆) δ 10.99 (s, 1 H), 8.73 (s, 1 H), 8.01 (s, 1 H), 7.43- 7.09 (m, 4 H), 6.83 (dd, J = 16.7, 10.6 Hz, 1 H), 6.14 (dd, J = 16.7, 2.4 Hz, 1 H), 5.70 (dd, J = 10.5, 2.4 Hz, 1 H), 5.46-5.36 (m, 1 H), 4.86-4.78 (m, 1 H), 4.52-4.46 (m, 1 H), 3.98 (s, 3 H), 2.98 (t, J = 7.4 Hz, 2 H), 2.77 (t, J = 7.4 Hz, 2 H), 2.42-2.24 (m, 2 H), 2.11-1.96 (m, 2 H), 1.82-1.64 (m, 8 H). |
| 183 | 1-(3-((4-((2,3- dihydro-1H-inden- 4-yl)amino)-7- methoxyquinazolin- 6-yl)oxy)-8- azabicyclo[3.2.1] octan-8-yl)prop-2-en- 1-one | 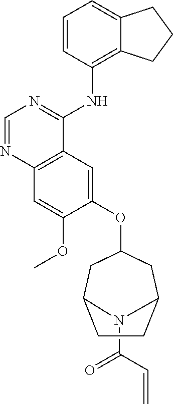 [SMILES: C=CC(=O)N1C2CCC1CC(Oc1cc3c(Nc4cccc5c4CCC5)ncnc3cc1OC)C2] | None | LC/MS (ESI, m/z): [M + 1]⁺ = 471.2. ¹H NMR (400 MHz, DMSO-d₆) δ 9.32 (s, 1 H), 8.31 (s, 1 H), 7.77 (s, 1 H), 7.30- 7.11 (m, 4 H), 6.76 (dd, J = 16.7, 10.3 Hz, 1 H), 6.21 (dd, J = 16.7, 2.4 Hz, 1 H), 5.72 (dd, J = 10.3, 2.4 Hz, 1 H), 5.05- 4.88 (m, 1 H), 4.61 (s, 2 H), 3.90 (s, 3 H), 2.94 (t, J = 7.4 Hz, 2 H), 2.74 (t, J = 7.4 Hz, 2 H), 2.29 (dd, J = 36.5, 5.9 Hz, 2 H), 2.08-1.76 (m, 6 H), 1.65 (t, J = 10.3 Hz, 1 H), 1.49 (t, J = 10.3 Hz, 1 H). |

### Example 184: Preparation of 1-(3-(4-((3,4-dichloro-2-fluorophenyl)amino)quinazolin-6-yl)piperidin-1-yl)prop-2-en-1-one (Compound 184)

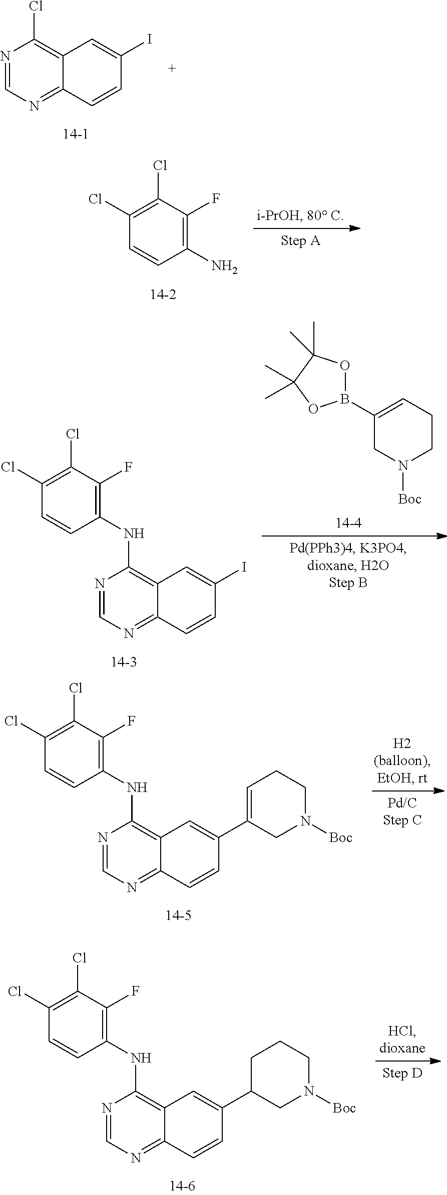 [SMILES: Clc1ncnc2ccc(I)cc12 ; Nc1ccc(Cl)c(Cl)c1F ; Fc1c(Nc2ncnc3ccc(I)cc23)ccc(Cl)c1Cl ; CC(C)(C)OC(=O)N1CCC=C(B2OC(C)(C)C(C)(C)O2)C1 ; CC(C)(C)OC(=O)N1CCC=C(c2ccc3ncnc(Nc4ccc(Cl)c(Cl)c4F)c3c2)C1 ; CC(C)(C)OC(=O)N1CCCC(c2ccc3ncnc(Nc4ccc(Cl)c(Cl)c4F)c3c2)C1]

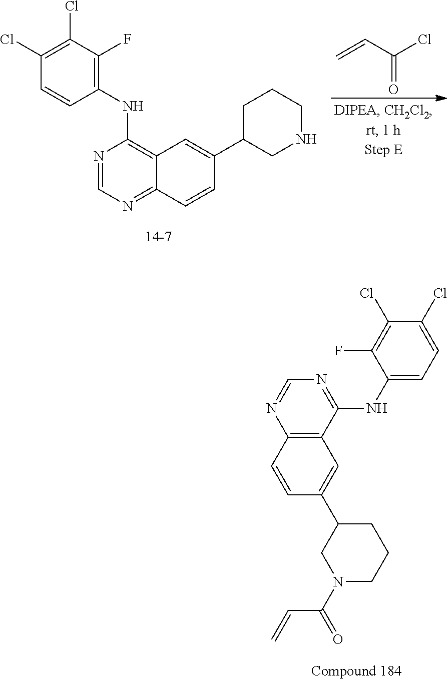

Step A: A mixture of 4-chloro-6-iodoquinazoline (1.6 g, 5.5 mmol) and 3,4-dichloro-2-fluoroaniline (1.09 g, 6.06 mmol) was slurry in i-PrOH (20 mL). The reaction mixture was stirred at 80° C. for 16 h. The solid was filtered to give the product N-(3,4-dichloro-2-fluorophenyl)-6-iodoquinazolin-4-amine as a grey solid (2.1 g, yield 88%). LC/MS (ESI, m/z): [M+H]⁺=434.0, 436.0. ¹H NMR (400 MHz, DMSO-d₆) δ 12.10 (br s, 1H), 9.35 (d, J=8.5 Hz, 1H), 8.94 (s, 1H), 8.40 (d, J=8.9 Hz, 1H), 7.82-7.79 (m, 1H), 7.69 (dd, J=8.8, 1.6 Hz, 7.63-7.58 (m, 1H).

Step B: A mixture of N-(3,4-dichloro-2-fluorophenyl)-6-iodoquinazolin-4-amine (434 mg, 1.0 mmol), tert-butyl 5-(4,4,5,5-tetramethyl-1,3,2-dioxaborolan-2-yl)-3,6-dihydropyridine-1(2H)-carboxylate (371 mg, 1.2 mmol) in dioxane (12 mL) and H₂O (3 mL) in a three neck flask. To the mixture was added Pd(PPh₃)₄(116 mg, 0.1 mmol), K₃PO₄ (636 mg, 3.0 mmol). The reaction mixture was exchanged with N₂ for three times and stirred at 90° C. under N₂ for 3 h. H₂O (20 mL) was added, and the mixture was extracted with EtOAc (40 mL×3). The combined organic layers were washed with brine, dried over anhydrous Na₂SO₄. The solid was filtered and the filtrate was concentrated to give a residue which was purified by column chromatography on silica gel (PE/EA=3/1) to give the product as an orange solid (451 mg, 92% yield). LC/MS (ESI, m/z): [M+H]⁺=489.1. ¹H NMR (400 MHz, CDCl₃) δ 8.78 (s, 1H), 8.61-8.28 (m, 1H), 7.98-7.91 (m, 2H), 7.85-7.71 (m, 2H), 7.36 (dd, J=9.0, 2.0 Hz, 1H), 6.42-6.39 (m, 1H), 4.40 (s, 2H), 3.62 (t, J=5.6 Hz, 2H), 2.43-2.38 (m, 2H), 1.52 (s, 9H).

Step C: To a solution of tert-butyl 5-(4-((3,4-dichloro-2-fluorophenyl)amino)quinazolin-6-yl)-3,6-dihydropyridine-1(2H)-carboxylate (265 mg, 0.54 mmol) in EtOH (10 mL) was added 10% Pd—C(70 mg). The reaction mixture was exchanged with H₂ for three times. The reaction mixture was stirred at room temperature under H₂ balloon for 16 h. Pd—C was filtered and the filtrate was concentrated to give a residue which was purified by column chromatography on silica gel (PE/EA=1/1) to give the product tert-butyl 3-(4-((3,4-dichloro-2-fluorophenyl)amino)quinazolin-6-yl)piperidine-1-carboxylate as a pale yellow solid (240 mg, yield 90%). LC/MS (ESI, m/z): [M+H]⁺=491.1, 493.1.

Step D: To a solution of tert-butyl 3-(4-((3,4-dichloro-2-fluorophenyl)amino)quinazolin-6-yl)piperidine-1-carboxylate (69 mg, 0.14 mmol) in MeOH (2 mL) was added HCl (0.7 mL) (4 M in dioxane) at room temperature. The reaction mixture was stirred at room temperature for 30 min. The solvent was removed under reduced pressure to give the crude product N-(3,4-dichloro-2-fluorophenyl)-6-(piperidin-3-yl)quinazolin-4-amine hydrochloride (60 mg) which was used directly in next step without any purification. LC/MS (ESI, m/z): [M+H]⁺=391.1, 393.0.

Step E: To a slurry of crude N-(3,4-dichloro-2-fluorophenyl)-6-(piperidin-3-yl)quinazolin-4-amine hydrochloride (60 mg, 0.14 mmol) in CH₂Cl₂ (2 mL) was added DIPEA (54 mg, 0.42 mmol). The reaction mixture was turned to a clear solution. To the reaction mixture was added acryloyl chloride (1.3 mL, 0.13 mmol) (0.1 M in CH₂Cl₂ at 0° C. The reaction mixture was stirred at room temperature for 1 h. H₂O (5 mL) was added, the mixture was extracted with CH₂Cl₂ (5 mL×3), washed with brine. The organic phase was dried over Na₂SO₄. The solid was filtered and the filtrate was concentrated to give a residue which was purified by column chromatography on silica gel (CH₂Cl₂/MeOH=15/1) to give 1-(3-(4-((3,4-dichloro-2-fluorophenyl)amino)quinazolin-6-yl)piperidin-1-yl)prop-2-en-1-one (Compound 184) as a white solid (43 mg, yield 69%). LC/MS (ESI, m/z): [M+H]⁺=445.0. H NMR showed two pair of signals. ¹H NMR (400 MHz, CDCl₃, a=0.3, b=0.7) δ 8.78 (s, 1H), 8.52 (t, J=7.8 Hz, 0.3 Ha), 8.42 (t, J=8.2 Hz, 0.7 Hb), 7.96-7.86 (m, 2H), 7.77-7.71 (m, 2H), 7.35 (d, J=8.9 Hz, 1H), 6.64 (dd, J=16.4, 10.4 Hz, 1H), 6.33 (d, J=16.6 Hz, 1H), 5.73 (d, J=10.1 Hz, 1 H), 4.79 (d, J=10.7 Hz, 0.3 Ha), 4.49 (d, J=2.9 Hz, 0.7 Hb), 4.15 (d, J=10.6 Hz, 0.3 Ha), 3.91 (d, J=12.5 Hz, 0.7 Hb), 3.41-3.22 (m, 2H), 3.08-2.94 (m, 1H), 2.21-2.14 (m, 1H), 2.07-1.76 (m, 3H).

### Example 185: Preparation of 1-(3-(4-((3,4-dichloro-2-fluorophenyl)amino)-7-methoxyquinazolin-6-yl)piperidin-1-yl)prop-2-en-1-one (Compound 185)

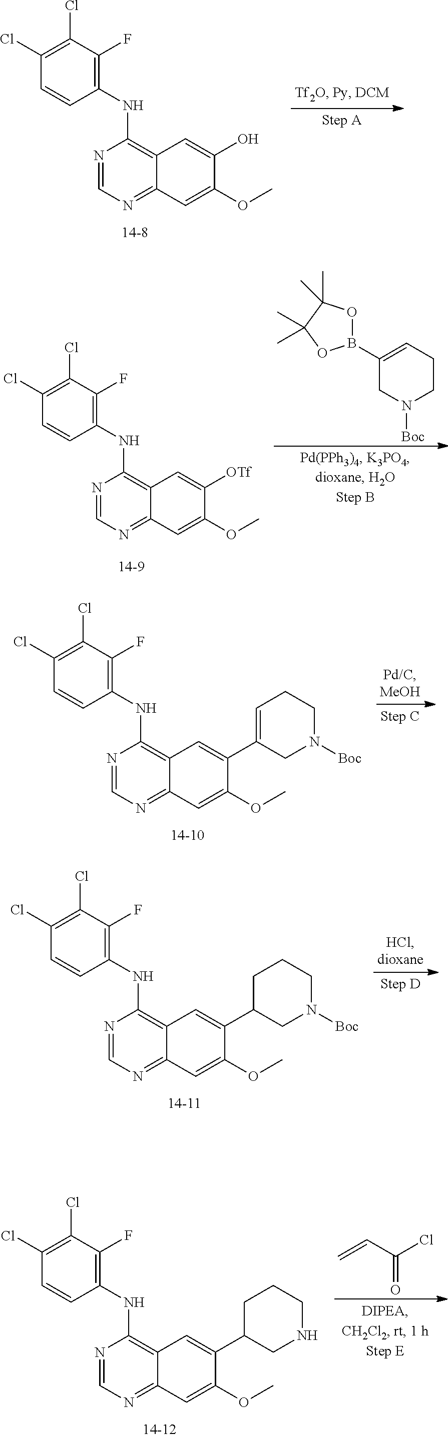

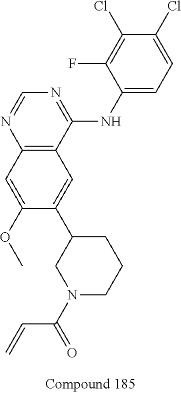 [SMILES: C=CC(=O)N1CCCC(c2cc3c(Nc4ccc(Cl)c(Cl)c4F)ncnc3cc2OC)C1]

Step A: To a solution of 4-((3,4-dichloro-2-fluorophenyl)amino)-7-methoxyquinazolin-6-ol (708 mg, 2 mmol), pyridine (320 mg, 4 mmol) in DCM (10 mL) was added Tf₂O (846 mg, 3 mmol) at 0° C. The mixture was stirred at room temperature for 16 hours. The reaction mixture was purified by column (PE:EA=2:1) to give 4-((3,4-dichloro-2-fluorophenyl)amino)-7-methoxyquinazolin-6-yl-trifluoromethanesulfonate (580 mg, 60% yield) as a white solid. LC/MS (ESI, m/z): [M+H]⁺=486.2.

Step B: To a solution of 4-((3,4-dichloro-2-fluorophenyl)amino)-7-methoxyquinazolin-6-yl trifluoromethanesulfonate (580 mg, 1.2 mmol) and tert-butyl 3-(4,4,5,5-tetramethyl-1,3,2-dioxaborolan-2-yl)-5,6-dihydropyridine-1(2H)-carboxylate (443 mg, 1.44 mmol), K₃PO₄ (763 mg, 3 mmol) in dioxane/H₂O (4:1, 15 mL) was added Pd(PPh₃)₄(139 mg, 0.12 mmol). The reaction mixture was exchanged with N₂ for three times. The mixture was heated to 90° C. for 16 hours under N₂ balloon. The reaction mixture was cooled to room temperature. To the mixture was filtered and washed with MeOH. The organic layer was concentrated and purified by column (PE:EA=2:1) to give tert-butyl 3-(4-((3,4-dichloro-2-fluorophenyl)amino)-7-methoxyquinazolin-6-yl)-5,6-dihydropyridine-1(2H)-carboxylate (440 mg, 70% yield) as a yellow solid. LC/MS (ESI, m/z): [M+H]⁺=519.1.

Step C: To a solution of tert-butyl 3-(4-((3,4-dichloro-2-fluorophenyl)amino)-7-methoxyquinazolin-6-yl)-5,6-dihydropyridine-1(2H)-carboxylate (170 mg, 0.33 mmol) in MeOH (5 mL) was added Pd/C (200 mg) under N₂. The reaction mixture was exchanged with H₂ and stirred at rt for 16 hours under 150 psi of H₂. The mixture was filtered and washed with MeOH. The organic layer was concentrated and purified by column (PE:EA=1:1) to give tert-butyl 3-(4-((3,4-dichloro-2-fluorophenyl)amino)-7-methoxyquinazolin-6-yl)piperidine-1-carboxylate (100 mg, 57% yield) as a brown oil. LC/MS (ESI, m/z): [M+H]⁺=521.1.

Step D: tert-butyl 3-(4-((3,4-dichloro-2-fluorophenyl)amino)-7-methoxyquinazolin-6-yl)piperidine-1-carboxylate (100 mg, 0.192 mmol) was added HCl (2 mL, 8 mmol) (4 M in dioxane). The reaction mixture was stirred at room temperature for 1 h. The solvent was concentrated to give the crude product which is used directly in next step without any purification.

Step E was completed in a similar manner as described in Example 184 step E to afford 1-(3-(4-((3,4-dichloro-2-fluorophenyl)amino)-7-methoxyquinazolin-6-yl)piperidin-1-yl)prop-2-en-1-one (Compound 185). LC/MS (ESI, m/z): [M+1]⁺=475.2, 477.2. ¹H NMR (400 MHz, DMSO-d₆) δ 9.97 (d, J=6.0 Hz, 1H), 8.45 (s, 1H), 8.30 (s, 1H), 7.77-7.64 (m, 1H), 7.59 (s, 2H), 7.23 (s, 1H), 6.88-6.82 (m, 1H), 6.13 (dd, J=16.8, 2.2 Hz, 1H), 5.75-5.68 (m, 1H), 4.61 (dd, J=30.4 Hz, 1H), 4.26-4.09 (m, 1H), 4.07-3.91 (m, 3H), 3.12-3.00 (m, 2H), 2.09-1.76 (m, 4H).

Examples 186-187 were prepared using similar procedures as described in the preceding Examples.

| EX. | | Compound | | |
|---|---|---|---|---|
| NO. | IUPAC Name | structure | salt | Compound characterization |
| 186 | 1-(5-(4-((3,4- dichloro-2- fluorophenyl)amino)- 7- methoxyquinazolin- 6-yl)-3,6- dihydropyridin- 1(2H)-yl)prop-2-en- 1-one | 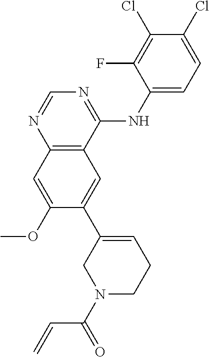 [SMILES: C=CC(=O)N1CCC=C(c2cc3c(Nc4ccc(Cl)c(Cl)c4F)ncnc3cc2OC)C1] | TFA | LC/MS (ESI, m/z): [M + 1]+ = 473.0, 475.0. ¹H NMR (400 MHz, DMSO-d₆) δ 10.81 (br s, 1 H), 8.71 (s, 1 H), 8.39 (s, 1 H), 7.66-7.46 (m, 2 H), 7.29 (s, 1 H), 6.92-6.88 (m, 1 H), 6.21-5.98 (m, 2 H), 5.73 (d, J = 10.3 Hz, 1 H), 4.41 (d, J = 8.5 Hz, 2 H), 3.99 (s, 3 H), 3.78-3.72 (m, 2 H), 2.38-2.33 (m, 2 H). |
| 187 | 1-(5-(4-((3,4- dichloro-2- fluorophenyl)amino) quinazolin-6-yl)-3,6- dihydropyridin- 1(2H)-yl)prop-2-en- 1-one | 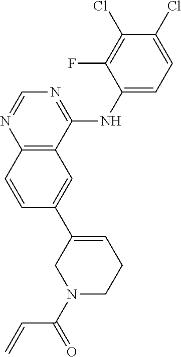 [SMILES: C=CC(=O)N1CCC=C(c2ccc3ncnc(Nc4ccc(Cl)c(Cl)c4F)c3c2)C1] | None | LC/MS (ESI, m/z): [M + 1]⁺ = 443.0. ¹H NMR (400 MHz, DMSO-d₆) δ 10.24-10.06 (m, 1 H), 8.51 (s, 1 H), 8.43 (s, 1 H), 8.08 (d, J = 7.7 Hz, 1 H), 7.80 (d, J = 8.7 Hz, 1 H), 7.61 (s, 2 H), 7.18-6.82 (m, 1 H), 6.60 (s, 1 H), 6.19 (t, J= 15.5 Hz, 1 H), 5.74 (d, J = 12.1 Hz, 1 H), 4.62 (s, 2 H), 3.75 (d, J = 4.9 Hz, 2 H), 2.42-2.37 (m, 2 H). |

### Example 188: Preparation of N-(3,4-dichloro-2-fluorophenyl)-6-(1-(vinylsulfonyl)piperidin-3-yl)quinazolin-4-amine (Compound 188)

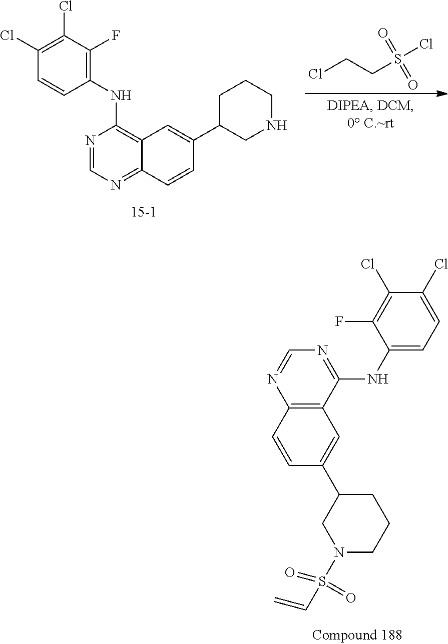 [SMILES: Fc1c(Nc2ncnc3ccc(C4CCCNC4)cc23)ccc(Cl)c1Cl ; O=S(=O)(Cl)CCCl ; C=CS(=O)(=O)N1CCCC(c2ccc3ncnc(Nc4ccc(Cl)c(Cl)c4F)c3c2)C1]

To a solution of N-(3,4-dichloro-2-fluorophenyl)-6-(piperidin-3-yl)quinazolin-4-amine (39 mg, 0.1 mmol) in DCM (2 mL) was added DIPEA (39 mg, 0.3 mmol) and 2-chloroethane-1-sulfonyl chloride (18 mg, 0.11 mmol) at 0° C. The reaction mixture was stirred at room temperature for 4 h. The solvent was concentrated to give a residue which was purified by column chromatography on silica gel (CH₂Cl₂/MeOH=15/1) to give N-(3,4-dichloro-2-fluorophenyl)-6-(1-(vinylsulfonyl)piperidin-3-yl)quinazolin-4-amine (Compound 188) as a white solid (6 mg, yield 13%). LC/MS (ESI, m/z): [M+H]⁺=481.0, 483.0. ¹H NMR (400 MHz, CDCl₃) δ 8.79 (s, 1H), 8.47 (dd, J=9.0, 8.1 Hz, 1H), 7.93 (d, J=8.7 Hz, 1H), 7.88 (d, J=1.2 Hz, 1H), 7.73 (dd, J=8.6, 1.8 Hz, 1H), 7.71-7.68 (m, 1H), 7.35 (dd, J=9.0, 2.0 Hz, 1H), 8.47 (dd, J=16.6, 9.8 Hz, 1H), 8.27 (d, J=16.6 Hz, 1H), 8.06 (d, J=10.0 Hz, 1H), 3.76 (dd, J=11.8, 3.6 Hz, 1H), 3.64 (dt, J=11.3, 3.0 Hz, 1H), 3.19-3.12 (m, 1H), 3.02 (dd, J=11.4, 10.0 Hz, 1H), 2.95-2.90 (m, 1H), 2.13-2.09 (m, 1H), 1.90-1.79 (m, 3H).

### Example 189: Preparation of 2-((3-(4-((3,4-dichloro-2-fluorophenyl)amino)quinazolin-6-yl)azetidin-1-yl)sulfonyl)ethan-1-ol (Compound 189)

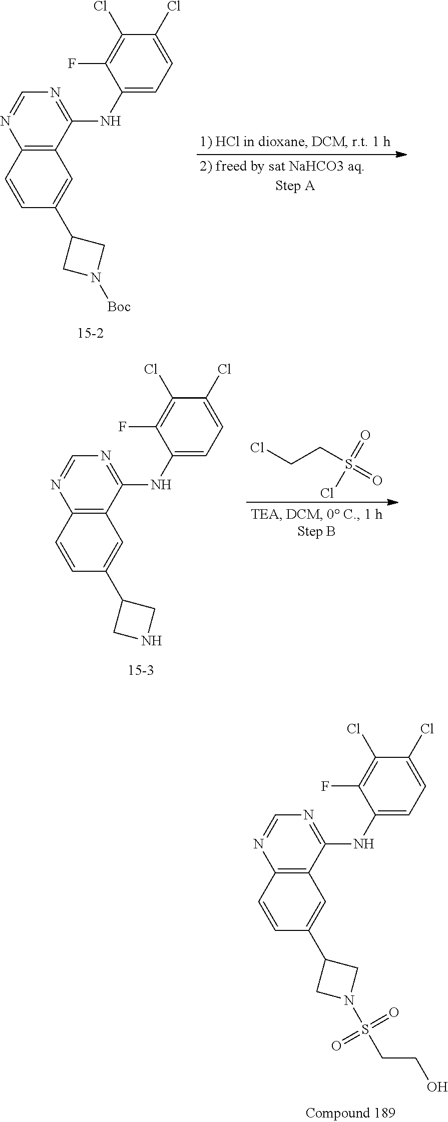

Step A: To a solution of tert-butyl 3-(4-((3,4-dichloro-2-fluorophenyl)amino)quinazolin-6-yl)azetidine-1-carboxylate (150 mg, 0.32 mmol) in DCM (3 mL) was added 4 M HCl in dioxane (1 mL). The reaction mixture was stirred at room temperature for 1 h. The mixture was added saturate NaHCO₃ to pH=8˜9. The mixture was extracted with DCM (10 mL×3), the combined organic layers were washed by brine, dried over Na₂SO₄. The solid was filtered and the filtrate was concentrated to give the crude product (72 mg, yield 62%) which was used directly without any purification. LC/MS (ESI, m/z): [M+H]⁺=363.1, 365.1.

Step B: To a solution of 6-(azetidin-3-yl)-N-(3,4-dichloro-2-fluorophenyl)quinazolin-4-amine (72 mg, 0.2 mmol) in DCM (2 mL) was added TEA (61 mg, 0.6 mmol) and 2-chloroethane-1-sulfonyl chloride (19 mg, 0.3 mmol) at 0° C. The reaction mixture was stirred at room temperature for 4 h. The solvent was concentrated to give a residue which was purified by Prep-HPLC (CH₃CN and H₂O with 0.01% TFA as mobile phase) to give 2-((3-(4-((3,4-dichloro-2-fluorophenyl)amino)quinazolin-6-yl)azetidin-1-yl)sulfonyl)ethan-1-ol (Compound 189) as a white solid (16 mg, yield 17%). LC/MS (ESI, m/z): [M+H]⁺=471.0, 473.0. ¹H NMR (400 MHz, DMSO-d₆) δ 10.05 (s, 1H), 8.53 (s, 1H), 8.39 (s, 1H), 7.99-7.78 (m, 2H), 7.61 (s, 2 H), 4.60-4.30 (m, 1H), 4.28-3.91 (m, 2H), 3.46 (t, J=6.4 Hz, 2H), 2.76 (t, J=6.7 Hz, 2H).

Examples 190-193 were prepared using similar procedures as described in the preceding Examples.

| EX. | | Compound | | |
|---|---|---|---|---|
| NO. | IUPAC Name | structure | salt | Compound characterization |
| 190 | N-(3,4-dichloro- 2-fluorophenyl)- 6-(1- (vinylsulfonyl)- 1,2,5,6- tetrahydropyridin- 3-yl)quinazolin- 4-amine | 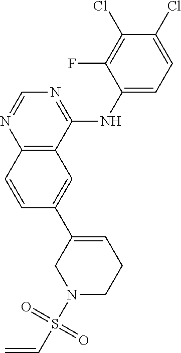 [SMILES: C=CS(=O)(=O)N1CCC=C(c2ccc3ncnc(Nc4ccc(Cl)c(Cl)c4F)c3c2)C1] | None | LC/MS (ESI, m/z): [M + 1]⁺ = 479.0. ¹H NMR (400 MHz, CDCl₃) δ 8.79 (s, 1 H), 8.43 (t, J = 8.5 Hz, 1 H), 7.94 (d, J = 8.8 Hz, 1 H), 7.86 (dd, J = 8.8, 1.7 Hz, 1 H), 7.75 (s, 1 H), 7.36 (dd, J = 9.0, 2.0 Hz, 1 H), 6.54 (dd, J = 16.6, 9.9 Hz, 1 H), 6.40-6.27 (m, 2 H), 6.08 (d, J = 9.9 Hz, 1 H), 4.20 (d, J = 2.0 Hz, 2 H), 3.45 (t, J = 5.8 Hz, 2 H), 2.57-2.49 (m, 2 H). |
| 191 | N-(3-((4-((3,4- dichloro-2- fluorophenyl) amino)-7- methoxyquinazolin- 6- yl)oxy)cyclobutyl) ethenesulfonamide | 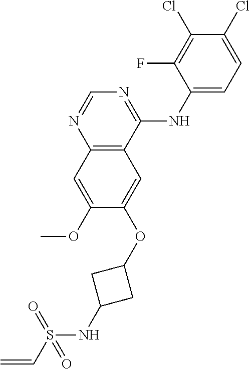 [SMILES: C=CS(=O)(=O)NC1CC(Oc2cc3c(Nc4ccc(Cl)c(Cl)c4F)ncnc3cc2OC)C1] | None | LC/MS (ESI, m/z): [M + 1]⁺ = 513.0, 515.0. HPLC and H NMR showed the ratio of diastereomers cis/trans is about 1/1. ¹H NMR (400 MHz, DMSO-d₆) δ 9.72 (d, J = 22.0 Hz, 1 H), 8.40 (s, 1 H), 7.84 (dd, J = 24.1, 8.1 Hz, 1 H), 7.59 (s, 2 H), 7.50 (d, J = 14.7 Hz, 1 H), 7.23 (d, J = 1.9 Hz, 1 H), 6.78-6.70(m, 1 H), 6.02 (dd, J = 16.8 Hz, 2.0 Hz, 2 H), 4.96-4.89 (m, 0.5 H, trans), 4.54-4.48 (m, 0.5 H, cis), 3.93-3.87 (m, 0.5 H, trans), 3.94 (s, 3 H), 3.49-3.39 (m, 0.5 H, cis) 3.11-2.99 (m, 2 H), 2.13-2.03 (m, 2 H). |
| 192 | N-(3,4-dichloro- 2-fluorophenyl)- 7-methoxy-6-((1- (vinylsulfonyl) piperidin-4- yl)oxy)quinazolin- 4-amine | 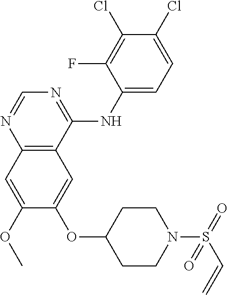 [SMILES: C=CS(=O)(=O)N1CCC(Oc2cc3c(Nc4ccc(Cl)c(Cl)c4F)ncnc3cc2OC)CC1] | None | LC/MS (ESI, m/z): [M + 1]⁺ = 527.1, 529.1. ¹H NMR (400 MHz, DMSO-d₆) δ 9.65 (s, 1 H), 8.40 (s, 1 H), 7.85 (s, 1 H), 7.71-7.39 (m, 2 H), 7.24 (s, 1 H), 6.89 (dd, J = 16.5, 10.0 Hz, 1 H), 6.18 (dd, J = 23.9, 13.3 Hz, 2 H), 4.82- 4.49 (m, 1 H), 3.95 (s, 3 H), 3.40-3.32 (m, 2 H), 3.20-3.02 (m, 2 H), 2.14-2.05 (m, 2 H), 1.93-1.85 (m, 2 H). |
| 193 | (3,4-dichloro-2- fluorophenyl)(7- methoxy-6-((1- (vinylsulfonyl) piperidin-4- yl)oxy)quinazolin- 4-yl)methanone | 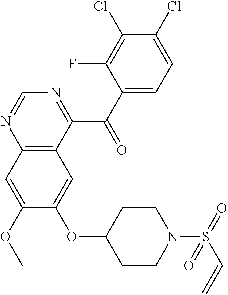 [SMILES: C=CS(=O)(=O)N1CCC(Oc2cc3c(C(=O)c4ccc(Cl)c(Cl)c4F)ncnc3cc2OC)CC1] | None | LC/MS (ESI, m/z): [M + H]⁺ = 540.0, 542.0. ¹H NMR (400 MHz, DMSO-d₆) δ 9.19 (s, 1 H), 7.86 (dd, J = 8.5, 7.2 Hz, 1 H), 7.82- 7.71 (m, 2 H), 7.55 (s, 1 H), 6.88 (dd, J = 16.5, 10.0 Hz, 1 H), 6.17 (dd, J = 23.1, 13.3 Hz, 2 H), 4.83-4.72 (m, 1 H), 4.05 (s, 3 H), 3.37-3.28 (m, 2 H), 3.13-3.01 (m, 2 H), 2.13-2.01 (m, 2 H), 1.89-1.72 (m, 2 H). |

### Example 194: Preparation of 4-((4-((3,4-dichloro-2-fluorophenyl)amino)-7-methoxyuinazolin-6-yl)oxy)piperidine-1-carbonitrile (Compound 194)

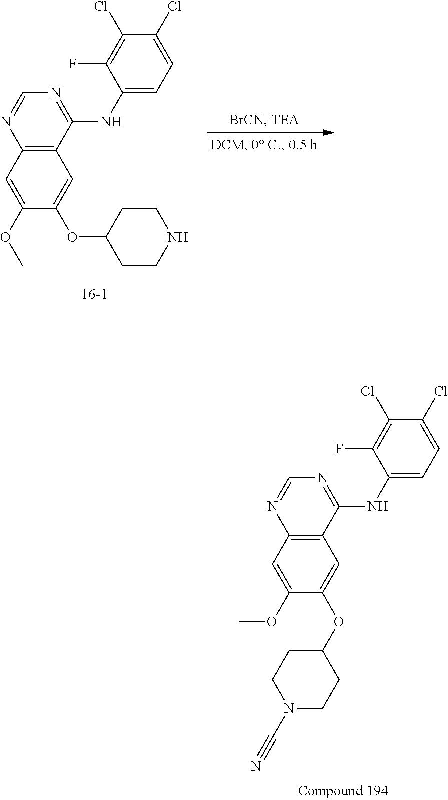 [SMILES: COc1cc2ncnc(Nc3ccc(Cl)c(Cl)c3F)c2cc1OC1CCNCC1 ; COc1cc2ncnc(Nc3ccc(Cl)c(Cl)c3F)c2cc1OC1CCN(C#N)CC1]

A solution of N-(3,4-dichloro-2-fluorophenyl)-7-methoxy-6-(piperidin-4-yloxy)quinazolin-4-amine (100 mg, 0.23 mmol) in DCM was added TEA (63 mg, 0.5 mmol), the mixture was stirred at 0° C. for 0.5 h, then BrCN (24 mg, 0.23 mmol) was added in and the mixture was stirred from 0° C.˜r.t. for 0.5 h, the mixture was then quenched by water, extracted with DCM, the combined organic layer was concentrated in vacuo and the residue was purified by column chromatography to give the 4-((4-((3,4-dichloro-2-fluorophenyl)amino)-7-methoxyquinazolin-6-yl)oxy)piperidine-1-carbonitrile (Compound 194) as a yellow solid (14.3 mg, 14% yield) as a yellow oil. LC/MS (ESI, m/z): [M+H]⁺=462.2. ¹H NMR (400 MHz, DMSO-d₆) δ 9.65 (s, 1H), 8.40 (s, 1H), 7.86 (s, 1H), 7.64-7.55 (m, 2H), 7.25 (s, 1H), 4.72-4.63 (m, 1H), 3.96 (s, 3H), 3.51-3.42 (m, 2H), 3.28-3.18 (m, 2H), 2.15-2.04 (m, 2H), 1.86-1.75 (m, 2H).

Examples 195-198 were prepared using similar procedures as described in the preceding Examples.

| EX. | | Compound | | |
|---|---|---|---|---|
| NO. | IUPAC Name | structure | salt | Compound characterization |
| 195 | 3-(4-((3,4- dichloro-2- fluorophenyl) amino)-7- methoxy- quinazolin-6- yl)azetidine-1- carbonitrile | 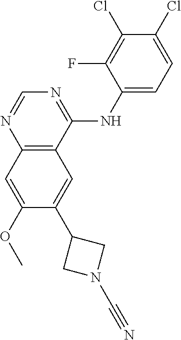 [SMILES: COc1cc2ncnc(Nc3ccc(Cl)c(Cl)c3F)c2cc1C1CN(C#N)C1] | None | LC/MS (ESI, m/z): [M + H]⁺ = 418.0, 420.0. ¹H NMR (400 MHz, DMSO-d₆) δ 9.93 (s, 1 H), 8.47 (s, 1 H), 8.35 (s, 1 H), 7.61 (d, J = 4.2 Hz, 2 H), 7.23 (s, 1 H), 4.55 (t, J = 8.1 Hz, 2 H), 4.39 (t, J = 7.3 Hz, 2 H), 4.28-4.20 (m, 1 H), 3.95 (s, 3 H). |
| 196 | N-(3-((4-((3,4- dichloro-2- fluorophenyl) amino)-7- methoxy- quinazolin-6- yl)oxy) cyclobutyl)-N- methylcyanamide | 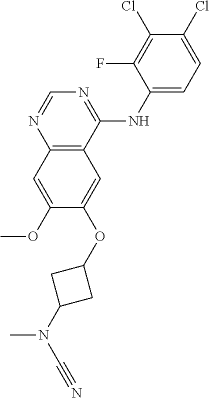 [SMILES: COc1cc2ncnc(Nc3ccc(Cl)c(Cl)c3F)c2cc1OC1CC(N(C)C#N)C1] | None | LC/MS (ESI, m/z): [M + H]⁺ = 462.1, 464.1. ¹H NMR (400 MHz, DMSO-d₆) δ 9.68 (s, 1 H), 8.39 (s, 1 H), 7.67-7.52 (m, 2 H), 7.50 (s, 1 H), 7.24 (s, 1 H), 5.13-4.80 (m, 1 H), 3.97 (s, 3 H), 3.95- 3.77 (m, 1 H), 2.82 (s, 3 H), 2.73-2.65 (m, 2 H), 2.50-2.32 (m, 2 H). |
| 197 | 5-(4-((3,4- dichloro-2- fluorophenyl) amino)-7- methoxy- quinazolin- 6-yl)-3,6- dihydropyridine- 1(2H)- carbonitrile | 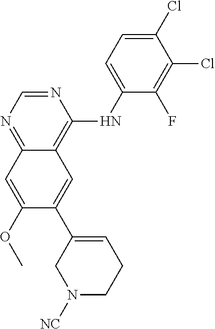 [SMILES: COc1cc2ncnc(Nc3ccc(Cl)c(Cl)c3F)c2cc1C1=CCCN(C#N)C1] | None | LC/MS (ESI, m/z): [M + H]⁺ = 444.0, 446.0. ¹H NMR (400 MHz, DMSO-d₆) δ 10.53 (br s, 1 H), 8.63 (s, 1 H), 8.34 (s, 1 H), 7.64-7.58 (m, 2 H), 7.24 (s, 1 H), 6.14 (s, 1 H), 4.10 (d, J = 1.7 Hz, 2 H), 3.98 (s, 3 H), 2.69-2.64 (m, 1 H), 2.53-2.50 (m, 2 H), 2.33-2.31 (m, 2 H). |
| 198 | 3-((4-((3,4- dichloro-2- fluorophenyl) amino)-7- methoxy- quinazolin-6- yl)oxy) piperidine-1- carbonitrile | 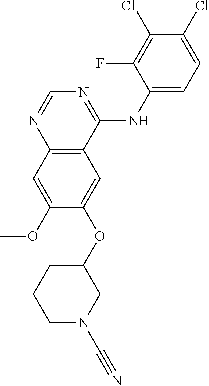 [SMILES: COc1cc2ncnc(Nc3ccc(Cl)c(Cl)c3F)c2cc1OC1CCCN(C#N)C1] | None | LC/MS (ESI, m/z): [M + H]⁺ = 462.1, 464.1. ¹H NMR (400 MHz, DMSO-d₆) δ 9.67 (s, 1 H), 8.40 (s, 1 H), 7.89 (s, 1 H), 7.61-7.57 (m, 2 H), 7.25 (s, 1 H), 4.66-4.63 (m, 1 H), 3.95 (s, 3 H), 3.57 (dd, J = 12.7, 3.0 Hz, 1 H), 3.42-3.32 (m, 3 H), 2.00-1.52 (m, 4 H). |

### Example 199: Preparation of 9-((1-acryloylpiperidin-4-yl)oxy)-3-(3,4-dichloro-2-fluorophenyl)-8-methoxy-1H-pyrimido[4,5,6-de]quinazolin-2(3H)-one (Compound 199)

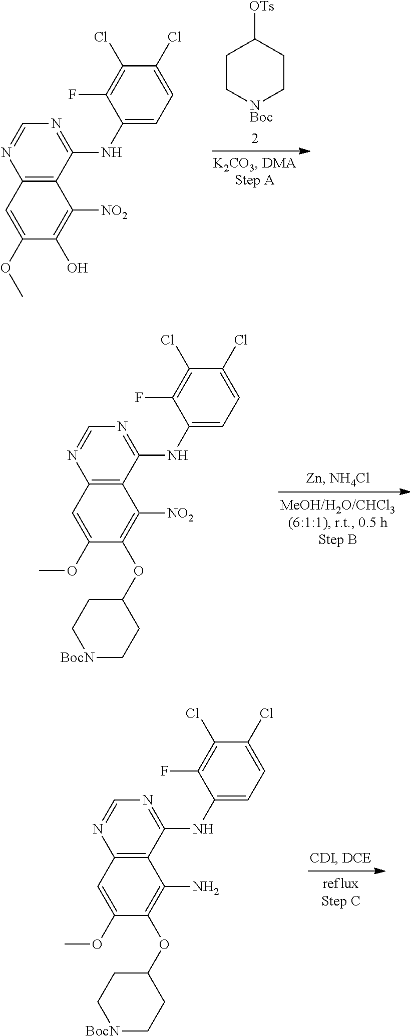 [SMILES: COc1cc2ncnc(Nc3ccc(Cl)c(Cl)c3F)c2c([N+](=O)[O-])c1O ; Cc1ccc(S(=O)(=O)OC2CCN(C(=O)OC(C)(C)C)CC2)cc1 ; COc1cc2ncnc(Nc3ccc(Cl)c(Cl)c3F)c2c([N+](=O)[O-])c1OC1CCN(C(=O)OC(C)(C)C)CC1 ; COc1cc2ncnc(Nc3ccc(Cl)c(Cl)c3F)c2c(N)c1OC1CCN(C(=O)OC(C)(C)C)CC1]

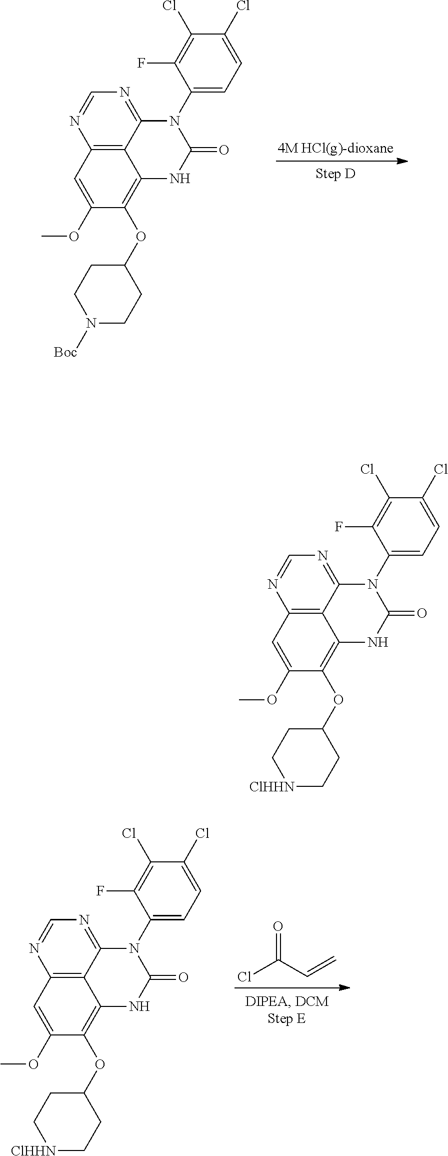

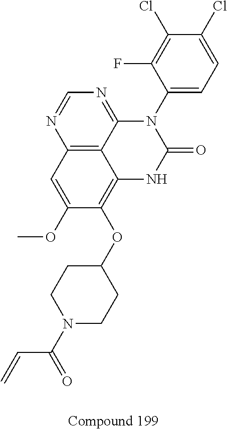 [SMILES: C=CC(=O)N1CCC(Oc2c(OC)cc3ncnc4c3c2NC(=O)N4c2ccc(Cl)c(Cl)c2F)CC1]

Step A: A mixture of 4-((3,4-dichloro-2-fluorophenyl)amino)-7-methoxy-5-nitroquinazolin-6-ol (0.5 g, 1.25 mmol), tert-butyl 4-(tosyloxy)piperidine-1-carboxylate (0.67 g, 1.88 mmol), K₂CO₃ (519 mg, 5.25 mmol) in DMA (20 mL) was degassed with nitrogen, heated to 130° C. and stirred for 2 hours under nitrogen atmosphere. Then the mixture was quenched by water (50 mL), extracted with EtOAc. The combined organic layers were washed with brine, dried over anhydrous sodium sulfate, filtered and concentrated in vacuum. The residue was purified by column chromatography (PE/EA) to give tert-butyl 4-((4-((3,4-dichloro-2-fluorophenyl)amino)-7-methoxy-5-nitroquinazolin-6-yl)oxy)piperidine-1-carboxylate (1.17 g, 79% yield) as a brown oil. LC/MS (ESI, m/z): [M+H]⁺=582.1.

Step B: A mixture of tert-butyl 4-((4-((3,4-dichloro-2-fluorophenyl)amino)-7-methoxy-nitroquinazolin-6-yl)oxy)piperidine-1-carboxylate (60 mg, 105.6 umol), Zn (69 mg, 1.06 mmol), NH₄Cl (56 mg, 1.06 mmol) in MeOH/H₂O/CHCl₃(12 mL/2 mL/2 mL) was stirred at r.t. for 1 hour. Then the mixture was quenched by water, extracted with DCM. The combined organic layers were washed with brine, dried over anhydrous sodium sulfate, filtered and concentrated in vacuum to give tert-butyl 4-((5-amino-4-((3,4-dichloro-2-fluorophenyl)amino)-7-methoxyquinazolin-6-yl)oxy)piperidine-1-carboxylate (57 mg, 100% yield) as a yellow solid. LC/MS (ESI, m/z): [M+H]⁺=552.1.

Step C: A mixture of tert-butyl 4-((5-amino-4-((3,4-dichloro-2-fluorophenyl)amino)-7-methoxyquinazolin-6-yl)oxy)piperidine-1-carboxylateas a yellow solid (57 mg, 105.6 umol), CDI (178.4 mg, 1.1 mmol) in DCE (5 mL) was heated to 90° C. and stirred for 2 hours under nitrogen atmosphere. The reaction was cooled to r.t and quenched by water, extracted with DCM. The combined organic layers were washed with brine, dried over anhydrous sodium sulfate, filtered and concentrated in vacuo to give tert-butyl 4-((3-(3,4-dichloro-2-fluorophenyl)-8-methoxy-2-oxo-2,3-dihydro-1H-pyrimido[4,5,6-de]quinazolin-9-yl)oxy)piperidine-1-carboxylate (61 mg, 98% yield) as a yellow solid. LC/MS (ESI, m/z): [M+H]⁺=578.1.

Step D: tert-butyl 4-((3-(3,4-dichloro-2-fluorophenyl)-8-methoxy-2-oxo-2,3-dihydro-1H-pyrimido[4,5,6-de]quinazolin-9-yl)oxy)piperidine-1-carboxylate (128 mg, 0.221 mmol) in 4 M HCl in dioxane (5 mL) was stirred for 1 hours at r.t. The reaction was then concentrated in vacuum to give 3-(3,4-dichloro-2-fluorophenyl)-8-methoxy-9-(piperidin-4-yloxy)-1H-Pyrimido[4,5,6-de]quinazolin-2(3H)-one hydrochloride (100 mg, 88% yield) as a yellow solid. LC/MS (ESI, m/z): [M+H]⁺=

Step E: A mixture of 3-(3,4-dichloro-2-fluorophenyl)-8-methoxy-9-(piperidin-4-yloxy)-1H-pyrimido[4,5,6-de]quinazolin-2(3H)-one hydrochloride (100 mg, 0.194 mmol), acryloyl chloride (17 mg, 0.194 mmol), DIPEA (100 mg, 0.78 mmol) in DCM (10 mL) was stirred for 0.5 h at r.t. The reaction was cooled to r.t and quenched by water, extracted with DCM. The combined organic layers were washed with brine, dried over anhydrous sodium sulfate, filtered and concentrated in vacuum. The residue was purified via reverse phase column chromatography (CH₃CN/H₂O) to give 9-((1-acryloylpiperidin-4-yl)oxy)-3-(3,4-dichloro-2-fluorophenyl)-8-methoxy-1H-pyrimido[4,5,6-de]quinazolin-2(3H)-one (Compound 199) (37 mg, 36% yield) as a white solid. LC/MS (ESI, m/z): [M+H]⁺=532.1, 534.0. ¹H NMR (400 MHz, DMSO) δ 11.22 (s, 1H), 8.49 (s, 1H), 7.76-7.70 (m, 2H), 7.03 (s, 1H), 6.86 (dd, J=16.6, 10.4 Hz, 1H), 6.12 (dd, J=16.7, 2.4 Hz, 1H), 5.69 (dd, J=10.4, 2.4 Hz, 1H), 4.56-4.47 (m, 1H), 4.36 (d, J=12.6 Hz, 1H), 4.08 (d, J=12.1 Hz, 1H), 4.00 (s, 3H), 3.16-3.07 (m, 1H), 2.78 (t, J=10.9 Hz, 1H), 1.93-1.85 (m, 2H), 1.84-1.76 (m, 2H).

### Example 200: Preparation of 9-((1-acryloylpiperidin-4-yl)oxy)-3-(3,4-dichloro-2-fluorophenyl)-8-methoxy-1-methyl-1H-pyrimido[4,5,6-del]quinazolin-2(3H)-one (Compound 200)

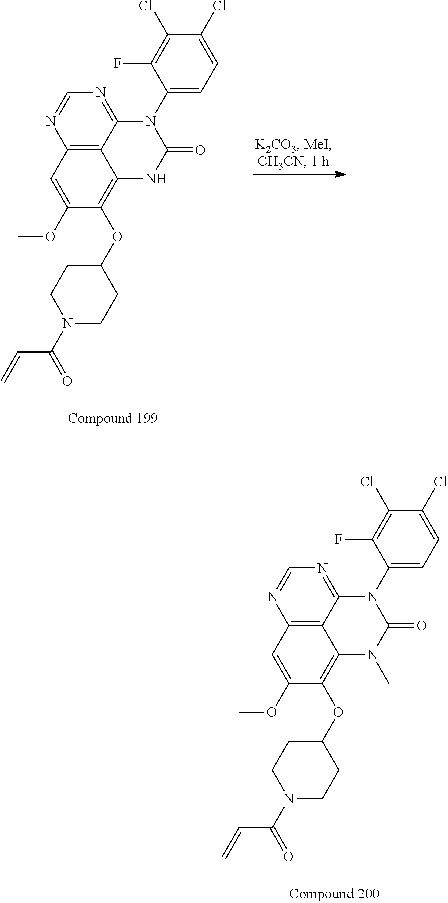 [SMILES: C=CC(=O)N1CCC(Oc2c(OC)cc3ncnc4c3c2NC(=O)N4c2ccc(Cl)c(Cl)c2F)CC1 ; C=CC(=O)N1CCC(Oc2c(OC)cc3ncnc4c3c2N(C)C(=O)N4c2ccc(Cl)c(Cl)c2F)CC1]

A mixture of 9-((1-acryloylpiperidin-4-yl)oxy)-3-(3,4-dichloro-2-fluorophenyl)-8-methoxy-1H-pyrimido[4,5,6-de]quinazolin-2(3H)-one (7 mg, 13 umol), CH₃I (3.7 mg, 26 umol) and K₂CO₃ (5.4 mg, 39 umol) in CH₃CN (1 mL) was stirred for 0.5 h at room temperature. The reaction was cooled to room temperature and quenched by water, extracted with EtOAc. The combined organic layers were washed with brine, dried over anhydrous sodium sulfate, filtered and concentrated in vacuum. The residue was purified via reverse phase column chromatography (CH₃CN/H₂O) to give 9-((1-acryloylpiperidin-4-yl)oxy)-3-(3,4-dichloro-2-fluorophenyl)-8-methoxy-1-methyl-1H-pyrimido[4,5,6-de]quinazolin-2(3H)-one (Compound 200) (5 mg, 70% yield) as a white solid. LC/MS (ESI, m/z): [M+H]⁺=546.2, 548.2. ¹H NMR (400 MHz, CDCl₃) δ 8.62 (s, 1H), 7.52-7.47 (m, 2H), 7.23-7.21 (m, 1H), 6.59 (dd, J=16.8, 10.7 Hz, 1H), 6.32 (dd, J=16.9, 1.7 Hz, 1H), 5.75 (dd, J=10.6, 1.5 Hz, 1H), 4.63-4.50 (m, 1 H), 4.35-4.25 (m, 1H), 4.11 (s, 3H), 4.07-4.00 (m, 1H), 3.85 (s, 3H), 2.90-2.82 (m, 2H), 2.10-2.01 (m, 2H), 1.81-1.73 (m, 2H).

### Example 201: Preparation of rel-(R)-9-((1-acryloylpiperidin-3-yl)oxy)-3-(3,4-dichloro-2-fluorophenyl)-8-methoxy-1H-pyrimido[4,5,6-de]quinazolin-2(3H)-one (Compound 201A) and rel-(R)-9-((1-acryloylpiperidin-3-yl)oxy)-3-(3,4-dichloro-2-fluorophenyl)-8-methoxy-1H-pyrimido[4,5,6-de]quinazolin-2(3H)-one (Compound 201B)

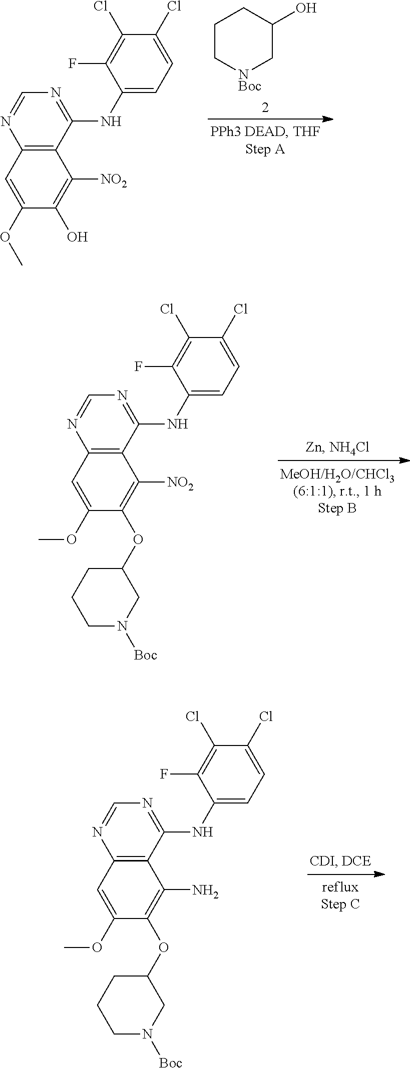

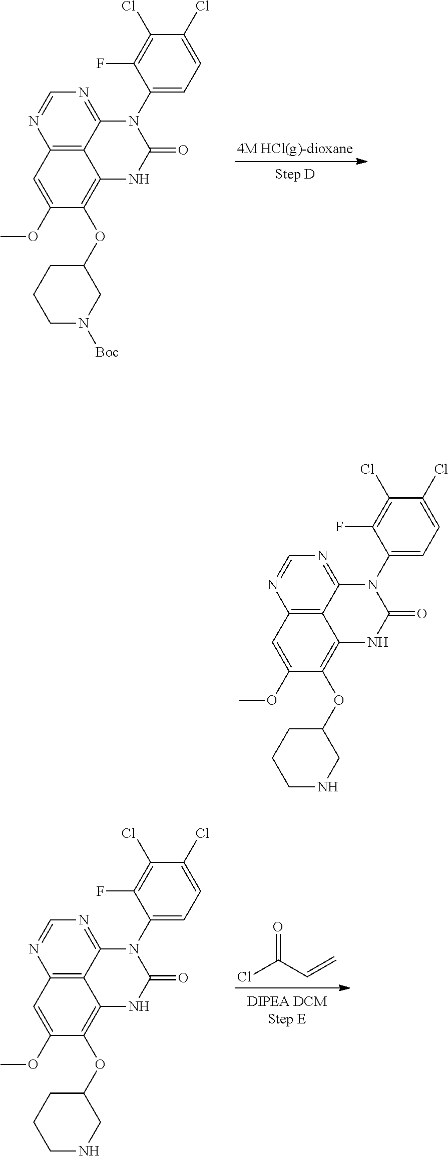

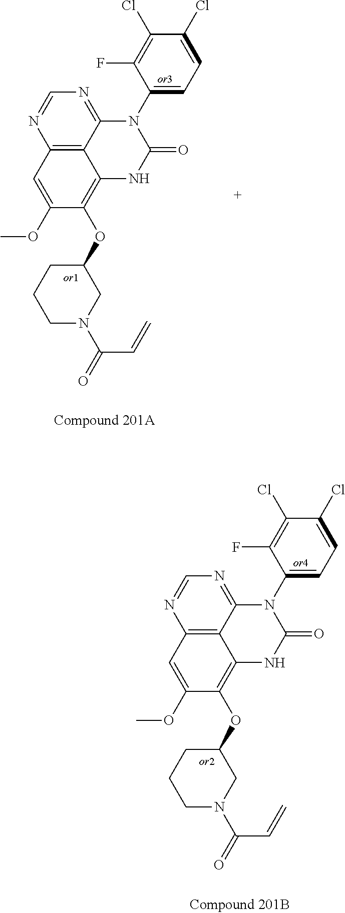 [SMILES: C=CC(=O)N1CCC[C@@H](Oc2c(OC)cc3ncnc4c3c2NC(=O)N4c2ccc(Cl)c(Cl)c2F)C1 ; C=CC(=O)N1CCC[C@@H](Oc2c(OC)cc3ncnc4c3c2NC(=O)N4c2ccc(Cl)c(Cl)c2F)C1]

Compound 201A and Compound 201B were prepared using similar procedures as described in Example 199. In Step C, two diastereomer pairs were separated by column chromatography on silica gel and the syntheses completed to give Compound 201A and Compound 201B (stereochemistry arbitrarily assigned). Compound 201A: LC/MS (ESI, m/z): [M+H]⁺=532.1. ¹H NMR (400 MHz, DMSO-d₆) δ 11.11 (d, J=46.8 Hz, 1H), 8.45 (s, 1H), 7.76-7.67 (m, 2H), 7.01 (s, 1H), 6.87-6.73 (m, 1H), 6.15-6.06 (m, 1H), 5.71-5.62 (m, 1H), 4.24-3.98 (m, 5H), 3.73-3.54 (m, 2H), 3.36-3.02 (m, 1H), 2.07-1.46 (m, 4H). Compound 201B: LC/MS (ESI, m/z): [M+H]⁺=532.1. ¹H NMR (400 MHz, DMSO-d₆) δ 11.19 (d, J=30.8 Hz, 1H), 8.48 (s, 1H), 7.76-7.68 (m, 2H), 7.03 (s, 1H), 6.86-6.77 (m, 1H), 6.14-6.10 (m, 1H), 5.71-5.86 (m, 1H), 4.15-3.99 (m, 5H), 3.78-2.97 (m, 3H), 2.08-1.31 (m, 4H).

### Example 202: Preparation of rel-(R)-9-((1-acryloylpiperidin-3-yl)oxy)-3-(3,4-dichloro-2-fluorophenyl)-8-methoxy-1-methyl-1H-pyrimido[4,5,6-de]quinazolin-2(3H)-one (Compound 202A) and rel-(R)-9-((1-acryloylpiperidin-3-yl)oxy)-3-(3,4-dichloro-2-fluorophenyl)-8-methoxy-1-methyl-1H-pyrimido[4,5,6-de]quinazolin-2(3H)-one (Compound 202B)

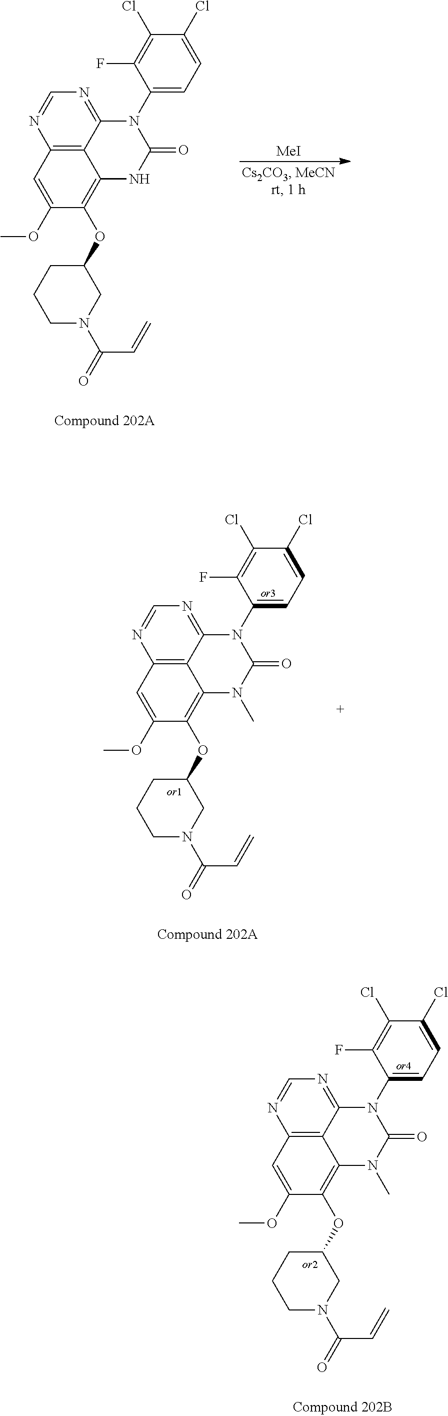 [SMILES: "MeI" ; C=CC(=O)N1CCC[C@@H](Oc2c(OC)cc3ncnc4c3c2NC(=O)N4c2ccc(Cl)c(Cl)c2F)C1 ; C=CC(=O)N1CCC[C@@H](Oc2c(OC)cc3ncnc4c3c2N(C)C(=O)N4c2ccc(Cl)c(Cl)c2F)C1 ; C=CC(=O)N1CCC[C@H](Oc2c(OC)cc3ncnc4c3c2N(C)C(=O)N4c2ccc(Cl)c(Cl)c2F)C1]

Compound 202A and Compound 202B were prepared using similar procedures as described in Example 199. Compound 202A and Compound 202B were separated by prep-HPLC (CH₃CN and H₂O with 0.01% TFA as mobile phase) to two pair of diastereomers (stereochemistry arbitrarily assigned). Compound 202A: LC/MS (ESI, m/z): [M+H]⁺=546.1. ¹H NMR (400 MHz, CDCl₃) δ 8.61 (s, 1H), 7.52-7.31 (m, 2H), 7.26-7.22 (m, 1H), 6.60-6.53 (m, 1H), 6.34-6.26 (m, 1H), 5.74-5.70 (m, 1H), 4.45-4.41 (m, 2H), 4.09 (s, 3H), 3.83 (d, J=11.6 Hz, 3H), 3.65-3.15 (m, 3H), 2.12-1.43 (m, 4H). Compound 202B: LC/MS (ESI, m/z): [M+H]⁺=546.0. ¹H NMR (400 MHz, CDCl₃) δ 8.64 (s, 1H), 7.60-7.46 (m, 2H), 7.35-7.21 (m, 1 H), 6.59-6.52 (m, 1H), 6.34-6.21 (m, 1H), 5.74-5.70 (m, 1H), 4.33-3.95 (m, 5H), 3.83 (d, J=12.8 Hz, 3H), 3.63-3.20 (m, 3H), 2.12-1.53 (m, 4H).

### Example 203: Preparation of 9-(1-acryloyl-1,2,3,6-tetrahydropyridin-4-yl)-3-(3,4-dichloro-2-fluorophenyl)-8-methoxy-1H-pyrimido[4,5,6-de]quinazolin-2(3H)-one (Compound 203)

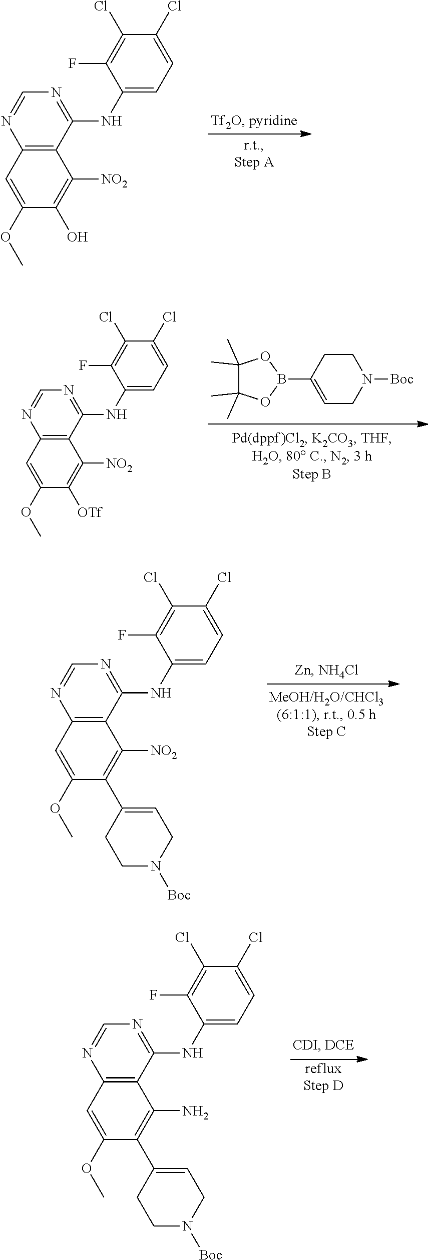

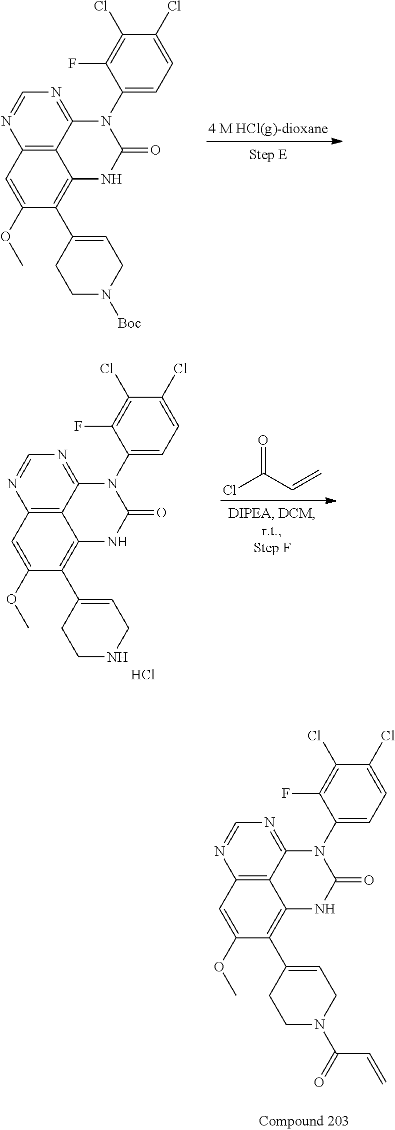

Step A: To a stirred solution of 4-((3,4-dichloro-2-fluorophenyl)amino)-7-methoxy-5-nitroquinazolin-6-ol (1.3 g, 3.26 mmol) in anhydrous CH₂Cl₂ (10 mL) was added anhydrous pyridine (0.91 mL, 11.23 mmol) at 0° C. and stirred for 30 min then Tf₂O (1.11 mL, 6.52 mmol) was added at same temperature under nitrogen atmosphere. The reaction mixture was stirred for 4 h at room temperature, then cooled to 0° C. and quenched with 1N HCl (till pH of the reaction mixture becomes neutral) to remove excess pyridine. The organic layer was separated and the aqueous layer was extracted with DCM (2×10 mL). the combined organic layers were washed with water (5 mL), brine (5 mL), dried with Na₂SO₄, and concentrated under reduced pressure, and the residue was purified by silica gel column chromatography (Hexanes/EtOAc) to afford 4-((3,4-dichloro-2-fluorophenyl)amino)-7-methoxy-5-nitroquinazolin-6-yl trifluoromethanesulfonate as white solid (1.21 g, 69.94% yield). LC/MS (ESI, m/z): [M+H]⁺=530.0.

Step B: To a solution of tert-butyl 4-((3,4-dichloro-2-fluorophenyl)amino)-7-methoxy-5-nitroquinazolin-6-yl trifluoromethanesulfonate (1.21 g, 2.3 mmol), K₂CO₃ (944 mg, 6.84 mmol) and PdCl₂(dppf) (170 mg, 230 umol) in 1.4-dioxane:H₂O (20 mL:2 mL). The reaction was heated to 90° C. and stirred for 18 hours under nitrogen atmosphere. The reaction was cooled to r.t and quenched by water, extracted with EtOAc (20 mL×3). The combined organic layers were washed with brine, dried over anhydrous sodium sulfate, filtered and concentrated in vacuum. The residue was purified by column chromatography (PE/EA) to give tert-butyl 4-(4-((3,4-dichloro-2-fluorophenyl)amino)-7-methoxy-5-nitroquinazolin-6-yl)-3,6-dihydropyridine-1(2H)-carboxylate (900 mg, 70% yield) as a brown solid. LC/MS (ESI, m/z): [M+H]⁺=564.1.

Steps C, D, E, and F were completed in a similar manner as described in Example 199 steps B, C, D, and E to afford 9-(1-acryloyl-1,2,3,6-tetrahydropyridin-4-yl)-3-(3,4-dichloro-2-fluorophenyl)-8-methoxy-1H-pyrimido[4,5,6-de]quinazolin-2(3H)-one (Compound 203). LC/MS (ESI, m/z): [M+H]⁺=514.0, 516.0. ¹H NMR (400 MHz, MeOD-d₄) δ 8.55 (s, 1H), 7.59 (dd, J=8.8, 1.7 Hz, 1H), 7.54-7.42 (m, 1H), 6.96 (s, 1H), 6.88-6.73 (m, 1H), 6.27 (dd, J=16.6, 5.3 Hz, 1H), 5.87-5.78 (m, 2H), 4.41-4.35 (m, 1H), 4.20-4.10 (m, 2H), 4.00 (s, 3 H), 3.87-3.78 (m, 1H), 2.62-2.54 (m, 1H), 2.33-2.25 (m, 1H).

### Example 204: Preparation of 1-(4-((4-((3-ethynylphenyl)amino)-7-methoxy-5-nitroquinazolin-6-yl)oxy)piperidin-1-yl)prop-2-en-1-one (Compound 204)

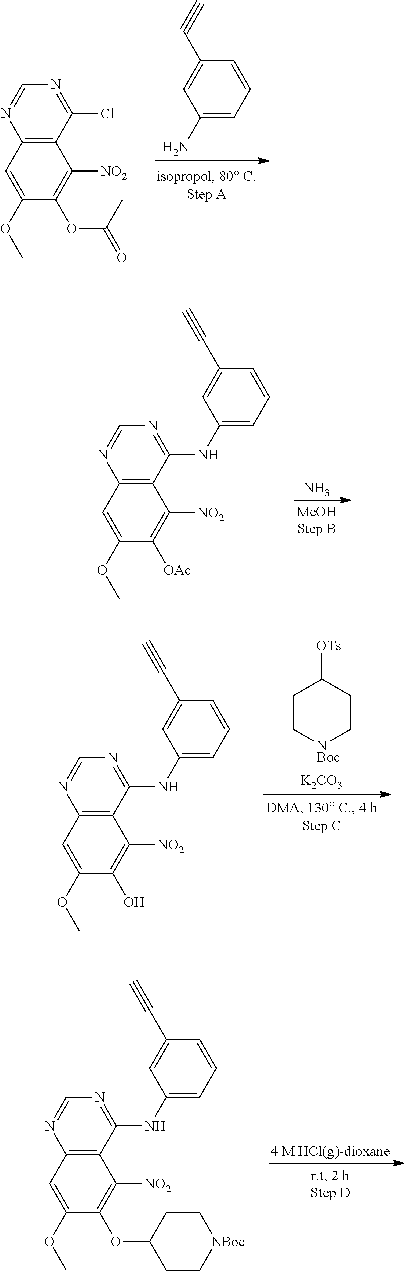

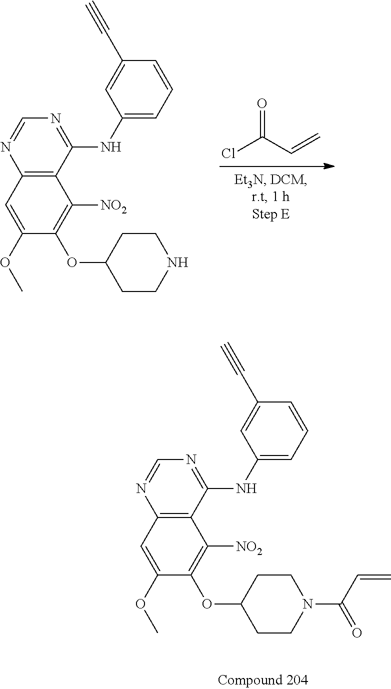

Step A: A mixture of 4-chloro-7-methoxy-5-nitroquinazolin-6-yl acetate (2.5 g, 8.39 mmol) and 3-ethynylaniline (980 mg, 8.39 mmol) in i-PrOH (10 mL) was stirred at 80° C. for 1 h. The reaction mixture was cooled to room temperature and a precipitate was filtered. The filter cake was washed with i-PrOH and dried to give the crude compound which was triturated with EA to afford 4-((3-ethynylphenyl)amino)-7-methoxy-5-nitroquinazolin-6-yl acetate (3 g, 95% yield) as a yellow solid. LC/MS (ESI, m/z): [M+H]⁺=379.1.

Step B: To a solution of 4-((3-ethynylphenyl)amino)-7-methoxy-5-nitroquinazolin-6-yl acetate (3 g, 7.94 mmol) in MeOH (20 mL) was added ammonium hydroxide (10 mL). The mixture was stirred at r.t. for 2 h and then concentrated in vacuum. The crude compound was triturated with EA to afford 4-((3-ethynylphenyl)amino)-7-methoxy-5-nitroquinazolin-6-ol (2.2 g, 83% yield) as a yellow solid. LC/MS (ESI, m/z): [M+1]⁺=337.1.

Step C: A mixture of methyl 4-((3-ethynylphenyl)amino)-7-methoxy-5-nitroquinazolin-6-ol (2 g, 5.95 mmol), tert-butyl 4-(tosyloxy)piperidine-1-carboxylate (4.26 g, 12 mmol) and K₂CO₃ (1.66 g, 12 mmol) in DMA (10 mL) was warmed to 130° C. and stirred for 4 h. The reaction mixture was cooled to room temperature, then poured into water and extracted with EA (3×100 mL). The combined organic layers were washed with water (100 mL), brine (100 mL), dried over Na₂SO₄, filtered and concentrated in vacuum. The residue was purified by flash chromatography eluting with EA/PE=32% to give tert-butyl 4-((4-((3-ethynylphenyl)amino)-7-methoxy-5-nitroquinazolin-6-yl)oxy)piperidine-1-carboxylate (710 mg, 23% yield) as a yellow solid. LC/MS (ESI, m/z): [M+H]⁺=520.0.

Step D: To a solution of tert-butyl 4-((4-((3-ethynylphenyl)amino)-7-methoxy-5-nitroquinazolin-6-yl)oxy)piperidine-1-carboxylate (200 mg, 0.385 mmol) in DCM (2 mL) was added 4M HCl solution in dioxane. The mixture was stirred at room temperature for 2 h. The reaction mixture was concentrated in vacuum. The residue was purified by prep HPLC to give N-(3-ethynylphenyl)-7-methoxy-5-nitro-6-(piperidin-4-yloxy)quinazolin-4-amine hydrochloride (180 mg). LC/MS (ESI, m/z): [M+H]⁺=420.1. ¹H NMR (400 MHz, DMSO-d₆) δ 8.63 (br. s., 1 H), 8.40 (br. s., 1H), 7.84 (br. s., 1H), 7.36-7.29 (m, 1H), 7.26 (br. s., 1H), 7.14 (d, J=7.5 Hz, 1H), 6.92 (br. s., 2H), 4.75-4.53 (m, 1H), 4.15 (s, 1H), 4.00 (s, 3H), 3.27-3.11 (m, 2 H), 3.11-2.94 (m, 2H), 2.11-1.94 (m, 2H), 1.87-1.69 (m, 2H).

Step E: To a mixture of N-(3-ethynylphenyl)-7-methoxy-5-nitro-6-(piperidin-4-yloxy)quinazolin-4-amine hydrochloride (80 mg, 0.191 mmol) and Et₃N (38.6 mg, 0.382 mmol) in DCM (10 mL) was added acryloyl chloride (19 mg, 0.21 mmol), The mixture was stirred at RT for 1 hour. The reaction mixture was quenched by MeOH and concentrated in vacuum. The residue was purified by prep-HPLC to afford 1-(4-((4-((3-ethynylphenyl)amino)-7-methoxy-5-nitroquinazolin-6-yl)oxy)piperidin-1-yl)prop-2-en-1-one (Compound 204) (26.9 mg, 30% yield) as a yellow solid. LC/MS (ESI, m/z): [M+H]⁺=474.0. ¹H NMR (400 MHz, DMSO-d₆) δ 8.00-7.70 (m, 1H), 7.34-7.29 (m, 1H), 7.24 (br. s., 1H), 7.14 (d, J=7.6 Hz, 1H), 7.06-6.87 (m, 2H), 6.82 (dd, J=10.4, 16.7 Hz, 1H), 6.09 (dd, J=2.4, 16.8 Hz, 1H), 5.67 (dd, J=2.4, 10.4 Hz, 1H), 4.70-4.61 (m, 1H), 4.14 (s, 1H), 4.01 (s, 3H), 3.96-3.88 (m, 1H), 3.86-3.77 (m, 1H), 3.38-3.28 (m, 1H), 3.17 (t, J=9.8 Hz, 1H), 1.95-1.82 (m, 2H), 1.59-1.42 (m, 2 H).

Examples 205-216 were prepared using similar procedures as described in the preceding Examples.

| EX. | | Compound | | |
|---|---|---|---|---|
| NO. | IUPAC Name | structure | salt | Compound characterization |
| 205 | 9-((1- acryloylpyrrolidin- 3-yl)oxy)- 3-(3,4-dichloro- 2- fluorophenyl)- 8-methoxy-1H- pyrimido[4,5,6- de]quinazolin- 2(3H)-one | 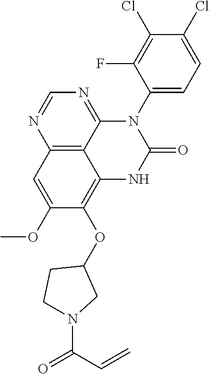 [SMILES: C=CC(=O)N1CCC(Oc2c(OC)cc3ncnc4c3c2NC(=O)N4c2ccc(Cl)c(Cl)c2F)C1] | None | LC/MS (ESI, m/z): [M + H]⁺ = 518.0, 520.0. ¹H NMR (400 MHz, DMSO-d₆) δ 11.38 (t, J = 9.7 Hz, 1 H), 8.46 (s, 1 H), 7.79-7.61 (m, 2 H), 7.02 (s, 1 H), 6.72-6.43 (m, 1 H), 6.16 (ddd, J = 16.7, 4.8, 2.4 Hz, 1 H), 5.68 (ddd, J = 12.9, 7.9, 2.6 Hz, 1 H), 5.00 (d, J = 15.5 Hz, 1 H), 3.95 (d, J = 3.3 Hz, 3 H), 3.90-3.83 (m, 2 H), 3.82-3.74 (m, 2 H), 2.10-2.01 ( m, 2 H). |
| 206 | 9-(1-acryloyl- 1,2,3,6- tetrahydropyridin- 4-yl)-3-(3,4- dichloro-2- fluorophenyl)- 8-methoxy-1- methyl-1H- pyrimido[4,5,6- de]quinazolin- 2(3H)-one | 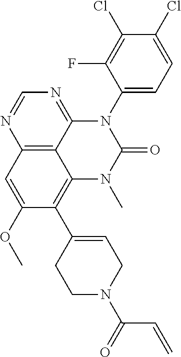 [SMILES: C=CC(=O)N1CC=C(c2c(OC)cc3ncnc4c3c2N(C)C(=O)N4c2ccc(Cl)c(Cl)c2F)CC1] | None | LC/MS (ESI, m/z): [M + H]⁺ = 528.2, 530.2. ¹H NMR (400 MHz, DMSO-d₆) δ 8.52 (s, 1 H), 7.75 (dd, J = 8.6, 3.6 Hz, 1 H), 7.67 (dd, J = 19.3, 10.9 Hz, 1 H), 7.04 (s, 1 H), 6.96-6.73 (m, 1 H), 6.17 (d, J = 16.8 Hz, 1 H), 5.73 (dd, J = 10.4, 2.4 Hz, 1 H), 5.69-5.53 (m, 1 H), 4.35- 4.15 (m, 2 H), 3.94 (s, 3 H), 3.82-3.72 (m, 2 H), 3.39 (s, 3 H), 2.80-2.65 (m, 1 H), 2.40-2.30 (m, 1 H). |
| 207 | N-(3,4- dichloro-2- fluorophenyl)- 7-methoxy-5- nitro-6- (piperidin-4- yloxy)quinazolin- 4-amine hydrochloride | 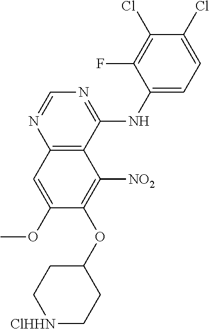 | HCl | LC/MS (ESI, m/z): [M + 1]+ = 482.0, 484.0. ¹H NMR (400 MHz, DMSO-d₆) δ 8.52 (s, 1 H), 8.33 (s, 1 H), 7.90 (s, 1 H), 7.39 (d, J = 8.2 Hz, 1 H), 7.24 (s, 1 H), 6.91 (t, J = 8.5 Hz, 1 H), 4.71- 4.56 (m, 1 H), 4.01 (s, 3 H), 3.19 (s, 2 H), 3.04 (s, 2 H), 2.01 (s, 2 H), 1.78 (dt, J = 18.1, 7.1 Hz, 2 H). |
| 208 | 1-(4-((4-((3,4- dichloro-2- fluorophenyl) amino)-7- methoxy-5- nitroquinazolin- 6- yl)oxy)piperidin- 1-yl)prop-2- en-1-one | 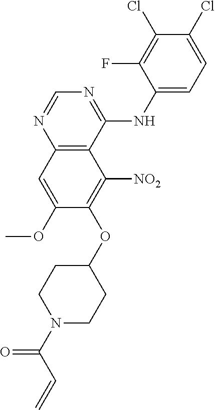 [SMILES: C=CC(=O)N1CCC(Oc2c(OC)cc3ncnc(Nc4ccc(Cl)c(Cl)c4F)c3c2[N+](=O)[O-])CC1] | None | LC/MS (ESI, m/z): [M + H]⁺ = 536.0, 538.0. ¹H NMR (400 MHz, CDCl₃) δ 8.71 (s, 1 H), 7.99 (s, 1 H), 7.70 (m, 1 H), 7.60 (s, 1 H), 7.33 (dd, J = 8.8, 1.8 Hz, 1 H) 6.59 (dd, J = 16.8, 10.6 Hz, 1 H), 6.30 (dd, J = 16.8, 1.6 Hz, 1 H), 5.73 (dd, J = 10.6, 1.6 Hz, 1 H), 4.86-4.79 (m, 1 H), 4.13 (s, 3 H), 33.99-3.87 (m, 1 H), 3.83-3.74 (m, 1 H), 3.62-3.42 (m, 2 H), 2.02-1.82 (m, 4 H). |
| 209 | N-(3,4- dichloro-2- fluorophenyl)- 7-methoxy-5- nitro-6- (piperidin-3- yloxy)quinazolin- 4-amine | 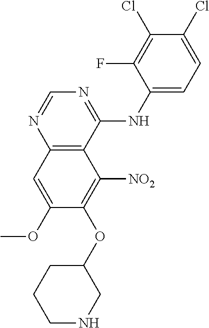 [SMILES: COc1cc2ncnc(Nc3ccc(Cl)c(Cl)c3F)c2c([N+](=O)[O-])c1OC1CCCNC1] | None | LC/MS (ESI, m/z): [M + H]⁺ = 484.0. ¹H NMR (400 MHz, DMSO-d₆) δ 8.74 (d, J = 3.6 Hz, 1 H), 8.63 (d, J = 2 Hz, 1 H), 7.92 (s, 1 H), 7.39 (d, J = 8.4 Hz, 1 H), 7.26 (d, J = 2 Hz, 1 H), 6.91 (t, J = 2 Hz, 1 H), 4.51 (s, 1 H), 4.02 (s, 3 H), 3.40-3.36 (m, 1 H), 3.15-2.98 (m, 3 H), 1.93- 1.86 (m, 2 H), 1.73-1.62 (m, 2 H). |
| 210 | 1-(3-((4-((3,4- dichloro-2- fluorophenyl) amino)-7- methoxy-5- nitroquinazolin- 6- yl)oxy)piperidin- 1-yl)prop-2- en-1-one | 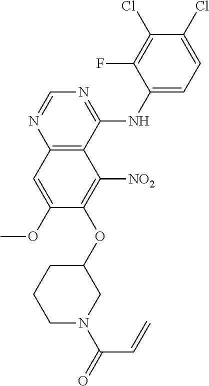 [SMILES: C=CC(=O)N1CCCC(Oc2c(OC)cc3ncnc(Nc4ccc(Cl)c(Cl)c4F)c3c2[N+](=O)[O-])C1] | None | LC/MS (ESI, m/z): [M + H]⁺ = 536.0. ¹H NMR (400 MHz, CDCl₃) δ 8.71 (s, 1 H), 7.99 (s, 1 H), 7.80 (s, 1 H), 7.33 (dd, J = 1.6 Hz, J = 8.8 Hz, 1 H), 6.58 (dd, J = 10.4 Hz, J = 16.8 Hz, 1 H), 6.31-6.22 (m, 1 H), 5.72 (dd, J = 1.6 Hz, J = 10.8 Hz, 1 H), 4.71-4.55 (m, 1 H), 4.30-4.02 (m, 1 H), 4.14 (s, 3 H), 3.66-3.18 (m, 3 H), 2.07-1.26 (m, 4 H). |
| 211 | 9-((1- acryloylpiperidin- 4-yl)oxy)-3- (3- ethynylphenyl)- 8-methoxy-1H- pyrimido[4,5,6- de]quinazolin- 2(3H)-one | 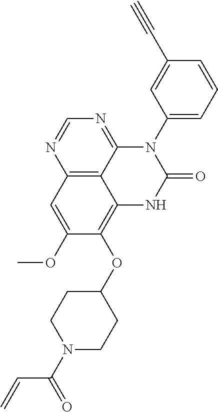 [SMILES: C#Cc1cccc(N2C(=O)Nc3c(OC4CCN(C(=O)C=C)CC4)c(OC)cc4ncnc2c34)c1] | None | LC/MS (ESI, m/z): [M + H]⁺ = 470.0. ¹H NMR (400 MHz, DMSO-d₆) δ 10.92 (s, 1 H), 8.38 (s, 1 H), 7.59-7.49 (m, 3 H), 7.49-7.43 (m, 1 H), 6.95 (s, 1 H), 6.85 (dd, J = 10.4, 16.6 Hz, 1 H), 6.12 (dd, J = 2.4, 16.7 Hz, 1 H), 5.69 (dd, J = 2.4, 10.4 Hz, 1 H), 4.51-4.41 (m, 1 H), 4.40- 4.31 (m, 1 H), 4.28 (s, 1 H), 4.08 (d, J = 13.9 Hz, 1 H), 3.98 (s, 3 H), 3.13 (t, J = 12.1 Hz, 1 H), 2.79 (t, J = 11.3 Hz, 1 H), 1.90-1.80 (m, 4 H). |
| 212 | 9-((1- acryloylpiperidin- 4-yl)oxy)-3- (3- ethynylphenyl)- 8-methoxy-1- methyl-1H- pyrimido[4,5,6- de]quinazolin- 2(3H)-one | 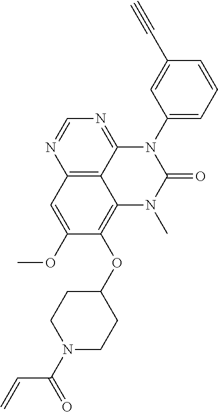 [SMILES: C#Cc1cccc(N2C(=O)N(C)c3c(OC4CCN(C(=O)C=C)CC4)c(OC)cc4ncnc2c34)c1] | None | LC/MS (ESI, m/z): [M + H]⁺ = 484.0. ¹H NMR (400 MHz, MeOD-d₄) δ 8.56 (s, 1 H), 7.64- 7.58 (m, 1 H), 7.54 (t, J = 7.8 Hz, 1 H), 7.50 (t, J = 1.5 Hz, 1 H), 7.42-7.33 (m, 1 H), 7.09 (s, 1 H), 6.79 (dd, J = 10.7, 16.8 Hz, 1 H), 6.20 (dd, J = 1.9, 16.8 Hz, 1 H), 5.75 (dd, J = 1.9, 10.6 Hz, 1 H), 4.48-4.34 (m, 2 H), 4.16-4.06 (m, 4 H), 3.83 (s, 3 H), 3.62 (s, 1 H), 3.29- 3.21 (m, 1 H), 2.98 (t, J = 11.3 Hz, 1 H), 2.14- 1.98 (m, 2 H), 1.84-1.66 (m, 2 H). |
| 213 | N-(3- ethynylphenyl)- 7-methoxy-5- nitro-6- (piperidin-4- yloxy)quinazolin- 4-amine | 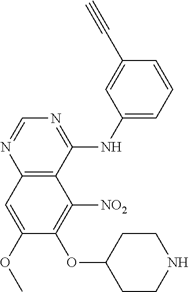 [SMILES: C#Cc1cccc(Nc2ncnc3cc(OC)c(OC4CCNCC4)c([N+](=O)[O-])c23)c1] | HCl | LC/MS (ESI, m/z): [M + H]⁺ = 420.1. ¹H NMR (400 MHz, DMSO-d₆) δ 8.63 (br. s., 1 H), 8.40 (br. s., 1 H), 7.84 (br. s., 1 H), 7.36-7.29 (m, 1 H), 7.26 (br. s., 1 H), 7.14 (d, J = 7.5 Hz, 1 H), 6.92 (br. s., 2 H), 4.75-4.53 (m, 1 H), 4.15 (s, 1 H), 4.00 (s, 3 H), 3.27-3.11 (m, 2 H), 3.11- 2.94 (m, 2 H), 2.11-1.94 (m, 2 H), 1.87-1.69 (m, 2 H). |
| 214 | 9-(1- acryloylpiperidin- 4-yl)-3-(2- fluorophenyl)- 8-methoxy-1H- pyrimido[4,5,6- de]quinazolin- 2(3H)-one | 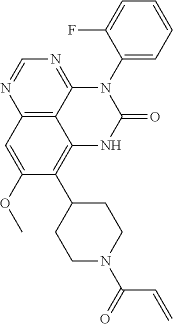 [SMILES: C=CC(=O)N1CCC(c2c(OC)cc3ncnc4c3c2NC(=O)N4c2ccccc2F)CC1] | None | LC/MS (ESI, m/z): [M + H]⁺ = 448.0. ¹H NMR (400 MHz, MeOD-d₄) δ 8.38 (s, 1 H), 7.51 (ddd, J = 30.1, 14.2, 6.6 Hz, 2 H), 7.35 (dd, J = 16.5, 8.6 Hz, 2 H), 6.92 (s, 1 H), 6.84 (dd, J = 16.8, 10.6 Hz, 1 H), 6.24 (dd, J = 16.8, 2.0 Hz, 1 H), 5.77 (dd, J = 10.6, 2.0 Hz, 1 H), 4.70 (d, J = 12.6 Hz, 1 H), 4.24 (d, J = 12.0 Hz, 1 H), 3.96 (s, 3 H), 3.51-3.36 (m, 2 H), 2.88 (t, J = 12.4 Hz, 1 H), 2.46-2.32 (m, 2 H), 2.09-1.96 (m, 2 H). |
| 215 | 9-((1- acryloylpiperidin- 4-yl)oxy)-3- (3,4- dichlorophenyl)- 8-methoxy- 1H- pyrimido[4,5,6- de]quinazolin- 2(3H)-one | 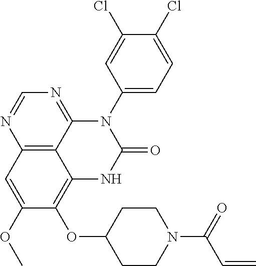 [SMILES: C=CC(=O)N1CCC(Oc2c(OC)cc3ncnc4c3c2NC(=O)N4c2ccc(Cl)c(Cl)c2)CC1] | None | LC/MS (ESI, m/z): [M + H]⁺ = 514.0. ¹H NMR (400 MHz, DMSO-d₆) δ 11.26 (br s, 1 H), 8.55 (s, 1 H), 7.84 (d, J = 2.2 Hz, 1 H), 7.82 (s, 1 H), 7.49 (dd, J = 8.4, 2.4 Hz, 1 H), 7.02 (d, J = 3.4 Hz, 1 H), 6.86 (dd, J = 16.8, 10.4 Hz, 1 H), 6.12 (dd, J = 16.8, 2.4 Hz, 1 H), 5.69 (dd, J = 10.4, 2.4 Hz, 1 H), 4.53-4.448 (m, 1 H), 4.35 (d, J = 13.4 Hz, 1 H), 4.08 (d, J = 13.6 Hz, 1 H), 4.01 (s, 3 H), 3.18-3.02 (m, 1 H), 2.89-2.73 (m, 1 H), 1.89-1.79 (m, 4 H). |
| 216 | 9-((1- acryloylazetidin- 3-yl)oxy)-3- (3,4-dichloro-2- fluorophenyl)- 8-methoxy-1H- pyrimido[4,5,6- de]quinazolin- 2(3H)-one | 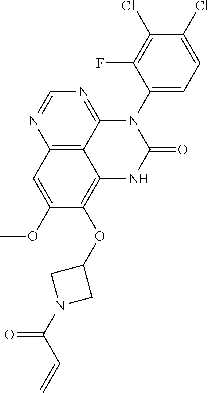 | None | LC/MS (ESI, m/z): [M + H]⁺ = 512.1, 514.1. ¹H NMR (400 MHz, DMSO-d₆) δ 10.97 (s, 1 H), 8.40 (s, 1 H), 7.85-7.77 (m, 2 H), 7.48 (dd, J = 8.4, 2.4 Hz, 1 H), 6.97 (s, 1 H), 4.56 (s, 1 H), 4.49 (dt, J = 9.8, 5.0 Hz, 1 H), 4.53-4.45 (m, 2 H), 3.98 (s, 3 H), 3.29-3.28 (s, 2 H), 1.90- 1.75 (m, 4 H). |

### Example 217: Preparation of 1-(4-((4-(3,4-dichloro-2-fluorophenyl)-8-methoxy-5-methyl-4H-pyrido[2,3,4-de]quinazolin-7-yl)oxy)piperidin-1yl)prop-2-en-1-one (Compound 217)

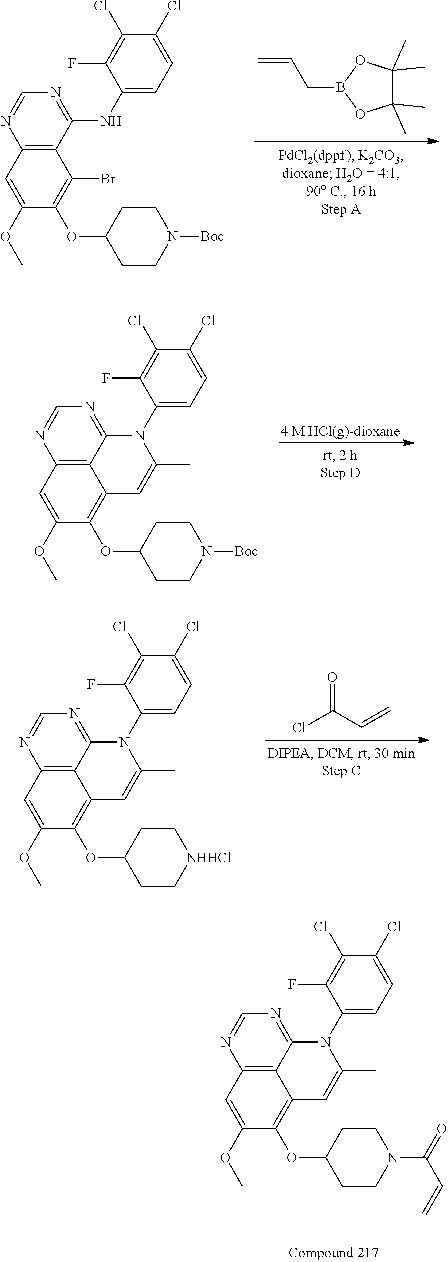

Step A: To a solution of tert-butyl 4-((5-bromo-4-((3,4-dichloro-2-fluorophenyl)amino)-7-methoxyquinazolin-6-yl)oxy)piperidine-1-carboxylate (150 mg, 244 umol), 2-allyl-4,4,5,5-tetramethyl-1,3,2-dioxaborolane (403 mg, 2.4 mmol), K₂CO₃ (101 mg, 732 umol) and PdCl₂(dppf) (36 mg, 48.8 umol) in 1.4-dioxane:H₂O (10 mL:1 mL). The reaction was heated to 90° C. and stirred for 18 hours under nitrogen atmosphere. The reaction was cooled to r.t and quenched by water, extracted with EtOAc (20 mL×3). The combined organic layers were washed with brine, dried over anhydrous sodium sulfate, filtered and concentrated in vacuum. The residue was purified via reverse phase column chromatography (CH₃CN/H₂O) to give tert-butyl 4-((4-(3,4-dichloro-2-fluorophenyl)-8-methoxy-5-methyl-4H-pyrido[2,3,4-de]quinazolin-7-yl)oxy)piperidine-1-carboxylate (43 mg, 31% yield) as a brown solid. LC/MS (ESI, m/z): [M+H]⁺=575.1.

Step B: tert-butyl 4-((4-(3,4-dichloro-2-fluorophenyl)-8-methoxy-5-methyl-4H-pyrido[2,3,4-de]quinazolin-7-yl)oxy)piperidine-1-carboxylate (45 mg, 78 umol) in 4 M HCl in dioxane (3 mL) was stirred for 1 hours at r.t. The reaction was then concentrated in vacuum to give 4-(3,4-dichloro-2-fluorophenyl)-8-methoxy-5-methyl-7-(piperidin-4-yloxy)-4H-pyrido[2,3,4-de]quinazoline hydrochloride (40 mg, 100% yield) as a yellow solid. LC/MS (ESI, m/z): [M+H]⁺=475.1.

Step C: A mixture of 4-(3,4-dichloro-2-fluorophenyl)-8-methoxy-5-methyl-7-(piperidin-4-yloxy)-4H-pyrido[2,3,4-de]quinazoline hydrochloride (33 mg, 64 umol), acryloyl chloride (6.95 mg, 76.8 umol), DIPEA (33 mg, 256 umol) in DCM (3 mL) was stirred for 0.5 h at r.t. The reaction was cooled to r.t and quenched by water, extracted with DCM. The combined organic layers were washed with brine, dried over anhydrous sodium sulfate, filtered and concentrated in vacuum. The residue was purified via reverse phase column chromatography (CH₃CN/H₂O) to give 1-(4-((4-(3,4-dichloro-2-fluorophenyl)-8-methoxy-5-methyl-4H-pyrido[2,3,4-de]quinazolin-7-yl)oxy)piperidin-1yl)prop-2-en-1-one (Compound 217) (8 mg, 23.5 yield) as a white solid. LC/MS (ESI, m/z): [M+H]⁺=529.1, 531.2. ¹H NMR (400 MHz, DMSO) δ 8.46 (s, 1H), 7.83 (dd, J=8.8, 1.6 Hz, 1H), 7.74 (t, J=8.2 Hz, 1H), 7.10 (s, 1H), 7.00 (s, 1H), 6.84 (ddd, J=14.2, 10.5, 3.7 Hz, 1H), 6.12 (dd, J=16.7, 2.4 Hz, 1H), 5.69 (dd, J=10.4, 2.4 Hz, 1 H), 4.43-4.35 (m, 1H), 4.19-4.11 (m, 2H), 4.02 (s, 3H), 3.33-3.25 (m, 1H), 3.12-3.02 (m, 1H), 2.10 (s, 3H), 1.97-1.89 (m, 2H), 1.72-1.64 (m, 2H).

### Example 218: Preparation of 1-(4-((4-(3,4-dichloro-2-fluorophenyl)-8-methoxy-4H-pyrido[2,3,4-de]quinazolin-7-yl)oxy) piperidin-1-yl)prop-2-en-1-one (Compound 218)

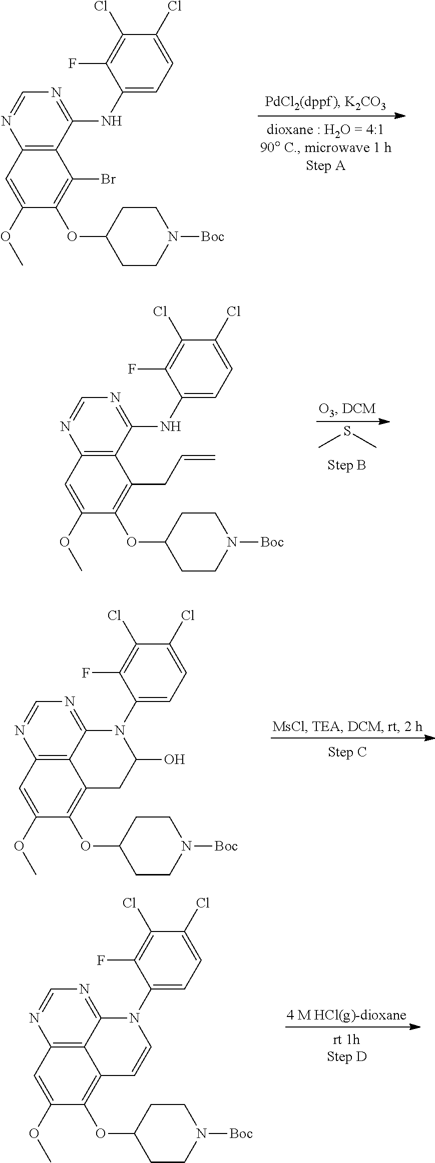

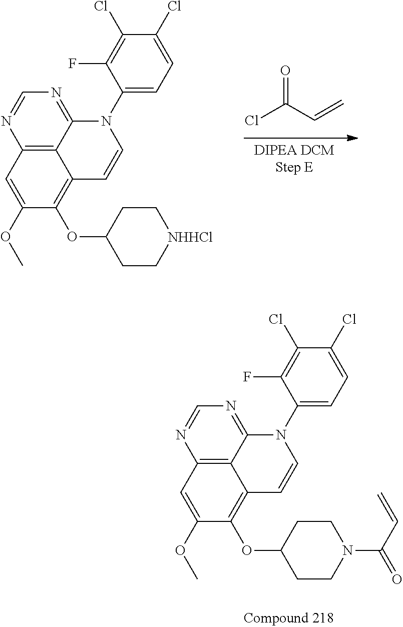

Step A: To a solution of tert-butyl 4-((5-bromo-4-((3,4-dichloro-2-fluorophenyl)amino)-7-methoxyquinazolin-6-yl)oxy)piperidine-1-carboxylate (400 mg, 651 umol), 2-allyl-4,4,5,5-tetramethyl-1,3,2-dioxaborolane (1.09 g, 6.51 mol), K₂CO₃ (269.5 mg, 1.95 mmol) and PdCl₂(dppf) (95 mg, 130 umol) in 1.4-dioxane:H₂O (9 mL:3 mL). The reaction was heated to 90° C. in microwave under nitrogen atmosphere for 1 hour. The reaction was cooled to r.t and quenched by water, extracted with EtOAc (20 mL×3). The combined organic layers were washed with brine, dried over anhydrous sodium sulfate, filtered and concentrated in vacuum. The residue was purified via reverse phase column chromatography (CH₃CN/H₂O) to give tert-butyl 4-((5-allyl-4-((3,4-dichloro-2-fluorophenyl)amino)-7-methoxyquinazolin-6-yl)oxy)piperidine-1-carboxylate (180.4 mg, 48% yield) as a brown solid. LC/MS (ESI, m/z): [M+H]⁺=577.2.

Step B: To a solution of tert-butyl 4-((5-bromo-4-((3,4-dichloro-2-fluorophenyl)amino)-7-methoxyquinazolin-6-yl)oxy)piperidine-1-carboxylate (50 mg, 88 umol) in MeOH (5 mL). The reaction was cooled to −78° C. and bubbled with ozone for 0.5 h until the reaction mixture turned to blue solution. The reaction mixture was stirred under nitrogen atmosphere for 10 min and dimethyl sulfide (1 mL) was added. The mixture was stirred for 5 min and warmed to rt and stirred overnight. The mixture was concentrated in vacuum and purified by column chromatography (CH₃CN/H₂O) to give tert-butyl 4-((4-(3,4-dichloro-2-fluorophenyl)-5-hydroxy-8-methoxy-5,6-dihydro-4H-pyrido[2,3,4-de]quinazolin-7-yl)oxy)piperidine-1-carboxylate (35 mg, 75% yield) as a brown solid. LC/MS (ESI, m/z): [M+H]⁺=579.1.

Step C: To a solution of tert-butyl 4-((4-(3,4-dichloro-2-fluorophenyl)-5-hydroxy-8-methoxy-5,6-dihydro-4H-pyrido[2,3,4-de]quinazolin-7-yl)oxy)piperidine-1-carboxylate (30 mg, 51.8 umol) in DCM (3 mL). Et₃N (52 mg, 518 umol) and MsCl (41.5 mg, 363 umol) were added. The reaction was stirred at room temperature for 4 hours and quenched by water, extracted with DCM. The combined organic layers were washed with brine, dried over anhydrous sodium sulfate, filtered and concentrated in vacuum. The residue was purified via reverse phase column chromatography (CH₃CN/H₂O) to give tert-butyl 4-((4-(3,4-dichloro-2-fluorophenyl)-8-methoxy-4H-pyrido[2,3,4-de]quinazolin-7-yl)oxy)piperidine-1-carboxylate (12 mg, 41%) as a white solid. LC/MS (ESI, m/z): [M+H]⁺=561.1.

Step D: tert-butyl 4-((4-(3,4-dichloro-2-fluorophenyl)-8-methoxy-4H-pyrido[2,3,4-de]quinazolin-7-yl)oxy)piperidine-1-carboxylate (12 mg, 21.3 umol) in 4 M HCl in dioxane (2 mL) was stirred for 1 hours at r.t. The reaction was then concentrated in vacuum to give 4-(3,4-dichloro-2-fluorophenyl)-8-methoxy-7-(piperidin-4-yloxy)-4H-pyrido[2,3,4-de]quinazoline hydrochloride (9 mg, 85% yield) as a yellow solid. LC/MS (ESI, m/z): [M+H]⁺=461.1.

Step E: A mixture of 4-(3,4-dichloro-2-fluorophenyl)-8-methoxy-7-(piperidin-4-yloxy)-4H-pyrido[2,3,4-de]quinazoline hydrochloride (9 mg, 18 umol), acryloyl chloride (1.96 mg,21.6 umol), DIPEA (7 mg, 54 umol) in DCM (2 mL) was stirred for 0.5 h at r.t. The reaction was cooled to r.t and quenched by water, extracted with DCM. The combined organic layers were washed with brine, dried over anhydrous sodium sulfate, filtered and concentrated in vacuum. The residue was purified via reverse phase column chromatography (CH₃CN/H₂O) to give 1-(4-((4-(3,4-dichloro-2-fluorophenyl)-8-methoxy-4H-pyrido[2,3,4-de]quinazolin-7-yl)oxy) piperidin-1-yl)prop-2-en-1-one (Compound 218) TFA salt (3 mg, 31% yield) as a white solid. LC/MS (ESI, m/z): [M+H]f=515.2, 517.2. ¹H NMR (400 MHz, CDCl₃) δ 8.39 (s, 1H), 7.90 (s, 1H), 7.55-7.43 (m, 1H), 7.33-7.27 (m, 1H), 7.02-6.97 (m, 1H), 6.95 (d, J=7.6 Hz, 1H), 6.61 (dd, J=16.8, 10.6 Hz, 1H), 6.31 (dd, J=16.8, 1.7 Hz, 1H), 5.73 (dd, J=10.6, 1.7 Hz, 1H), 4.55-4.45 (m, 1H), 4.35-4.25 (m, 1H), 4.12 (s, 3H), 4.00-3.91 (m, 1H), 3.40-3.20 (m, 2H), 2.04-1.94 (m, 2H), 1.85-1.73 (m, 2H).

### Example 219: Preparation of 4-((3,4-dichloro-2-fluorophenyl)amino)-7-methoxy-5-nitroquinazolin-6-ol (Compound 219)

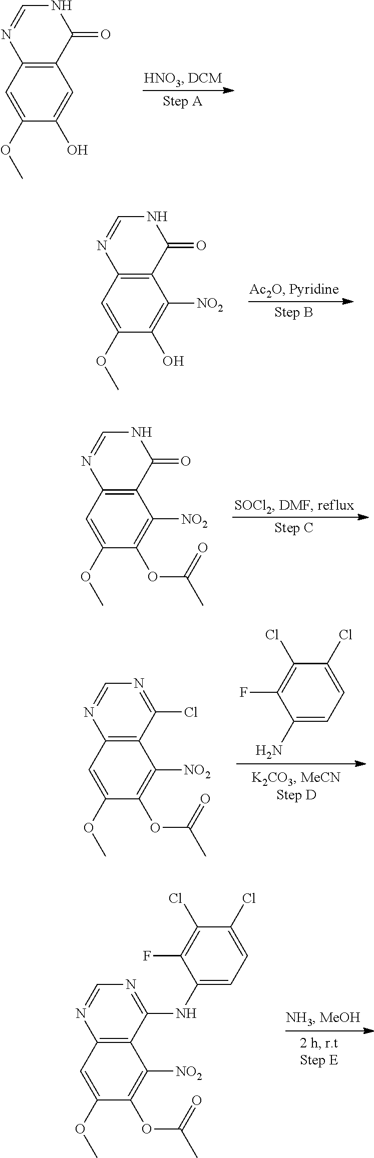

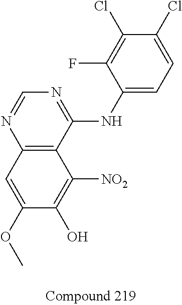 [SMILES: COc1cc2ncnc(Nc3ccc(Cl)c(Cl)c3F)c2c([N+](=O)[O-])c1O]

Step A: To a solution of fuming nitric acid (16 mL) was slowly added to 6,7-dimethoxy-3,4-dihydroquinazolin-4-one (10 g, 52 mmol) in DCM (100 mL). The reaction mixture was heated at rt for 18 hours, then allowed to cool and diluted with water. The resulting precipitate was collected by filtration, washed with water and dried to give 7-methoxy-5-nitro-4-oxo-3,4-dihydroquinazolin-6-yl acetate (1 Ig, 89% yield). LC/MS (ESI, m/z): [M+H]⁺=238.0.

Step B: 6-hydroxy-7-methoxy-5-nitroquinazolin-4(3H)-one (12 g, 50.4 mmol) was added to acetic anhydride (100 mL) and Pyridine (20 mL) was added with stirring. Heated at 100° C. for 4 hours. After cooling to room temperature, add ice water quenching reaction. The precipitated white solid was 7-methoxy-5-nitro-4-oxo-3,4-dihydroquinazolin-6-yl acetate (8 g, 57%). LC/MS (ESI, m/z): [M+H]⁺=280.0.

Step C: To a solution of 7-methoxy-5-nitro-4-oxo-3,4-dihydroquinazolin-6-yl acetate (3.0 g, 10.74 mmol, 1.00 equiv), thionyl chloride (30 mL), N,N-dimethylformamide (0.1 mL). The resulting solution was stirred for 1.5 h at 85° C. The resulting solution was evaporated in vacuum, extracted with EtOAc (3×100 mL) and the organic layers were combined and dried over Na2SO4. The solid was filtered and the filtrate was concentrated under reduced pressure to afford 4-chloro-7-methoxy-5-nitroquinazolin-6-yl acetate (3 g, 94%) as a brown solid. LC/MS (ESI, m/z): [M+H]⁺=298.0.

Step D: A mixture of 4-chloro-7-methoxy-5-nitroquinazolin-6-yl acetate (825 mg, 2.8 mmol), 3,4-dichloro-2-fluoroaniline (499 mg, 2.77 mmol) in isopropanol was degassed with nitrogen, heated to 80° C. and stirred for 1 hour under nitrogen atmosphere. The reaction was cooled to r.t, filtered and concentrated in vacuum to give 4-((3,4-dichloro-2-fluorophenyl)amino)-7-methoxy-5-nitroquinazolin-6-yl acetate (1.1 g, 90% yield) as a brown solid. LC/MS (ESI, m/z): [M+H]⁺=441.0.

Step E: A solution of 4-((3,4-dichloro-2-fluorophenyl)amino)-7-methoxy-5-nitroquinazolin-6-yl acetate (1.1 g, 2.5 mmol) in MeOH (10 mL) was slowly added ammonium hydroxide at RT. After addition, the reaction mixture was stirred at RT for 2 h. The reaction was then filtered and the cake was dried to give 4-((3,4-dichloro-2-fluorophenyl)amino)-7-methoxy-5-nitroquinazolin-6-ol (Compound 219) (800 mg, 80% yield) as an orange solid. LC/MS (ESI, m/z): [M+H]⁺=398.8, 400.8. ¹H NMR (400 MHz, DMSO-d₆) δ 8.37 (s, 1H), 8.31 (s, 1H), 7.77 (t, J=8.8 Hz, 1H), 7.42 (d, J=8.5 Hz, 1H), 6.79 (s, 1H), 3.83 (s, 3H).

### Example 220: Preparation of (E)-3-(3,4-dichloro-2-fluorophenyl)-9-((1-(4-(dimethylamino)but-2-enoyl)piperidin-4-yl)oxy)-8-methoxy-1H-pyrimido[4,5,6-de]quinazolin-2(3H)-one (Compound 220)

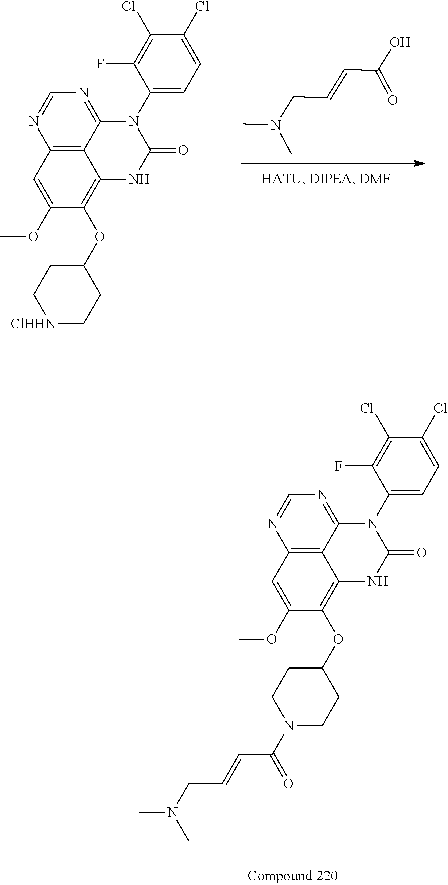

To a solution of 3-(3,4-dichloro-2-fluorophenyl)-8-methoxy-9-(piperidin-4-yloxy)-1H-pyrimido[4,5,6-de]quinazolin-2(3H)-one hydrochloride (60 mg, 0.12 mmol) in DMF (3 mL) was added HATU (68.4 mg, 0.18 mmol) and DIPEA (46 mg, 0.36 mmol), (E)-4-(dimethylamino)but-2-enoic acid (23 mg, 0.18 mmol). The reaction mixture was stirred at room temperature overnight. The mixture was purified by prep-HPLC (CH₃CN and H₂O with 0.01% TFA as mobile phase) to give the product (E)-3-(3,4-dichloro-2-fluorophenyl)-9-((1-(4-(dimethylamino)but-2-enoyl)piperidin-4-yl)oxy)-8-methoxy-1H-pyrimido[4,5,6-de]quinazolin-2(3H)-one (Compound 220) as a TFA salt (22 mg, 32% yield) as a white solid. LC/MS (ESI, m/z): [M+H]⁺=589.1, 591.1. ¹H NMR (400 MHz, DMSO-d₆) δ 11.16 (s, 1H), 9.91 (br s, 1 H), 8.47 (s, 1H), 7.71 (dt, J=16.3, 8.7 Hz, 2H), 7.10-6.82 (m, 2H), 6.65-6.45 (m, 1H), 4.50-4.06 (m, 2H), 4.09-3.93 (m, 4H), 3.92-3.85 (m, 2H), 2.83-2.66 (m, 6H), 1.92-1.84 (m, 2H), 1.83-1.75 (m, 2H).

### Example 221: Preparation of (E)-3-(3,4-dichloro-2-fluorophenyl)-9-((1-(4-(dimethylamino)but-2-enoyl)piperidin-4-yl)oxy)-8-methoxy-1-methyl-11H-pyrimido[4,5,6-de]quinazolin-2(3H)-one (Compound 221)

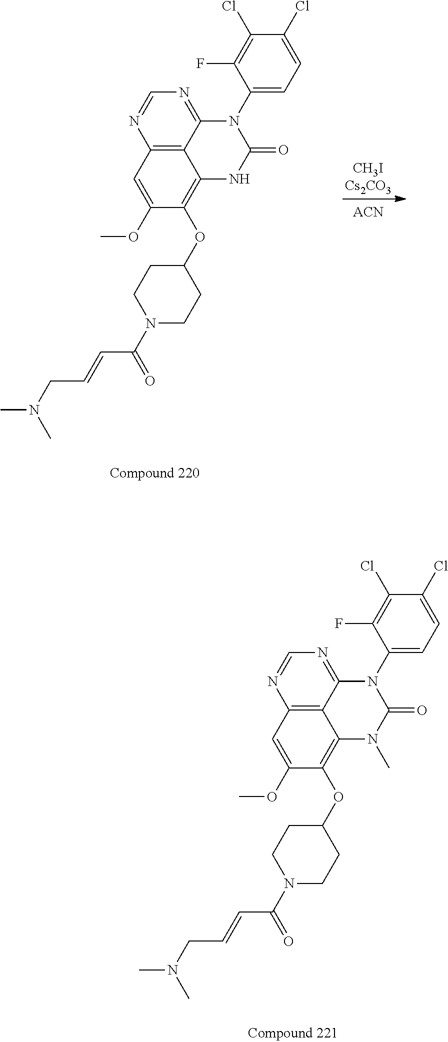 [SMILES: COc1cc2ncnc3c2c(c1OC1CCN(C(=O)/C=C/CN(C)C)CC1)NC(=O)N3c1ccc(Cl)c(Cl)c1F ; "ACN" ; COc1cc2ncnc3c2c(c1OC1CCN(C(=O)/C=C/CN(C)C)CC1)N(C)C(=O)N3c1ccc(Cl)c(Cl)c1F]

To a solution of (E)-3-(3,4-dichloro-2-fluorophenyl)-9-((1-(4-(dimethylamino)but-2-enoyl)piperidin-4-yl)oxy)-8-methoxy-1H-pyrimido[4,5,6-de]quinazolin-2(3H)-one (15 mg, 0.026 mmol) in CH₃CN (2 mL) was added Cs₂CO₃ (17 mg, 0.051 mmol) and Mel (0.15 mL, 0.039 mmol) (0.1 M in CH₃CN). The reaction mixture was stirred at room temperature for 4 h. H₂O (5 mL) was added. The mixture was extracted with EtOAc (10 mL×3), washed with brine, dried over Na₂SO₄. The solid was filtered and the filtrate was concentrated to give a residue which was purified by prep-HPLC (CH3CN and H2O with 0.01% TFA as mobile phase) to give the product (E)-3-(3,4-dichloro-2-fluorophenyl)-9-((1-(4-(dimethylamino)but-2-enoyl)piperidin-4-yl)oxy)-8-methoxy-1-methyl-1H-pyrimido[4,5,6-de]quinazolin-2(3H)-one (Compound 221) 2,2,2-trifluoroacetate as a white solid. (7 mg, 39% yield). LC/MS (ESI, m/z): [M+H]⁺=603.0. ¹H NMR (400 MHz, CD₃OD) δ 8.51 (d, J=9.0 Hz, 1H), 7.59 (d, J=8.8 Hz, 1H), 7.46 (dd, J=16.1, 8.3 Hz, 1H), 7.18-6.95 (m, 2H), 6.81-6.73 (m, 1H), 4.50-4.34 (m, 2H), 4.16-4.01 (m, 6H), 3.82 (s, 3H), 3.19-2.98 (m, 8H), 2.18-2.00 (m, 2H), 1.86-1.68 (m, 2H).

Examples 222-227 were prepared using similar procedures as described in the preceding Examples.

| EX. | | Compound | | |
|---|---|---|---|---|
| NO. | IUPAC Name | structure | salt | Compound characterization |
| 222 | 9-((1-(but-2- ynoyl)piperidin- 4-yl)oxy)-3-(3,4- dichloro-2- fluorophenyl)-8- methoxy-1H- pyrimido[4,5,6- de]quinazolin- 2(3H)-one | 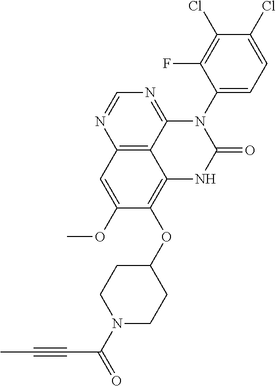 [SMILES: CC#CC(=O)N1CCC(Oc2c(OC)cc3ncnc4c3c2NC(=O)N4c2ccc(Cl)c(Cl)c2F)CC1] | None | LC/MS (ESI, m/z): [M + H]⁺ = 544.0, 546.0. ¹H NMR (400 MHz, DMSO-d₆) δ11.20 (s, 1 H), 8.48 (s, 1 H), 7.79-7.61 (m, 2 H), 7.03 (s, 1 H), 4.51-4.45 (m, 1 H), 4.25-4.22 (m, 2 H), 4.00 (s, 3 H), 3.29-3.11 (m, 1 H), 2.82 (t, J = 12.2 Hz, 1 H), 2.03 (s, 3 H), 2.02-1.72 (m, 4 H). |
| 223 | 9-((1-(but-2- ynoyl)piperidin- 4-yl)oxy)-3-(3,4- dichloro-2- fluorophenyl)-8- methoxy-1- methyl-1H- pyrimido[4,5,6- de]quinazolin- 2(3H)-one | 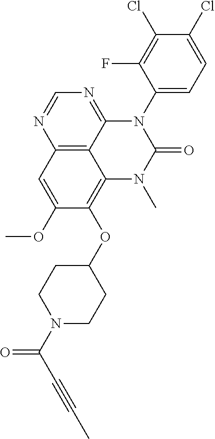 [SMILES: CC#CC(=O)N1CCC(Oc2c(OC)cc3ncnc4c3c2N(C)C(=O)N4c2ccc(Cl)c(Cl)c2F)CC1] | None | LC/MS (ESI, m/z): [M + 1]+ = 558.0, 560.0. ¹H NMR (400 MHz, CD3OD) δ 8.52 (s, 1 H), 7.59 (d, J = 8.8 Hz, 1 H), 7.53-7.39 (m, 1 H), 7.10 (s, 1 H), 4.43-4.26 (m, 3 H), 4.08 (s, 3 H), 3.83 (s, 3 H), 2.95 (d, J = 13.6 Hz, 1 H), 2.04 (s, 2 H), 2.03 (s, 3 H), 1.81-1.64 (m, 2 H). |
| 224 | 9-((1-(but-2- ynoyl)piperidin- 4-yl)oxy)-3-(3- ethynylphenyl)- 8-methoxy-1H- pyrimido[4,5,6- de]quinazolin- 2(3H)-one | 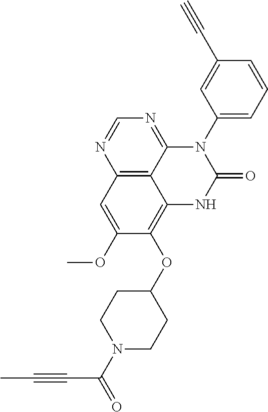 [SMILES: C#Cc1cccc(N2C(=O)Nc3c(OC4CCN(C(=O)C#CC)CC4)c(OC)cc4ncnc2c34)c1] | None | LC/MS (ESI, m/z): [M + H]⁺ = 482.0. ¹H NMR (400 MHz, DMSO-d₆) δ 11.10 (s, 1 H), 8.49 (s, 1 H), 7.60-7.51 (m, 3 H), 7.50- 7.44 (m, 1 H), 6.97 (s, 1 H), 4.53-4.42 (m, 1 H), 4.29 (s, 1 H), 4.23 (d, J = 13.3 Hz, 2 H), 4.00 (s, 3 H), 3.27-3.16 (m, 1 H), 2.90-2.76 (m, 1 H), 2.02 (s, 3 H), 1.97- 1.82 (m, 3 H), 1.82-1.68 (m, 1 H). |
| 225 | 3-(3,4-dichloro- 2-fluorophenyl)- 8-methoxy-9-((1- (vinylsulfonyl) piperidin-4- yl)oxy)-1H- pyrimido[4,5,6- de]quinazolin- 2(3H)-one | 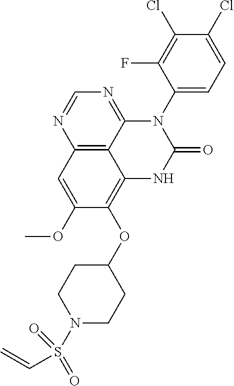 | None | LC/MS (ESI, m/z): [M + 17]⁺ = 584.2. ¹H NMR (400 MHz, CDCl₃) δ 8.62 (s, 1 H), 8.17 (s, 1 H), 7.62-7.42 (m, 1 H), 7.29 (d, J = 7.9 Hz, 1 H), 7.23 (s, 1 H), 6.46 (dd, J= 16.6, 9.9 Hz, 1 H), 6.28 (d, J = 16.6 Hz, 1 H), 6.07 (d, J = 9.9 Hz, 1 H), 4.46 (m,1 H), 4.06 (s, 3 H), 3.70-3.60 (m, 2 H) , 2.96 (t, J = 9.8 Hz, 2 H), 2.15-2.05(m, 2 H), 1.94-1.87 (m, 2 H). |
| 226 | 3-(3,4- dichlorophenyl)- 8-methoxy-9-((1- (vinylsulfonyl) piperidin-4- yl)oxy)-1H- pyrimido[4,5,6- de]quinazolin- 2(3H)-one | 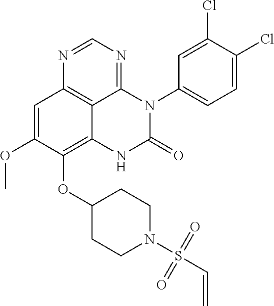 [SMILES: C=CS(=O)(=O)N1CCC(Oc2c(OC)cc3ncnc4c3c2NC(=O)N4c2ccc(Cl)c(Cl)c2)CC1] | None | LC/MS (ESI, m/z): [M + H]⁺ = 550.0, 552.0. ¹H NMR (400 MHz, DMSO-d₆) δ 8.58 (s, 1 H), 8.00 (s, 1 H), 7.66 (d, J = 8.4 Hz, 1 H), 7.48 (d, J = 2.4 Hz, 1 H), 7.25- 7.20 (m, 1 H), 7.00 (d, J = 4.9 Hz, 1 H), 6.46 (dd, J = 16.8, 10.0 Hz, 1 H), 6.28 (d, J = 16.8 Hz, 1 H), 6.07 (d, J = 9.6 Hz, 1 H), 4.46-4.39 (m, 1 H), 4.03 (s, 3 H), 3.69- 3.60 (m, 2 H), 3.02-2.92 (m, 2 H), 2.12- 2.08 (m, 2 H), 1.96-1.83 (m, 2 H). |
| 227 | 3-(3,4- dichlorophenyl)- 8-methoxy-9-((1- propioloylpiperidin- 4-yl)oxy)-1H- pyrimido[4,5,6- de]quinazolin- 2(3H)-one | 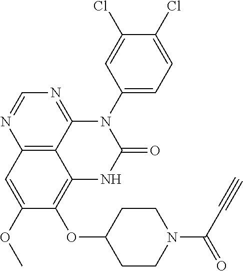 [SMILES: C#CC(=O)N1CCC(Oc2c(OC)cc3ncnc4c3c2NC(=O)N4c2ccc(Cl)c(Cl)c2)CC1] | None | LC/MS (ESI, m/z): [M + H]⁺ = 512.1, 514.1. ¹H NMR (400 MHz, DMSO-d₆) δ 10.97 (s, 1 H), 8.40 (s, 1 H), 7.85-7.77 (m, 2 H), 7.48 (dd, J = 8.4, 2.4 Hz, 1 H), 6.97 (s, 1 H), 4.56 (s, 1 H), 4.49 (dt, J = 9.8, 5.0 Hz, 1 H), 4.53-4.45 (m, 2 H), 3.98 (s, 3 H), 3.29-3.28 (s, 2 H), 1.90-1.75 (m, 4 H). |

### Example 228: Preparation of N-(4-amino-5-(quinolin-3-yl)-8,9-dihydro-7H-pyrimido[5′,4′:4,5]pyrrolo[2,1-b][1,3]oxazin-8-yl)acrylamide (Compound 228)

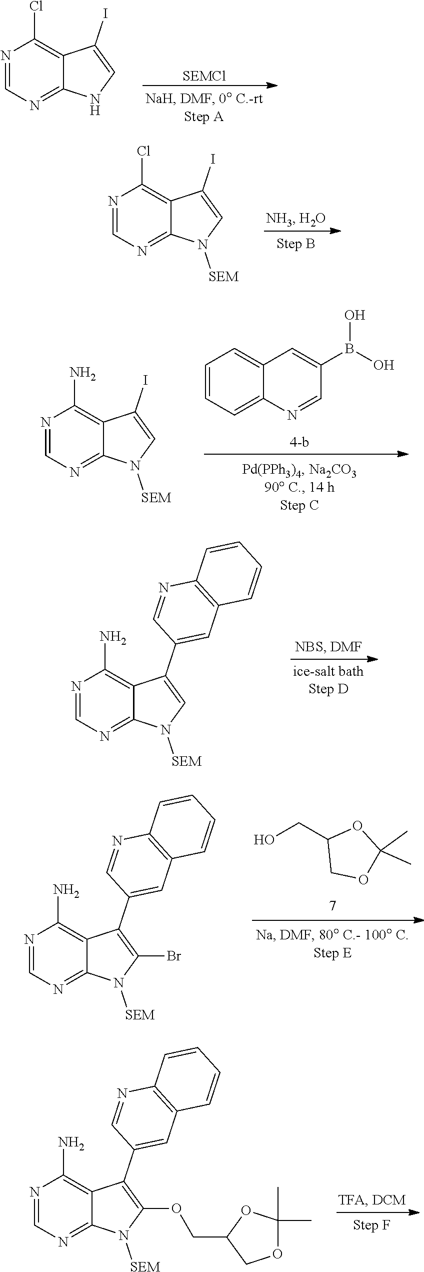

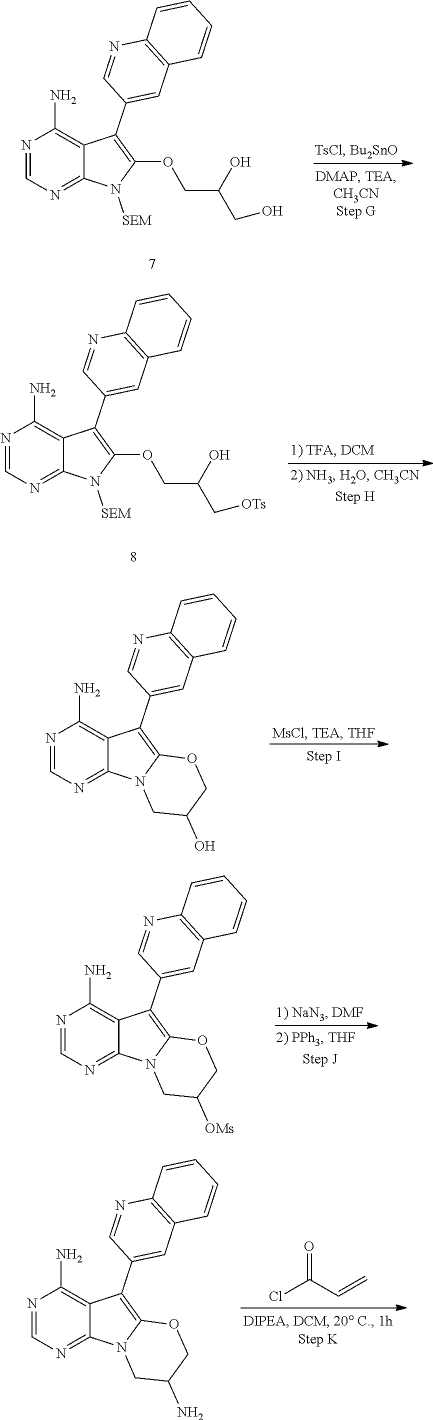

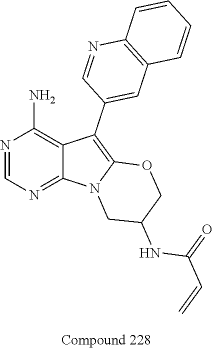 [SMILES: C=CC(=O)NC1COc2c(-c3cnc4ccccc4c3)c3c(N)ncnc3n2C1]

Step A: To a mixture of NaH (3.43 g, 86.0 mmol) in DMF (30 mL) were added dropwise a solution of 4-chloro-5-iodo-7H-pyrrolo[2,3-d]pyrimidine (16 g, 57.2 mmol) in DMF (100 ml) at 0° C. under N₂. The reaction mixture was stirred at the same temperature for 1 hour and then a solution of SEMCI (10 g, 60.1 mmol) in DMF (100 ml) was added to the mixture. The resulting mixture was stirred at the room temperature for 2 hr. The reaction solution was quenched with water (100 mL) and then extracted with EA (100 mL×3). The combined organic layers were washed with brine. The organic layer was dried over Na₂SO₄, filtered and concentrated to give a residue which was purified by silica gel with PE/EA=4/1 to give 4-chloro-5-iodo-7-((2-(trimethylsilyl)ethoxy) methyl)-7H-pyrrolo[2,3-d]pyrimidine as a white solid (15 g, 36.6 mmol, 64% yield). LC/MS (ESI, m/z): [M+H]⁺=410.1.

Step B: To a mixture of 4-chloro-5-iodo-7-((2-(trimethylsilyl)ethoxy) methyl)-7H-pyrrolo [2,3-d]pyrimidine (11 g, 26.8 mmol) in 1.4-dioxane (30 mL) was added NH₃·H₂O (150 mL). The reaction mixture was stirred at 120° C. for 16 hr. then the mixture was concentrated under reduced pressure to give 5-iodo-7-((2-(trimethylsilyl) ethoxy)methyl)-7H-pyrrolo[2,3-d]pyrimidin-4-amine as a white solid (10.5 g, 26.8 mmol, 100% yield). LC/MS (ESI, m/z): [M+H]⁺=391.1.

Step C: To a mixture of 5-iodo-7-((2-(trimethylsilyl)ethoxy)methyl)-7H-pyrrolo[2,3-d]pyrimidin-4-amine (12 g, 30.7 mmol), quinolin-3-ylboronic acid (6.387 g, 36.9 mmol), and Na₂CO₃ (6.523 g, 61.5 mmol) in 1,4-dioxane (100 mL) and H₂O (50 mL) were added Pd(PPh₃)₄ (3.555 g, 3.07 mmol), and the reaction mixture was stirred at 100° C. for 16 hours under N₂. The mixture was cooled to room temperature, then extracted with EA (60×3 mL), the organic layers were washed with brine, dried over anhydrous Na₂SO₄, concentrated under reduced pressure to give a residue which was purified by silica gel with PE/EA=1/1 to give 5-(quinolin-3-yl)-7-((2-(trimethylsilyl) ethoxy)methyl)-7H-pyrrolo[2,3-d]pyrimidin-4-amine as a yellow solid (10 g, 25.5 mmol, 83% yield). LC/MS (ESI, m/z): [M+H]⁺=392.1. ¹H NMR (400 MHz, DMSO-d₆) δ 9.10 (d, J=2 Hz, 1H), 8.4 (d, J=2 Hz, 1H), 8.29 (s, 1H), 8.14-8.08 (m, 2H), 7.84-7.79 (m, 1H), 7.74 (s, 1H), 7.71-7.67 (m, 1H), 6.45 (s, 2H), 5.65 (s, 2H), 3.65 (t, J=8 Hz, 2H), 0.93 (t, J=8 Hz, 2H), 0 (s, 9H).

Step D: To a solution of 5-(quinolin-3-yl)-7-((2-(trimethylsilyl)ethoxy)methyl)-7H-pyrrolo [2,3-d]pyrimidin-4-amine (10 g, 25.5 mmol) in DMF (100 mL) was added NBS (5.0 g, 28.4 mmol) in an ice-salt bath, and the reaction mixture was stirred at the same temperature for 1 hr. The mixture was diluted with water (100 mL) and then extracted with EA (100×3 mL). The organic layers were washed with water and brine, dried over anhydrous Na₂SO₄, concentrated under reduced pressure to give a residue which was purified by silica gel with EA to give 6-bromo-5-(quinolin-3-yl)-7-((2-(trimethylsilyl)ethoxy)methyl)-7H-pyrrolo[2,3-d]pyrimidin-4-amine as a yellow solid (8.3 g, 17.6 mmol, 70% yield). LC/MS (ESI, m/z): [M+H]⁺=470.1, 472.1. ¹H NMR (400 MHz, DMSO-d₆) δ 8.97 (d, J=2.4 Hz, 1H), 8.45 (d, J=2 Hz, 1H), 8.28 (s, 1H), 8.15-8.12 (m, 2H), 7.89-7.85 (m, 1H), 7.74-7.70 (m, 1H), 6.37 (s, 2H), 5.70 (s, 2 H), 3.67 (t, J=7.6 Hz, 2H), 0.93 (t, J=8 Hz, 2H), 0 (s, 9H).

Step E: A mixture of Na (980 mg, 42.6 mmol) and (2,2-dimethyl-1,3-dioxolan-4-yl)methanol (40 mL) was stirred at 80° C. for 30 min under N₂, a solution of 6-bromo-5-(quinolin-3-yl)-7-((2-(trimethylsilyl)ethoxy)methyl)-7H-pyrrolo[2,3-d]pyrimidin-4-amine (2 g, 4.26 mmol) in DMF (20 mL) was added to this mixture, the resulting solution was stirred at 100° C. for 16 hr. The mixture was cooled to room temperature, quenched with water, and extracted with EA (50×3 mL). The organic layers were washed with water and brine, dried over anhydrous Na₂SO₄, and concentrated under reduced pressure to give a residue. The residue was purified by silica gel with EA to give 6-((2,2-dimethyl-1,3-dioxolan-4-yl)methoxy)-5-(quinolin-3-yl)-7-((2-(trimethylsilyl)ethoxy)methyl)-7H-pyrrolo[2,3-d]pyrimidin-4-amine as a yellow solid (1.5 g, 2.87 mmol, 67% yield). ¹H NMR (400 MHz, CDCl₃) δ 9.40 (s, 1H), 8.79 (s, 1H), 8.30 (d, J=8.4 Hz, 1H), 8.19 (s, 1H), 8.06 (d, J=8 Hz, 1H), 7.97-7.94 (m, 1H), 7.84-7.80 (m, 1H), 5.64 (s, 2H), 4.26-4.22 (m, 1H), 4.07-4.04 (m, 1H), 3.96-3.88 (m, 2H), 3.73 (t, J=8 Hz, 2H), 3.60-3.56 (m, 1H), 1.147 (s, 6H), 0.97 (t, J=8.8 Hz, 2H), 0 (s, 9H). LC/MS (ESI, m/z): [M+H]⁺=522.1.

Step F: To a solution of 6-((2,2-dimethyl-1,3-dioxolan-4-yl)methoxy)-5-(quinolin-3-yl)-7-((2-(trimethylsilyl)ethoxy)methyl)-7H-pyrrolo[2,3-d]pyrimidin-4-amine (1.4 g, 2.68 mmol) in DCM (15 mL) was added TFA (3.0 g, 26.8 mmol). The reaction mixture was stirred at the room temperature for 4 hr. The mixture was neutralized with Na₂CO₃, then concentrated under reduced pressure. The residue was dissolved in MeOH (20 mL), stirred at the room temperature for 20 min, then filtered and concentrated under reduced pressure to give 3-((4-amino-5-(quinolin-3-yl)-7-((2-(trimethylsilyl)ethoxy) methyl)-7H-pyrrolo[2,3-d]pyrimidin-6-yl)oxy)propane-1,2-diol as a yellow solid (1.2 g, 2.68 mmol, 100% yield). LC/MS (ESI, m/z): [M+H]⁺=482.1. ¹H NMR (400 MHz, DMSO-d₆) δ 9.03 (d, J=2 Hz, 1H), 8.41 (d, J=1.6 Hz, 1H), 8.21 (s, 1H), 8.11-8.07 (m, 2H), 7.83-7.79 (m, 1H), 7.69-7.66 (m, 1H), 6.03 (s, 2H), 5.58 (s, 2H), 4.98 (d, J=4.8 Hz, 1H), 4.60 (t, J=5.6 Hz, 1H), 3.93-3.90 (m, 1H), 3.84-3.80 (m, 1H), 3.71-3.64 (m, 3H), 3.34-3.29 (m, 2H), 0.92 (t, J=8 Hz, 2H), 0 (s, 9H).

Step G: A mixture of 3-((4-amino-5-(quinolin-3-yl)-7-((2-(trimethylsilyl)ethoxy)methyl)-7H-pyrrolo[2,3-d]pyrimidin-6-yl)oxy)propane-1,2-diol (1.1 g, 2.28 mmol), Bu₂SnO (114 mg, 0.45 mmol), TEA (634 mL, 4.57 mmol) and DMAP (56 mg, 0.45 mmol) in CH₃CN (10 mL) was stirred at the room temperature for 10 min. To this mixture was added a solution of TsCl (480 mg, 2.51 mmol) in CH₃CN (4 mL). The resulting reaction mixture was stirred at the room temperature for 1 hr. The mixture was concentrated under reduced pressure to give a residue which was purified by silica gel with PE/EA=1/1 to give 3-((4-amino-5-(quinolin-3-yl)-7-((2-(trimethylsilyl)ethoxy)methyl)-7H-pyrrolo[2,3-d]pyrimidin-6-yl)oxy)-2-hydroxypropyl 4-methylbenzenesulfonate as a yellow solid (1.0 g, 1.57 mmol, 75% yield). LC/MS (ESI, m/z): [M+H]⁺=636.1. ¹H NMR (400 MHz, DMSO-d₆) δ 8.99 (d, J=2.4 Hz, 1H), 8.39 (d, J=2 Hz, 1H), 8.23 (s, 1H), 8.15-8.12 (m, 1H), 8.09-8.07 (m, 1H), 7.87-7.83 (m, 1H), 7.74-7.71 (m, 3H), 7.46-7.44 (m, 2H), 6.06 (s, 2H), 5.53-5.50 (m, 3H), 4.04-4.02 (m, 1H), 3.95-3.82 (m, 4H), 3.65 (t, J=8 Hz, 2H), 2.44 (s, 3H), 0.86 (t, J=8.4 Hz, 2 H), 0 (s, 9H).

Step H: To a solution of 3-((4-amino-5-(quinolin-3-yl)-7-((2-(trimethylsilyl)ethoxy)methyl)-7H-pyrrolo[2,3-d]pyrimidin-6-yl)oxy)-2-hydroxypropyl 4-methylbenzenesulfonate (1.0 g, 1.57 mmol) in DCM (10 mL) was added TFA (10 mL). The mixture was stirred at 80° C. for 16 hours and then concentrated under reduced pressure. The residue was dissolved in CH₃CN (10 mL). To this solution was added NH₃·H₂O (480 mg, 2.51 mmol). The resulting solution was stirred at the room temperature for 1 hr. The mixture was concentrated under reduced pressure to give a residue which was purified by silica gel with MeOH/DCM=1/5 to give 4-amino-5-(quinolin-3-yl)-8,9-dihydro-7H-pyrimido[5′,4′:4,5]pyrrolo[2,1-b][1,3]oxazin-8-ol as a yellow solid (400 mg, 1.20 mmol, 76% yield). LC/MS (ESI, m/z): [M+H]⁺=334.1. ¹H NMR (400 MHz, DMSO-d₆) δ 8.99 (d, J=1.6 Hz, 1H), 8.19 (d, J=1.6 Hz, 1H), 8.12 (s, 1H), 8.02-7.97 (m, 2H), 7.72-7.68 (m, 1H), 7.61-7.57 (m, 1H), 6.00 (s, 2H), 5.66 (d, J=2 Hz, 1H), 4.42-4.32 (m, 3H), 4.19 (s, 2H).

Step I: To a mixture of 4-amino-5-(quinolin-3-yl)-8,9-dihydro-7H-pyrimido[5′,4′:4,5]pyrrolo[2,1-b][1,3]oxazin-8-ol (300 mg, 0.90 mmol) and TEA (0.25 mL, 1.80 mmol) in THE (4 mL) was added MsCl (0.077 mL, 0.99 mmol). The mixture was stirred at the room temperature for 1 hour, and then concentrated under reduced pressure to give a residue which was purified by prep-TLC with MeOH/DCM=1/7 to give 4-amino-5-(quinolin-3-yl)-8,9-dihydro-7H-pyrimido[5′,4′:4,5]pyrrolo[2,1-b][1,3]oxazin-8-yl methanesulfonate as a yellow solid (136 mg, 0.33 mmol, 36.7% yield). LC/MS (ESI, m/z): [M+H]⁺=412.1.

Step J: To a solution of 4-amino-5-(quinolin-3-yl)-8,9-dihydro-7H-pyrimido[5′,4′:4,5]pyrrolo [2,1-b][1,3]oxazin-8-yl methanesulfonate (136 mg, 0.33 mmol) in DMF (2 mL) was added NaN₃ (64 mg, 0.99 mmol). The mixture was heated at 80° C. for 16 hr. The mixture was cooled to room temperature, diluted with water (5 mL), and extracted with EA (10×3 mL). The organic layers were washed with water and brine, dried over anhydrous Na₂SO₄, concentrated under reduced pressure. The residue was dissolved in THE (2 mL) and H₂O (0.4 mL). To this mixture was added PPh3 (241 mg, 0.92 mmol). The resulting mixture was stirred at 65° C. for 1 hour, and then concentrated under reduced pressure to give a residue which was purified by prep-TLC with MeOH/DCM=1/5 to give 5-(quinolin-3-yl)-8,9-dihydro-7H-pyrimido [5′,4′:4,5]pyrrolo[2,1-b][1,3]oxazine-4,8-diamine as a yellow solid (40 mg, 0.12 mmol, 36% yield). LC/MS (ESI, m/z): [M+H]⁺=333.1. ¹H NMR (400 MHz, CD₃CN) δ 9.09 (d, J=2.4 Hz, 1H), 8.24 (d, J=2 Hz, 1H), 8.19 (s, 1H), 8.09-8.07 (m, 1H), 7.96-7.94 (m, 1H), 7.75-7.71 (m, 1H), 7.64-7.60 (m, 1H), 5.21 (s, 2H), 4.42-4.33 (m, 2H), 4.18-4.14 (m, 1 H), 3.98-3.93 (m, 1H), 3.66-3.61 (m, 1H).

Step K: To a mixture of 5-(quinolin-3-yl)-8,9-dihydro-7H-pyrimido[5′,4′:4,5]pyrrolo[2,1-b][1,3]oxazine-4,8-diamine (20 mg, 0.06 mmol), TEA (12 mg, 0.12 mmol) in DCM (1 mL) was added acryloyl chloride (6 mg, 0.06 mmol). The mixture was stirred at room temperature for 1 hr. The mixture was concentrated under reduced pressure to give a residue which was purified by prep-HPLC to give N-(4-amino-5-(quinolin-3-yl)-8,9-dihydro-7H-pyrimido[5′,4′:4,5]pyrrolo[2,1-b][1,3]oxazin-8-yl)acrylamide (Compound 228) as a yellow solid (3 mg, 0.007 mmol, 13% yield). 1H NMR (400 MHz, CD3CN) δ 9.04 (d, J=2.4 Hz, 1H), 8.35 (d, J=2 Hz, 1H), 8.15 (s, 1H), 8.12-8.10 (m, 1H), 7.99-7.97 (m, 1H), 7.80-7.75 (m, 1H), 7.66-7.62 (m, 1H), 7.11-7.10 (m, 1H), 6.18-6.05 (m, 2H), 5.57-5.54 (m, 1H), 4.64-4.63 (m, 1H), 4.44-4.29 (m, 4H). LC/MS (ESI, m/z): [M+H]⁺=387.1.

### Example 229: Preparation of 1-(4-((4-((3,4-dichloro-2-fluorophenyl)amino)-7H-pyrrolo[2,3-d]Pyrimidin-6-yl)(hydroxy)methyl)piperidin-1-yl)prop-2-en-1-one (Compound 229)

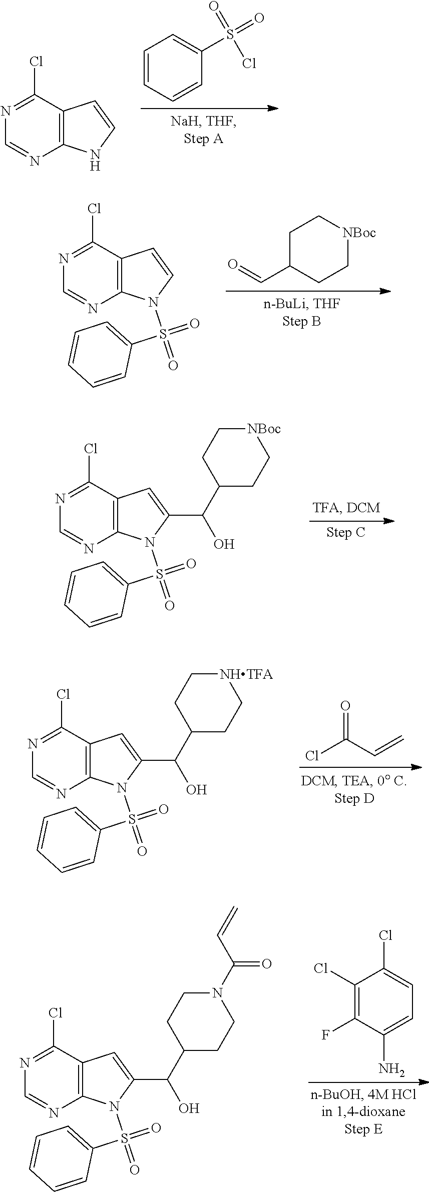

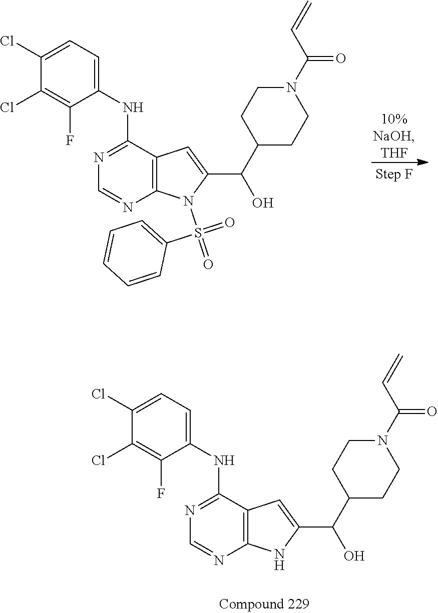 [SMILES: C=CC(=O)N1CCC(C(O)c2cc3c(Nc4ccc(Cl)c(Cl)c4F)ncnc3n2S(=O)(=O)c2ccccc2)CC1 ; C=CC(=O)N1CCC(C(O)c2cc3c(Nc4ccc(Cl)c(Cl)c4F)ncnc3[nH]2)CC1]

Step A: To a mixture of NaH (5.2 g, 13.0 mmol) in THF (40 mL) was added dropwise a solution of 4-chloro-7H-pyrrolo[2,3-d]pyrimidine (10 g, 6.51 mmol) in THF (60 mL). The reaction mixture was stirred at 0° C. for 1 hr and then benzenesulfonyl chloride (16.6 mL) was added dropwise to this mixture. The resulting mixture was stirred at the room temperature for 16 hr and then the reaction solution was quenched with water (100 mL). The mixture was extracted with EA (100 mL×3) and the combined organic layers were washed with brine. The organic layer was dried over Na₂SO₄, filtered and concentrated to give 4-chloro-7-(phenylsulfonyl)-7H-pyrrolo[2,3-d]pyrimidine as a white solid (18.5 g, 62.98 mmol, 96.8% yield). LC/MS (ESI, m/z): [M+H]⁺=294.1. ¹H NMR (400 MHz, DMSO-d₆) δ 8.86 (s, 1H), 8.22-8.17 (m, 3H), 7.83 (t, J=4 Hz, 1H), 7.80-7.68 (m, 2H), 6.99 (d, J=4 Hz, 1H).

Step B: To a solution of 4-chloro-7-(phenylsulfonyl)-7H-pyrrolo[2,3-d]pyrimidine (4.126 g, 14.0 mmol) in THF (40 mL) was added n-BuLi (13.2 mL, 21.1 mmol) at −78° C. under N₂. The reaction solution was stirred at the same temperature for 1 hr. Then a solution of tert-butyl 4-formylpiperidine-1-carboxylate (3.9 g, 18.3 mmol) in THE (20 mL) was added dropwise to this mixture. The resulting reaction solution was stirred at −78° C. for 1 hr. The reaction solution was quenched with saturated aqueous NH₄Cl (50 mL) and the mixture was extracted with EA (20 mL×3). The combined organic layers were washed with brine, dried over Na₂SO₄, filtered and concentrated to give a residue which was purified by silica gel with PE/EA=3/1 to give tert-butyl 4-((4-chloro-7-(phenylsulfonyl)-7H-pyrrolo[2,3-d]pyrimidin-6-yl)(hydroxy)methyl)piperidine-1-carboxylate as a light yellow solid (5.23 g, 10.3 mmol, 73% yield). ¹H NMR (400 MHz, DMSO-d₆) δ 8.77 (s, 1H), 8.14-8.11 (m, 2H), 7.79-7.75 (m, 1 H), 7.68-7.64 (m, 2H), 6.84 (s, 1H), 5.80 (d, J=12 Hz, 1H), 5.32 (t, J=4 Hz, 1H), 4.06-3.99 (m, 2H), 2.53-2.50 (m, 2H), 2.03-1.96 (m, 1H), 1.44-1.37 (m, 13H). LC/MS (ESI, m/z): [M+H]⁺=507.1.

Step C: To a solution of tert-butyl 4-((4-chloro-7-(phenylsulfonyl)-7H-pyrrolo[2,3-d]pyrimidin-6-yl) (hydroxy)methyl)piperidine-1-carboxylate (2.0 g, 3.95 mmol) in DCM (10 mL) was added TFA (10 mL). The reaction solution was stirred at the room temperature for 16 hr, then concentrated to give crude (4-chloro-7-(phenylsulfonyl)-7H-pyrrolo[2,3-d]pyrimidin-6-yl)(piperidin-4-yl)methanol as a purple solid (2 g, 3.94 mmol, 100% yield). LC/MS (ESI, m/z): [M+H]⁺=407.1.

Step D: To a solution of (4-chloro-7-(phenylsulfonyl)-7H-pyrrolo[2,3-d]pyrimidin-6-yl)(piperidin-4-yl) methanol (400 mg, 0.76 mmol) and TEA (0.853 mL, 6.15 mmol) in DCM (5 mL) was added acryloyl chloride (70 mg, 0.76 mmol) at 0° C. The reaction solution was stirred at 0° C. for 1 h and then the reaction solution was quenched with water (10 mL). The mixture was extracted with EA (10 mL×3). The combined organic layers were washed with brine, dried over Na₂SO₄, filtered and concentrated to give a residue which was purified by silica gel with EA to give 1-(4-((4-chloro-7-(phenylsulfonyl)-7H-pyrrolo[2,3-d]pyrimidin-6-yl)(hydroxy)methyl)piperidin-1-yl)prop-2-en-1-one as a purple solid (160 mg, 0.347 mmol 43.4% yield). LC/MS (ESI, m/z): [M+H]⁺=461.1. ¹H NMR (400 MHz, DMSO-d₆) δ 8.78 (s, 1H), 8.15-8.13 (m, 2H), 7.80-7.76 (m, 1H), 7.69-7.65 (m, 2H), 6.87-6.77 (m, 2H), 6.10 (dd, J=16 Hz, 1H), 5.84 (t, J=5.2 Hz, 1H), 5.67 (d, J=10.4 Hz, 1H), 5.34 (t, J=4.8 Hz, 1H), 4.55 (t, J=15.2 Hz, 1H), 4.11 (t, J=16.8 Hz, 1H), 3.00 (dt, J=66.3, 12.2 Hz, 1H), 2.56 (dt, J=66.8, 12.1 Hz, 1H), 2.17-2.10 (m, 1H), 1.54-1.51 (m, 2H), 1.39-1.23 (m, 1H).

Step E: To a solution of 1-(4-((4-chloro-7-(phenylsulfonyl)-7H-pyrrolo[2,3-d]pyrimidin-6-yl)(hydroxy) methyl)piperidin-1-yl)prop-2-en-1-one (160 mg, 0.347 mmol) and 3, 4-dichloro-2-fluoroaniline (94 mg, 0.521 mmol) in n-BuOH (3 mL) was added HCl (0.043 mL, 0.173 mmol, 4M in 1, 4-dioxane). The reaction solution was stirred at 80° C. for 2 h. The reaction solution was then cooled to room temperature, diluted with water (5 mL), and extracted with EA (5 mL×3). The combined organic layers were washed with brine, dried over Na₂SO₄, filtered and concentrated to give a residue which was purified by prep-TLC with EA to give 1-(4-((4-((3,4-dichloro-2-fluorophenyl)amino)-7-(phenylsulfonyl)-7H-pyrrolo[2,3-d]pyrimidin-6-yl)(hydroxy)methyl)piperidin-1-yl)prop-2-en-1-one as a white solid (60 mg, 0.099 mmol, 28.9% yield). LC/MS (ESI, m/z): [M+H]⁺=604.1. ¹H NMR (400 MHz, DMSO-d₆) δ 9.77 (s, 1H), 8.29 (s, 1H), 8.10-8.07 (m, 2H), 7.75-7.61 (m, 4H), 7.52 (dd, J=8.8 Hz, 1H), 7.09 (s, 1H), 6.85-6.76 (m, 1H), 6.09 (dd, J=16.4 Hz, 1H), 5.70-5.64 (m, 2H), 5.31 (s, 1H), 4.55-4.47 (m, 1H), 4.16-4.10 (m, 1H), 2.99 (dt, J=52.4, 12.4 Hz, 1H), 2.56 (dt, J=49.6, 12.8 Hz, 1 H), 2.06-2.04 (m, 1H), 1.73-1.70 (m, 1H), 1.52-1.49 (m, 2H), 1.35-1.29 (m, 1H).

Step F: To a solution of 1-(4-((4-((3,4-dichloro-2-fluorophenyl)amino)-7-(phenylsulfonyl)-7H-pyrrolo [2,3-d]pyrimidin-6-yl)(hydroxy)methyl)piperidin-1-yl)prop-2-en-1-one (30 mg, 0.049 mmol) in THE (1 mL) was added NaOH (1 mL, 10% in water). The reaction solution was stirred at 65° C. for 16 h. The reaction solution was concentrated and purified by prep-HPLC to give 1-(4-((4-((3,4-dichloro-2-fluorophenyl)amino)-7H-pyrrolo[2,3-d]pyrimidin-6-yl)(hydroxy)methyl)piperidin-1-yl)prop-2-en-1-one (Compound 229) as a white solid (1.3 mg, 0.0028 mmol 5.65% yield). LC/MS (ESI, m/z): [M+H]⁺=464.2. ¹H NMR (400 MHz, MeOD-d₄) δ 8.24 (s, 1H), 7.64 (t, J=8 Hz, 1H), 7.52 (dd, J=8.8 Hz, 1H), 6.79-6.69 (m, 1H), 6.61 (s, 1H), 6.16 (d, J=16.8 Hz, 1H), 5.70 (dd, J=10 Hz, 1H), 5.34 (t, J=4.8 Hz, 1H), 4.63-4.55 (m, 2H), 4.19-4.10 (m, 1H), 3.14-3.06 (m, 1H), 2.72-2.64 (m, 1H), 2.09-2.05 (m, 3H), 1.65-1.61 (m, 2H).

### Example 230: Preparation of 1-(4-(butoxy(4-((3,4-dichloro-2-fluorophenyl)amino)-7H-pyrrolo[2,3-d]pyrimidin-6-yl)methyl)piperidin-1-yl)prop-2-en-1-one (Compound 230)

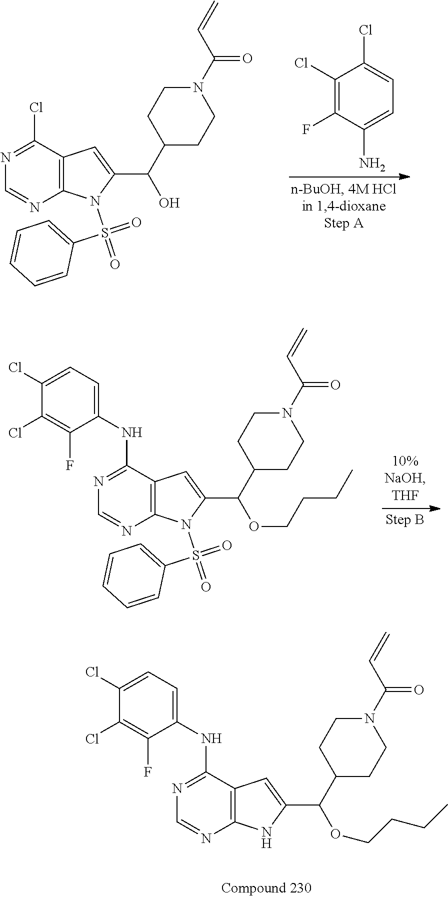 [SMILES: C=CC(=O)N1CCC(C(O)c2cc3c(Cl)ncnc3n2S(=O)(=O)c2ccccc2)CC1 ; Nc1ccc(Cl)c(Cl)c1F ; C=CC(=O)N1CCC(C(OCCCC)c2cc3c(Nc4ccc(Cl)c(Cl)c4F)ncnc3n2S(=O)(=O)c2ccccc2)CC1 ; C=CC(=O)N1CCC(C(OCCCC)c2cc3c(Nc4ccc(Cl)c(Cl)c4F)ncnc3[nH]2)CC1]

Step A: To a solution of 1-(4-((4-chloro-7-(phenylsulfonyl)-7H-pyrrolo[2,3-d]pyrimidin-6-yl)(hydroxy) methyl)piperidin-1-yl)prop-2-en-1-one (50 mg, 0.108 mmol) and 3,4-dichloro-2-fluoroaniline (20 mg, 0.108 mmol) in n-BuOH (2 mL) was added HCl (0.013 mL, 0.054 mmol, 4M in 1,4-dioxane). The reaction solution was stirred at 80° C. for 16 h. The reaction solution was cooled to room temperature, diluted with water (5 mL), and extracted with EA (5 mL×3). The combined organic layers were washed with brine, dried over Na₂SO₄, filtered and concentrated to give a residue which was purified by prep-TLC with EA to give the 1-(4-(butoxy(4-((3,4-dichloro-2-fluorophenyl)amino)-7-(phenylsulfonyl)-7H-pyrrolo [2,3-d]pyrimidin-6-yl)methyl)piperidin-1-yl)prop-2-en-1-one as a white solid (50 mg, 0.075 mmol 70.4% yield). LC/MS (ESI, m/z): [M+H]⁺=660.1.

Step B: To a solution of 1-(4-(butoxy(4-((3,4-dichloro-2-fluorophenyl)amino)-7-(phenylsulfonyl)-7H-pyrrolo[2,3-d]pyrimidin-6-yl)methyl)piperidin-1-yl)prop-2-en-1-one (50 mg, 0.049 mmol) in THF (1 mL) was added NaOH (1 mL, 10% in water). The reaction solution was stirred at 65° C. for 16 h. The reaction solution was concentrated and purified by prep-HPLC to give 1-(4-(butoxy(4-((3,4-dichloro-2-fluorophenyl)amino)-7H-pyrrolo[2,3-d]pyrimidin-6-yl)methyl)piperidin-1-yl)prop-2-en-1-one (Compound 230) as a white solid (20 mg, 0.038 mmol 51.2% yield). LC/MS (ESI, m/z): [M+H]⁺=520.1. ¹H NMR (400 MHz, CDCl₃) δ 11.38 (br s, 1H), 8.35 (s, 1H), 7.65 (s, 1H), 7.38 (dd, J=7.6 Hz, 1H), 6.59-6.50 (m, 1H), 6.25 (d, J=12.8 Hz, 1H), 5.80 (br s, 1H), 5.68 (d, J=13.6 Hz, 1H), 4.73-4.62 (m, 1H), 4.11-3.96 (m, 2H), 3.39-3.26 (m, 2H), 3.07-2.94 (m, 1H), 2.58-2.50 (m, 1H), 2.07-1.89 (m, 2H), 1.55-1.30 (m, 7H), 0.88 (t, J=7.2 Hz, 3H).

### Example 231: Preparation of 1-(4-((4-((3,4-dichloro-2-fluorophenyl)amino)-7H-pyrrolo[2,3-d]pyrimidin-6-yl)(hydroxy)methyl)piperidin-1-yl)-3-methoxypropan-1-one (Compound 231)

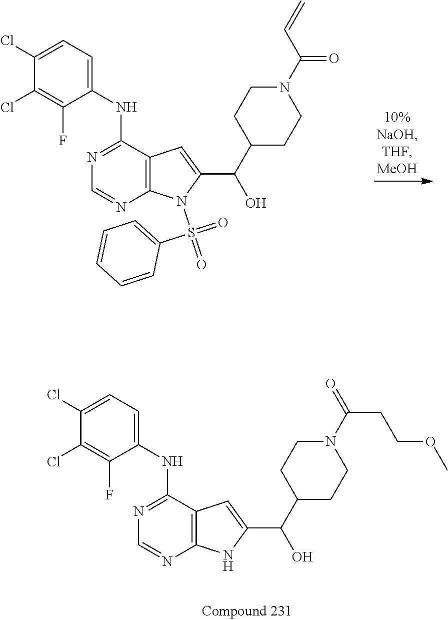 [SMILES: C=CC(=O)N1CCC(C(O)c2cc3c(Nc4ccc(Cl)c(Cl)c4F)ncnc3n2S(=O)(=O)c2ccccc2)CC1 ; COCCC(=O)N1CCC(C(O)c2cc3c(Nc4ccc(Cl)c(Cl)c4F)ncnc3[nH]2)CC1]

To a solution of 1-(4-((4-((3,4-dichloro-2-fluorophenyl)amino)-7-(phenylsulfonyl)-7H-pyrrolo[2,3-d]pyrimidin-6-yl)(hydroxy)methyl)piperidin-1-yl)prop-2-en-1-one (30 mg, 0.049 mmol) in THF (1 mL) and MeOH (1 mL) was added NaOH (1 mL, 10% in water). The reaction solution was stirred at 65° C. for 1 h. The reaction solution was concentrated and purified by prep-HPLC to give 1-(4-((4-((3,4-dichloro-2-fluorophenyl)amino)-7H-pyrrolo[2,3-d]pyrimidin-6-yl)(hydroxy)methyl)piperidin-1-yl)-3-methoxypropan-1-one (Compound 231) as a white solid (1.5 mg, 0.005 mmol 10.1% yield). LC/MS (ESI, m/z): [M+H]⁺=496.2, 498.2. ¹H NMR (400 MHz, MeOD-d₄) δ 8.25 (s, 1H), 7.63-7.53 (m, 2H), 6.62 (d, J=2 Hz, 1H), 4.63-4.53 (m, 2 H), 4.08-3.99 (m, 1H), 3.65-3.61 (m, 2H), 3.30 (s, 3H), 3.13-3.01 (m, 1H), 2.70-2.54 (m, 3H), 2.04-1.88 (m, 2H), 1.58-1.49 (m, 1H), 1.37-1.29 (m, 3H).

### II. Biological Data

### Example 232: EC50 Assay

4,000 Ba/F3 stable cells/90 μL/well were plated onto 96-well cell culture plate (Grenier, Cat.No. 655180) and incubated at 37° C. with a humidified atmosphere of 5% CO₂ for 2 h. Then the cells were treated with 10 μL compounds at serial titration. The final DMSO concentration is 0.1%. The cells treated with 0.1% DMSO (Vetec, Cat.No.V900090) as a positive control (Max), and medium with 0.1% DMSO was as a negative control (Min). Incubated the cells at 37° C. for 72 h. 10 μL the cell proliferation reagent CCK-8 (Dojindo, Cat.No. CK04 1000T) was added into each well and incubated the plate at 37° C. for 4 hours, the GD value was measured by SPECTRAmax plus (Molecular Devices) at 450 nm. Data processing with Graphpad 7.0.

| | Ba/F3-EGFR | Ba/F3-EGFR | Ba/F3-EGFR | Ba/F3-EGFR | Ba/F3-HER2 | Ba/F3-EGFR |
|---|---|---|---|---|---|---|
| EX. | V769- | D770-N771 | A763- | H773- | A775- | L858R- |
| NO. | D770insASV | insSVD | Y764insFQEA | V774insNPH | G776insYVMA | T790M |
| 1 | A | A | A | A | A | A |
| 2 | C | C | C | C | C | C |
| 3 | A | A | A | A | A | B |
| 4 | A | B | A | A | A | B |
| 5 | A | B | A | B | B | B |
| 6 | A | B | A | A | A | B |
| 7 | B | C | A | B | C | A |
| 8 | B | B | A | B | B | C |
| 9 | C | C | A | C | B | C |
| 10 | A | B | A | B | B | C |
| 11 | A | B | A | A | B | C |
| 12 | C | C | C | C | C | C |
| 13 | B | C | A | B | C | C |
| 14 | A | C | A | A | C | A |
| 15 | A | B | A | B | C | B |
| 16 | A | A | A | A | A | A |
| 17 | A | A | A | A | A | A |
| 18 | A | A | A | A | A | A |
| 19 | A | A | A | A | A | A |
| 20 | A | C | A | B | B | B |
| 21 | A | A | A | A | A | A |
| 22 | C | C | B | C | C | C |
| 23 | A | A | A | A | B | A |
| 24 | C | C | B | C | C | C |
| 25 | A | A | A | A | A | A |
| 26 | A | B | A | B | B | B |
| 27 | A | B | A | A | C | B |
| 28 | A | B | A | A | B | B |
| 29 | A | B | A | A | B | A |
| 30 | A | A | A | A | A | A |
| 31 | A | A | A | A | B | A |
| 32 | A | A | A | A | A | A |
| 33 | A | A | A | A | A | A |
| 34 | B | B | A | A | A | C |
| 35 | C | C | C | C | C | C |
| 37 | A | B | A | B | B | B |
| 38 | B | C | A | B | C | C |
| 39 | B | C | A | C | C | C |
| 40 | A | B | A | B | B | B |
| 41 | C | C | B | C | C | C |
| 42 | A | B | A | B | B | C |
| 43 | B | B | A | C | B | C |
| 44 | A | B | A | A | B | B |
| 45 | C | C | A | C | C | C |
| 46 | A | B | A | A | A | A |
| 47 | A | A | A | A | B | A |
| 48 | A | B | A | B | B | A |
| 49 | A | A | A | A | B | A |
| 50 | A | C | A | B | B | C |
| 51 | B | C | A | B | C | A |
| 52 | A | A | A | A | B | A |
| 53 | A | A | A | A | A | A |
| 54 | A | A | A | A | A | A |
| 56 | A | B | A | A | B | B |
| 57 | A | A | A | A | A | A |
| 58 | A | B | A | A | A | A |
| 59 | A | B | A | A | B | A |
| 60 | A | B | A | A | B | A |
| 61 | A | A | A | A | B | A |
| 62 | B | B | A | B | B | B |
| 63 | A | A | A | A | B | A |
| 64 | A | A | A | A | A | A |
| 66 | A | B | A | A | A | A |
| 67 | C | C | B | C | C | C |
| 68 | C | C | C | C | C | C |
| 69 | C | C | C | C | C | C |
| 70 | C | C | C | C | C | C |
| 71 | C | C | C | C | C | C |
| 72 | A | A | A | A | B | B |
| 73 | A | B | A | B | B | C |
| 74 | A | B | A | A | A | B |
| 75 | B | C | A | B | B | B |
| 76 | A | B | A | B | B | C |
| 77 | B | C | A | B | B | C |
| 78 | A | C | A | B | B | C |
| 79 | A | C | A | B | B | C |
| 80 | C | C | A | C | C | C |
| 81 | A | A | A | B | B | B |
| 82 | A | B | A | B | B | B |
| 83 | A | B | A | A | B | B |
| 84 | B | C | A | C | C | C |
| 85 | B | C | A | C | C | C |
| 86 | C | C | B | C | C | C |
| 87 | C | C | B | C | C | C |
| 88 | C | C | B | C | C | C |
| 89 | C | C | B | C | C | C |
| 90 | A | C | A | B | C | C |
| 91 | B | B | A | B | B | C |
| 92 | A | B | A | A | A | B |
| 94 | C | C | A | C | C | C |
| 95 | C | C | C | C | C | C |
| 96 | C | C | C | C | C | C |
| 97 | C | C | C | C | C | C |
| 98 | C | C | B | C | C | C |
| 99 | A | B | A | A | A | A |
| 100 | A | B | A | B | B | B |
| 101 | B | C | A | C | C | C |
| 102 | A | A | A | A | A | A |
| 103 | A | A | A | A | A | A |
| 104 | A | B | A | A | B | B |
| 105 | A | A | A | A | A | A |
| 106 | A | A | A | A | A | B |
| 107 | B | B | A | B | C | B |
| 108 | C | C | A | B | C | C |
| 109 | C | C | B | C | C | C |
| 110 | A | B | A | A | A | B |
| 111 | A | B | A | A | A | B |
| 112 | B | C | A | B | A | B |
| 113 | B | C | A | B | A | C |
| 114 | B | B | A | B | A | A |
| 115 | C | C | A | C | A | B |
| 116 | A | B | A | A | A | B |
| 117 | C | C | B | C | C | B |
| 118 | C | C | A | C | B | C |
| 119 | C | C | A | C | C | C |
| 120 | A | A | A | A | B | B |
| 121 | B | B | A | B | B | B |
| 122 | B | B | A | B | C | B |
| 123 | A | B | A | A | B | B |
| 124 | C | C | A | C | C | C |
| 125 | B | B | A | B | B | B |
| 126 | C | C | C | C | C | C |
| 127 | B | C | A | C | C | C |
| 129 | C | C | C | C | C | C |
| 130 | A | C | A | B | B | C |
| 131 | A | A | A | A | A | C |
| 132 | B | B | A | B | B | C |
| 133 | A | A | A | A | A | A |
| 134 | C | C | B | C | A | C |
| 135 | A | B | A | B | B | B |
| 136 | C | C | B | C | C | C |
| 137 | B | C | A | C | B | C |
| 138 | A | B | A | B | A | B |
| 139 | C | C | A | C | C | C |
| 140 | C | C | C | C | C | C |
| 141 | B | C | A | B | B | B |
| 142 | A | C | A | B | A | B |
| 143 | A | C | A | B | B | B |
| 144 | C | C | B | C | C | C |
| 145 | B | C | A | B | C | B |
| 146 | A | B | A | B | B | B |
| 147 | C | C | C | C | C | C |
| 148 | A | A | A | A | B | A |
| 149 | A | A | A | A | A | A |
| 150 | B | C | A | B | C | C |
| 151 | C | C | C | C | C | C |
| 152 | B | B | A | B | A | B |
| 153 | A | B | A | B | B | B |
| 154 | C | C | B | C | C | C |
| 155 | C | C | A | C | B | B |
| 156 | A | B | A | A | A | B |
| 157 | A | C | A | B | A | B |
| 158 | B | C | A | B | C | A |
| 159 | B | C | A | B | B | B |
| 160 | A | C | A | B | C | B |
| 161 | B | C | A | B | C | B |
| 162 | A | C | A | B | B | A |
| 164 | A | B | A | B | B | A |
| 165 | B | C | A | B | C | B |
| 166 | A | C | A | A | A | B |
| 167 | B | C | A | B | B | A |
| 168 | A | B | A | A | A | C |
| 169 | A | B | A | A | A | B |
| 170 | B | C | A | B | B | A |
| 171 | A | B | A | A | A | C |
| 172 | A | B | A | B | A | C |
| 173 | A | C | A | B | C | C |
| 174 | C | C | C | C | C | C |
| 175 | A | B | A | A | B | B |
| 176 | A | A | A | A | A | A |
| 177 | A | A | A | A | A | A |
| 178 | A | B | A | A | A | A |
| 179 | A | B | A | A | A | A |
| 180 | A | A | A | A | A | A |
| 181 | A | A | A | A | A | A |
| 182 | B | C | A | B | C | B |
| 183 | A | B | A | A | B | A |
| 184 | A | B | A | B | A | C |
| 185 | A | B | A | A | B | A |
| 186 | A | B | A | A | B | C |
| 187 | A | B | A | B | B | B |
| 188 | A | B | A | A | B | A |
| 190 | A | A | A | A | B | A |
| 191 | A | A | A | A | C | A |
| 192 | A | C | A | A | B | A |
| 193 | B | C | A | B | C | B |
| 194 | A | C | A | B | C | C |
| 195 | C | C | B | C | B | B |
| 196 | A | B | A | B | B | C |
| 197 | C | C | B | C | C | C |
| 198 | B | C | A | B | C | C |
| 199 | B | B | A | B | B | B |
| 200 | B | C | A | B | C | C |
| 205 | C | C | B | C | C | C |
| 206 | B | C | A | C | B | C |
| 207 | B | C | A | B | B | B |
| 208 | C | C | B | C | C | C |
| 215 | C | C | A | C | C | C |
| 216 | A | A | A | A | C | A |
| 217 | A | B | A | B | C | B |
| 218 | A | B | A | A | C | B |
| 225 | A | A | A | A | C | A |
| 226 | B | B | A | A | A | A |
| 227 | B | B | A | A | B | A |
| 228 | B | C | A | C | C | A |

where A: EC50 ≤ 200 nM, B: 200 nM < EC50 ≤ 1000 nM, and C: EC50 > 1000 nM

We claim:

1. A compound of Formula (I), or a pharmaceutically acceptable salt or solvate thereof: 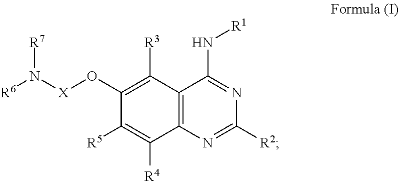
wherein:
X is C₃₋₈cycloalkylene optionally substituted with one, two, three, or four R⁸;
R¹ is selected from C₃₋₈cycloalkyl, C₂₋₉heterocycloalkyl, C₆₋₁₀aryl, and C₁₋₉heteroaryl, wherein C₃₋₈cycloalkyl, C₂₋₉heterocycloalkyl, C₆₋₁₀aryl, and C₁₋₉heteroaryl, are optionally substituted with one, two, three, or four R⁹;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from 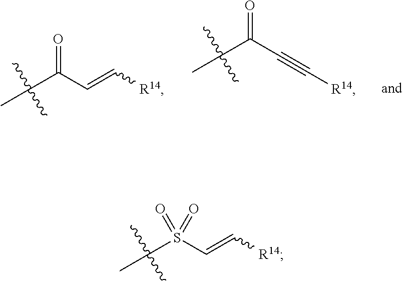
R⁷ is selected from hydrogen, C₁₋₆alkyl, and C₁₋₆haloalkyl;
each R⁸ is independently selected from halogen, oxo, C₁₋₆alkyl, C₁₋₆haloalkyl, C₃₋₆cycloalkyl, C₂₋₉heterocycloalkyl, —OR¹⁰, —SR¹⁰, —N(R¹⁰)(R¹¹), —C(O)OR¹⁰, —C(O)R¹³, —C(O)N(R¹⁰)(R¹¹), —S(O)₂R¹³, —S(O)₂N(R¹⁰)(R¹¹)—, wherein C₁₋₆alkyl, C₃₋₆cycloalkyl, and C₂₋₉heterocycloalkyl are optionally substituted with one, two, or three groups selected from halogen, C₁₋₆alkyl, C₁₋₆haloalkyl, —OR¹⁰, and —N(R¹⁰)(R¹¹);
each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹°)(R¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

2. The compound of claim 1, or a pharmaceutically acceptable salt or solvate thereof, wherein X is cyclobutylene optionally substituted with one, two, three, or four R⁸.

3. The compound of claim 1, or a pharmaceutically acceptable salt or solvate thereof, wherein X is cyclopentylene optionally substituted with one, two, three, or four R⁸.

4. The compound of claim 1, or a pharmaceutically acceptable salt or solvate thereof, wherein X is cyclohexylene optionally substituted with one, two, three, or four R⁸.

5. The compound of claim 1, or a pharmaceutically acceptable salt or solvate thereof, having the structure of Formula (Ia):

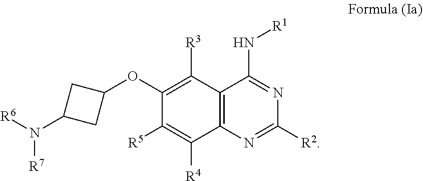

6. The compound of any one of claims 1-5, or a pharmaceutically acceptable salt or solvate thereof, wherein R⁷ is hydrogen.

7. The compound of any one of claims 1-5, or a pharmaceutically acceptable salt or solvate thereof, wherein R⁷ is C₁₋₆alkyl.

8. A compound of Formula (II), or a pharmaceutically acceptable salt or solvate thereof: 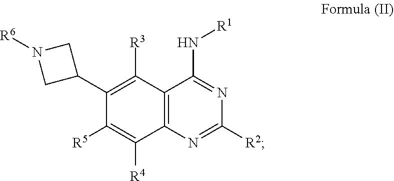
wherein:
R¹ is selected from C₃₋₈cycloalkyl, C₂₋₉heterocycloalkyl, C₆₋₁₀aryl, and C₁₋₉heteroaryl, wherein C₃₋₈cycloalkyl, C₂₋₉heterocycloalkyl, C₆₋₁₀aryl, and C₁₋₉heteroaryl, are optionally substituted with one, two, three, or four R⁹;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from 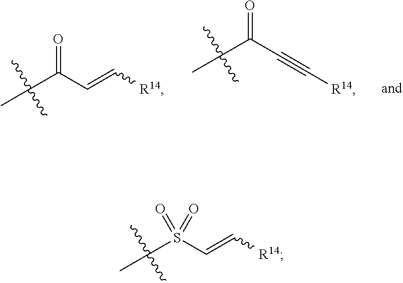
each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

9. The compound of any one of claims 1-8, or a pharmaceutically acceptable salt or solvate thereof, wherein R¹ is C₆₋₁₀aryl optionally substituted with one, two, three, or four R⁹.

10. The compound of any one of claims 1-9, or a pharmaceutically acceptable salt or solvate thereof, wherein R¹ is phenyl optionally substituted with one, two, three, or four R⁹.

11. The compound of any one of claims 1-8, or a pharmaceutically acceptable salt or solvate thereof, wherein R¹ is C₁₋₉heteroaryl optionally substituted with one, two, three, or four R⁹.

12. The compound of any one of claims 1-11, or a pharmaceutically acceptable salt or solvate thereof, wherein each R⁹ is independently selected from halogen, C₁₋₆alkyl, and C₁₋₆haloalkyl; or two R⁹ substituents are combined to form a cycloalkyl or heterocycloalkyl ring.

13. The compound of any one of claims 1-12, or a pharmaceutically acceptable salt or solvate thereof, wherein each R⁹ is independently selected from halogen.

14. The compound of any one of claims 1-8, or a pharmaceutically acceptable salt or solvate thereof, wherein R¹ is unsubstituted C₆₋₁₀aryl.

15. The compound of any one of claims 1-8, or a pharmaceutically acceptable salt or solvate thereof, wherein R¹ is unsubstituted C₁₋₉heteroaryl.

16. A compound of Formula (III), or a pharmaceutically acceptable salt or solvate thereof: 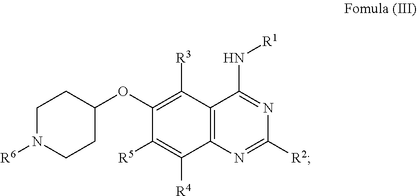
wherein:
R¹ is selected from naphthyl and C₁₋₉heteroaryl, wherein naphthyl and C₁₋₉heteroaryl are optionally substituted with one, two, three, or four R⁹, and C₁₋₉heteroaryl is not indazolyl;
R², R³, R⁴, and R⁵ are each independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
R⁶ is selected from 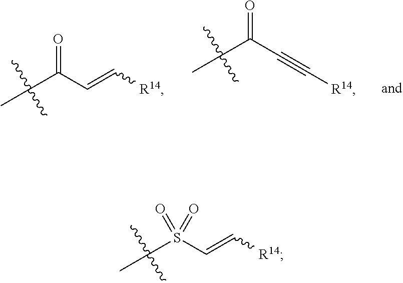
each R⁹ is independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —C(O)N(R¹⁰)(R¹¹), —N(R¹²)C(O)R¹³, —S(O)₂R¹³, —S(O)₂N(R¹⁰)(R¹¹)—, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, —N(R¹⁰)(R¹¹), and —C(O)OR¹⁰; or two R⁹ substituents are combined to form a cycloalkyl or heterocycloalkyl ring;
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
R¹⁴ is selected from hydrogen and C₁₋₆alkyl optionally substituted with —N(R¹⁰)(R¹¹).

17. The compound of claim 16, or a pharmaceutically acceptable salt or solvate thereof, wherein R¹ is naphthyl optionally substituted with one, two, three, or four R⁹.

18. The compound of claim 17, or a pharmaceutically acceptable salt or solvate thereof, wherein R¹ is unsubstituted naphthyl.

19. The compound of claim 16, or a pharmaceutically acceptable salt or solvate thereof, wherein R¹ is C₁₋₉heteroaryl optionally substituted with one, two, three, or four R⁹.

20. The compound of claim 19, or a pharmaceutically acceptable salt or solvate thereof, wherein R¹ is unsubstituted C₁₋₉heteroaryl.

21. The compound of any one of claims 1-20, or a pharmaceutically acceptable salt or solvate thereof, wherein R⁵ is selected from hydrogen, halogen, C₁₋₆alkyl, C₁₋₆haloalkyl, and —OR¹⁰.

22. The compound of any one of claims 1-21, or a pharmaceutically acceptable salt or solvate thereof, wherein R⁵ is —OR¹⁰.

23. The compound of any one of claims 1-22, or a pharmaceutically acceptable salt or solvate thereof, wherein R⁵ is —OR¹⁰ and R¹⁰ is C₁₋₆alkyl.

24. The compound of any one of claims 1-21, or a pharmaceutically acceptable salt or solvate thereof, wherein R⁵ is hydrogen.

25. The compound of any one of claims 1-24, or a pharmaceutically acceptable salt or solvate thereof, wherein R⁴ is hydrogen.

26. The compound of any one of claims 1-25, or a pharmaceutically acceptable salt or solvate thereof, wherein R³ is hydrogen.

27. The compound of any one of claims 1-26, or a pharmaceutically acceptable salt or solvate thereof, wherein R² is hydrogen.

28. The compound of any one of claims 1-27, or a pharmaceutically acceptable salt or solvate thereof, wherein R⁶ is

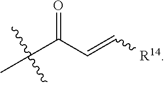

29. The compound of any one of claims 1-27, or a pharmaceutically acceptable salt or solvate thereof, wherein R⁶ is

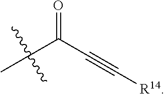

30. The compound of any one of claims 1-27, or a pharmaceutically acceptable salt or solvate thereof, wherein R⁶ is

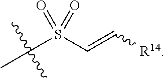

31. The compound of any one of claims 1-30, or a pharmaceutically acceptable salt or solvate thereof, wherein R¹⁴ is hydrogen.

32. The compound of any one of claims 1-30, or a pharmaceutically acceptable salt or solvate thereof, wherein R¹⁴ is C₁₋₆alkyl optionally substituted with —N(R¹⁰)(R¹¹).

33. The compound of any one of claims 1-30, or a pharmaceutically acceptable salt or solvate thereof, wherein R¹⁴ is unsubstituted C₁₋₆alkyl.

34. The compound of any one of claims 1-30, or a pharmaceutically acceptable salt or solvate thereof, wherein R¹⁴ is C₁₋₆alkyl substituted with —N(R¹⁰)(R¹¹).

35. The compound of claim 34, or a pharmaceutically acceptable salt or solvate thereof, wherein R¹⁴ is C₁₋₆alkyl substituted with —N(R¹⁰)(R¹¹), R¹⁰ is C₁₋₆alkyl and R¹¹ is C₁₋₆alkyl.

36. A compound selected from:
1) 1-(3-(4-((3,4-dichloro-2-fluorophenyl)amino)-7-methoxyquinazolin-6-yl)azetidin-1-yl)prop-2-en-1-one
3) 1-(3-(4-((3,4-dichloro-2-fluorophenyl)amino)-7-methoxyquinazolin-6-yl)pyrrolidin-1-yl)prop-2-en-1-one
4) 1-(3-(7-methoxy-4-(naphthalen-1-ylamino)quinazolin-6-yl)azetidin-1-yl)prop-2-en-1-one
5) 1-(3-(7-methoxy-4-(naphthalen-2-ylamino)quinazolin-6-yl)azetidin-1-yl)prop-2-en-1-one
6) 1-(3-(4-((3-chloro-4-fluorophenyl)amino)-7-methoxyquinazolin-6-yl)azetidin-1-yl)prop-2-en-1-one
7) 1-(3-(4-((4-chloronaphthalen-1-yl)amino)-7-methoxyquinazolin-6-yl)azetidin-1-yl)prop-2-en-1-one
8) 1-(3-(4-((3,4-dichloro-2-fluorophenyl)amino)quinazolin-6-yl)azetidin-1-yl)prop-2-en-1-one
9) 1-(4-(4-((3,4-dichloro-2-fluorophenyl)amino)quinazolin-6-yl)piperidin-1-yl)prop-2-en-1-one
10) 1-(4-(4-((3,4-dichloro-2-fluorophenyl)amino)-7-methoxyquinazolin-6-yl)piperidin-1-yl)prop-2-en-1-one
11) 1-(3-(4-((1H-indol-5-yl)amino)-7-methoxyquinazolin-6-yl)azetidin-1-yl)prop-2-en-1-one
13) 1-(3-(4-((4-fluorobenzyl)amino)-7-methoxyquinazolin-6-yl)azetidin-1-yl)prop-2-en-1-one
14) 1-(3-(4-((4-fluoronaphthalen-1-yl)amino)-7-methoxyquinazolin-6-yl)azetidin-1-yl)prop-2-en-1-one
15) 1-(3-(4-((3-chlorobenzyl)amino)-7-methoxyquinazolin-6-yl)azetidin-1-yl)prop-2-en-1-one
16) 1-(3-(4-((3-chloro-2,4-difluorophenyl)amino)-7-methoxyquinazolin-6-yl)azetidin-1-yl)prop-2-en-1-one
17) 1-(3-(4-((3,4-dichloro-2-fluorophenyl)amino)-7-(2-methoxyethoxy)quinazolin-6-yl)azetidin-1-yl)prop-2-en-1-one
18) 1-(3-(4-((3,4-dichloro-2-fluorophenyl)amino)-7-ethoxyquinazolin-6-yl)azetidin-1-yl)prop-2-en-1-one
19) 1-(3-(4-((3,4-dichloro-2-fluorophenyl)amino)-7-(2-hydroxyethoxy)quinazolin-6-yl)azetidin-1-yl)prop-2-en-1-one
20) 1-(3-(4-((3,4-dichloro-2-fluorophenyl)amino)-7-(2,2,2-trifluoroethoxy)quinazolin-6-yl)azetidin-1-yl)prop-2-en-1-one
21) 1-(3-(4-((3,4-dichloro-2-fluorophenyl)amino)-7-(2-(4-methylpiperazin-1-yl)ethoxy)quinazolin-6-yl)azetidin-1-yl)prop-2-en-1-one
22) (S)-1-(3-(4-((3,4-dichloro-2-fluorophenyl)amino)-7-((tetrahydrofuran-3-yl)oxy)quinazolin-6-yl)azetidin-1-yl)prop-2-en-1-one
23) 1-(3-(4-((3,4-dichloro-2-fluorophenyl)amino)-7-(difluoromethoxy)quinazolin-6-yl)azetidin-1-yl)prop-2-en-1-one
24) (S)-1-(3-(4-((3,4-dichloro-2-fluorophenyl)amino)-7-((tetrahydrofuran-3-yl)oxy)quinazolin-6-yl)azetidin-1-yl)prop-2-en-1-one
25) 1-(3-(4-((3,4-dichloro-2-fluorophenyl)amino)-7-(oxetan-3-yloxy)quinazolin-6-yl)azetidin-1-yl)prop-2-en-1-one
26) 1-(3-(4-((3,4-dichloro-2-fluorophenyl)amino)-7-(2-(dimethylamino)ethoxy)quinazolin-6-yl)azetidin-1-yl)prop-2-en-1-one
27) 1-(3-(4-((3,4-dichloro-2-fluorophenyl)amino)-7-((tetrahydro-2H-pyran-4-yl)oxy)quinazolin-6-yl)azetidin-1-yl)prop-2-en-1-one
28) 1-(3-(4-((3,4-dichloro-2-fluorophenyl)amino)-7-(2-morpholinoethoxy)quinazolin-6-yl)azetidin-1-yl)prop-2-en-1-one
29) (R)-1-(3-(4-((3,4-dichloro-2-fluorophenyl)amino)-7-((tetrahydrofuran-3-yl)oxy)quinazolin-6-yl)azetidin-1-yl)prop-2-en-1-one
30) 1-(3-(4-((3,4-dichloro-2-fluorophenyl)amino)-7-(3-morpholinopropoxy)quinazolin-6-yl)azetidin-1-yl)prop-2-en-1-one
31) 1-(3-(4-((3,4-dichloro-2-fluorophenyl)amino)-7-(3-(4-methylpiperazin-1-yl)propoxy)quinazolin-6-yl)azetidin-1-yl)prop-2-en-1-one
32) 1-(3-(4-((3,4-dichloro-2-fluorophenyl)amino)-7-(methoxy-d3)quinazolin-6-yl)azetidin-1-yl)prop-2-en-1-one
33) (S)-1-(3-(4-((3-chloro-2,4-difluorophenyl)amino)-7-((tetrahydrofuran-3-yl)oxy)quinazolin-6-yl)azetidin-1-yl)prop-2-en-1-one
34)N-((4-((3,4-dichloro-2-fluorophenyl)amino)-6,7-dihydrofuro[3,2-g]quinazolin-6-yl)methyl)acrylamide
37) (S)-1-(3-(4-((3,4-dichloro-2-fluorophenyl)amino)-7-((tetrahydrofuran-3-yl)oxy)quinazolin-6-yl)azetidin-1-yl)-2-fluoroprop-2-en-1-one
38) 1-(3-(4-((3,4-dichloro-2-fluorophenyl)amino)-7-methoxyquinazolin-6-yl)azetidin-1-yl)-2-fluoroprop-2-en-1-one
39) 1-(3-(4-((3,4-dichloro-2-fluorophenyl)amino)-7-(methoxy-d3)quinazolin-6-yl)azetidin-1-yl)-2-fluoroprop-2-en-1-one
40)N-((1s,3s)-3-((4-((3,4-dichloro-2-fluorophenyl)amino)-7-methoxyquinazolin-6-yl)oxy)cyclobutyl)-2-fluoroacrylamide
41) 1-(3-(4-((4-(benzyloxy)-3-chloro-2-fluorophenyl)amino)-7-methoxyquinazolin-6-yl)azetidin-1-yl)prop-2-yn-1-one
42) (E)-N-(3-((4-((3,4-dichloro-2-fluorophenyl)amino)-7-methoxyquinazolin-6-yl)oxy)cyclobutyl)-4-(dimethylamino)but-2-enamide
43)N-(3-((4-((3,4-dichloro-2-fluorophenyl)amino)-7-methoxyquinazolin-6-yl)oxy)cyclobutyl)but-2-ynamide
44)N-((1r,3r)-3-((4-((2,3-dihydro-1H-inden-4-yl)amino)-7-methoxyquinazolin-6-yl)oxy)cyclobutyl)propiolamide
45)N-((1s,3s)-3-((4-((2,3-dihydro-1H-inden-4-yl)amino)-7-methoxyquinazolin-6-yl)oxy)cyclobutyl)propiolamide
46)N-(3-((7-methoxy-4-(naphthalen-1-ylamino)quinazolin-6-yl)oxy)cyclobutyl)propiolamide
47)N-((1s,3s)-3-((7-methoxy-4-(naphthalen-1-ylamino)quinazolin-6-yl)oxy)cyclobutyl)propiolamide
48)N-((1r,3r)-3-((7-methoxy-4-(naphthalen-2-ylamino)quinazolin-6-yl)oxy)cyclobutyl)propiolamide
49)N-(3-((4-((3,4-dichloro-2-fluorophenyl)amino)-7-methoxyquinazolin-6-yl)oxy)cyclobutyl)propiolamide
50) 1-(4-((4-((3,4-dichloro-2-fluorophenyl)amino)-7-methoxyquinazolin-6-yl)oxy)piperidin-1-yl)-2-fluoroprop-2-en-1-one
51) 1-(4-((4-(3,4-dichloro-2-fluorobenzoyl)-7-methoxyquinazolin-6-yl)oxy)piperidin-1-yl)prop-2-yn-1-one
52) 1-(4-((4-((3,4-dichloro-2-fluorophenyl)amino)-7-methoxyquinazolin-6-yl)oxy)piperidin-1-yl)prop-2-yn-1-one
53) 1-(4-((7-methoxy-4-(naphthalen-2-ylamino)quinazolin-6-yl)oxy)piperidin-1-yl)prop-2-yn-1-one
54) 1-(4-((7-methoxy-4-(naphthalen-1-ylamino)quinazolin-6-yl)oxy)piperidin-1-yl)prop-2-yn-1-one
56) 1-(3-((4-((2,3-dihydro-1H-inden-4-yl)amino)-7-methoxyquinazolin-6-yl)oxy)-8-azabicyclo[3.2.1]octan-8-yl)prop-2-yn-1-one
57) 1-(3-((7-methoxy-4-(naphthalen-1-ylamino)quinazolin-6-yl)oxy)-9-azabicyclo[3.3.1]nonan-9-yl)prop-2-yn-1-one
58) 1-(3-((7-methoxy-4-(naphthalen-2-ylamino)quinazolin-6-yl)oxy)-9-azabicyclo[3.3.1]nonan-9-yl)prop-2-yn-1-one
59) 1-(3-((7-methoxy-4-(naphthalen-2-ylamino)quinazolin-6-yl)oxy)-8-azabicyclo[3.2.1]octan-8-yl)prop-2-yn-1-one
60) 1-(3-((7-methoxy-4-(naphthalen-1-ylamino)quinazolin-6-yl)oxy)-8-azabicyclo[3.2.1]octan-8-yl)prop-2-yn-1-one
61) 1-(3-(4-((3,4-dichloro-2-fluorophenyl)amino)quinazolin-6-yl)piperidin-1-yl)prop-2-yn-1-one
62) 1-(5-(4-((3,4-dichloro-2-fluorophenyl)amino)quinazolin-6-yl)-3,6-dihydropyridin-1(2H)-yl)prop-2-yn-1-one
63) 1-(5-(4-((3,4-dichloro-2-fluorophenyl)amino)-7-methoxyquinazolin-6-yl)-3,6-dihydropyridin-1(2H)-yl)prop-2-yn-1-one
64) 1-(4-((7-methoxy-4-(naphthalen-2-ylamino)quinazolin-6-yl)oxy)piperidin-1-yl)prop-2-yn-1-one
66)N-((1s,3s)-3-((4-((3,4-dichloro-2-fluorophenyl)amino)-7-methoxyquinazolin-6-yl)oxy)cyclobutyl)acrylamide
67)N-(3-((7-methoxy-4-((7-methyl-2,3-dihydro-1H-inden-4-yl)amino)quinazolin-6-yl)oxy)cyclobutyl)acrylamide
72)N-(3-((4-((3,4-dichloro-2-fluorophenyl)amino)-7-methoxyquinazolin-6-yl)oxy)cyclobutyl)-N-methylacrylamide
73)N-(3-((4-((3,4-dichlorophenyl)amino)-7-methoxyquinazolin-6-yl)oxy)cyclobutyl)acrylamide
74)N-(3-((4-((3-chloro-2-fluorophenyl)amino)-7-methoxyquinazolin-6-yl)oxy)cyclobutyl)acrylamide
75)N-(3-((4-((2,4-dichloro-3-fluorophenyl)amino)-7-methoxyquinazolin-6-yl)oxy)cyclobutyl)acrylamide
76)N-(3-((4-((2,3-dichloro-4-fluorophenyl)amino)-7-methoxyquinazolin-6-yl)oxy)cyclobutyl)acrylamide
77)N-(3-((4-((2-fluorophenyl)amino)-7-methoxyquinazolin-6-yl)oxy)cyclobutyl)acrylamide
78)N-(3-((4-((4-chloro-2-fluorophenyl)amino)-7-methoxyquinazolin-6-yl)oxy)cyclobutyl)acrylamide
79)N-((1r,3r)-3-((4-((3,4-dichloro-2-fluorophenyl)amino)-7-methoxyquinazolin-6-yl)oxy)cyclobutyl)acrylamide
80)N-(3-((7-methoxy-4-(naphthalen-2-ylamino)quinazolin-6-yl)oxy)cyclobutyl)acrylamide
81)N-(3-((7-methoxy-4-(naphthalen-1-ylamino)quinazolin-6-yl)oxy)cyclobutyl)acrylamide
82)N-((1s,3s)-3-((4-((3,4-dichlorophenyl)amino)-7-methoxyquinazolin-6-yl)oxy)cyclobutyl)acrylamide
83)N-((1s,3s)-3-((7-methoxy-4-(naphthalen-1-ylamino)quinazolin-6-yl)oxy)cyclobutyl)acrylamide
84)N-(3-((4-((4-fluorobenzyl)amino)-7-methoxyquinazolin-6-yl)oxy)cyclobutyl)acrylamide
85)N-(3-((4-((3-chloro-4-fluorophenyl)amino)-7-methoxyquinazolin-6-yl)oxy)cyclobutyl)acrylamide
86)N-(3-((4-((3-chloro-4-cyanophenyl)amino)-7-methoxyquinazolin-6-yl)oxy)cyclobutyl)acrylamide
87)N-(3-((4-((3-cyano-4-fluorophenyl)amino)-7-methoxyquinazolin-6-yl)oxy)cyclobutyl)acrylamide
88)N-(3-((4-((2,3-dihydro-1H-inden-4-yl)amino)-7-methoxyquinazolin-6-yl)oxy)cyclobutyl)acrylamide
89)N-(3-((7-methoxy-4-((5,6,7,8-tetrahydronaphthalen-1-yl)amino)quinazolin-6-yl)oxy)cyclobutyl)acrylamide
90)N-((1s,4s)-4-((4-((3,4-dichloro-2-fluorophenyl)amino)-7-methoxyquinazolin-6-yl)oxy)cyclohexyl)acrylamide
91)N-((1r,4r)-4-((4-((3,4-dichloro-2-fluorophenyl)amino)-7-methoxyquinazolin-6-yl)oxy)cyclohexyl)acrylamide
92)N-(3-((4-((3,4-dichloro-2-fluorophenyl)amino)-7-methoxyquinazolin-6-yl)amino)cyclobutyl)acrylamide
94) 6-((1-acryloylpiperidin-4-yl)oxy)-N-(4-fluorobenzyl)-7-methoxyquinazoline-4-carboxamide
98)N-(6-((1-acryloylpiperidin-4-yl)oxy)-7-methoxyquinazolin-4-yl)-4-fluorobenzamide
99)N-((1R,3s)-3-((4-((3,4-dichloro-2-fluorophenyl)amino)-7-(((S)-tetrahydrofuran-3-yl)oxy)quinazolin-6-yl)oxy)cyclobutyl)acrylamide
100)N-((1s,3s)-3-((4-((3,4-dichloro-2-fluorophenyl)amino)-7-(2-hydroxyethoxy)quinazolin-6-yl)oxy)cyclobutyl)acrylamide
101)N-((1s,3s)-3-((4-((3,4-dichloro-2-fluorophenyl)amino)-7-(2-(dimethylamino)ethoxy)quinazolin-6-yl)oxy)cyclobutyl)acrylamide
102)N-((1s,3s)-3-((4-((3,4-dichloro-2-fluorophenyl)amino)-7-(methoxy-d3)quinazolin-6-yl)oxy)cyclobutyl)acrylamide
103)N-((1s,3s)-3-((4-((3,4-dichloro-2-fluorophenyl)amino)-7-(methylamino)quinazolin-6-yl)oxy)cyclobutyl)acrylamide
104)N-((1s,3s)-3-((4-((3,4-dichloro-2-fluorophenyl)amino)-7-(oxetan-3-yloxy)quinazolin-6-yl)oxy)cyclobutyl)acrylamide
105)N-((1s,3s)-3-((4-((3,4-dichloro-2-fluorophenyl)amino)-7-ethoxyquinazolin-6-yl)oxy)cyclobutyl)acrylamide
106) 1-(4-((4-((3-chlorobenzyl)amino)-7-methoxyquinazolin-6-yl)oxy)piperidin-1-yl)prop-2-en-1-one
107) 1-(4-((4-((3-chlorobenzyl)oxy)-7-methoxyquinazolin-6-yl)oxy)piperidin-1-yl)prop-2-en-1-one
108) 1-(4-((4-((4-chlorobenzyl)amino)-7-methoxyquinazolin-6-yl)oxy)piperidin-1-yl)prop-2-en-1-one
109) 1-(4-((4-((2,4-dichlorobenzyl)amino)-7-methoxyquinazolin-6-yl)oxy)piperidin-1-yl)prop-2-en-1-one
110) 1-(4-((4-((2-chlorobenzyl)amino)-7-methoxyquinazolin-6-yl)oxy)piperidin-1-yl)prop-2-en-1-one
111) 1-(4-((4-((3-fluorobenzyl)amino)-7-methoxyquinazolin-6-yl)oxy)piperidin-1-yl)prop-2-en-1-one
112) 1-(4-((4-((2-fluorobenzyl)amino)-7-methoxyquinazolin-6-yl)oxy)piperidin-1-yl)prop-2-en-1-one
113) 1-(4-((4-((4-chloro-3-fluorobenzyl)amino)-7-methoxyquinazolin-6-yl)oxy)piperidin-1-yl)prop-2-en-1-one
114) 1-(4-((4-((3-chloro-4-fluorobenzyl)amino)-7-methoxyquinazolin-6-yl)oxy)piperidin-1-yl)prop-2-en-1-one
115) 1-(4-((4-((3,4-dichlorobenzyl)amino)-7-methoxyquinazolin-6-yl)oxy)piperidin-1-yl)prop-2-en-1-one
116) 1-(4-((4-((3-chloro-2-fluorobenzyl)amino)-7-methoxyquinazolin-6-yl)oxy)piperidin-1-yl)prop-2-en-1-one
117) 1-(4-((4-((3,5-dichlorobenzyl)amino)-7-methoxyquinazolin-6-yl)oxy)piperidin-1-yl)prop-2-en-1-one
118) 1-(4-((4-((5-chloro-2-fluorobenzyl)amino)-7-methoxyquinazolin-6-yl)oxy)piperidin-1-yl)prop-2-en-1-one
119) 1-(4-((4-((2,5-dichlorobenzyl)amino)-7-methoxyquinazolin-6-yl)oxy)piperidin-1-yl)prop-2-en-1-one
120) 1-(4-((4-((3-bromobenzyl)amino)-7-methoxyquinazolin-6-yl)oxy)piperidin-1-yl)prop-2-en-1-one
121) 1-(4-((7-methoxy-4-((3-methylbenzyl)amino)quinazolin-6-yl)oxy)piperidin-1-yl)prop-2-en-1-one
122) 1-(4-((4-((3-chloro-5-fluorobenzyl)amino)-7-methoxyquinazolin-6-yl)oxy)piperidin-1-yl)prop-2-en-1-one
123) 1-(4-((4-(benzylamino)-7-methoxyquinazolin-6-yl)oxy)piperidin-1-yl)prop-2-en-1-one
124) 3-(((6-((1-acryloylpiperidin-4-yl)oxy)-7-methoxyquinazolin-4-yl)amino)methyl)benzonitrile
125) 1-(4-((4-((3-iodobenzyl)amino)-7-methoxyquinazolin-6-yl)oxy)piperidin-1-yl)prop-2-en-1-one
127) 2-((6-((1-acryloylpiperidin-4-yl)oxy)-7-methoxyquinazolin-4-yl)amino)-2-(3-chlorophenyl)acetamide
130) 3-((6-((1-acryloylpiperidin-4-yl)oxy)-7-methoxyquinazolin-4-yl)amino)-3-(3-chlorophenyl) propanamide
131) 1-(4-((4-((1-(3-chlorophenyl)-3-hydroxypropyl)amino)-7-methoxyquinazolin-6-yl)oxy)piperidin-1-yl)prop-2-en-1-one
132) 1-(3-((4-((3,4-dichloro-2-fluorophenyl)amino)-7-methoxyquinazolin-6-yl)oxy)piperidin-1-yl)prop-2-en-1-one
133) 1-(4-((4-((3,4-dichloro-2-fluorophenyl)amino)-7-methoxyquinazolin-6-yl)oxy)piperidin-1-yl)prop-2-en-1-one
134) 1-(4-((7-methoxy-4-((1-(methylsulfonyl)indolin-5-yl)amino)quinazolin-6-yl)oxy)piperidin-1-yl)prop-2-en-1-one
135) 1-(4-((7-methoxy-4-(quinolin-3-ylamino)quinazolin-6-yl)oxy)piperidin-1-yl)prop-2-en-1-one
136) 1-(4-((4-((5-chlorobenzo[d]oxazol-2-yl)amino)-7-methoxyquinazolin-6-yl)oxy)piperidin-1-yl)prop-2-en-1-one
137) 1-(4-((4-((1H-indazol-6-yl)amino)-7-methoxyquinazolin-6-yl)oxy)piperidin-1-yl)prop-2-en-1-one
138) 1-(4-((7-methoxy-4-((1-methyl-1H-indazol-6-yl)amino)quinazolin-6-yl)oxy)piperidin-1-yl)prop-2-en-1-one
139) 1-(4-((4-((1H-indazol-7-yl)amino)-7-methoxyquinazolin-6-yl)oxy)piperidin-1-yl)prop-2-en-1-one
141) 1-(4-((7-methoxy-4-(quinolin-7-ylamino)quinazolin-6-yl)oxy)piperidin-1-yl)prop-2-en-1-one
142) 1-(4-((4-(benzo[d]thiazol-5-ylamino)-7-methoxyquinazolin-6-yl)oxy)piperidin-1-yl)prop-2-en-1-one
143) 1-(4-((7-methoxy-4-(quinolin-6-ylamino)quinazolin-6-yl)oxy)piperidin-1-yl)prop-2-en-1-one
144) 1-(4-((7-methoxy-4-(quinoxalin-6-ylamino)quinazolin-6-yl)oxy)piperidin-1-yl)prop-2-en-1-one
145) 1-(4-((4-(benzo[d]thiazol-6-ylamino)-7-methoxyquinazolin-6-yl)oxy)piperidin-1-yl)prop-2-en-1-one
146) 1-(4-((7-methoxy-4-(quinolin-8-ylamino)quinazolin-6-yl)oxy)piperidin-1-yl)prop-2-en-1-one
148) 1-(4-((7-methoxy-4-(naphthalen-2-ylamino)quinazolin-6-yl)oxy)piperidin-1-yl)prop-2-en-1-one
149) 1-(4-((7-methoxy-4-(naphthalen-1-ylamino)quinazolin-6-yl)oxy)piperidin-1-yl)prop-2-en-1-one
150) 1-(4-((4-((1H-indazol-5-yl)amino)-7-methoxyquinazolin-6-yl)oxy)piperidin-1-yl)prop-2-en-1-one
152) 1-(4-((4-(benzo[d][1,3]dioxol-5-ylamino)-7-methoxyquinazolin-6-yl)oxy)piperidin-1-yl)prop-2-en-1-one
153) 1-(4-((4-((2,3-dihydro-1H-inden-5-yl)amino)-7-methoxyquinazolin-6-yl)oxy)piperidin-1-yl)prop-2-en-1-one
154) 1-(4-((4-((5-chlorobenzo[d]oxazol-2-yl)amino)-7-methoxyquinazolin-6-yl)oxy)piperidin-1-yl)prop-2-en-1-one
155) 1-(4-((7-methoxy-4-((2-methylbenzo[d]thiazol-5-yl)amino)quinazolin-6-yl)oxy)piperidin-1-yl)prop-2-en-1-one
156) 1-(4-((4-((2,3-dihydro-1H-inden-4-yl)amino)-7-methoxyquinazolin-6-yl)oxy)piperidin-1-yl)prop-2-en-1-one
157) 1-(4-((4-(benzo[d]thiazol-5-ylamino)-7-methoxyquinazolin-6-yl)oxy)piperidin-1-yl)prop-2-en-1-one
158) 1-(4-((7-methoxy-4-(quinolin-2-ylamino)quinazolin-6-yl)oxy)piperidin-1-yl)prop-2-en-1-one
159) 1-(4-((4-(isoquinolin-3-ylamino)-7-methoxyquinazolin-6-yl)oxy)piperidin-1-yl)prop-2-en-1-one
160) 1-(4-((4-((1-chloronaphthalen-2-yl)amino)-7-methoxyquinazolin-6-yl)oxy)piperidin-1-yl)prop-2-en-1-one
161) 1-(4-((4-(isoquinolin-1-ylamino)-7-methoxyquinazolin-6-yl)oxy)piperidin-1-yl)prop-2-en-1-one
162) 1-(4-((7-methoxy-4-(quinolin-4-ylamino)quinazolin-6-yl)oxy)piperidin-1-yl)prop-2-en-1-one
164) 1-(4-((4-((4-chloronaphthalen-1-yl)amino)-7-methoxyquinazolin-6-yl)oxy)piperidin-1-yl)prop-2-en-1-one
165) 1-(4-((4-((1H-indol-4-yl)amino)-7-methoxyquinazolin-6-yl)oxy)piperidin-1-yl)prop-2-en-1-one
166) 1-(4-((4-((1H-indol-7-yl)amino)-7-methoxyquinazolin-6-yl)oxy)piperidin-1-yl)prop-2-en-1-one
167) 1-(4-((7-methoxy-4-((4-methylnaphthalen-1-yl)amino)quinazolin-6-yl)oxy)piperidin-1-yl)prop-2-en-1-one
168) 1-(4-((4-(indolin-5-ylamino)-7-methoxyquinazolin-6-yl)oxy)piperidin-1-yl)prop-2-en-1-one
169) 1-(4-((4-((1H-indol-5-yl)amino)-7-methoxyquinazolin-6-yl)oxy)piperidin-1-yl)prop-2-en-1-one
170) 1-(4-((7-methoxy-4-((1-methyl-1H-indol-5-yl)amino)quinazolin-6-yl)oxy)piperidin-1-yl)prop-2-en-1-one
171) 1-(4-((4-(indolin-5-ylamino)-7-methoxyquinazolin-6-yl)oxy)piperidin-1-yl)prop-2-en-1-one
172) 1-(4-((4-((4-fluorobenzyl)amino)-7-methoxyquinazolin-6-yl)oxy)piperidin-1-yl)prop-2-en-1-one
173) (R)-1-(4-((4-((1-(4-fluorophenyl)ethyl)amino)-7-methoxyquinazolin-6-yl)oxy)piperidin-1-yl)prop-2-en-1-one
175) 1-(4-((4-(3,4-dichloro-2-fluorobenzoyl)-7-methoxyquinazolin-6-yl)oxy)piperidin-1-yl)prop-2-en-1-one
176) 1-(3-((4-((3,4-dichloro-2-fluorophenyl)amino)-7-methoxyquinazolin-6-yl)oxy)-9-azabicyclo[3.3.1]nonan-9-yl)prop-2-en-1-one
177) 1-(3-((4-((3,4-dichloro-2-fluorophenyl)amino)-7-methoxyquinazolin-6-yl)oxy)-8-azabicyclo[3.2.1]octan-8-yl)prop-2-en-1-one
178) 1-(3-((7-methoxy-4-(naphthalen-1-ylamino)quinazolin-6-yl)oxy)-9-azabicyclo[3.3.1]nonan-9-yl)prop-2-en-1-one
179) 1-(3-((7-methoxy-4-(naphthalen-2-ylamino)quinazolin-6-yl)oxy)-9-azabicyclo[3.3.1]nonan-9-yl)prop-2-en-1-one
180) 1-(3-((7-methoxy-4-(naphthalen-2-ylamino)quinazolin-6-yl)oxy)-8-azabicyclo[3.2.1]octan-8-yl)prop-2-en-1-one
181) 1-(3-((7-methoxy-4-(naphthalen-1-ylamino)quinazolin-6-yl)oxy)-8-azabicyclo[3.2.1]octan-8-yl)prop-2-en-1-one
182) 1-(3-((4-((2,3-dihydro-1H-inden-4-yl)amino)-7-methoxyquinazolin-6-yl)oxy)-9-azabicyclo[3.3.1]nonan-9-yl)prop-2-en-1-one
183) 1-(3-((4-((2,3-dihydro-1H-inden-4-yl)amino)-7-methoxyquinazolin-6-yl)oxy)-8-azabicyclo[3.2.1]octan-8-yl)prop-2-en-1-one
184) 1-(3-(4-((3,4-dichloro-2-fluorophenyl)amino)quinazolin-6-yl)piperidin-1-yl)prop-2-en-1-one
185) 1-(3-(4-((3,4-dichloro-2-fluorophenyl)amino)-7-methoxyquinazolin-6-yl)piperidin-1-yl)prop-2-en-1-one
186) 1-(5-(4-((3,4-dichloro-2-fluorophenyl)amino)-7-methoxyquinazolin-6-yl)-3,6-dihydropyridin-1(2H)-yl)prop-2-en-1-one
187) 1-(5-(4-((3,4-dichloro-2-fluorophenyl)amino)quinazolin-6-yl)-3,6-dihydropyridin-1(2H)-yl)prop-2-en-1-one
188)N-(3,4-dichloro-2-fluorophenyl)-6-(1-(vinylsulfonyl)piperidin-3-yl)quinazolin-4-amine
190)N-(3,4-dichloro-2-fluorophenyl)-6-(1-(vinylsulfonyl)-1,2,5,6-tetrahydropyridin-3-yl)quinazolin-4-amine
191)N-(3-((4-((3,4-dichloro-2-fluorophenyl)amino)-7-methoxyquinazolin-6-yl)oxy)cyclobutyl)ethenesulfonamide
192)N-(3,4-dichloro-2-fluorophenyl)-7-methoxy-6-((1-(vinylsulfonyl)piperidin-4-yl)oxy)quinazolin-4-amine
193) (3,4-dichloro-2-fluorophenyl)(7-methoxy-6-((1-(vinylsulfonyl)piperidin-4-yl)oxy)quinazolin-4-yl)methanone
194) 4-((4-((3,4-dichloro-2-fluorophenyl)amino)-7-methoxyquinazolin-6-yl)oxy)piperidine-1-carbonitrile
195) 3-(4-((3,4-dichloro-2-fluorophenyl)amino)-7-methoxyquinazolin-6-yl)azetidine-1-carbonitrile
196)N-(3-((4-((3,4-dichloro-2-fluorophenyl)amino)-7-methoxyquinazolin-6-yl)oxy)cyclobutyl)-N-methylcyanamide
197) 5-(4-((3,4-dichloro-2-fluorophenyl)amino)-7-methoxyquinazolin-6-yl)-3,6-dihydropyridine-1(2H)-carbonitrile
198) 3-((4-((3,4-dichloro-2-fluorophenyl)amino)-7-methoxyquinazolin-6-yl)oxy)piperidine-1-carbonitrile
199) 9-((1-acryloylpiperidin-4-yl)oxy)-3-(3,4-dichloro-2-fluorophenyl)-8-methoxy-1H-pyrimido[4,5,6-de]quinazolin-2(3H)-one
200) 9-((1-acryloylpiperidin-4-yl)oxy)-3-(3,4-dichloro-2-fluorophenyl)-8-methoxy-1-methyl-1H-pyrimido[4,5,6-de]quinazolin-2(3H)-one
205) 9-((1-acryloylpyrrolidin-3-yl)oxy)-3-(3,4-dichloro-2-fluorophenyl)-8-methoxy-1H-pyrimido[4,5,6-de]quinazolin-2(3H)-one
206) 9-(1-acryloyl-1,2,3,6-tetrahydropyridin-4-yl)-3-(3,4-dichloro-2-fluorophenyl)-8-methoxy-1-methyl-1H-pyrimido[4,5,6-de]quinazolin-2(3H)-one
207)N-(3,4-dichloro-2-fluorophenyl)-7-methoxy-5-nitro-6-(piperidin-4-yloxy)quinazolin-4-amine hydrochloride
208) 1-(4-((4-((3,4-dichloro-2-fluorophenyl)amino)-7-methoxy-5-nitroquinazolin-6-yl)oxy)piperidin-1-yl)prop-2-en-1-one
215) 9-((1-acryloylpiperidin-4-yl)oxy)-3-(3,4-dichlorophenyl)-8-methoxy-1H-pyrimido[4,5,6-de]quinazolin-2(3H)-one
216) 9-((1-acryloylazetidin-3-yl)oxy)-3-(3,4-dichloro-2-fluorophenyl)-8-methoxy-1H-pyrimido[4,5,6-de]quinazolin-2(3H)-one
217) 1-(4-((4-(3,4-dichloro-2-fluorophenyl)-8-methoxy-5-methyl-4H-pyrido[2,3,4-de]quinazolin-7-yl)oxy)piperidin-1yl)prop-2-en-1-one
218) 1-(4-((4-(3,4-dichloro-2-fluorophenyl)-8-methoxy-4H-pyrido[2,3,4-de]quinazolin-7-yl)oxy) piperidin-1-yl)prop-2-en-1-one
225) 3-(3,4-dichloro-2-fluorophenyl)-8-methoxy-9-((1-(vinylsulfonyl)piperidin-4-yl)oxy)-1H-pyrimido[4,5,6-de]quinazolin-2(3H)-one
226) 3-(3,4-dichlorophenyl)-8-methoxy-9-((1-(vinylsulfonyl)piperidin-4-yl)oxy)-1H-pyrimido[4,5,6-de]quinazolin-2(3H)-one
227) 3-(3,4-dichlorophenyl)-8-methoxy-9-((1-propioloylpiperidin-4-yl)oxy)-1H-pyrimido[4,5,6-de]quinazolin-2(3H)-one; and
228)N-(4-amino-5-(quinolin-3-yl)-8,9-dihydro-7H-pyrimido[5′,4′:4,5]pyrrolo[2,1-b][1,3]oxazin-8-yl)acrylamide

37. A pharmaceutical composition comprising a compound of any one of claims 1-36, or a pharmaceutically acceptable salt or solvate thereof, and at least one pharmaceutically acceptable excipient.

38. A method of treating cancer in an individual, comprising administering to the individual a compound of any one of claims 1-36, or a pharmaceutically acceptable salt or solvate thereof.

39. The method of claim 38, wherein the cancer is characterized by the overexpression of EGFR/Erb-B2 or mutations of EGFR/Erb-B2.

40. The method of claim 38 or claim 39, wherein the cancer comprises exon 20 insertions.

41. The method of any one of claims 38-40, wherein the cancer is lung cancer.

42. The method of any one of claims 38-41, wherein the cancer is non-small-cell lung cancer.
